SEQUENCE LISTING (37 sequences)

SEQ ID NO: 1 - protein, 12 aa, Homo sapiens
KDNIKHVPGGGS

SEQ ID NO: 2 - protein, 13 aa, Artificial Sequence
  source: /note=“Description of Artificial Sequence Synthetic peptide”
CKDNIKHVPGGGS

SEQ ID NO: 3 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 8 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 9 - protein, 7 aa, Homo sapiens
KHQPGGG

SEQ ID NO: 10 - protein, 6 aa, Homo sapiens
HQPGGG

SEQ ID NO: 11 - protein, 7 aa, Homo sapiens
KHVPGGG

SEQ ID NO: 12 - protein, 6 aa, Homo sapiens
HVPGGG

SEQ ID NO: 13 - protein, 7 aa, Homo sapiens
HHKPGGG

SEQ ID NO: 14 - protein, 6 aa, Homo sapiens
HKPGGG

SEQ ID NO: 15 - protein, 7 aa, Homo sapiens
THVPGGG

SEQ ID NO: 16 - protein, 6 aa, Homo sapiens
HVPGGG

SEQ ID NO: 17 - protein, 29 aa, Homo sapiens
DLSKVTSKCGSLGNIHHKPGGGQVEVKSE

SEQ ID NO: 18 - protein, 30 aa, Homo sapiens
SKIGSLDNITHVPGGGNKKIETHKLTFREN

SEQ ID NO: 19 - protein, 12 aa, Homo sapiens
LDNITHVPGGGN

SEQ ID NO: 20 - RNA, 22 nt, Homo sapiens
ugagguaguagguuguauaguu

SEQ ID NO: 21 - RNA, 23 nt, Homo sapiens
uacccuguagauccgaauuugug

SEQ ID NO: 22 - RNA, 23 nt, Homo sapiens
guccaguuuucccaggaaucccu

SEQ ID NO: 23 - RNA, 23 nt, Homo sapiens
agcagcauuguacagggcuauga

SEQ ID NO: 24 - RNA, 23 nt, Homo sapiens
caacggaaucccaaaagcagcug

SEQ ID NO: 25 - RNA, 22 nt, Homo sapiens
uuauaauacaaccugauaagug

SEQ ID NO: 26 - RNA, 22 nt, Homo sapiens
uucaaguaauccaggauaggcu

SEQ ID NO: 27 - RNA, 23 nt, Homo sapiens
agcagcauuguacagggcuauca

SEQ ID NO: 28 - RNA, 22 nt, Homo sapiens
uagcagcacauaaugguuugug

SEQ ID NO: 29 - RNA, 22 nt, Homo sapiens
ucguaccgugaguaauaaugcg

SEQ ID NO: 30 - RNA, 21 nt, Homo sapiens
caagucacuagugguuccguu

SEQ ID NO: 31 - RNA, 23 nt, Homo sapiens
uaaggugcaucuagugcagauag

SEQ ID NO: 32 - RNA, 21 nt, Homo sapiens
aucacauugccagggauuucc

SEQ ID NO: 33 - RNA, 21 nt, Homo sapiens
uucaaguaauucaggauaggu

SEQ ID NO: 34 - RNA, 22 nt, Homo sapiens
uagcuuaucagacugauguuga

SEQ ID NO: 35 - protein, 5 aa, Artificial Sequence
  source: /note=“Description of Artificial Sequence Synthetic peptide”
EAAAK

SEQ ID NO: 36 - protein, 4 aa, Artificial Sequence
  source: /note=“Description of Artificial Sequence Synthetic peptide”
GGGS

SEQ ID NO: 37 - protein, 15 aa, Artificial Sequence
  source: /note=“Description of Artificial Sequence Synthetic peptide”
GGGGSGGGGSGGGGS

(19) United States
(12) Patent Application Publication
(10) Pub. No.: US 2022/0402979 A1
(43) Pub. Date: Dec. 22, 2022

(54) BIOMARKERS AND TREATMENTS OF ALZHEIMER'S DISEASE AND MILD COGNITIVE IMPAIRMENT

(71) Applicant: AXON NEUROSCIENCE SE, Lamaca (CY)

(72) Inventors: Michal NOVAK, Bratislava (SK); Eva KONTSEKOVÁ, Senec (SK); Branislav KOVACECH, Bratislava (SK); Norbert ZILKA, Bratislava (SK)

(21) Appl. No.: 17/640,961

(22) PCT Filed: Sep. 8, 2020
(86) PCT No.: PCT/IB2020/000728

§ 371 Date: Mar. 7, 2022

Related U.S. Application Data
(60) Provisional application No. 62/897,940, filed on Sep. 9, 2019.
(60) Provisional application No. 63/003,585, filed on Apr. 1, 2020.

(51) Int. Cl. C07K 14/47; A61P 25/28

(52) U.S. Cl. CPC C07K 14/47; A61P 25/28; A61K 38/195

(57) ABSTRACT

The disclosure provides immunogenic peptides, compositions, means, and methods for treating Alzheimer's disease or mild cognitive impairment. The disclosure further provides means and methods for diagnosing patients, selecting patients for treatment, and/or evaluating the efficacy of treatment for Alzheimer's disease or mild cognitive impairment.

### CROSS-REFERENCE

This application claims the benefit of priority to U.S. Provisional Patent Application No. 62/897,940, filed Sep. 9, 2019, and U.S. Provisional Patent Application No. 63/003,585, filed Apr. 1, 2020; the entire contents of each of which are incorporated herein by reference.

### FIELD

The present disclosure relates to immunogenic peptides, compositions, means, and methods for treating Alzheimer's disease or mild cognitive impairment. The present disclosure further features means and methods for diagnosing and/or selecting patients for treatment for Alzheimer's disease or mild cognitive impairment, as well as means and methods for assessing the efficacy of treatment for Alzheimer's disease or mild cognitive impairment.

### BACKGROUND

Alzheimer's disease (AD) is a progressive neurodegenerative disorder that can destroy higher brain structures, such as those involved in memory and cognition. The disease leads to deficits in cognitive function and declines in memory, learning, language, and in the ability to perform intentional and purposeful movements. There is a need for effective methods and compositions for treatment and prophylaxis of AD.

AD is typically characterized histologically by the presence of extraneuronal plaques and intracellular and extracellular neurofibrillary tangles in the brain. Plaques are composed mainly of β amyloid (Aβ), whereas tangles comprise pathological forms of tau, such as pathological tau conformers and their aggregates. A recognized role for tau in AD pathology has been demonstrated in numerous studies. For example, Braak showed that the closest correlate for AD neurodegeneration was the presence of tau tangles, and not of amyloid plaques (Braak, H., et al. Neuropathological staging of Alzheimer-related changes. Acta Neuropathol 82:239-259 (1991)). A recognized role for tau in AD pathology has been demonstrated in numerous studies.

Tau belongs to a family of intrinsically disordered proteins, characterized by the absence of a rigid three-dimensional structure in their physiological environment (Zilka et al., 2008) However, tau truncation and hyperphosphorylation can cause pathological transformations from an intrinsically disordered state to multiple soluble and insoluble misdisordered structures, including paired helical filaments (PHFs) and other aggregates (Wischik et al., 1988a; Wischik et al., 1988b; Novak et al., 1993; Skrabana et al., 2006; Zilka et al., 2008; Kovacech et al., 2010). These structural changes lead to a toxic gain of function, to a loss of physiological function of the native protein, or both (Zilka et al., 2008; Kovacech et al., 2010).

Although tau appears to play a pathological role in the clinical manifestation of AD, the development of drugs that work against tau has been slow, in part due to tau's importance in physiologic microtubule dynamics and to its complex biology (Dickey and Petrucelli, 2006). However, an increased understanding of the molecular mechanisms underlying the pathological transformations of tau has opened up the possibility of specifically targeting pathological modifications of tau for therapeutic purposes. As a result, a number of therapeutic approaches that directly or indirectly target the tau cascade have emerged (for review articles, see, e.g. Dickey and Petrucelli, 2006; Schneider and Mandelkow, 2008; Zilka et al., 2008), including compounds that prevent or reverse tau aggregation (Wischik et al., 1996; Necula et al. 2005; Pickhardt et al., 2005; Taniguchi et al., 2005a; Larbig et al., 2007) small-molecule type drugs that inhibit tau kinases or activate tau phosphatases (Iqbal and Grundke-Iqbal, 2004; Noble et al., 2005; Iqbal and Grundke-Iqbal, 2007), microtubule stabilizing drugs (Zhang et al., 2005), drugs that facilitate the proteolytic degradation of misfolded tau proteins (Dickey et al., 2005, Dickey et al. 2006; Dickey and Petrucelli, 2006), and immunosuppresive drugs (Zilka et al., 2008), as well as immunotherapeutic strategies including active and passive immunization (Schneider and Mandelkow et al., 2008; Zilka et al., 2008; Tabira, T. Immunization Therapy for Alzheimer disease: A Comprehensive Review of Active Immunization Strategies. Tohoku J. Exp. Med., 220: 95-106 (2010)).

International Publication No. WO2013/041962 by Novak et al., herein incorporated by reference in its entirety, describes the discovery of four regions of tau that promote tau-tau aggregation in AD, as well as vaccines and antibodies that prevent tau aggregation by binding to those four regions.

An active immunization approach (i.e., one in which the patient's body itself generates immunity against the target) was found to be effective in clearing Aβ deposits and reversing neuropathological lesions in several APP-transgenic mouse studies of AD (see, e.g. Schenk et al., 1999; Janus et al., 2000; Morgan et al., 2000; Sigurdsson et al., 2001). Recently, active immunotherapy with a phosphorylated tau epitope (Tau 379-408 [P-Ser 396, 404]) reduced the extent of aggregated tau in the brain and slowed the progression of the behavioral phenotype in mouse models of tau tangle pathology (Asuni et al., 2007; Boutajangout et al. 2010; US2008/0050383; US/2010/00316564). Treated animals produced anti-tau antibodies, which were detected in the brain and colocalized with antibodies that recognized pathological tau (Asuni et al., 2007). This immunotherapeutic approach was substantially more effective in the early stages of functional impairments in the animals (5 months) than at later stages (8 months), suggesting that clearance of early-stage pathological tau can be of therapeutic benefit (Asuni et al., 2007; Zilka et al., 2008). Indeed, there is awareness that not all tau is susceptible or perhaps even suitable for disruption and clearance. Some have suggested that disrupting tau aggregates could increase the abundance of toxic intermediate species, while others have suggested that detectable tau aggregates are not necessarily toxic and can even play a protective role (Lee et al., 2005).

Thus, although immunotherapeutic approaches to target tau have shown pre-clinical promise, there is still a need for validated therapeutics and methods that produce improved, lasting benefits.

### SUMMARY OF THE DISCLOSURE

In some embodiments, the disclosure relates to a method for diagnosing a patient suffering from Alzheimer's Disease (AD) or mild cognitive impairment, the method comprising: a) detecting the presence and/or amount of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p in the patient or a sample from the patient; b) wherein the presence of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p and/or an altered amount relative to an amount in a control sample or threshold indicates Alzheimer's Disease in the subject; and c) diagnosing the presence or absence of Alzheimer's Disease in the patient based on step (b). In some embodiments, detecting the presence and/or amount of one or more of the above mRNA biomarkers comprises: a) obtaining a cerebrospinal fluid (CSF), serum, or blood plasma sample from the patient; b) optionally extracting one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p from the CSF, serum, or blood plasma of the patient; c) optionally performing cDNA synthesis; and d) detecting the presence and/or amount of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p, e.g., using quantitative polymerase chain reaction (qPCR). In some embodiments, the control sample is from a healthy individual or a patient with Alzheimer's Disease.

In some embodiments, the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, or hsa-miR-21-5p in the patient is 1, 2, 3, 4, 5, 6, 7, 8, 9, 10, 15, 20, 25, 50, 100, or more fold different than in the control sample. In some embodiments, the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, or hsa-miR-21-5p in the patient is 10%, 20%, 30%, 40%, 50%, 60%, 70%, 80%, 90%, or 100%, less than in the control sample. In some embodiments, the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, or hsa-miR-21-5p in the patient is 10%, 20%, 30%, 40%, 50%, 60%, 70%, 80%, 90%, 100%, 200%, or 500% greater than in the control sample. In some embodiments, diagnosis of a patient is carried out in vitro.

In some embodiments, the presence and/or an altered amount of at least 2, at least 3, at least 4, at least 5, at least 6, or at least 7 or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p relative to the control sample or threshold indicates Alzheimer's Disease in the subject. In some embodiments, the presence and/or amount of one or more of hsa-let-7a-5p, hsa-miR-15a-5p, hsa-miR191-5p, and hsa-miR-23a-3p is detected. In some embodiments, a patient diagnosed with Alzheimer's Disease or mild cognitive impairment is administered one or more doses of an immunogenic composition comprising an immunogenic peptide comprising SEQ ID NO: 1 or SEQ ID NO: 2.

In some embodiments, the disclosure relates to a method for diagnosing a patient suffering from Alzheimer's Disease (AD) or mild cognitive impairment, the method comprising: a) detecting the presence and/or amount of one or more metabolites, e.g., 2,4-dihydroxybutanoic acid, a phospholipid, phosphatidylcholine, sphingomyelin, or sterol, in blood plasma, serum, or cerebrospinal fluid (CSF) of the patient; b) wherein the presence of one or more metabolites and/or an altered amount relative to an amount in a control sample or threshold indicates Alzheimer's Disease in the subject; and c) diagnosing the presence or absence of Alzheimer's Disease in the patient based on step (b). In some embodiments, detecting the presence and/or amount of one or more metabolites comprises: a) obtaining a cerebrospinal fluid (CSF), serum, or blood plasma sample from the patient; b) extracting one or more metabolites from the CSF, serum, or blood plasma of the patient; and c) determining the amount of one or more metabolites, e.g., using mass spectrometry. In some embodiments, the mass spectrometry is time-of-flight mass spectrometry or tandem mass spectrometry/mass spectrometry. In some embodiments, the control sample is from a healthy individual or a patient with Alzheimer's Disease.

In some embodiments, the amount of metabolites in the sample from the patient is 1, 2, 3, 4, 5, 6, 7, 8, 9, 10, 15, 20, 25, 50, 100, or more fold different than in the control sample. In some embodiments, the amount of metabolites in the sample from the patient is 10%, 20%, 30%, 40%, 50%, 60%, 70%, 80%, 90%, 100%, 200%, or 500% greater than in the control sample. In some embodiments, the amount of metabolites in the sample from the patient is 10%, 20%, 30%, 40%, 50%, 60%, 70%, 80%, 90%, or 100% less than in the control sample. In some embodiments, diagnosis of a patient is carried out in vitro.

In some embodiments, the presence and/or an altered amount of at least 2, at least 3, at least 4, at least 5, at least 6, or at least 7 or more metabolites relative to the control sample or threshold indicates Alzheimer's Disease in the subject. In some embodiments, a patient diagnosed with Alzheimer's Disease or mild cognitive impairment is administered one or more doses of an immunogenic composition comprising an immunogenic peptide comprising SEQ ID NO: 1 or SEQ ID NO: 2.

In some embodiments, the disclosure relates to a method of treating Alzheimer's Disease (AD) or mild cognitive impairment, comprising: a) detecting the presence and/or amount of one or more of hsa-let-7a-5p, hsa-miR-15a-5p, hsa-miR191-5p, and hsa-miR-23a-3p in a sample from a patient; b) selecting the patient for treatment when the sample from the patient has one or more of hsa-let-7a-5p, hsa-miR-15a-5p, hsa-miR191-5p, and hsa-miR-23a-3p or an altered level of one or more of hsa-let-7a-5p, hsa-miR-15a-5p, hsa-miR191-5p, and hsa-miR-23a-3p relative to a control sample or threshold; and c) selecting the patient for treatment by administering one or more doses of an immunogenic composition comprising an immunogenic peptide comprising SEQ ID NO: 1 or SEQ ID NO: 2.

In some embodiments, the amount of hsa-let-7a-5p, hsa-miR-15a-5p, hsa-miR191-5p, and/or hsa-miR-23a-3p in the sample from the patient is 1, 2, 3, 4, 5, 6, 7, 8, 9, 10, 15, 20, 25, 50, 100, or more fold different than in the control sample. In some embodiments, the amount of hsa-let-7a-5p and/or hsa-miR-15a-5p in the sample from the patient is 1, 2, 3, 4, 5, 6, 7, 8, 9, 10, 15, 20, 25, 50, 100, or more fold less than in the control sample. In some embodiments, the amount of hsa-miR-15a-5p in the sample from the patient is 1, 2, 3, 4, 5, 6, 7, 8, 9, 10, 15, 20, 25, 50, 100, or more fold less than in the control sample. In some embodiments, the amount of hsa-miR191-5p and/or hsa-miR-23a-3p in the sample from the patient is 1, 2, 3, 4, 5, 6, 7, 8, 9, 10, 15, 20, 25, 50, 100, or more fold greater than in the control sample. In some embodiments, the amount of hsa-miR-23a-3p in the sample from the patient is 1, 2, 3, 4, 5, 6, 7, 8, 9, 10, 15, 20, 25, 50, 100, or more fold greater than in the control sample. In some embodiments, the presence and/or an altered amount of at least 2, at least 3, or at least 4 of hsa-let-7a-5p, hsa-miR-15a-5p, hsa-miR191-5p, and hsa-miR-23a-3p relative to the control sample or threshold indicates Alzheimer's Disease in the subject. In some embodiments, the presence and/or an altered amount of at least 2, at least 3, or at least 4 of hsa-let-7a-5p, hsa-miR-15a-5p, hsa-miR191-5p, and hsa-miR-23a-3p relative to the control sample or threshold indicates that the subject is selected for treatment with one or more doses of an immunogenic composition comprising an immunogenic peptide comprising SEQ ID NO: 1 or SEQ ID NO: 2.

In some embodiments, the disclosure relates to a method of treating Alzheimer's Disease (AD) or mild cognitive impairment, the method comprising: a) detecting the presence and/or amount of neurofilament light chain (NfL) in a sample from a patient; b) selecting the patient for treatment when the sample from the patient contains the NfL and/or an altered (e.g., increased) amount of the NfL relative to a control sample or threshold; and c) treating the patient by administering one or more doses of an immunogenic composition comprising an immunogenic peptide comprising SEQ ID NO: 1 or SEQ ID NO: 2.

In some embodiments, the amount of NfL in the patient is 1, 2, 3, 4, 5, 6, 7, 8, 9, 10, 15, 20, 25, 50, 100, or more fold higher than in the control sample. In some embodiments, the amount of NfL in the sample from the patient is more than a threshold of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 pg/ml. In some embodiments, the amount of NfL in the patient is 10%, 20%, 30%, 40%, 50%, 60%, 70%, 80%, 90%, or 100% greater than in the control sample. In some embodiments, the control sample is a plasma sample. In some embodiments, the sample from the patient is a plasma sample. In some embodiments, the disclosure relates to a method of treating Alzheimer's Disease (AD) or mild cognitive impairment, the method comprising: a) determining a Mini-Mental State Examination (MMSE) score for the patient; b) comparing the score from step a) to a threshold score; b) selecting the patient for treatment wherein the patient has a MMSE score above the threshold; and d) treating the patient by administering one or more doses of an immunogenic composition comprising an immunogenic peptide comprising SEQ ID NO: 1 or SEQ ID NO: 2.

In some embodiments, the MMSE score of the patient is at least 29, at least 28, at least 27, at least 26, at least 25, at least 24, at least 23, at least 22, at least 21, at least 20, at least 19, at least 18, at least 17, at least 16, at least 15, at least 14, at least 13, or at least 12. In some embodiments, the MMSE score of the patient is at or greater than the threshold of 29, 28, 27, 26, 25, 24, 23, 22, 21, 20, 19, 18, 17, 16, 15, 14, 13, or 12. In some embodiments, the MMSE score of the patient is 24-26. In some embodiments, the disclosure relates to a method of treating Alzheimer's Disease (AD) or mild cognitive impairment, comprising: a) determining the genotype for each of the two alleles of the ApoE gene present in the patient; b) selecting the patient for treatment when the patient has at least one ApoE-ε4 allele; and c) treating the patient by administering one or more doses of an immunogenic composition comprising an immunogenic peptide comprising SEQ ID NO: 1 or SEQ ID NO: 2. In some embodiments, the patient has two c4 alleles. In some embodiments, the disclosure relates to a method of treating Alzheimer's Disease (AD) or mild cognitive impairment, comprising: a) detecting the presence and/or amount of neurogranin in a sample from a patient; b) selecting the patient for treatment when the patient or sample from the patient contains neurogranin and/or an increased amount relative to a control sample or threshold; and c) treating the patient by administering one or more doses of an immunogenic composition comprising an immunogenic peptide comprising SEQ ID NO: 1 or SEQ ID NO: 2.

In some embodiments, the amount of neurogranin in the sample from the patient is 1, 2, 3, 4, 5, 6, 7, 8, 9, 10, 15, 20, 25, 50, 100, or more fold different than in the control sample. In some embodiments, the amount of neurogranin in the sample from the patient is greater than a threshold of 1, 2, 3, 4, 5, 6, 7, 8, 9, 10, 15, 20, 25, 50, or 100 pg/ml. In some embodiments, the amount of neurogranin in the sample from the patient is 10%, 20%, 30%, 40%, 50%, 60%, 70%, 80%, 90%, or 100% less than in the control sample. In some embodiments, the amount of neurogranin in the sample from the patient is 10%, 20%, 30%, 40%, 50%, 60%, 70%, 80%, 90%, or 100% greater than in the control sample.

In some embodiments, the disclosure relates to a method of treating a patient suffering from Alzheimer's Disease (AD) or mild cognitive impairment, the method comprising: a) determining the presence and/or amount of one or more of hsa-let-7a-5p, hsa-miR-15a-5p, hsa-miR191-5p, and hsa-miR-23a-3p in a sample from the patient; b) administering one or more doses of an immunogenic composition comprising an immunogenic peptide comprising SEQ ID NO: 1 or SEQ ID NO: 2; c) after administering the one or more doses, determining the presence and/or amount of one or more of hsa-let-7a-5p, hsa-miR-15a-5p, hsa-miR191-5p, and hsa-miR-23a-3p in the patient; d) comparing the presence and/or amount from step (c) after treatment to the presence and/or amount from step (a) before treatment, wherein an altered amount of the one or more of hsa-let-7a-5p, hsa-miR-15a-5p, hsa-miR191-5p, and hsa-miR-23a-3p indicates treatment efficacy; and e) administering one or more additional doses of the immunogenic composition when the patient demonstrates treatment efficacy in step (d).

In some embodiments, decreased amounts of hsa-let-7a-5p and/or hsa-miR-15a-5p after treatment indicates efficacy. In some embodiments, about 1, 2, 3, 4, 5, 6, 7, 8, 9, 10, 15, 20, 25, 50, 100, or more fold reduction of hsa-let-7a-5p and/or hsa-miR-15a-5p after treatment indicates efficacy. In some embodiments, increased amounts of hsa-miR191-5p and/or hsa-miR-23a-3p after treatment indicates efficacy. In some embodiments, about 1, 2, 3, 4, 5, 6, 7, 8, 9, 10, 15, 20, 25, 50, 100, or more fold increase of hsa-miR191-5p and/or hsa-miR-23 after treatment indicates efficacy.

In some embodiments, the patient has been treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of the immunogenic peptide in step (b). In some embodiments, the patient is treated with at least 2, 3, 4, 5, 6, 7, 8, 9, 10, 11, 12, 13, 14, or 15 additional doses of the immunogenic peptide in step (e).

In some embodiments, treating the patient comprises administering doses at 2-week intervals, 3-week intervals, 4-week intervals, 5-week intervals, 6-week intervals, 7-week intervals, 8-week intervals, 9-week intervals, 10-week intervals, 11-week intervals, 12-week intervals, 13-week intervals, 14-week intervals, 15-week intervals, 20-week intervals, 25-week intervals, 30-week intervals, 35-week intervals, or 40-week intervals.

In some embodiments, the disclosure relates to a method of treating a patient suffering from Alzheimer's Disease (AD) or mild cognitive impairment, the method comprising: a) determining the presence and/or amount of NfL in the patient; b) administering one or more doses of an immunogenic composition comprising an immunogenic peptide comprising SEQ ID NO: 1 or SEQ ID NO: 2; c) after administering the one or more doses, determining the presence and/or amount of NfL in the patient; d) comparing the presence and/or amount from step (c) after treatment to the presence and/or amount from step a before treatment, wherein an altered amount of NfL indicates treatment efficacy; and e) administering one or more additional doses of the immunogenic composition when the patient demonstrates treatment efficacy in step (d).

In some embodiments, a stable or decreased amount of NfL after treatment indicates efficacy. In some embodiments, an amount of NfL that does not increase by more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after treatment indicates efficacy. In some embodiments, an amount of NfL that decreases by more than about 30%, 25%, 20%, 15%, 14%, 13%, 12%, 11%, 10%, 9%, 8%, 7%, 6%, 5%, 4%, 3%, 2%, or 1% after treatment indicates efficacy. In some embodiments, an amount of NfL that does not increase by more than about 6 pg/mL, 5 pg/mL 4 pg/mL, 3 pg/mL or 2 pg/mL after treatment indicates efficacy. In some embodiments, the amount of NfL in a patient has increased by no more than about 3.2 pg/mL two years following administration of the one or more doses of the immunogenic composition compared to baseline. In some embodiments, the amount of NfL in a patient has increased about 1 to about 3.2 pg/mL two years following administration of the one or more doses of the immunogenic composition compared to baseline. In some embodiments, two years following administration of the one or more doses of the immunogenic composition, the increase in NfL levels in a patient administered the one or more doses of the immunogenic composition is about 10%, about 20%, about 30%, about 40%, about 50%, about 60%, about 70%, about 80%, about 90%, or about 100% less compared to the increase in NfL levels in an age-matched patient who has not received one or more doses of the immunogenic composition. In some embodiments, two years following administration of the one or more doses of the immunogenic composition, the increase in NfL levels in a patient administered the one or more doses of the immunogenic composition is about 10%, about 20%, about 30%, about 40%, about 50%, about 60%, about 70%, about 80%, about 90%, or about 100% less compared to the increase in NfL levels in an age-matched patient administered a placebo.

In some embodiments, the patient has been treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of the immunogenic peptide in step (b). In some embodiments, the patient is treated with at least 2, 3, 4, 5, 6, 7, 8, 9, 10, 11, 12, 13, 14, or 15 additional doses of the immunogenic peptide in step (e). In some embodiments, the one or more doses of the immunogenic composition comprises at least 6 doses. In further embodiments, the one or more doses of the immunogenic composition comprises 6 monthly doses followed by at least 5 boosters.

In some embodiments, treating the patient comprises administering doses at 2-week intervals, 3-week intervals, 4-week intervals, 5-week intervals, 6-week intervals, 7-week intervals, 8-week intervals, 9-week intervals, 10-week intervals, 11-week intervals, 12-week intervals, 13-week intervals, 14-week intervals, 15-week intervals, 20-week intervals, 25-week intervals, 30-week intervals, 35-week intervals, or 40-week intervals.

In some embodiments, the disclosure relates to a method of treating a patient suffering from Alzheimer's Disease (AD) or mild cognitive impairment, the method comprising: a) determining the presence and/or amount of neurogranin in the patient; b) administering one or more doses of an immunogenic composition comprising an immunogenic peptide comprising SEQ ID NO: 1 or SEQ ID NO: 2; c) after administering the one or more doses, determining the presence and/or amount of neurogranin in the patient; d) comparing the presence and/or amount from step (c) after treatment to the presence and/or amount from step a before treatment, wherein an altered amount of neurogranin indicates treatment efficacy; and e) administering one or more additional doses of the immunogenic composition when the patient demonstrates treatment efficacy in step (d).

In some embodiments, a stable or decreased amount of neurogranin after treatment indicates efficacy. In some embodiments, an amount of neurogranin that does not increase by more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after treatment indicates efficacy. In some embodiments, an amount of neurogranin that decreases by more than about 30%, 25%, 20%, 15%, 14%, 13%, 12%, 11%, 10%, 9%, 8%, 7%, 6%, 5%, 4%, 3%, 2%, or 1% after treatment indicates efficacy. In some embodiments, an amount of neurogranin that does not increase by more than about 20 pg/mL, 18 pg/mL, 16 pg/mL, 14 pg/mL, 12 pg/mL, 10 pg/mL, 8 pg/mL, 6 pg/mL, 5 pg/mL 4 pg/mL, 3 pg/mL or 2 pg/mL after treatment indicates efficacy.

In some embodiments, the patient has been treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of the immunogenic peptide in step (b). In some embodiments, the patient is treated with at least 2, 3, 4, 5, 6, 7, 8, 9, 10, 11, 12, 13, 14, or 15 additional doses of the immunogenic peptide in step (e).

In some embodiments, treating the patient comprises administering doses at 2-week intervals, 3-week intervals, 4-week intervals, 5-week intervals, 6-week intervals, 7-week intervals, 8-week intervals, 9-week intervals, 10-week intervals, 11-week intervals, 12-week intervals, 13-week intervals, 14-week intervals, 15-week intervals, 20-week intervals, 25-week intervals, 30-week intervals, 35-week intervals, or 40-week intervals.

In some embodiments, the disclosure relates to a method of treating a patient suffering from Alzheimer's Disease (AD) or mild cognitive impairment, the method comprising: a) determining a Mini-Mental State Examination (MMSE) score for the patient; b) comparing the score from step (a) to a threshold score, wherein optionally the threshold score is 20-26; c) selecting the patient for treatment wherein the patient has a MMSE score above the threshold; d) administering one or more doses of an immunogenic composition comprising an immunogenic peptide comprising SEQ ID NO: 1 or SEQ ID NO: 2; e) after administering the one or more doses, determining the MMSE score of the patient; f) comparing the MMSE score from step (e) after treatment to the MMSE score from step (a) before treatment, wherein an altered MMSE score indicates treatment efficacy; and g) administering one or more additional doses of the immunogenic composition when the patient demonstrates treatment efficacy in step (f). In some embodiments, the disclosure relates to a method of treating a patient suffering from Alzheimer's Disease (AD) or mild cognitive impairment, the method comprising: a) determining an AD biomarker signature (e.g. one or more of total tau protein>400 pg/mL; pT181 tau protein>60 pg/mL; Aβ42<600 pg/mL; and Aβ42:Aβ40 ratio<0.089) in the patient; b) administering one or more doses of an immunogenic composition comprising an immunogenic peptide comprising SEQ ID NO: 1 or SEQ ID NO: 2; c) after administering the one or more doses, determining an AD biomarker signature in the patient; d) comparing the biomarker signature from step (c) after treatment to the biomarker signature from step (a) before treatment, wherein an altered biomarker signature indicates treatment efficacy; and e) administering one or more additional doses of the immunogenic composition when the patient demonstrates treatment efficacy in step (d).

In some embodiments, pT181 decreases by at least 8.1 pg/mL two years following administration of the one or more doses of the immunogenic composition compared to baseline. In some embodiments, total tau decreases by at least 71.8 pg/mL two years following administration of the one or more doses of the immunogenic composition compared to baseline. In some embodiments, pT217 decreases by at least 69.2 pg/mL two years following administration of the one or more doses of the immunogenic composition compared to baseline. In some embodiments, Aβ40 decreases by at least 888 pg/mL two years following administration of the one or more doses of the immunogenic composition compared to baseline. In some embodiments, Aβ42 remains stable two years following administration of the one or more doses of the immunogenic composition compared to baseline (e.g., no more than a 1%, 5%, 10%, 15%, 20%, or 25% change compared to baseline). In some embodiments, Aβ42 remains stable two years following administration of the one or more doses of the immunogenic composition compared to baseline (e.g., a change, e.g., a decrease, of about 25 pg/mL, about 20 pg/mL, about 15 pg/mL, about 10 pg/mL, or about 5 pg/mL compared to baseline). In some embodiments, two years following administration of the one or more doses of the immunogenic composition, Aβ42 levels in the patient have decreased by about 20 pg/mL compared to baseline. In some embodiments, pT181 decreases by at least 8.1 pg/mL, pT217 decreases by at least 69.2 pg/mL, total tau decreases by at least 71.8 pg/mL, and Aβ40 decreases by at least 888 pg/mL two years following administration of the one or more doses of the immunogenic composition compared to baseline. In some embodiments, pT181 decreases by at least 8.1 pg/mL, pT217 decreases by at least 69.2 pg/mL, total tau decreases by at least 71.8 pg/mL, Aβ40 decreases by at least 888 pg/mL, and Aβ40 remains stable two years following administration of the one or more doses of the immunogenic composition compared to baseline. In some embodiments, the change in an AD biomarker signature following administration of the one or more doses of the immunogenic composition compared to baseline is calculated using the analysis of covariance (ANCOVA) model.

In some embodiments, pT181 decreases after two years following administration of the one or more doses of the immunogenic composition (compared to baseline). For instance, pT181 may decrease by at least about 2 to about 15 pg/mL, about 5 to about 15 pg/mL, about 2 to about 10 pg/mL, or about 5 to about 10 pg/mL two years following administration of the one or more doses of the immunogenic composition compared to baseline. In some embodiments, pT181 decreases by at least about 5 to about 10 pg/mL two years following administration of the one or more doses of the immunogenic composition compared to baseline.

In some embodiments, total tau decreases after two years following administration of the one or more doses of the immunogenic composition (compared to baseline). For instance, total tau may decrease by at least about 40 to about 100 pg/mL, about 50 to about 90 pg/mL, about 55 to about 85 pg/mL, about 60 to about 80 pg/mL, or about 65 to about 75 pg/mL two years following administration of the one or more doses of the immunogenic composition compared to baseline. In some embodiments, total tau decreases by at least about 65 to about 75 pg/mL two years following administration of the one or more doses of the immunogenic composition compared to baseline.

In some embodiments, pT217 decreases after two years following administration of the one or more doses of the immunogenic composition (compared to baseline). For instance, pT217 may decrease by at least about 40 to about 100 pg/mL, about 50 to about 90 pg/mL, about 55 to about 85 pg/mL, about 60 to about 80 pg/mL, or about 65 to about 75 pg/mL two years following administration of the one or more doses of the immunogenic composition compared to baseline. In some embodiments, pT217 decreases by at least about 65 to about 75 pg/mL two years following administration of the one or more doses of the immunogenic composition compared to baseline.

In some embodiments, Aβ40 decreases after two years following administration of the one or more doses of the immunogenic composition (compared to baseline). For instance, Aβ40 decreases by at least about 700 to about 1100 pg/mL, about 750 to about 1050 pg/mL, about 800 to about 1000 pg/mL, or about 850 to about 950 pg/mL two years following administration of the one or more doses of the immunogenic composition compared to baseline. In some embodiments, Aβ40 decreases by at least about 850 to about 950 pg/mL two years following administration of the one or more doses of the immunogenic composition compared to baseline. In some embodiments, Aβ42 remains stable two years following administration of the one or more doses of the immunogenic composition compared to baseline (e.g., no more than a 1%, 5%, 10%, 15%, 20%, or 25% change compared to baseline). In some embodiments, Aβ42 remains stable two years following administration of the one or more doses of the immunogenic composition compared to baseline (e.g., a decrease of about 25 pg/mL, about 20 pg/mL, about 15 pg/mL, about 10 pg/mL, or about 5 pg/mL compared to baseline). In some embodiments, pT181 decreases by at least about 5 to about 10 pg/mL, pT217 decreases by at least about 65 to about 75 pg/mL, total tau decreases by at least about 65 to about 75 pg/mL, and Aβ40 decreases by at least about 850 to about 950 pg/mL two years following administration of the one or more doses of the immunogenic composition compared to baseline.

In some embodiments, pT181 decreases by at least about 5 to about 10 pg/mL, pT217 decreases by at least about 65 to about 75 pg/mL, total tau decreases by at least about 65 to about 75 pg/mL, Aβ40 decreases by at least about 850 to about 950 pg/mL, and Aβ40 remains stable two years following administration of the one or more doses of the immunogenic composition compared to baseline. In some embodiments, the change in an AD biomarker signature following administration of the one or more doses of the immunogenic composition compared to baseline is calculated using the analysis of covariance (ANCOVA) model.

In some embodiments, the disclosure relates to a method of treating a patient suffering from Alzheimer's Disease (AD) or mild cognitive impairment, the method comprising: a) determining a volume of medial temporal lobe atrophy as assessed according to a brain MRI in the patient; b) administering one or more doses of an immunogenic composition comprising an immunogenic peptide comprising SEQ ID NO: 1 or SEQ ID NO: 2 if medial temporal lobe atrophy is above a threshold (e.g., Scheltens score of ≥2); c) after administering the one or more doses, determining a volume of temporal lobe atrophy in the patient; d) comparing temporal lobe atrophy assessed in step (c) after treatment to temporal lobe atrophy assessed in step (a) before treatment, wherein an altered temporal lobe atrophy indicates treatment efficacy; and e) administering one or more additional doses of the immunogenic composition when the patient demonstrates treatment efficacy in step (d).

In some embodiments, the disclosure relates to a method of treating Alzheimer's Disease (AD) or mild cognitive impairment, comprising administering one or more doses of an immunogenic composition comprising an immunogenic peptide comprising SEQ ID NO: 1 or SEQ ID NO: 2 to a patient expressing one or more of hsa-let-7a-5p, hsa-miR-15a-5p, hsa-miR191-5p, and hsa-miR-23a-3p in a sample. In some embodiments, the amount of hsa-let-7a-5p, hsa-miR-15a-5p, hsa-miR191-5p, and/or hsa-miR-23a-3p in the patient is about 1, 2, 3, 4, 5, 6, 7, 8, 9, 10, 15, 20, 25, 50, 100, or more fold different relative to a control sample or threshold. In some embodiments, the amount of hsa-miR191-5p and/or hsa-miR-23a-3p in the sample from the patient is about 1, 2, 3, 4, 5, 6, 7, 8, 9, 10, 15, 20, 25, 50, 100, or more fold greater than in a control sample or threshold. In some embodiments, the amount of hsa-let-7a-5p and/or hsa-miR-15a-5p in the sample from the patient is about 1, 2, 3, 4, 5, 6, 7, 8, 9, 10, 15, 20, 25, 50, 100, or more fold less than in a control sample or threshold. In some embodiments, the control sample is from a healthy individual or a patient with Alzheimer's Disease. In some embodiments, the control sample is blood plasma or CSF.

In some embodiments, the disclosure relates to a method of treating Alzheimer's Disease (AD) or mild cognitive impairment, comprising administering one or more doses of an immunogenic composition comprising an immunogenic peptide comprising SEQ ID NO: 1 or SEQ ID NO: 2 to a patient having NfL in a sample. In some embodiments, the amount of NfL in the patient is less than a threshold of 10, 20, 30, 40, 50, 60, 70, 80, 90, or 100 pg/ml. In some embodiments, the amount of NfL in the patient is greater than 20 pg/ml.

In some embodiments, the disclosure relates to a method of treating Alzheimer's Disease (AD) or mild cognitive impairment, comprising administering one or more doses of an immunogenic composition comprising an immunogenic peptide comprising SEQ ID NO: 1 or SEQ ID NO: 2 to a patient having an MMSE score of greater than 23. In some embodiments, the MMSE score of the patient is 26, 27, 28, 29, or 30.

In some embodiments, the disclosure relates to a method of treating Alzheimer's Disease (AD) or mild cognitive impairment, comprising administering one or more doses of an immunogenic composition comprising an immunogenic peptide comprising SEQ ID NO: 1 or SEQ ID NO: 2 to a patient having at least one c4 allele of the ApoE gene.

In some embodiments, the disclosure relates to a method of treating a patient suffering from Alzheimer's Disease (AD) or mild cognitive impairment, the method comprising: a) determining the presence and/or amount of one or more metabolites in the patient; b) administering one or more doses of an immunogenic composition comprising an immunogenic peptide comprising SEQ ID NO: 1 or SEQ ID NO: 2; c) after administering the one or more doses, determining the presence and/or amount of one or more metabolites in the patient; d) comparing the presence and/or amount from step (c) after treatment to the presence and/or amount from step a before treatment, wherein an altered amount of neurogranin indicates treatment efficacy; and e) administering one or more additional doses of the immunogenic composition when the patient demonstrates treatment efficacy in step (d). In some embodiments, an altered amount of one or more metabolites after treatment indicates efficacy. In some embodiments, an amount of the one or more metabolites that increases by more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after treatment indicates efficacy. In some embodiments, an amount of the one or more metabolites that decreases by more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after treatment indicates efficacy. In some embodiments, an altered amount of at least 2, at least 3, at least 4, at least 5, at least 6 or at least 7 metabolites after treatment indicates efficacy. In some embodiments, the patient has been treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of the immunogenic peptide in step (b). In some embodiments, the patient is treated with at least 2, 3, 4, 5, 6, 7, 8, 9, 10, 11, 12, 13, 14, or 15 additional doses of the immunogenic peptide in step (e). In some embodiments, treating the patient comprises administering doses at 2-week intervals, 3-week intervals, 4-week intervals, 5-week intervals, 6-week intervals, 7-week intervals, 8-week intervals, 9-week intervals, 10-week intervals, 11-week intervals, 12-week intervals, 13-week intervals, 14-week intervals, 15-week intervals, 20-week intervals, 25-week intervals, 30-week intervals, 35-week intervals, or 40-week intervals.

In some embodiments, the disclosure relates to a method for selecting a patient suffering from Alzheimer's Disease to treat with an immunogenic composition comprising an immunogenic peptide comprising SEQ ID NO: 1 or SEQ ID NO: 2, comprising: a) detecting the presence and/or amount of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p in the patient or a sample from the patient; b) comparing to a control sample or threshold, wherein the presence of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p and/or an altered amount relative to an amount in a control sample or threshold indicates Alzheimer's Disease in the subject; and c) selecting the patient for treatment based on step (b). In some embodiments, the disclosure relates to a method for selecting a patient suffering from Alzheimer's Disease to treat with an immunogenic composition comprising an immunogenic peptide comprising SEQ ID NO: 1 or SEQ ID NO: 2, comprising: a) detecting the presence and/or amount of 2,4-dihydroxybutanoic acid, a phospholipid, phosphatidylcholine, sphingomyelin, and/or sterol, in blood plasma, serum, or cerebrospinal fluid (CSF) of the patient; b) comparing to a control sample or threshold, wherein the presence of one or more metabolites and/or an altered amount relative to an amount in a control sample or threshold indicates Alzheimer's Disease in the subject; and c) selecting the patient for treatment based on step (b).

In some embodiments, the disclosure relates to a method of treating a patient suffering from Alzheimer's Disease or mild cognitive impairment, the method comprising administering to the patient one or more doses of an immunogenic composition comprising an immunogenic peptide comprising SEQ ID NO: 1 or SEQ ID NO: 2, wherein the immunogenic composition is administered in an amount effective to yield an antibody titre of at least 100 ng/mL of antibodies against pathological tau. In some embodiments, the disclosure relates to a method of treating a patient suffering from Alzheimer's Disease or mild cognitive impairment, the method comprising administering to the patient one or more doses of an immunogenic composition comprising an immunogenic peptide comprising SEQ ID NO: 1 or SEQ ID NO: 2, wherein the immunogenic composition is administered in an amount effective to yield an antibody titre of at least 100 ng/mL of antibodies against p108 tau peptide. In some embodiments, the disclosure relates to a method of treating a patient suffering from Alzheimer's Disease or mild cognitive impairment, the method comprising administering to the patient one or more doses of an immunogenic composition comprising an immunogenic peptide comprising SEQ ID NO: 1 or SEQ ID NO: 2, wherein the immunogenic composition is administered in an amount effective to yield an antibody titre of at least 1000 ng/mL of antibodies against p108 tau peptide. In some embodiments, the disclosure relates to a method of treating a patient suffering from Alzheimer's Disease or mild cognitive impairment, the method comprising administering to the patient one or one more doses of an immunogenic composition comprising an immunogenic peptide comprising SEQ ID NO: 1 or SEQ ID NO: 2, wherein the immunogenic composition is administered in an amount effective to yield a calculated area under the curve (AUC) of titre for antibodies (e.g., IgG, IgM antibodies) that bind p108 tau peptide of at least 100,000. In some embodiments, the antibodies are IgG antibodies. In some embodiments, the method comprises administering an initial dosing regimen of six doses of the immunogenic peptide or composition, followed by at least five boosters. In some embodiments, the number of administered boosters is fix, six, or seven.

In some embodiments, the immunogenic peptide induces at least one antibody characterized by a Kd against pathological tau of about 10 nM or less. In some embodiments, the immunogenic peptide induces at least one antibody characterized by a Kd against pathological tau of about 4.2 nM or less (e.g., about 4.2 nM to about 0.01 nM). In some embodiments, the immunogenic peptide induces at least one antibody characterized by a Kd against pathological tau of about 1 nM or less (e.g., about 1 nM to about 0.01 nM). In some embodiments, pathological tau comprises tau151-391/4R.

In some embodiments, the method comprises an initial dosing regimen comprising administering a dose of the immunogenic composition once per month for e.g., six months.

In some embodiments, the method further comprises administering one or more boosters following the initial dosing regimen. In some embodiments, the method comprises administering at least five (e.g., five, six, seven, etc.) boosters.

In some embodiments, if the titre of antibodies against pathological tau falls below 100 ng/mL, the method further comprises administering one or more boosters following the initial dosing regimen in an amount effective to restore the titre of antibodies against pathological tau to at least 100 ng/mL, wherein optionally, following administration of the one or more boosters, the average titre of antibodies against pathological tau is at least 100 ng/mL for at least two years measured from the initial dosing regimen (e.g., of six doses of the immunogenic peptide). In some embodiments, the one or more boosters are administered once every three months. In some embodiments, the method comprises administering at least five boosters. In some embodiments, the method comprises administering at least six boosters. In some embodiments, the method comprises administering at least seven boosters. In some embodiments, the patient suffers from Alzheimer's Disease.

In some embodiments, if the titre of antibodies against p108 tau peptide falls below 100 ng/mL, the method further comprises administering one or more boosters following the initial dosing regimen in an amount effective to restore the titre of antibodies against p108 tau peptide to at least 100 ng/mL, wherein optionally, following administration of the one or more boosters, the average titre of antibodies against p108 tau peptide is at least 100 ng/mL for at least two years measured from the initial dosing regimen (e.g., of six doses of the immunogenic peptide). In some embodiments, if the titre of antibodies against p108 tau peptide falls below 1000 ng/mL, the method further comprises administering one or more boosters following the initial dosing regimen in an amount effective to restore the titre of antibodies against p108 tau peptide to at least 1000 ng/mL, wherein optionally, following administration of the one or more boosters, the average titre of antibodies against p108 tau peptide is at least 1000 ng/mL for at least two years measured from the initial dosing regimen (e.g., of six doses of the immunogenic peptide). In some embodiments, the one or more boosters are administered once every three months. In some embodiments, the method comprises administering at least five boosters. In some embodiments, the method comprises administering at least six boosters. In some embodiments, the method comprises administering at least seven boosters. In some embodiments, the patient suffers from Alzheimer's Disease.

In some embodiments, if the calculated area under the curve (AUC) of titre for antibodies (e.g., IgG, IgM antibodies) that bind p108 tau peptide falls below 100,000, the method further comprises administering one or more boosters following the initial dosing regimen in an amount effective to restore the calculated AUC of titre for antibodies (e.g., IgG, IgM antibodies) that bind p108 tau peptide to at least 100,000, wherein optionally, following administration of the one or more boosters, the average calculated AUC of titre for antibodies (e.g., IgG, IgM antibodies) that bind p108 tau peptide is at least 100,000 for at least two years measured from the initial dosing regimen (e.g., of six doses of the immunogenic peptide). In some embodiments, the one or more boosters are administered once every three months. In some embodiments, the method comprises administering at least five boosters. In some embodiments, the method comprises administering at least six boosters. In some embodiments, the method comprises administering at least seven boosters. In some embodiments, the patient suffers from Alzheimer's Disease.

In some embodiments, the immunogenic composition is administered in an amount effective to stabilize or reduce accumulation of neurofilament light chain in plasma, relative to baseline accumulation of neurofilament light chain in plasma. In some embodiments, the patient has stable neurofilament light chain concentration (e.g., no more than a 1%, 5%, 10%, 15%, 20%, or 25% increase in neurofilament light chain concentration) in plasma, relative to baseline accumulation, following the initial dosing regimen. In some embodiments, the patient has reduced accumulation of neurofilament light chain in plasma, relative to baseline accumulation, following the initial dosing regimen. In some embodiments, the method further comprises administering one or more boosters following the initial dosing regimen. In some embodiments, if the patient has an increased accumulation (e.g., more than about a 25% increase) of neurofilament light chain in plasma relative to baseline accumulation following the initial dosing regimen, the method further comprises administering one or more boosters following the initial dosing regimen in an amount effective to stabilize or reduce accumulation of neurofilament light chain in plasma, wherein optionally, following administration of the one or more boosters, the patient has stable neurofilament light chain concentration or reduced accumulation of neurofilament light chain in plasma for at least two years measured from the initial dosing regimen. In some embodiments, the one or more boosters are administered once every three months. In some embodiments, the method comprises administering at least five boosters.

In some embodiments, following administration of the immunogenic composition (e.g., following administration of the initial dosing regimen): if the patient is 50-67 years of age, the concentration of neurofilament light chain in plasma increases by no more than 2 pg/mL, preferably by no more than 1.8 pg/mL, for at least two years; or if the patient is 68-85 years of age, the concentration of neurofilament light chain in plasma increases by no more than 2.3 pg/mL for at least two years. In some embodiments, the method further comprises administering one or more boosters following the initial dosing regimen if: during the two years following the initial dosing regimen, the average concentration of neurofilament light chain in plasma increases by more than 2 pg/mL (e.g., if the patient is 50-67 years of age), or by more than 2.3 pg/mL (e.g., if the patient is 58-85 years of age); wherein the one or more boosters are administered in an amount effective to maintain an average increase in neurofilament light chain in plasma of no more than 2 pg/mL (e.g., if the patient is 50-67 years of age) or no more than 2.3 pg/mL (e.g., if the patient is 58-85 years of age). In some embodiments, the one or more boosters are administered once every three months. In some embodiments, the method comprises administering at least five boosters.

In some embodiments, the immunogenic composition is administered in an amount effective to stabilize or reduce total tau (t-tau) levels in CSF, relative to t-tau levels in CSF before administration of the immunogenic composition. In some embodiments, the patient has stable t-tau levels in CSF following the initial dosing regimen, relative to t-tau levels before administration of the initial dosing regimen. In some embodiments, the patient has reduced t-tau levels in CSF following the initial dosing regimen, relative to t-tau levels before administration of the initial dosing regimen. In some embodiments, by two years following administration of the immunogenic composition (e.g., by two years following administration of the initial dosing regimen), the concentration of t-tau in CSF is decreased by at least 2 ng/L, optionally by at least 2.5 ng/L, and further optionally by at least 2.7 ng/L.

In some embodiments, the method further comprises administering one or more boosters following the initial dosing regimen. In some embodiments, if the concentration of t-tau in CSF has not decreased by at least 2 ng/L, optionally by at least 2.5 ng/L, or further optionally by at least 2.7 ng/mL after the initial dosing regimen, e.g., by two years after the initial dosing regimen, the method further comprises administering one or more boosters following the initial dosing regimen in an amount effective to stabilize or reduce t-tau levels in CSF, wherein optionally, following administration of the one or more boosters, the patient has stable or reduced t-tau levels for at least two years measured from the initial dosing regimen or from the date of booster administration, wherein, further optionally, by two years after the initial dosing regimen or two years after booster administration, the concentration of t-tau in CSF is decreased by at least 2 ng/L, optionally by at least 2.5 ng/L, and further optionally by at least 2.7 ng/L. In some embodiments, the one or more boosters are administered once every three months. In some embodiments, the method comprises administering at least five boosters.

In some embodiments, the immunogenic composition is administered in an amount effective to inhibit spreading of pathological tau. In some embodiments, pathological tau comprises tau151-391/4R.

In some embodiments, the immunogenic composition is effective to inhibit spreading of pathological tau following the initial dosing regimen. In some embodiments, the method further comprises administering one or more boosters following the initial dosing regimen. In some embodiments, spreading of pathological tau increases, the method further comprises administering one or more boosters following the initial dosing regimen in an amount effective to inhibit spreading of pathological tau, wherein optionally, after administration of the one or more boosters, spreading of pathological tau is inhibited for at least two years measured from the initial dosing regimen. In some embodiments, the one or more boosters are administered once every three months. In some embodiments, the method comprises administering at least five boosters.

In some embodiments, the immunogenic composition is administered in an amount effective to a) reduce tau protein phosphorylated at threonine-181 (pT181 tau) levels in CSF, relative to pT181 tau levels in CSF before administration of the immunogenic composition; and/or b) reduce tau protein phosphorylated at threonine-217 (pT217 tau) levels in CSF, relative to pT217 tau levels in CSF before administration of the immunogenic composition. In some embodiments, the patient has reduced pT181 tau levels and/or reduced pT217 levels in CSF following the initial dosing regimen. In some embodiments, by two years following administration of the immunogenic composition, a) the concentration of pT181 tau in CSF is decreased by at least 5 ng/L, optionally by at least 5.3 ng/L over the concentration before administration of the immunogenic composition; and/or b) the concentration of pT217 tau in CSF is decreased by at least 30 ng/L, optionally by at least 34 ng/L over the concentration before administration of the immunogenic composition.

In some embodiments, the method further comprises administering one or more boosters following the initial dosing regimen. In some embodiments, if the patient has increased pT181 tau levels and/or increased pT217 levels in CSF following the initial dosing regimen, the method further comprises administering one or more boosters following the initial dosing regimen in an amount effective to reduce pT181 tau levels and/or reduce pT217 levels in CSF, wherein optionally, following administration of the one or more boosters, the patient has reduced pT181 tau levels and/or reduced pT217 levels in CSF for at least two years measured from the initial dosing regimen. In some embodiments, if by two years following the initial dosing regimen, the concentration of pT181 tau in CSF is not decreased by at least 5 ng/L (optionally by at least 5.3 ng/L) and/or the concentration of pT217 tau in CSF is not decreased by at least 30 ng/L (optionally by at least 34 ng/L), the method further comprises administering one or more boosters following the initial dosing regimen in an amount effective to decrease the concentration of pT181 tau in CSF by at least 5 ng/L (optionally by at least 5.3 ng/L) and/or decrease the concentration of pT217 tau in CSF by at least 30 ng/L (optionally by at least 34 ng/L). In some embodiments, the one or more boosters are administered once every three months. In some embodiments, the method comprises administering at least five boosters.

In some embodiments, the immunogenic composition is administered in an amount effective to halt white matter degradation in the patient's fornix and/or genu corpus callosum, relative to white matter degradation before administration of the immunogenic composition. In some embodiments, white matter degradation is measured by fractional anisotropy or mean diffusivity.

In some embodiments, the immunogenic composition is administered in an amount effective to increase fractional anisotropy in white matter. In some embodiments, the increase in fractional anisotropy in white matter is maintained or continues to improve (e.g., increases further) for at least two years after administration of the immunogenic composition. In some embodiments, the immunogenic composition is administered in an amount effective to decrease mean diffusivity in white matter. In some embodiments, the decrease in mean diffusivity in white matter is maintained or continues to improve (e.g., decreases further) for at least two years after administration of the immunogenic composition. In some embodiments, increase in fractional anisotropy is detected for fornix. In some embodiments, decrease in mean diffusivity is detected for fornix. Without being limited by theory, fractional anisotropy (FA) reflects the degree of water diffusion directionality and tends to decrease over time in AD patients, while mean diffusivity (MD) describes the overall diffusion in each of the principal directions and tends to increase over time in AD patients.

In some embodiments, the immunogenic composition is administered in an amount effective to halt axonal degeneration, relative to axonal degradation before administration of the immunogenic composition. In some embodiments, axonal degeneration is measured by fractional anisotropy or mean diffusivity. In some embodiments, the immunogenic composition is administered in an amount effective to increase fractional anisotropy in axons. In some embodiments, the increase in fractional anisotropy in axons is maintained or continues to improve (e.g., increases further) for at least two years after administration of the immunogenic composition. In some embodiments, the immunogenic composition is administered in an amount effective to decrease mean diffusivity in axons. In some embodiments, the decrease in mean diffusivity in axons is maintained or continues to improve (e.g., decreases further) for at least two years after administration of the immunogenic composition. In some embodiments, white matter degradation is halted and/or axonal degradation is halted after administration of the initial dosing regimen.

In some embodiments, the method further comprises administering one or more boosters following the initial dosing regimen. In some embodiments, if the patient has increased white matter degradation and/or increased axonal degradation following the initial dosing regimen, the method further comprises administering one or more boosters following the initial dosing regimen in an amount effective to halt white matter degeneration and/or halt axonal degeneration, wherein optionally, following administration of the one or more boosters, white matter degradation is halted and/or axonal degradation is halted for at least two years measured from the initial dosing regimen. In some embodiments, the one or more boosters are administered once every three months. In some embodiments, the method comprises administering at least five boosters.

In some embodiments, the immunogenic composition is administered in an amount effective to prevent hippocampal atrophy (e.g., effective to prevent a decrease in hippocampal volume relative to hippocampal volume before administration of the immunogenic composition). In some embodiments, the method comprises administering an initial dosing regimen comprising administering the immunogenic composition once per month for six months, optionally followed by one or more boosters. In some embodiments, if the patient's hippocampal volume is reduced following an initial dosing regimen comprising administering the immunogenic composition once per month for six months, relative to the patient's hippocampal volume before administration of the initial dosing regimen, the method further comprises administering one or more boosters in an amount effective to prevent further hippocampal atrophy, wherein optionally the one or more boosters are administered in an amount effective to prevent further hippocampal atrophy for at least two years.

In some embodiments, the patient is 50-67 years of age and, by two years following administration of the immunogenic composition, the patient's hippocampal volume is reduced by no more than 10%, preferably by no more than 8%, relative to the patient's hippocampal volume before administration of the immunogenic composition.

In some embodiments, if the patient's hippocampal volume is reduced by more than 10% following an initial dosing regimen comprising administering the immunogenic composition once per month for six months, relative to the patient's hippocampal volume before administration of the initial dosing regimen, the method further comprises administering one or more boosters in an amount effective to prevent further hippocampal atrophy (e.g., to prevent further loss of hippocampal volume of more than 10%), optionally for at least two years. In some embodiments, the one or more boosters are administered once every three months. In some embodiments, the method comprises administering at least five boosters.

In some embodiments, the immunogenic composition is administered in an amount effective to prevent cortical atrophy (e.g., effective to prevent a decrease in cortical volume relative to cortical volume before administration of the immunogenic composition). In some embodiments, the method comprises administering an initial dosing regimen comprising administering the immunogenic composition once per month for six months, optionally followed by one or more boosters.

In some embodiments, if the patient's cortical volume is reduced following an initial dosing regimen comprising administering the immunogenic composition once per month for six months, relative to the patient's cortical volume before administration of the initial dosing regimen, the method further comprises administering one or more boosters in an amount effective to prevent further cortical atrophy, wherein optionally the one or more boosters are administered in an amount effective to prevent further cortical atrophy for at least two years.

In some embodiments, the patient is 50-67 years of age and, by two years following administration of the immunogenic composition, the patient's cortical volume is reduced by no more than 5%, preferably by no more than 4%, relative to the patient's cortical volume before administration of the immunogenic composition. In some embodiments, if the patient's cortical volume is reduced by more than 5% following an initial dosing regimen comprising administering the immunogenic composition once per month for six months, relative to the patient's cortical volume before administration of the initial dosing regimen, the method further comprises administering one or more boosters in an amount effective to prevent further cortical atrophy (e.g., to prevent further loss of cortical volume of more than 5%), optionally for at least two years. In some embodiments, the one or more boosters are administered once every three months. In some embodiments, the method comprises administering at least five boosters.

In some embodiments, atrophy (e.g., volume) is measured by MRI volumetry.

In some embodiments, the patient is 50-67 years of age and the immunogenic composition is administered in an amount effective to slow the patient's rate of cognitive decline. In some embodiments, the method comprises administering an initial dosing regimen comprising administering the immunogenic composition once per month for six months, optionally followed by one or more boosters. In some embodiments, the patient's rate of cognitive decline is measured using the Clinical Dementia Rating Scale Sum of Boxes (CDR-SB) test, the Alzheimer's Disease Cooperative Study Mild Cognitive Impairment activities of Daily Living (ADCS MCI ADL) questionnaire, the Mini-Mental State Examination (MMSE), and/or a cognitive battery comprising the Cogstate International Shopping List Task, the Cogstate One Card Learning and One Card Back Tasks, the Letter Fluency Test and Category Fluency Test, and/or the Digit Symbol Coding test.

In some embodiments, by two years following administration of the immunogenic composition (e.g., following administration of the initial dosing regimen), the patient's CDR-SB test score is increased by no more than 4, relative to the patient's CDR-SB test score before administration of the immunogenic composition. In some embodiments, by two years following administration of the immunogenic composition (e.g., following administration of the initial dosing regimen), the patient's MMSE score is reduced by no more than 7 points, preferably by no more than 6.5 points, relative to the patient's MMSE score before administration of the immunogenic composition. In some embodiments, by two years following administration of the immunogenic composition (e.g., following administration of the initial dosing regimen), the patient's ADCS MCI ADL questionnaire score is reduced by no more than 14 points, preferably by no more than 13.5 points, relative to the patient's ADCS MCI ADL questionnaire score before administration of the immunogenic composition.

In some embodiments, the method further comprises administering one or more boosters if: a) the patient's CDR-SB score increases by more than 4 relative to the patient's CDR-SB test score before administration of the immunogenic composition (e.g., the initial dosing regimen); b) the patient's MMSE score reduces by more than 6.5 points or by more than 7 points relative to the patient's MMSE score before administration of the immunogenic composition (e.g., the initial dosing regimen); and/or c) the patient's ADCS MCI ADL questionnaire score reduces by more than 13.5 points or by more than 14 points relative to the patient's ADCS MCI ADL questionnaire score before administration of the immunogenic composition (e.g., the initial dosing regimen). In some embodiments, the one or more boosters are administered in an amount effective to prevent: a) an increase in the patient's CDR-SB score of more than 4 relative to the patient's CDR-SB test score before administration of the immunogenic composition (e.g., the initial dosing regimen); b) a reduction in the patient's MMSE score of more than 6.5 points or more than 7 points relative to the patient's MMSE score before administration of the immunogenic composition (e.g., the initial dosing regimen); and/or c) a reduction in the patient's ADCS MCI ADL questionnaire score of more than 13.5 points or more than 14 points relative to the patient's ADCS MCI ADL questionnaire score before administration of the immunogenic composition (e.g., the initial dosing regimen). In some embodiments, the one or more boosters are administered once every three months. In some embodiments, the method comprises administering at least five boosters.

For any of the above embodiments: In some embodiments, the immunogenic peptide further comprises a carrier protein or peptide. In some embodiments, the carrier comprises at least one of serum albumin, keyhole limpet hemocyanin, an immunoglobulin molecule, thyroglobulin, ovalbumin, tetanus toxoid, and/or a toxoid from another pathogenic bacterium, such as diphtheria, E. coli, V. cholera, or H. pylori, an attenuated toxin derivative, a cytokine such as IL-1, IL-1α or IL-1β peptide, IL-2, IFNγ, IL-10, GM-CSF, or a chemokine, such as MIP1α, MIP1β, or RANTES. In some embodiments, the carrier comprises keyhole limpet hemocyanin (KLH) or a fragment thereof. In some embodiments, the KLH or fragment thereof is coupled to SEQ ID NO: 1 or SEQ ID NO: 2 via a maleimide linker. In some embodiments, the maleimide linker comprises GMBS. In some embodiments, the maleimide linker is Sulfo-GMBS. In some embodiments, a dose comprises about 20-50 μg, e.g., at least about 20 μg, at least about 30 μg, at least about 40 μg, or at least about 50 μg of the immunogenic peptide. In some embodiments, each dose comprises about 40 μg of the immunogenic peptide. In some embodiments, the immunogenic composition further comprises an adjuvant, e.g, an aluminum compound. In some embodiments, the aluminum compound comprises aluminum hydroxide (Al(OH)₃). In some embodiments, the adjuvant comprises about 0.1 mg to about 10 mg aluminum (Al³⁺), optionally, about 0.5 mg aluminum (Al³⁺). In some embodiments, the immunogenic composition further comprises a phosphate buffer, optionally in a volume of about 0.3 mL. In some embodiments, the immunogenic composition comprises an immunogenic peptide comprising SEQ ID NO: 2 coupled via a maleimide linker (e.g., Sulfo-GMBS) to KLH, about 0.5 mg aluminum (Al³⁺), and a phosphate buffer, optionally in a volume of about 0.3 mL. In some embodiments, treating the patient comprises administering 5 doses, 6 doses, 7 doses, 8 doses, 9 doses, 10 doses, 11 doses, 12 doses, 13 doses, 14 doses, 15 doses, 20 doses, 25 doses, 50 doses or 100 doses of the immunogenic peptide. In some embodiments, treating the patient comprises administering doses at 2-week intervals, 3-week intervals, 4-week intervals, 5-week intervals, 6-week intervals, 7-week intervals, 8-week intervals, 9-week intervals, 10-week intervals, 11-week intervals, 12-week intervals, 13-week intervals, 14-week intervals, 15-week intervals, 20-week intervals, 25-week intervals, 30-week intervals, 35-week intervals, or 40-week intervals. In some embodiments, treating the patient comprises administering one or more (e.g., 6) doses of the immunogenic composition at 4-week intervals followed by one or more (e.g., 5, 6) doses of the immunogenic composition at 20-week intervals. In some embodiments, the control sample is from a healthy individual or a patient with Alzheimer's Disease. In some embodiments, the control sample is a CSF sample or a blood plasma sample. In some embodiments, the immunogenic peptide consists of SEQ ID NO: 1 or SEQ ID NO: 2. In some embodiments, the immunogenic peptide consists of SEQ ID NO: 1 and 1, 2, 3, 4, or 5 additional residues at the N and/or C terminus, wherein the additional residues are identical to those immediately adjacent to SEQ ID NO: 1 when aligned against tau 2N4R. In some embodiments, the immunogenic peptide consists of SEQ ID NO: 2 and 1, 2, 3, 4, or 5 additional residues at the N and/or C terminus, wherein the additional residues are identical to those immediately adjacent to SEQ ID NO: 2 when aligned against tau 2N4R.

For any of the above embodiments: In some embodiments, the one or more boosters comprise an immunogenic peptide comprising SEQ ID NO: 1 or SEQ ID NO: 2. In some embodiments, the immunogenic peptide further comprises a carrier protein or peptide. In some embodiments, the carrier comprises at least one of serum albumin, keyhole limpet hemocyanin, an immunoglobulin molecule, thyroglobulin, ovalbumin, tetanus toxoid, and/or a toxoid from another pathogenic bacterium, such as diphtheria, E. coli, V. cholera, or H. pylori, an attenuated toxin derivative, a cytokine such as IL-1, IL-1α or IL-1β peptide, IL-2, IFNγ, IL-10, GM-CSF, or a chemokine, such as MIP1α, MIP1β, or RANTES. In some embodiments, the carrier comprises keyhole limpet hemocyanin (KLH) or a fragment thereof. In some embodiments, the KLH or fragment thereof is coupled to SEQ ID NO: 1 or SEQ ID NO: 2 via a maleimide linker. In some embodiments, the maleimide linker comprises GMBS. In some embodiments, the maleimide linker is Sulfo-GMBS. In some embodiments, the booster further comprises an adjuvant, e.g., an aluminum compound. In some embodiments, the aluminum compound comprises aluminum hydroxide (Al(OH)₃). In some embodiments, the adjuvant comprises about 0.1 mg to about 10 mg aluminum (Al³⁺), optionally, about 0.5 mg aluminum (Al³⁺). In some embodiments, the booster further comprises a phosphate buffer, optionally in a volume of about 0.3 mL. In some embodiments, the booster comprises an immunogenic peptide comprising SEQ ID NO: 2 coupled via a maleimide linker (e.g., Sulfo-GMBS) to KLH, about 0.5 mg aluminum (Al³⁺), and a phosphate buffer, optionally in a volume of about 0.3 mL. In some embodiments, a dose of the booster comprises at least about 20 μg, at least about 30 μg, at least about 40 μg, or at least about 50 μg of the immunogenic peptide, optionally about 20 to 50 μg of the immunogenic peptide. In some embodiments, a dose of the booster comprises about 40 μg of the immunogenic peptide. In some embodiments, the booster is identical in composition to the immunogenic composition administered as part of an initial dosing regimen. In some embodiments, the booster is administered at the same dose as the immunogenic composition administered as part of an initial dosing regimen. In some embodiments, the immunogenic peptide consists of SEQ ID NO: 1 or SEQ ID NO: 2. In some embodiments, the immunogenic peptide consists of SEQ ID NO: 1 and 1, 2, 3, 4, or 5 additional residues at the N and/or C terminus, wherein the additional residues are identical to those immediately adjacent to SEQ ID NO: 1 when aligned against tau 2N4R. In some embodiments, the immunogenic peptide consists of SEQ ID NO: 2 and 1, 2, 3, 4, or 5 additional residues at the N and/or C terminus, wherein the additional residues are identical to those immediately adjacent to SEQ ID NO: 2 when aligned against tau 2N4R.

For any of the above embodiments: In some embodiments, the patient has a diagnosis of probable Alzheimer's Disease according to the revised 2011 NIA-AA criteria. In some embodiments, the patient has a diagnosis of probable Alzheimer's Disease according to the revised 2018 NIA-AA criteria. In some embodiments, the patient has a total Mini-Mental State Examination score of ≥20 and ≤26. In some embodiments, the patient has a brain MRI finding consistent with a diagnosis of Alzheimer's Disease. In some embodiments, the patient has a medial temporal lobe atrophy as assessed on brain MRI and according to a Scheltens score of ≥2 (rated on a scale of 0-4 on the more atrophied side). In some embodiments, the patient has a positive AD biomarker signature in the CSF (e.g., one or more of total tau protein>400 pg/mL; pT181 tau protein>60 pg/mL; Aβ42<600 pg/mL; and Aβ42:Aβ40 ratio<0.089). In some embodiments, the patient is 50-85 years of age, e.g., 50-67 years of age, 50-70 years of age, 68-85 years of age, or 71-85 years of age. In some embodiments, the patient is 50-67 years of age. In some embodiments, the patient is 50-70 years of age. In some embodiments, the patient is 68-85 years of age. In some embodiments, the patient has received a stable therapy with an acetylcholinesterase inhibitor for at least 3 months before the start of treatment. In some embodiments, the patient has received a stable dose of memantine treatment for at least 3 months before the start of treatment. In some embodiments, the patient is male. In some embodiments, before administration of the immunogenic peptide, the patient has an Aβ₄₂ concentration of less 600 pg/mL in cerebrospinal fluid (CSF), a phosphorylated tau (p-tau) T181 concentration of greater than 60 pg/mL in CSF, and a total tau (t-tau) concentration of greater than 400 pg/mL in CSF. In some embodiments, prior to treatment, the patient is less than 80 years old, has a plasma neurofilament light chain concentration of greater than 10 pg/mL, has detectable tau protein in CSF, and lacks micro-hemorrhages, beginning or large confluent hemispheric deep white matter lesions (Fazekas grade 2 or 3), severe hippocampal atrophy (Scheltens score of 4), and hallucinations. In some embodiments, diagnosis of a patient is carried out in vitro.

In some embodiments, the patient has a diagnosis of Alzheimer's Disease. In some embodiments, the patient has Alzheimer's Disease. In some embodiments, the patient has a diagnosis of mild cognitive impairment. In some embodiments, the patient has mild cognitive impairment.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1: Distribution of total MMSE score in patient population at the time of the screening visit.

FIG. 2: Comparison of total MMSE for each patient at V01 and V05.

FIG. 3: Mean change in CDR-SB (50-70 and 71-85 years old) compared to baseline over the course of the study.

FIG. 4: Mean change in CDR-SB (50-67 and 68-85 years old) compared to baseline over the course of the study.

FIG. 5: Mean change in MMSE (50-70 and 71-85 years old) compared to baseline over the course of the study.

FIG. 6: Mean change in MMSE (50-67 and 68-85 years old) compared to baseline over the course of the study.

FIG. 7: Mean change in ADCS-MCI-ADL 24 (50-70 and 71-85 years old) compared to baseline over the course of the study.

FIG. 8: Mean change in ADCS-MCI-ADL 24 (50-67 and 68-85 years old) compared to baseline over the course of the study.

FIG. 9: Levels of IgG and IgM titers compared to AADvac1 peptide.

FIG. 10: Levels of anti-KLH antibody titer.

FIG. 11: Antibody responses to AADvac1

FIG. 12: Distribution of antibodies to tau151-391/4R and AADvac1 after treatment.

FIG. 13: IgG titers at V08 and area-under-the-curve comparison between younger (50-70 years old) and older (71-85 years old) subpopulations.

FIG. 14: Correlation of IgG area-under-the-curve at V16 with age of patient.

FIG. 15: IgG titers at V08 and area-under-the-curve comparison between younger (50-67 years old) and older (68-85 years old) subpopulations.

FIG. 16: Levels of pT217 and pT181 tau in patients with different neurological conditions.

FIG. 17: Change in pT217 tau levels from V02 to V16.

FIG. 18: Change in pT181 tau levels from V02 to V16.

FIG. 19: Change in total tau, pT217 tau, and pT181 tau levels from V02 to V16 in patients who were positive for pT181 tau.

FIG. 20: Distribution of NfL in the patient population at baseline.

FIG. 21: Change in NfL levels from V02 to V16.

FIG. 22: Change in NfL levels from V02 to V16 in patient age groups (51-70 and 71-85 years old).

FIG. 23: Change in NfL levels from V02 to V16 in patient age groups (51-67 and 68-85 years old).

FIG. 24: Change in tau levels from V02 to V16.

FIG. 25: Change in neurogranin levels from V02 to V16.

FIG. 26: Change in ratio of Aβ1-42:Aβ1-40 and Aβ1-42 and Aβ1-40 levels from V02 to V16.

FIG. 27: Scheltens atrophy score and left hippocampal volume distributions in patients at the screening visit.

FIG. 28: Comparison of left and right hippocampus volume at the screening visit.

FIG. 29: Distribution of left and right hippocampus volume in patient population.

FIG. 30: Distribution of Fazekas white matter lesion score in the patient population at screening visit.

FIG. 31: Distribution of micro-hemorrhages at screening visit.

FIG. 32: Change in brain volume in regions of interest in younger subgroup (<71 years old) at V16.

FIG. 33: Change in brain volume in regions of interest (cortical volume longitudinal (CVL), temporal lobe volume longitudinal (TLVL), and lateral ventricle longitudinal (LVVL)) in younger subgroup (<68 years old) at V16.

FIG. 34: Change in brain volume in regions of interest (left hipoocampal volume longitudinal (LHVL), right hipoocampal volume longitudinal (RHVL), and whole brain volume (WBV)) in younger subgroup (<68 years old) at V16.

FIG. 35: Change in fractional anisotropy in different regions of the brain.

FIG. 35: Level of microRNAs in patients with AD compared to control.

FIG. 36: Change in levels of microRNAs in patients after treatment with AADvac1 or placebo measured at V11 or V16 compared to V02.

FIG. 37: MicroRNA levels measured at V11 or V16 compared to baseline.

FIG. 38: Levels of metabolites (acylcarnitines) in AD and control patients.

FIG. 39: Levels of metabolites (glycerophospholipids) in AD and control patients.

FIG. 40: Levels of metabolites (glycerophospholipids) in AD and control patients.

FIG. 41: Levels of metabolites (glycerophospholipids, sphingolipids and sugars) in AD and control patients.

FIG. 42: Levels of metabolites (amino acids and biogenic amines) in AD and control patients.

FIG. 43: Levels of peptides in AD and control patients.

FIGS. 44A-44I: Safety endpoint data for AADvac1 and placebo groups.

FIGS. 45A-45C: CSF biomarker (tau) levels in AADvac1 and placebo groups.

FIGS. 46A-46E: Diffusion Tensor Imaging (DTI) MRI results in AADvac1 and placebo groups.

FIGS. 47A-47D: CDR-SB score and cortical atrophy for AADvac1 therapy.

FIGS. 48A-48N: CDR-SB score, MMSE score, ADCS-MCI-ADS score, MRI volumetry, and plasma NfL levels for AADvac1 therapy.

FIGS. 49A-49D: Antibody response against tau peptide and pathological tau.

FIG. 50: Antibody response in elderly population.

FIGS. 51A-51B: Serum antibody affinity against pathological tau, and comparison to competitive tau monoclonal antibodies.

FIG. 52: AD biomarker (CSF) positive patient subgroup criteria and demographics.

FIGS. 53A-53P: Plot of immune response and treatment efficacy in AD biomarker (CSF) positive patient subgroup.

FIG. 54: AD-probable patient subgroup demographics.

FIGS. 55A-55J: Correlation between immune response and treatment efficacy in AD-probable patient subgroup.

### DETAILED DESCRIPTION

In order to better understand the disclosure, certain exemplary embodiments are discussed herein. In addition, certain terms are discussed to aid in the understanding.

Unless otherwise defined, all technical and scientific terms used herein have the same meaning as commonly understood by one of ordinary skill in the art to which this disclosure belongs. All references cited herein are incorporated by reference in their entireties. To the extent terms or discussion in references conflict with this disclosure, the latter shall control.

As used herein, the singular forms of a word also include the plural form of the word, unless the context clearly dictates otherwise; as examples, the terms “a,” “an,” and “the” are understood to be singular or plural. By way of example, “an element” means one or more element. The term “or” shall mean “and/or” unless the specific context indicates otherwise.

The term “comprising,” or variations such as “comprises,” will be understood to imply the inclusion of a stated element, integer or step, or group of elements, integers or steps, but not the exclusion of any other element, integer or step, or group of elements, integers or steps. Throughout the specification the word “consisting of,” or variations such as “consists of,” will be understood to imply the inclusion of a stated element, integer or step, or group of elements, integers or steps, and the exclusion of any other element, integer or step, or group of elements, integers or steps. Throughout the specification the word “consisting essentially of,” or variations such as “consists essentially of,” will be understood to imply the inclusion of a stated element, integer or step, or group of elements, integers or steps, and any other element, integer or step, or group of elements, integers or steps that do not materially affect the basic and novel characteristics of the disclosure and/or claim.

About can be understood as within +/−10%, e.g., +/−10%, 9%, 8%, 7%, 6%, 5%, 4%, 3%, 2%, 1%, 0.5%, 0.1%, 0.05%, or 0.01% of the stated value. When used in reference to a percentage value, “about” can be understood as within ±1% (e.g., “about 5%” can be understood as within 4%-6%) or ±0.5% (e.g., “about 5%” can be understood as within 4.5%-5.5%). Unless otherwise clear from the context, all numerical values provided herein are modified by the term “about.” All ranges used herein encompass the endpoints.

As used herein, the terms “treatment,” “treating,” and the like, refer to obtaining a desired pharmacologic and/or physiologic effect. The effect can be prophylactic in terms of completely or partially preventing a disease or symptom thereof from occurring in the first place and/or can be therapeutic in terms of at least a partial remediation, delaying, slowing, reducing, or reversing of one or more symptoms of a disease and/or an adverse effect attributable to the disease. As an example, the term “treatment” and the like, as used herein, encompasses any treatment of Alzheimer's disease (AD) or related tauopathies in a mammal, particularly in a human, and includes: (a) preventing the disease from occurring in a subject, e.g., a subject identified as predisposed to the disease or at risk of acquiring the disease but has not yet been diagnosed as having it; (b) delaying onset or progression of the disease, e.g., as compared to the anticipated onset or progression of the disease in the absence of treatment; (c) inhibiting the disease, e.g., arresting its development; and/or (d) relieving the disease, i.e., causing regression of the disease. In some embodiments, “treating” refers to administering e.g., subcutaneously, an effective dose, or effective multiple doses of a composition e.g., a composition comprising an immunogenic peptide as disclosed herein to an animal (including a human being) suspected of suffering or already suffering from AD or another tauopathy. It can also refer to reducing, eliminating, or at least partially arresting, as well as to exerting any beneficial effect, on one or more symptoms of the disease and/or associated with the disease and/or its complications.

“Prevention” refers to administration to a patient susceptible to, or otherwise at risk of, a particular disease. Anyone in the general population may be at risk for a tauopathy such as AD. In some embodiments, the subject is identified by one or more marker, e.g., one or more genetic marker, of being at increased risk or susceptibility for a tauopathy such as AD over the risk in the general population. “Tauopathy” refers to a disease associated with the formation of pathological tau. Some individuals have an increased, genetic risk for AD. In some embodiments, prevention as used herein includes eliminating or reducing the risk, or delaying the onset of disease in a patient, e.g., in a patient at increased risk of a tauopathy. Delay of onset or progression can be measured, e.g., based on standard times of disease progression in similar populations or individuals.

“Physiological tau” refers to any one of the 6 isoforms of normal human tau, namely: 2N4R (SEQ ID NO: 3), 1N4R (SEQ ID NO: 4), 2N3R (SEQ ID NO: 5), ON4R (SEQ ID NO: 6), 1N3R (SEQ ID NO: 7), and ON3R (SEQ ID NO: 8). Excluded from this definition are those that carry any one of the phosphorylation modifications associated with Alzheimer's disease and other tauopathies.

TABLE 1

| Name | SEQ ID NO: | Sequence |
|---|---|---|
| 2N4R | SEQ ID NO: 3 | MAEPRQEFEVMEDHAGTYGLGDRKDQGGYTMHQDQE |
| | | GDTDAGLKESPLQTPTEDGSEEPGSETSDAKSTPTAED |
| | | VTAPLVDEGAPGKQAAAQPHTEIPEGTTAEEAGIGDTPS |
| | | LEDEAAGHVTQARMVSKSKDGTGSDDKKAKGADGKTKI |
| | | ATPRGAAPPGQKGQANATRIPAKTPPAPKTPPSSGEPP |
| | | KSGDRSGYSSPGSPGTPGSRSRTPSLPTPPTREPKKVA |
| | | VVRTPPKSPSSAKSRLQTAPVPMPDLKNVKSKIGSTENL |
| | | KHQPGGGKVQIINKKLDLSNVQSKCGSKDNIKHVPGGG |
| | | SVQIVYKPVDLSKVTSKCGSLGNIHHKPGGGQVEVKSEK |
| | | LDFKDRVQSKIGSLDNITHVPGGGNKKIETHKLTFRENAK |
| | | AKTDHGAEIVYKSPVVSGDTSPRHLSNVSSTGSIDMVDS |
| | | PQLATLADEVSASLAKQGL |
| 1N4R | SEQ ID NO: 4 | MAEPRQEFEVMEDHAGTYGLGDRKDQGGYTMHQDQE |
| | | GDTDAGLKESPLQTPTEDGSEEPGSETSDAKSTPTAEA |
| | | EEAGIGDTPSLEDEAAGHVTQARMVSKSKDGTGSDDKK |
| | | AKGADGKTKIATPRGAAPPGQKGQANATRIPAKTPPAPK |
| | | TPPSSGEPPKSGDRSGYSSPGSPGTPGSRSRTPSLPTP |
| | | PTREPKKVAVVRTPPKSPSSAKSRLQTAPVPMPDLKNV |
| | | KSKIGSTENLKHQPGGGKVQIINKKLDLSNVQSKCGSKD |
| | | NIKHVPGGGSVQIVYKPVDLSKVTSKCGSLGNIHHKPGG |
| | | GQVEVKSEKLDFKDRVQSKIGSLDNITHVPGGGNKKIET |
| | | HKLTFRENAKAKTDHGAEIVYKSPVVSGDTSPRHLSNVS |
| | | STGSIDMVDSPQLATLADEVSASLAKQGL |
| 2N3R | SEQ ID NO: 5 | MAEPRQEFEVMEDHAGTYGLGDRKDQGGYTMHQDQE |
| | | GDTDAGLKESPLQTPTEDGSEEPGSETSDAKSTPTAED |
| | | VTAPLVDEGAPGKQAAAQPHTEIPEGTTAEEAGIGDTPS |
| | | LEDEAAGHVTQARMVSKSKDGTGSDDKKAKGADGKTKI |
| | | ATPRGAAPPGQKGQANATRIPAKTPPAPKTPPSSGEPP |
| | | KSGDRSGYSSPGSPGTPGSRSRTPSLPTPPTREPKKVA |
| | | VVRTPPKSPSSAKSRLQTAPVPMPDLKNVKSKIGSTENL |
| | | KHQPGGGKVQIVYKPVDLSKVTSKCGSLGNIHHKPGGG |
| | | QVEVKSEKLDFKDRVQSKIGSLDNITHVPGGGNKKIETH |
| | | KLTFRENAKAKTDHGAEIVYKSPVVSGDTSPRHLSNVSS |
| | | TGSIDMVDSPQLATLADEVSASLAKQGL |
| 0N4R | SEQ ID NO: 6 | MAEPRQEFEVMEDHAGTYGLGDRKDQGGYTMHQDQE |
| | | GDTDAGLKAEEAGIGDTPSLEDEAAGHVTQARMVSKSK |
| | | DGTGSDDKKAKGADGKTKIATPRGAAPPGQKGQANAT |
| | | RIPAKTPPAPKTPPSSGEPPKSGDRSGYSSPGSPGTPG |
| | | SRSRTPSLPTPPTREPKKVAVVRTPPKSPSSAKSRLQTA |
| | | PVPMPDLKNVKSKIGSTENLKHQPGGGKVQIINKKLDLS |
| | | NVQSKCGSKDNIKHVPGGGSVQIVYKPVDLSKVTSKCG |
| | | SLGNIHHKPGGGQVEVKSEKLDFKDRVQSKIGSLDNITH |
| | | VPGGGNKKIETHKLTFRENAKAKTDHGAEIVYKSPVVSG |
| | | DTSPRHLSNVSSTGSIDMVDSPQLATLADEVSASLAKQG |
| | | L |
| 1N3R | SEQ ID NO: 7 | MAEPRQEFEVMEDHAGTYGLGDRKDQGGYTMHQDQE |
| | | GDTDAGLKESPLQTPTEDGSEEPGSETSDAKSTPTAEA |
| | | EEAGIGDTPSLEDEAAGHVTQARMVSKSKDGTGSDDKK |
| | | AKGADGKTKIATPRGAAPPGQKGQANATRIPAKTPPAPK |
| | | TPPSSGEPPKSGDRSGYSSPGSPGTPGSRSRTPSLPTP |
| | | PTREPKKVAVVRTPPKSPSSAKSRLQTAPVPMPDLKNV |
| | | KSKIGSTENLKHQPGGGKVQIVYKPVDLSKVTSKCGSLG |
| | | NIHHKPGGGQVEVKSEKLDFKDRVQSKIGSLDNITHVPG |
| | | GGNKKIETHKLTFRENAKAKTDHGAEIVYKSPVVSGDTS |
| | | PRHLSNVSSTGSIDMVDSPQLATLADEVSASLAKQGL |
| 0N3R | SEQ ID NO: 8 | MAEPRQEFEVMEDHAGTYGLGDRKDQGGYTMHQDQE |
| | | GDTDAGLKAEEAGIGDTPSLEDEAAGHVTQARMVSKSK |
| | | DGTGSDDKKAKGADGKTKIATPRGAAPPGQKGQANAT |
| | | RIPAKTPPAPKTPPSSGEPPKSGDRSGYSSPGSPGTPG |
| | | SRSRTPSLPTPPTREPKKVAVVRTPPKSPSSAKSRLQTA |
| | | PVPMPDLKNVKSKIGSTENLKHQPGGGKVQIVYKPVDLS |
| | | KVTSKCGSLGNIHHKPGGGQVEVKSEKLDFKDRVQSKI |
| | | GSLDNITHVPGGGNKKIETHKLTFRENAKAKTDHGAEIVY |
| | | KSPVVSGDTSPRHLSNVSSTGSIDMVDSPQLATLADEVS |
| | | ASLAKQGL |

“Pathological tau” includes various pathological tau conformers and structures and encompasses all of the following: Tau Type IA, IB, IIA, and IIB, misordered, misdisordered tau (monomer, dimer, trimer, oligomer), misdisordered soluble tau, sarkosyl-insoluble tau, extracellular tau deposits, tau aggregates, paired helical filaments, neurofibrillary pathology, including neurofibrillary lesions, tangles, threads, fibrils, axonal spheriods, highly phosphorylated forms of truncated tau and of full-length tau, or any other form of tau associated with AD or another tauopathy.

“Linked” refers to attachment of a moiety to an immunogenic peptide, antibody, or compound. The moiety can be coupled, or complexed, or covalently or noncovalently attached. The moiety can be chemically crosslinked or expressed or synthesized as a fusion with the peptide or antibody. “Moiety” refers to any compound (e.g., any organic molecule, peptide, protein, nucleic acid, carrier, adjuvant, etc.) that is able to be attached to the immunogenic peptide, antibody, or binding protein. “Carrier” refers to an agent that may be linked to an immunogenic peptide to increase its size and help elicit an immune response. Without being bound by theory, in some cases small peptides may be less effective immunogens because they act as haptens that lack the necessary Th-cell epitopes and/or are captured with low efficiency by antigen presenting cells (APC). Thus, in some embodiments, a peptide immunogen can be linked to a suitable carrier to help elicit an immune response. In certain embodiments, suitable carriers include serum albumin, keyhole limpet hemocyanin, an immunoglobulin molecule, thyroglobulin, ovalbumin, tetanus toxoid, and/or a toxoid from another pathogenic bacterium, such as diphtheria, E. coli, V. cholera, or H. pylori, or an attenuated toxin derivative. Other carriers for stimulating or enhancing an immune response include a cytokine such as IL-1, IL-1α or IL-1β peptide, IL-2, IFNγ, IL-10, GM-CSF, and a chemokine, such as MIP1α, MIP1β or RANTES. Immunogenic peptides can also be linked to agents that enhance transport across tissues, as described in O'Mahony, WO 97/17613 and WO 97/17614. In some embodiments, an immunogenic peptide further comprises a carrier peptide or protein. In some embodiments, the carrier is keyhole limpet hemocyanin (KLH) or a fragment thereof, e.g., a fragment that provides for an enhanced immune response. In some embodiments, an immunogenic peptide comprises or consists of a peptide having an amino acid sequence of KDNIKHVPGGGS (SEQ ID NO: 1) coupled to KLH. In some embodiments, an immunogenic peptide comprises or consists of a peptide having a sequence of CKDNIKHVPGGGS (SEQ ID NO: 2) coupled to KLH.

“Immunogenic” refers to something that can elicit an immune response. The immune response can be antibody- or cell-mediated, or both.

“Adjuvant” refers to a substance that is capable of increasing, amplifying, or modulating the immune response to the accompanying peptide.

The term “isolated,” as used herein, shall mean removed from one or more components of the natural environment. For instance, a polynucleotide of genomic, cDNA, or synthetic origin or some combination thereof may be isolated if it (1) is not associated with all or a portion of a polynucleotide in which the “isolated polynucleotide” is found in nature, (2) is operably linked to a polynucleotide which it is not linked to in nature, (3) does not occur in nature as part of a larger sequence, and/or (4) is otherwise isolated from other components of its natural environment.

“Mild cognitive impairment” (MCI) as used herein, refers to the stage between the expected cognitive impairment of normal aging and cognitive impairment associated with dementia, e.g., Alzheimer's disease. In the context of this application the underlying cause of MCI is understood to be Alzheimer's disease (AD) pathology. MCI may be identified in a patient through an assessment of mental performance, such as but not limited to the Wechsler Memory Scale-revised, Short Test of Mental Status, the Montreal Cognitive Assessment (MoCA), or the Mini-Mental State Examination (MMSE). See Kokmen et al. A short test of mental status: description and preliminary results. Mayo Clinic Proceedings. 1987; 62; 281-88; Ziad et al. The Montreal Cognitive Assessment, MoCA: A Brief Screening Tool for Mild Cognitive Impairment. Journal of the American Geriatrics Society. 2005; 53(4):695-99; Hua et al., Tensor-based morphometry as a neuroimaging biomarker for Alzheimer's disease: an MRI study of 676 AD, MCI, and normal subjects. NeuroImage. 2008; 43(3):458-69; Wechsler, D., 1987. WMS-R Wechsler Memory Scale-Revised Manual. The Psychological Corporation. Harcourt Brace Jovanovich, Inc., New York. In some embodiments, MCI is assessed using the Alzheimer's Disease Cooperative Study Mild Cognitive Impairment activities of Daily Living (ADCS MCI ADL) inventory.

As used herein, “Alzheimer's disease” (AD) (used interchangeably herein with “Alzheimer's disease dementia” or “AD dementia”) and “mild cognitive impairment” (MCI) can both be caused by AD pathology. AD can be distinguished from MCI using the NIA-AA criteria (e.g., the ATN criteria of the NIA-AA research framework, see Jack et al., Alzheimer's & Dementia, (2018)) as well as neuropsychological examination(s) used to identify MCI (e.g., the Wechsler Memory Scale-revised, Short Test of Mental Status, the MoCA, and/or the MMSE).

Immunogenic Peptides

Disclosed herein are fragments of tau for use in inducing an immune response to pathological tau. In one aspect, disclosed herein are immunogenic peptides comprising one or more of the regions of tau protein. The immunogenic peptide may be derived from regions of tau protein involved in the formation of the core of paired helical filaments (PHFs) and that promote PHF assembly in vitro. In some embodiments, an immunogenic peptide comprises or consists of fragments from tau protein encompassing four therapeutic epitopes, comprising tau 267-273 (SEQ ID NO: 9), tau 298-304 (SEQ ID NO: 11), tau 329-335 (SEQ ID NO: 13), or tau 361-367 (SEQ ID NO: 15), numbered according to the longest human tau isoform tau 2N4R. In some embodiments, an immunogenic peptide comprises or consists of fragments from tau protein comprising tau 268-273 (SEQ ID NO: 10), tau 299-304 (SEQ ID NO: 12), tau 330-335 (SEQ ID NO: 14), or tau 362-367 (SEQ ID NO: 16), numbered according to the longest human tau isoform tau 2N4R. In some embodiments, an immunogenic peptide comprises or consists of fragments from tau protein comprising tau 314-342 (SEQ ID NO: 17), tau 352-380 (SEQ ID NO: 18), or tau 357-368 (SEQ ID NO: 19). In some embodiments, an immunogenic peptide comprises a peptide sequence of CKDNIKHVPGGGS (SEQ ID NO: 2). In some embodiments, an immunogenic peptide is a fragment of a pathologic tau protein that comprises or consists of CKDNIKHVPGGGS (SEQ ID NO: 2) or KDNIKHVPGGGS (SEQ ID NO: 1). In some embodiments, an immunogenic peptide is a fragment of a pathologic tau protein that comprises or consists of CKDNIKHVPGGGS (SEQ ID NO: 2) or KDNIKHVPGGGS (SEQ ID NO: 1) with up to 5, up to 4, up to 3, up to 2, or up to 1 mutation. In some embodiments, an immunogenic peptide is a fragment of a pathologic tau protein that comprises or consists of CKDNIKHVPGGGS (SEQ ID NO: 2) or KDNIKHVPGGGS (SEQ ID NO: 1) and further comprises 1, 2, 3, 4, or 5 additional amino acids, e.g., inserted at the ends and/or internally to the sequence. In some embodiments, the immunogenic peptide consists of SEQ ID NO: 1 and 1, 2, 3, 4, or 5 additional residues at the N and/or C terminus, wherein the additional residues are identical to those immediately adjacent to SEQ ID NO: 1 when aligned against tau 2N4R. In some embodiments, the immunogenic peptide consists of SEQ ID NO: 2 and 1, 2, 3, 4, or 5 additional residues at the N and/or C terminus, wherein the additional residues are identical to those immediately adjacent to SEQ ID NO: 2 when aligned against tau 2N4R. In some embodiments, an immunogenic peptide is a fragment of a pathologic tau protein that has at least 99%, 95%, 92%, 90%, 85%, 80%, 75%, 70%, 65%, 60%, 55%, or 50% sequence identity with

(SEQ ID NO: 2)
CKDNIKHVPGGGS
or
(SEQ ID NO: 1)
KDNIKHVPGGGS.

TABLE 2

| Name | SEQ ID NO | Sequence |
|---|---|---|
| tau 294-305 | SEQ ID NO: 1 | KDNIKHVPGGGS |
| C-tau 294-305 | SEQ ID NO: 2 | CKDNIKHVPGGGS |
| tau 267-273 | SEQ ID NO: 9 | KHQPGGG |
| tau 268-273 | SEQ ID NO: 10 | HQPGGG |
| tau 298-304 | SEQ ID NO: 11 | KHVPGGG |
| tau 299-304 | SEQ ID NO: 12 | HVPGGG |
| tau 329-335 | SEQ ID NO: 13 | HHKPGGG |
| tau 330-335 | SEQ ID NO: 14 | HKPGGG |
| tau 361-367 | SEQ ID NO: 15 | THVPGGG |
| tau 362-367 | SEQ ID NO: 16 | HVPGGG |
| tau 314-342 | SEQ ID NO: 17 | DLSKVTSKCGSLGNIHHKPGGGQVE |
| | | VKSE |
| tau 352-380 | SEQ ID NO: 18 | SKIGSLDNITHVPGGGNKKIETHKL |
| | | TFREN |
| tau 357-368 | SEQ ID NO: 19 | LDNITHVPGGGN |

In some embodiments, the immunogenic peptide comprising or consisting of a fragment of tau discussed above may be further modified to insert one or more mutations in the sequence while retaining the original structure and/or at least one function of the unmutated protein, e.g., the ability to fold and present an epitope in the unmutated protein and/or the ability to form a core of a PHF.

In some embodiments, the immunogenic peptide comprising or consisting of a fragment of tau discussed above may be attached (e.g., covalently or noncovalently) to a moiety, e.g., a carrier that provides an enhanced immune response (e.g., as compared to the immunogenic peptide in the absence of the carrier). Exemplary carriers include serum albumin, keyhole limpet hemocyanin (KLH), an immunoglobulin molecule, thyroglobulin, ovalbumin, tetanus toxoid, or a toxoid from another pathogenic bacterium, such as diphtheria, E. coli, V. cholera, or H. pylori, or an attenuated toxin derivative, a cytokine such as IL-1, IL-1α or IL-1β peptide, IL-2, IFNγ, IL-10, GM-CSF, and a chemokine, such as MIP1α, MIP1β or RANTES. In some embodiments, the carrier comprises KLH.

Without being bound by theory, small peptides may be less effective immunogens because they can act as haptens that lack the necessary Th-cell epitopes and/or that are captured with low efficiency by antigen presenting cells (APC). Thus, a peptide immunogen can be linked to a suitable carrier to help elicit an immune response. In some embodiments, a larger peptide, e.g., a portion of tau of at least 10, at least 15, at least 20, at least 25, at least 30, at least 40, at least 50, at least 60, at least 70, at least 80, at least 90, at least 100 amino acids in length, may provide for an improved immune response by serving as better immunogens. In some embodiments, an immunogenic peptide further comprises a carrier peptide or protein. In some embodiments, an immunogenic peptide is a fragment of a pathologic tau protein that comprises or consists of CKDNIKHVPGGGS (SEQ ID NO: 2) or KDNIKHVPGGGS (SEQ ID NO: 1) coupled to a carrier, e.g., KLH or a fragment thereof. In some embodiments, the carrier is keyhole limpet hemocyanin (KLH) or a fragment thereof, e.g., a fragment that provides for an enhanced immune response. In some embodiments, an immunogenic peptide comprises or consists of a peptide having a sequence of CKDNIKHVPGGGS (SEQ ID NO: 2) or KDNIKHVPGGGS (SEQ ID NO: 1) coupled to KLH. In some embodiments, an immunogenic peptide is a fragment of a pathologic tau protein that comprises or consists of CKDNIKHVPGGGS (SEQ ID NO: 2) or KDNIKHVPGGGS (SEQ ID NO: 1) with up to 5, up to 4, up to 3, up to 2, or up to 1 mutation, coupled to a carrier, e.g., KLH or a fragment thereof. In some embodiments, an immunogenic peptide is a fragment of a pathologic tau protein that comprises or consists of CKDNIKHVPGGGS (SEQ ID NO: 2) or KDNIKHVPGGGS (SEQ ID NO: 1) and further comprises 1, 2, 3, 4, or 5 additional amino acids, e.g., inserted at the ends and/or internally to the sequence, and coupled to a carrier, e.g., KLH or a fragment thereof. In some embodiments, the immunogenic peptide consists of SEQ ID NO: 1 and 1, 2, 3, 4, or 5 additional residues at the N and/or C terminus, wherein the additional residues are identical to those immediately adjacent to SEQ ID NO: 1 when aligned against tau 2N4R, and wherein the immunogenic peptide is coupled to a carrier, e.g., KLH or a fragment thereof. In some embodiments, the immunogenic peptide consists of SEQ ID NO: 2 and 1, 2, 3, 4, or 5 additional residues at the N and/or C terminus, wherein the additional residues are identical to those immediately adjacent to SEQ ID NO: 2 when aligned against tau 2N4R, and wherein the immunogenic peptide is coupled to a carrier, e.g., KLH or a fragment thereof.

In some embodiments, an immunogenic peptide is a fragment of a pathologic tau protein that has at least 99%, 95%, 92%, 90%, 85%, 80%, 75%, 70%, 65%, 60%, 55%, or 50% sequence identity with CKDNIKHVPGGGS (SEQ ID NO: 2) or KDNIKHVPGGGS (SEQ ID NO: 1) and coupled to a carrier, e.g., KLH or a fragment thereof. In some embodiments, the immunogenic peptide is AADvac1.

In some embodiments, the carrier is linked to the immunogenic peptide covalently. In some embodiments, the carrier is linked to the immunogenic peptide noncovalently. In some embodiments, the immunogenic peptide can be linked at the amino terminus, the carboxyl terminus, or at a site anywhere within the peptide (internally) to the carrier. In some embodiments, the immunogenic peptide is linked at its N terminus to the carrier or 1, 2, 3, 4, or 5 amino acids from the terminus. In some embodiments, the immunogenic peptide is linked at its C terminus to the carrier or 1, 2, 3, 4, or 5 amino acids from the terminus. In some embodiments, the immunogenic peptide is linked at an internal amino acid to the carrier.

Immunogenic agents can be linked to carriers by chemical crosslinking. Techniques for linking an immunogen to a carrier include the formation of disulfide linkages using N-succinimidyl-3-(2-pyridyl-thio) propionate (SPDP) and succinimidyl 4-(N-maleimidomethyl)cyclohexane-1-carboxylate (SMCC) (e.g., if the peptide lacks a sulfhydryl group, this can be provided by addition of a cysteine residue). In some embodiments, these reagents create a disulfide linkage between themselves and peptide cysteine resides on one protein and an amide linkage through the ε-amino on a lysine, or other free amino group in other amino acids. A variety of such disulfide/amide forming agents are described in Immun. Rev. 62, 185 (1982). Other bifunctional coupling agents that may be used include, e.g., those that form a thioether rather than a disulfide linkage. Many thioether-forming agents are commercially available and include reactive esters of 6-maleimidocaproic acid, 2-bromoacetic acid, and 2-iodoacetic acid, 4-(N-maleimidomethyl)cyclohexane-1-carboxylic acid. In some embodiments, the carboxyl groups can be activated by combining them with succinimide or 1-hydroxyl-2-nitro-4-sulfonic acid, sodium salt. In some embodiments, the immunogenic peptide or a fragment thereof is linked to a carrier by chemical crosslinking. In some embodiments, the carrier is conjugated to the peptide via N-succinimidyl-3-(2-pyridyl-thio) propionate (SPDP) or succinimidyl 4-(N-maleimidomethyl)cyclohexane-1-carboxylate (SMCC). In some embodiments, an immunogenic peptide comprising or consisting of a peptide sequence of KDNIKHVPGGGS (SEQ ID NO: 1) or CKDNIKHVPGGGS (SEQ ID NO: 2) is linked to a carrier or a fragment thereof, by N-succinimidyl-3-(2-pyridyl-thio) propionate (SPDP) or succinimidyl 4-(N-maleimidomethyl)cyclohexane-1-carboxylate (SMCC). In some embodiments, an immunogenic peptide comprising or consisting of a peptide sequence of KDNIKHVPGGGS (SEQ ID NO: 1) or CKDNIKHVPGGGS (SEQ ID NO: 2) is linked to KLH or a fragment thereof, by N-succinimidyl-3-(2-pyridyl-thio) propionate (SPDP) or succinimidyl 4-(N-maleimidomethyl)cyclohexane-1-carboxylate (SMCC).

In some embodiments, the carrier is conjugated to the immunogenic peptide by a crosslinker comprising N-[γ-maleimidobutyryloxy]succinimide ester (GMBS). In some embodiments, the crosslinker comprises or is GMBS. In some embodiments, the carrier is conjugated to the immunogenic peptide by a crosslinker comprising a derivative of GMBS. In some embodiments, the crosslinker is N-γ-maleimidobutyryl-oxysulfosuccinimide ester (Sulfo-GMBS).

In some embodiments, an immunogenic peptide comprising or consisting of a peptide sequence of KDNIKHVPGGGS (SEQ ID NO: 1) or CKDNIKHVPGGGS (SEQ ID NO: 2) is linked to a carrier or a fragment thereof, by a crosslinker, e.g., one comprising GMBS or a derivative thereof (e.g., Sulfo-GMBS). In some embodiments, an immunogenic peptide comprising or consisting of a peptide sequence of KDNIKHVPGGGS (SEQ ID NO: 1) or CKDNIKHVPGGGS (SEQ ID NO: 2) is linked to KLH or a fragment thereof, by a crosslinker comprising GMBS or a derivative thereof (e.g., Sulfo-GMBS).

Immunogenic peptides may be directly linked to carriers or indirectly, e.g., through a flexible linker such as a spacer amino acid sequence, e.g., rigid linkers like (EAAAK)ₙ, or flexible linkers like GGGS and (GGGGS)₃.

In some embodiments, immunogenic peptides can be expressed as fusion proteins with carriers. The immunogenic peptide can be linked at the amino terminus, the carboxyl terminus, or at a site anywhere within the peptide (internally) to the carrier, directly or through a spacer amino acid sequence, e.g., rigid linkers like (EAAAK)ₙ, or flexible linkers like GGGS and (GGGGS)₃.

In some embodiments, multiple repeats of the immunogenic peptide can be present in a fusion protein. In some embodiments, the immunogenic peptide is linked on the N terminus to a carrier protein. In some embodiments, the immunogenic peptide is linked on the C terminus to a carrier protein. In some embodiments, the immunogenic peptide is linked on an internal amino acid residue to a carrier protein. In some embodiments, an immunogenic peptide comprising or consisting of a peptide sequence of KDNIKHVPGGGS (SEQ ID NO: 1) or CKDNIKHVPGGGS (SEQ ID NO: 2) is linked on its N terminus to KLH. In some embodiments, an immunogenic peptide comprising a peptide sequence of KDNIKHVPGGGS (SEQ ID NO: 1) or CKDNIKHVPGGGS (SEQ ID NO: 2) is linked on its C terminus to KLH. In some embodiments, an immunogenic peptide comprising a peptide sequence of KDNIKHVPGGGS (SEQ ID NO: 1) or CKDNIKHVPGGGS (SEQ ID NO: 2) is linked by an internal amino acid to KLH. Examples of additional immunogenic peptides, carriers, and how to prepare them, may be found in PCT/IB2012/002246, which is herein incorporated by reference in its entirety.

Pharmaceutical Compositions

The immunogenic peptides disclosed herein may be prepared and/or formulated in pharmaceutical compositions, e.g., those suitable for administration to a subject, e.g., a human patient suffering from, at risk for, or otherwise in need of treatment for AD or another tauopathy. A pharmaceutical composition may comprise a therapeutic agent (e.g., an immunogenic peptide, as described above) and one or more of other pharmaceutically acceptable components such as excipients (solvents, adjuvants, and the like) and/or additional therapeutic agents (e.g. other agents that target pathologic tau or other proteins associated with AD and/or other tauopathies). A wide variety of pharmaceutically acceptable excipients are known in the art. E.g., A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7.sup.th ed., Lippincott, Williams, & Wilkins; and Handbook of Pharmaceutical Excipients (2000) A. H. Kibbe et al., eds., 3 ed. Amer. Pharmaceutical Assoc.

In some embodiments, a pharmaceutical composition disclosed herein comprises a pharmaceutically acceptable excipient and/or diluent. For instance, any vehicle used to formulate pharmaceutical compositions for animal or human administration may be used. The diluent may be selected so as not to affect the biological activity of the combination. Examples of such diluents are distilled water, physiological phosphate-buffered saline, Ringer's solutions, dextrose solution, and Hank's solution. The pharmaceutical compositions and formulations disclosed herein can also include other excipients, adjuvants, or nontoxic, nontherapeutic, nonimmunogenic stabilizers and the like. Examples of suitable pharmaceutical excipients are known in the art and include phosphate buffered saline solutions, water, emulsions, such as oil/water emulsions, various types of wetting agents, sterile solutions etc. Compositions comprising such excipients can be formulated by known conventional methods.

In some embodiments, the immunogenic peptides disclosed herein can be provided in pharmaceutical compositions suitable for subcutaneous administration. In some embodiments, the immunogenic peptides disclosed herein can be provided in pharmaceutical compositions suitable for intramuscular administration.

In various embodiments, the pharmaceutical compositions may be provided in formulations comprising one or more inactive ingredient and/or one or more additional active ingredient in addition to the immunogenic peptides. In some embodiments, the compositions of the disclosure can be formulated in formulations suitable for administration in a mammalian subject, e.g., a human, using components and techniques known in the art.

In some embodiments, a pharmaceutical composition disclosed herein comprises an immunogenic peptide and an adjuvant. In some embodiments, the adjuvant comprises an aluminum compound. In some embodiments, the adjuvant comprises aluminum hydroxide. In some embodiments, the adjuvant comprises about 0.1-1.0 mg aluminum (Al³⁺), e.g., about 0.1 mg, about 0.2 mg, about 0.3 mg, about 0.4 mg, about 0.5 mg, about 0.6 mg, about 0.7 mg, about 0.8 mg, about 0.9 mg, about 1.0 mg. In some embodiments, a pharmaceutical composition comprises an immunogenic peptide of sequence CKDNIKHVPGGGS (SEQ ID NO: 2) coupled to keyhole limpet hemocyanin and aluminum hydroxide. In some embodiments, the pharmaceutical composition is formulated in phosphate buffer. In some embodiments, the pharmaceutical composition comprises about 10-190 μg of an immunogenic peptide of sequence CKDNIKHVPGGGS (SEQ ID NO: 2) coupled to keyhole limpet hemocyanin, e.g., about 10 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, or about 190 μg. In some embodiments, the pharmaceutical composition comprises about 40 μg of an immunogenic peptide of sequence CKDNIKHVPGGGS (SEQ ID NO: 2) coupled to keyhole limpet hemocyanin, and 0.5 mg Al³⁺ in a phosphate buffer. In some embodiments, the pharmaceutical composition comprises about 160 μg of an immunogenic peptide of sequence CKDNIKHVPGGGS (SEQ ID NO: 2) coupled to keyhole limpet hemocyanin, and 0.5 mg Al³⁺ in a phosphate buffer. Descriptions of exemplary peptide vaccine structures and examples of additional formulations and how to prepare them, may be found in PCT/IB2012/002246, which is herein incorporated by reference in its entirety.

An adjuvant can be administered with an immunogen as a single composition, or can be administered before, concurrent with, or after administration of the immunogen. The immunogen and adjuvant can be packaged and supplied in the same vial or can be packaged in separate vials and mixed before use. In some embodiments, immunogen and adjuvant are packaged with a label, indicating the intended therapeutic application. If immunogen and adjuvant are packaged separately, the packaging may include instructions for mixing before use.

Uses of Immunogenic Compositions Comprising an Immunogenic Peptide

In various embodiments, disclosed herein are methods for treating a patient in need thereof, comprising administering an immunogenic peptide or pharmaceutical composition comprising the immunogenic peptide. The immunogenic peptide disclosed herein may be administered by parenteral, topical, intradermal, intravenous, oral, subcutaneous, intraperitoneal, intranasal or intramuscular means for prophylactic and/or therapeutic treatment. In some embodiments, the delivery is by subcutaneous delivery. In some embodiments, the delivery is by intramuscular delivery.

In some embodiments, the immunogenic peptide is delivered by subcutaneous delivery to the upper arm. In some embodiments, the immunogenic peptide is delivered by subcutaneous delivery to the abdomen. In some embodiments, the immunogenic peptide is delivered by subcutaneous delivery to the thigh. In some embodiments, the immunogenic peptide is delivered by subcutaneous delivery to the upper back. In some embodiments the immunogenic peptide is delivered by subcutaneous delivery to the buttock.

In some embodiments, the immunogenic peptide is delivery by intramuscular delivery to the deltoid muscle of the arm. In some embodiments, the immunogenic peptide is delivered by intramuscular delivery to the vastus lateralis muscle of the thigh. In some embodiments, the immunogenic peptide is delivered by intramuscular delivery to the ventrogluteal muscle of the hip. In some embodiments, the immunogenic peptide is delivered by intramuscular delivery to the dorsogluteal muscles of the buttocks

In various embodiments, the immunogenic peptide is administered one or more times, e.g., with the same dose used for each administration, with one or more intervals between vaccinations. In some embodiments, the immunogenic peptide is administered 1, 2, 3, 4, 5, 6, 7, 8, 9, 10, 11, 12, 13, 14, 15, 16, 17, 18, 19, or 20 or more times, e.g., with the same dose used for each administration. In some embodiments, the immunogenic peptide is administered at 1-week, 2-week, 3-week, 4-week, 5-week, 6-week, 7-week, 8-week, 9-week, 10-week, 11-week, 12-week, 1-month, 2-month, 3-month, 4-month, 5-month, 6-month, 7-month, 8-month, 9-month, 10-month, 11-month, 1-year, 2-year, 3-year, or 4-year intervals. In some embodiments, the immunogenic peptide is administered at one interval, and then administered at a different interval, e.g., 4-week intervals for 5 doses, then 6-month intervals for 5 doses, or 3-week intervals for 6 doses, then 2-month intervals for 4 doses. In some embodiments, the immunogenic peptide is administered at 6-week intervals for 6 doses, then 3-month interval for 5 doses, e.g., doses at weeks 0, 6, 12, 18, 24, 30, 42, 54, 66, 78, and 90. In some embodiments, the immunogenic peptide is administered at 4-week intervals for 6 doses, then 3-month intervals for 5 doses, e.g., doses at weeks 0, 4, 8, 12, 16, 20, 32, 44, 56, 68, and 80. In some embodiments, the immunogenic peptide is administered at 4-week intervals for 6 doses, then 14-week intervals for 5 doses, e.g., doses at weeks 0, 4, 8, 12, 16, 20, 34, 48, 62, 76, and 90.

In various embodiments, the methods and materials disclosed herein are indicated for and can be used in the treatment of Alzheimer's Disease or a related tauopathy, e.g., by subcutaneous or intramuscular administration to a patient showing the symptoms of the disease. In various embodiments, the methods and materials disclosed herein are used in the treatment of Alzheimer's Disease, e.g., by subcutaneous or intramuscular administration to a patient diagnosed with Alzheimer's Disease, e.g., using one of the biomarkers disclosed herein and/or by brain imaging, but where the patient is not yet showing the clinical symptoms of the disease. In some embodiments, diagnosis of a patient is carried out in vitro.

In various embodiments, one or more immunogenic peptides, e.g., peptides comprising sequence SEQ ID NO: 1 or SEQ ID NO: 2 disclosed herein, can be administered subcutaneously or intramuscularly to treat Alzheimer's Disease. In some embodiments, a method of treating AD or another tauopathy is provided, comprising administering one or more doses comprising an effective amount of a composition comprising a peptide as disclosed herein to a patient in need thereof. In some embodiments, an effective dose is a dose that partially or fully alleviates (i.e., eliminates or reduces), at least one symptom associated with the disorder/disease state being treated, that slows, delays, or prevents onset or progression to a disorder/disease state, that slows, delays, or prevents progression of a disorder/disease state, that diminishes the extent of disease, that reverses one or more symptom, that results in remission (partial or total) of disease, and/or that prolongs survival. In some embodiments, an effective dose is a dose that reduces or ameliorates the effects of AD, e.g., as indicated by a detectable change in one or more biomarkers. In some embodiments, an effective dose is a dose that reduces the amount of tau pathologies in the brain of a patient. Examples of disease states contemplated for treatment are set out herein. In some embodiments, the patient has AD, or is at risk of developing AD.

Examples of the immunogenic compositions that can be used in the methods disclosed herein include the immunogenic peptides and compositions disclosed in PCT/IB2012/002246, which is herein incorporated by reference in its entirety. In some embodiments, the immunogenic composition comprises AADvac1, which comprises the immunogenic peptide CKDNIKHVPGGGS (SEQ ID NO: 2) (e.g., about 40 μg of the peptide) coupled to keyhole limpet hemocyanin (KLH) via a maleimide linker, along with an aluminium hydroxide adjuvant (e.g., containing 0.5 mg Al³⁺) in a phosphate buffer solution (e.g. in a volume of about 0.3 mL).

In some embodiments, an immunogenic peptide or pharmaceutical composition as disclosed herein, e.g., AADvac1, is administered, e.g., subcutaneously, to a patient in need of treatment for Alzheimer's Disease. In some embodiments, the patient is 40-95 years of age. In some embodiments, the patient is 45-90 years of age. In some embodiments, the patient is 50-85 years of age. In some embodiments, the patient is 50-67 years of age. In some embodiments, the patient is 68-85 years of age. In some embodiments, the patient is 50-70 years of age. In some embodiments, the patient is 71-85 years of age. In some embodiments, the subject is identified as having probable AD by the revised National Institute on Aging-Alzheimer's Disease Association (NIA-AA) criteria.

The pre-screening of patients amenable to treatment is also contemplated, e.g., according to the methods of identifying AD disclosed herein, as well as the administration of treatment to patients identified according to criteria disclosed herein.

Methods of Diagnosis—Criteria

Diagnosis of a patient with AD for treatment with an immunogenic peptide or pharmaceutical composition disclosed herein, and assessment of AD severity in the patient, may include one or more of neurological or clinical tests of reflexes, coordination, eye movement, speech, memory, problem solving, attention, counting, and/or language. Thresholds and/or criteria for determining a diagnosis of AD based on these tests are well known in the art. Examples of diagnosis criteria of AD include but are not limited to the 2011 revised NIA-AA criteria, the 2018 revised NIAA-AA criteria, the revised National Institute of Neurological and Communicative Disorders and Stroke and the Alzheimer's Disease and Related Disorders Association (NINCDS-ADRDA) criteria, the International Working Group (IWG) criteria, and the revised IWG-2 criteria. Dubois et al., Lancet Neurology, (2014) 13(6): p 614-629; McKhann et al., Neurol., (1984) 34(7): 939-944; Jack et al., Alzheimer's & Dementia, (2018) 14: 535-562.

In some embodiments, the subject is identified as having AD by the revised NINCDS-ADRDA criteria. In some embodiments, the subject is identified as having AD by the IWG criteria. In some embodiments, the subject is identified as having AD by the revised IWG-2 criteria.

In some embodiments, diagnosis of a patient is carried out in vitro.

For example, one criterion for neuropathologic diagnosis of AD is the revised National Institute on Aging/Reagan Institute of the Alzheimer Association (NIA-AA) criteria. McKhann et al., Alzheimer's & Dementia, (2011) 7:263-269; Hyman et al., Alzheimer's & Dementia, (2012) 8:1-13. The NIA-AA provides criteria for diagnosing a patient having dementia with (1) probable AD dementia, (2) possible AD dementia, and (3) probable or possible AD with evidence of AD pathophysiological process. In particular, dementia is diagnosed when patients have symptoms that:
1) Interfere with the ability to function at work or at usual activities;
2) Represent a decline from previous levels of functioning and performing;
3) Are not explained by delirium or major psychiatric disorder;
4) Cognitive impairment is detected and diagnosed through a combination of
   a. history-taking from the patient and a knowledgeable informant and
   b. an objective cognitive assessment, either a “bedside” mental status examination or neuropsychological testing;
5) The cognitive or behavioral impairment involves a minimum of two of the following domains:
   a. Impaired ability to acquire and remember new information—symptoms include: repetitive questions or conversations, misplacing personal belongings, forgetting events or appointments, getting lost on a familiar route;
   b. Impaired reasoning and handling of complex tasks, poor judgment—symptoms include: poor understanding of safety risks, inability to manage finances, poor decision-making ability, inability to plan complex or sequential activities;
   c. Impaired visuospatial abilities—symptoms include inability to recognize faces or common objects or to find objects in direct view despite good acuity, inability to operate simple implements, or orient clothing to the body;
   d. Impaired language functions, speaking, reading, writing—symptoms include: difficulty thinking of common words while speaking, hesitations; speech, spelling, and writing errors.
   e. Changes in personality, behavior, or comportment—symptoms include: uncharacteristic mood fluctuations such as agitation, impaired motivation, initiative, apathy, loss of drive, social withdrawal, decreased interest in previous activities, loss of empathy, compulsive or obsessive behaviors, socially unacceptable behaviors.

In some embodiments, a person with a diagnosis of probable AD dementia meets the above criteria and also has the following characteristics:
A. Insidious onset. Symptoms have a gradual onset over months to years, not sudden over hours or days;
B. Clear-cut history of worsening of cognition by report or observation; and
C. The initial and most prominent cognitive deficits are evident on history and examination in one of the following categories.
   a. Amnestic presentation: It is the most common syndromic presentation of AD dementia. The deficits should include impairment in learning and recall of recently learned information. There should also be evidence of cognitive dysfunction in at least one other cognitive domain, as defined earlier in the text.
   b. Nonamnestic presentations:
      Language presentation: The most prominent deficits are in word-finding, but deficits in other cognitive domains should be present.
      Visuospatial presentation: The most prominent deficits are in spatial cognition, including object agnosia, impaired face recognition, simultanagnosia, and alexia. Deficits in other cognitive domains should be present.
      Executive dysfunction: The most prominent deficits are impaired reasoning, judgment, and problem solving. Deficits in other cognitive domains should be present.
D. The diagnosis of probable AD dementia should not be applied when there is evidence of (a) substantial concomitant cerebrovascular disease, defined by a history of a stroke temporally related to the onset or worsening of cognitive impairment; or the presence of multiple or extensive infarcts or severe white matter hyperintensity burden; or (b) core features of Dementia with Lewy bodies other than dementia itself; or (c) prominent features of behavioral variant frontotemporal dementia; or (d) prominent features of semantic variant primary progressive aphasia or nonfluent/agrammatic variant primary progressive aphasia; or (e) evidence for another concurrent, active neurological disease, or a non-neurological medical comorbidity or use of medication that could have a substantial effect on cognition.

In some embodiments, a person with possible AD may be a patient who (1) meets the core clinical criteria in terms of the nature of the cognitive deficits for AD dementia, but either has a sudden onset of cognitive impairment or demonstrates insufficient historical detail or objective cognitive documentation of progressive decline, or (2) has an etiologically mixed presentation that meets all core clinical criteria for AD dementia but has evidence of concomitant cerebrovascular disease, features of dementia with Lewy bodies other than dementia itself, or evidence for another neurological disease or a non-neurological medical comorbidity or medication use that could have a substantial effect on cognition.

In some embodiments, a person with probable or possible AD with evidence of AD pathophysiological process has additionally, positive test results based on biomarkers. These biomarkers may include low AB₄₂ in the cerebrospinal fluid (CSF), positive positron emission tomography (PET) amyloid and tau imaging, elevated CSF tau (both total tau and phosphorylated tau), decreased fluorodeoxyglucose (FDG) uptake on PET in temporoparietal cortex, and/or disproportionate atrophy on structural magnetic resonance imaging in medial, basal and lateral temporal lobe, and medial parietal cortex. In some embodiments, biomarker tests in a patient are carried out in vitro. In some embodiments, total tau is measured using an ELISA assay. In some embodiments, the ELISA assay is an Innotest hTAU Ag and phospho-tau (181P) ELISA assay. In some embodiments, pT181 tau is measured using an ELISA assay. In some embodiments, the ELISA assay is an Innotest hTAU Ag and phospho-tau (181P) ELISA assay. In some embodiments, pT217 tau is measured using an ELISA assay. In some embodiments, the ELISA assay is a digital ELISA assay. In some embodiments, Aβ40 is measured using an ELISA assay. In some embodiments, Aβ42 is measured using an ELISA assay. In some embodiments, one or more biomarkers are measured by another suitable immunoassay, such as, but not limited to, the Elecsys® assay. Lifke et al., Elecsys® Total-Tau and Phospho-Tau (181P) CSF assays: Analytical performance of the novel, fully automated immunoassays for quantification of tau proteins in human cerebrospinal fluid. Clinical Biochemistry, (2019) 72:30-38. In some embodiments, one or more biomarkers are measured using mass spectrometry. E.g., Pottiez et al., A mass spectrometry-based method to quantify in parallel Tau and amyloid β 1-42 in CSF for the diagnostic of Alzheimer's Disease. J. Proteome Res., (2017) 16(3):1228-1238; Russell et al., Comprehensive Quantitative Profiling of Tau and Phosphorylated Tau Peptides in Cerebrospinal Fluid by Mass Spectrometry Provides New Biomarker Candidates. Journal of Alzheimer's Disease, (2017) 55(1):303-313.

In some embodiments, the patient has a diagnosis of probable Alzheimer's disease, e.g., as determined by the 2011 revised National Institute on Aging-Alzheimer's Disease Association (NIA-AA) criteria. In some embodiments, the patient has a diagnosis of possible Alzheimer's disease, e.g., as determined by the 2011 revised National Institute on Aging-Alzheimer's Disease Association (NIA-AA) criteria. In some embodiments, the patient has a diagnosis of probable or possible AD with evidence of AD pathophysiological process, e.g., as determined by the 2011 revised National Institute on Aging-Alzheimer's Disease Association (NIA-AA) criteria. In some embodiments, the diagnosis is made based on clinical evaluation of symptoms. In some embodiments, the diagnosis is made based on the 2011 revised National Institute on Aging/Reagan Institute of the Alzheimer Association (NIA-AA) criteria. In some embodiments, the diagnosis is made based on a probable AD determination based on the 2011 revised NIA-AA criteria. In some embodiments, one or more alternative diagnosis methods are used. In some embodiments, the diagnosis is confirmed using at least one additional method.

Another criteria for neuropathologic diagnosis of AD that may be used in the methods disclosed herein is the updated National Institute on Aging/Reagan Institute of the Alzheimer Association (NIA-AA) research framework. Jack et al., Alzheimer's & Dementia, (2018) 14: 535-562. The updated NIA-AA research framework bases diagnosis not on clinical consequences of the disease, but focuses instead on diagnosis of AD using biomarkers in living persons. Patients are placed on an Alzheimer's continuum based on three biomarker groups: (a) biomarkers of Aβ plaques such as cortical amyloid PET ligand binding or low CSF Aβ₄₂ (labeled “A”), (b) biomarkers of fibrillar tau such as elevated CSF phosphorylated tau (P-tau) and cortical tau PET ligand binding (labeled “T”), and (c) biomarkers of neurodegeneration or neuronal injury such as CSF T-tau, FDG PET hypometabolism, and atrophy on MRI (labeled “N”). This sets up an AT(N) system of biomarker characterization, where only “A” and “T” are considered as potential biomarker definitions of AD. For example, an individual with biomarker evidence of Aβdeposition alone with a normal pathologic tau biomarker (i.e. A+ and T−) would be assigned the label of “Alzheimer's pathologic change.” On the other hand, a patient with biomarker evidence of both Aβ and pathologic tau would be assigned a label of “Alzheimer's Disease.” Examples of biomarker profiles and categories are shown in Table 3.

TABLE 3

| AT(N) Profiles | Biomarker Category | |
|---|---|---|
| A-T-(N)- | Normal AD biomarkers | |
| A+T-(N)- | Alzheimer’s pathologic change | Alzheimer's |
| A+T+(N)- | Alzheimer's Disease | continuum |
| A+T+(N)+ | Alzheimer's Disease | |
| A+T-(N)+ | Alzheimer's and concomitant | |
| | suspected non-Alzheimer’s | |
| | pathologic change | |

| | |
|---|---|
| A-T+(N)- | Non-AD pathologic change |
| A-T-(N)+ | Non-AD pathologic change |
| A-T+(N)+ | Non-AD pathologic change |

In some embodiments, a patient is diagnosed with Alzheimer's disease without any detectable clinical symptoms. In some embodiments, the patient has a diagnosis of Alzheimer's disease based solely on biomarkers. In some embodiments, the patient has a diagnosis of Alzheimer's disease or Alzheimer's continuum, e.g., as determined by the 2018 revised NIA-AA research framework. In some embodiments, the patient has a diagnosis of Alzheimer's pathologic change or Alzheimer's continuum, e.g., as determined by the 2018 revised NIA-AA research framework. In some embodiments, the patient has a diagnosis of Alzheimer's and concomitant suspected non-Alzheimer's pathologic change, or Alzheimer's continuum, e.g., as determined by the 2018 revised NIA-AA research framework. In some embodiments, the 2018 revised research framework described herein can be used in combination with one or more of the assays or methods described here.

In some embodiments, one or more tests for cognitive impairment are used in the diagnosis or evaluation of patients. An exemplary test for cognitive impairment that may be used with the methods disclosed herein is the Mini-Mental State Examination (MMSE), a questionnaire commonly used to screen for dementia. Folstein et al., J. Psychiatr. Res., (1975)12:189-198. The MMSE has a maximum score of 30 points, and ordinarily can be administered in 5-10 minutes. The questions are typically grouped into seven categories, each representing a different cognitive domain or function:

(a) Orientation to time (5 points) e.g., “What is the year? Season? Date? Day of the week? Month?”;

(b) Orientation to place (5 points), e.g., “Where are we now: State? County? Town/city? Hospital? Floor?”;

(c) Registration of three words (3 points), e.g., examiner names three unrelated objects clearly and slowly, then asks the patient to name all three of them. The patient's response is used for scoring. The examiner repeats them until patient learns all of them, if possible;

(d) Attention and Calculation (5 points), e.g., “I would like you to count backward from 100 by sevens.” (93, 86, 79, 72, 65, . . . ) Stop after five answers. Alternative: “Spell WORLD backwards.” (D-L-R-O-W);

(e) Recall of three words (3 points), e.g., “Earlier I told you the names of three things. Can you tell me what those were?”;

(f) Language (8 points), e.g., ask the patient to name two simple objects shown to them, “Repeat the phrase: ‘No ifs, ands, or buts,” or “Make up and write a sentence about anything”; and

(g) Visual Construction (1 point) “Please copy this picture.” (The examiner gives the patient a blank piece of paper and asks him/her to draw a symbol shown to him/her.

The patient's score on the MMSE may be adjusted for his/her education attainment. For example, a patient whose education levels are 7^{th} grade or lower may have cognitive impairment with a score of 22 or below, but a patient who has some college or higher may have cognitive impairment with a score of 26 or below. In some embodiments, a patient has a total MMSE score of less than 18, 19, 20, 21, 22, 23, 24, 25, 26, 27, 28, 29, or 30. In some embodiments, a patient has a total MMSE score of at least 15, at least 16, at least 17, at least 18, at least 19, at least 20, at least 21, at least 22, at least 23, at least 24, at least 25, at least 26, at least 27, at least 28, or at least 29. In some embodiments, a patient has a total MMSE score of at most 29, at most 28, at most 27, at most 26, at most 25, at most 24, at most 23, or at most 22. In some embodiments, a patient has a total MMSE score of more than 19 and less than 27. In some embodiments, a patient has a total MMSE score of more than 20 and less than 26.

In some embodiments, diagnosis of a patient is carried out in vitro.

Methods of Diagnosis—Biomarkers

In some embodiments, biomarkers may be used to identify and/or confirm a subject has AD. Biomarkers may be agents whose concentration, presence, and/or activity are associated with a disease, and may also be used to provide a diagnosis of AD. Biomarkers may be found in any part of the body, e.g., plasma, urine, cerebrospinal fluid (CSF). In some embodiments, CSF provides a source for AD biomarkers. In some embodiments, blood plasma provides a source for AD biomarkers. Protein biomarkers for AD diagnosis include, but are not limited to Aβ40, Aβ42, total-tau (t-tau), and phosphorylated-tau (p-tau), including tau species identified in serum, plasma or CSF, while biomarkers of inflammation and oxidative stress and urine-based biomarkers may also provide vital information on development and progression of AD. Blennow et al., Lancet Neurology, (2003) 2(10):605-613; Formichi et al., Journal of Cellular Physiology, (2006) 208(1):39-46; Vandermeeren et al., Journal of Neurochemistry. (1993) 61(5):1828-1834; Morikawa et al., Alcoholism: Clinical and Experimental Research, (1999) 23(4):575-577; Hu et al., American Journal of Pathology, (2002) 160(4):1269-1278; Hampel et al., Archives of General Psychiatry, (2004) 61(1):95-102; Koopman et al., Neurochemistry International, (2009) 55(4):214-218.

In some embodiments, an AD diagnosis and/or a determination that a patient is suitable for treatment as disclosed herein is made at least in part based on measurements of protein biomarkers. In some embodiments, the biomarker is tau protein in plasma. In some embodiments, the biomarker is tau protein in the cerebrospinal fluid (CSF). In some embodiments, the biomarker is total tau (t-tau) proteins in the CSF. In some embodiments, a patient suffering from Alzheimer's disease and/or suitable for treatment has total tau protein in the CSF of >400 pg/mL. e.g., >500 pg/mL, >600 pg/mL, >700 pg/mL, >800 pg/mL, >900 pg/mL, or >1000 pg/mL. In some embodiments, the biomarker is tau protein phosphorylated at threonine 181 (pT181 tau) in the CSF. In some embodiments, a patient suffering from Alzheimer's disease and/or suitable for treatment has pT181 tau in the CSF of >60 pg/mL. e.g., >70 pg/mL, >80 pg/mL, >90 pg/mL, >100 pg/mL, >120 pg/mL, or >150 pg/mL. In some embodiments, the biomarker is Aβ40 peptides in the CSF. In some embodiments, the biomarker is Aβ42 in the CSF. In some embodiments, a patient suffering from Alzheimer's disease and/or suitable for treatment has Aβ42 levels in the CSF of <600 pg/mL. e.g., <550 pg/mL, <500 pg/mL, <450 pg/mL, <400 pg/mL, <350 pg/mL, or <300 pg/mL. In some embodiments, the biomarker is the ratio of Aβ42:Aβ40 in the CSF. In some embodiments, a patient suffering from Alzheimer's disease and/or suitable for treatment has Aβ42:Aβ40 ratios in the CSF of <0.089. e.g., <0.08, <0.075, <0.07, <0.065, <0.06, <0.055, <0.05, <0.045, <0.04, <0.035, or <0.03. In some embodiments, the biomarkers comprise one or a combination of more than one of total tau proteins, pathological tau proteins, Aβ42 peptides, Aβ40 peptides, and ratio of Aβ42:Aβ40 in the CSF. In some embodiments, a patient has total tau protein>400 pg/mL, pT181 tau protein>60 pg/mL, Aβ42<600 pg/mL, and/or Aβ42:Aβ40 ratio<0.089.

Non-coding RNA, for example microRNA or lncRNA, may also be used as biomarkers for AD. In some embodiments, an AD diagnosis is made at least in part based on measurements of microRNA. Wu et al., Journal of Alzheimer's Disease, (2016) 49(3):755-766. MicroRNA (also abbreviated as miRNA) is a small non-coding RNA molecule (containing about 22 nucleotides) found in plants, animals and some viruses. Without being bound by any theory, miRNA function in RNA silencing and post-transcriptional regulation of gene expression. MiRNAs function via base-pairing with complementary sequences within mRNA molecules. Levels of miRNA may be dysregulated in disease states, and as such may be used as biomarkers. Exemplary microRNAs that may serve as biomarkers for AD are shown in Table 4. In some embodiments, an AD diagnosis is made at least in part based on measurements of microRNA biomarkers. In some embodiments, the biomarker is hsa-let-7a-5p. In some embodiments, the biomarker is hsa-miR-10a-5p. In some embodiments, the biomarker is hsa-miR-145-5p. In some embodiments, the biomarker is hsa-miR-103a-3p. In some embodiments, the biomarker is hsa-miR-191-5p. In some embodiments, the biomarker is hsa-miR-374a-5p. In some embodiments, the biomarker is hsa-miR-26a-5p. In some embodiments, the biomarker is hsa-miR-107. In some embodiments, the biomarker is hsa-miR-15a-5p. In some embodiments, the biomarker is hsa-miR-126-3p. In some embodiments, the biomarker is hsa-miR-224-5p. In some embodiments, the biomarker is hsa-miR-18a-5p. In some embodiments, the biomarker is hsa-miR-23a-3p. In some embodiments, the biomarker is hsa-miR-26b-5p. In some embodiments, the biomarker is hsa-miR-21-5p. In some embodiments, combinations of more than one microRNA biomarker are used.

TABLE 4
Exemplary MicroRNA biomarkers for AD

| miRNA | SEQ ID NO: | MiRBase NO: | Sequence |
|---|---|---|---|
| hsa-let-7a-5p | SEQ ID NO: | MIMAT0000062 | UGAGGUAGUAGGUUGUAUAGUU |
| | 20 | | |
| hsa-miR-10a-5p | SEQ ID NO: | MIMAT0000253 | UACCCUGUAGAUCCGAAUUUGUG |
| | 21 | | |
| hsa-miR-145-5p | SEQ ID NO: | MIMAT0000437 | GUCCAGUUUUCCCAGGAAUCCCU |
| | 22 | | |
| hsa-miR-103a- | SEQ ID NO: | MIMAT0000101 | AGCAGCAUUGUACAGGGCUAUGA |
| 3p | 23 | | |
| hsa-miR-191-5p | SEQ ID NO: | MIMAT0000440 | CAACGGAAUCCCAAAAGCAGCUG |
| | 24 | | |
| hsa-miR-374a- | SEQ ID NO: | MIMAT0000727 | UUAUAAUACAACCUGAUAAGUG |
| 5p | 25 | | |
| hsa-miR-26a-5p | SEQ ID NO: | MIMAT0000082 | UUCAAGUAAUCCAGGAUAGGCU |
| | 26 | | |
| hsa-miR-107 | SEQ ID NO: | MIMAT0000104 | AGCAGCAUUGUACAGGGCUAUGA |
| | 27 | | |
| hsa-miR-15a-5p | SEQ ID NO: | MIMAT0000068 | UAGCAGCACAUAAUGGUUUGUG |
| | 28 | | |
| hsa-miR-126-3p | SEQ ID NO: | MIMAT0000445 | UCGUACCGUGAGUAAUAAUGCG |
| | 29 | | |
| hsa-miR-224-5p | SEQ ID NO: | MIMAT0000281 | CAAGUCACUAGUGGUUCCGUU |
| | 30 | | |
| hsa-miR-18a-5p | SEQ ID NO: | MIMAT0000072 | UAAGGUGCAUCUAGUGCAGAUAG |
| | 31 | | |
| hsa-miR-23a-3p | SEQ ID NO: | MIMAT0000078 | AUCACAUUGCCAGGGAUUUCC |
| | 32 | | |
| hsa-miR-26b-5p | SEQ ID NO: | MIMAT0000083 | UUCAAGUAAUUCAGGAUAGGU |
| | 33 | | |
| hsa-miR-21-5p | SEQ ID NO: | MIMAT0000076 | UAGCUUAUCAGACUGAUGUUGA |
| | 34 | | |

Methods for measuring microRNA levels are known in the art, and include but are not limited to reverse-transcriptase quantitative PCR (RT-qPCR), PCR, or high-throughput sequencing.

In some embodiments, the amount of microRNA in a patient's blood plasma or CSF is compared to that from a control sample or a threshold. A control sample can be a negative control sample or a positive control sample, and can be derived from a healthy individual or a patient with AD. Comparisons may also be made to disease progression, or disease state at different time points or treatment courses. In some embodiments, a control sample is a sample of blood plasma or CSF obtained from an individual that has not been diagnosed with Alzheimer's disease. In some embodiments, a control sample is a sample of blood plasma or CSF obtained from an individual with Alzheimer's disease. In some embodiments, a threshold is determined based on an individual who has not been diagnosed with Alzheimer's disease. In some embodiments, a threshold is determined based on an individual who has Alzheimer's disease.

In some embodiments, a method for diagnosing a patient suffering from AD comprises: (a) detecting the presence and/or amount of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p, in the patient or a sample from the patient; (b) wherein the presence of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p and/or an altered amount relative to an amount in a control sample or threshold indicates Alzheimer's Disease in the subject; and (c) diagnosing the presence or absence of Alzheimer's Disease in the patient based on step (b). In some embodiments, the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p in the patient is 1, 2, 3, 4, 5, 6, 7, 8, 9, 10, 15, 20, 25, 50, 100, or more fold different than in the control sample.

In some embodiments, the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p in the patient is 1, 2, 3, 4, 5, 6, 7, 8, 9, 10, 15, 20, 25, 50, 100, or more fold greater than in the control sample. In some embodiments, the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p in the patient is 1, 2, 3, 4, 5, 6, 7, 8, 9, 10, 15, 20, 25, 50, 100, or more fold less than in the control sample. In some embodiments, the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p in the patient is about 10%, 20%, 30%, 40%, 50%, 60%, 70%, 80%, 90%, 100%, 200%, or 500% greater than in the control sample. In some embodiments, the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p in the patient is 10%, 20%, 30%, 40%, 50%, 60%, 70%, 80%, 90%, or 100% less than in the control sample. In some embodiments, presence and/or altered amount of at least 2, at least 3, at least 4, at least 5, at least 6, or at least 7 or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p relative to the control sample or threshold indicates Alzheimer's Disease in the subject.

In some embodiments, a method for diagnosing a patient suffering from AD comprises obtaining a CSF or blood plasma sample from the patient. In some embodiments, the method further comprises extracting one or more of the hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p from the CSF or blood plasma of the patient. In some embodiments, the method further comprises performing cDNA synthesis from the miRNAs. In some embodiments, detection the presence or amount of the one or more of the hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p is performed by qPCR.

In some embodiments, a patient suffering from Alzheimer's disease has hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p levels at least 1, 2, 3, 4, 5, 6, 7, 8, 9, 10, 15, 20, 25, 50, 100, or more fold higher than in a control sample. In some embodiments, a patient suffering from Alzheimer's disease has hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p levels at least 1, 2, 3, 4, 5, 6, 7, 8, 9, 10, 15, 20, 25, 50, 100, or more fold lower than in the control sample. In some embodiments, a patient suffering from Alzheimer's disease has hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p levels 10%, 20%, 30%, 40%, 50%, 60%, 70%, 80%, 90%, 100%, 200%, or 500% greater than in the control sample. In some embodiments, a patient suffering from Alzheimer's disease has hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and/or hsa-miR-21-5p levels 10%, 20%, 30%, 40%, 50%, 60%, 70%, 80%, 90%, or 100% less than in the control sample.

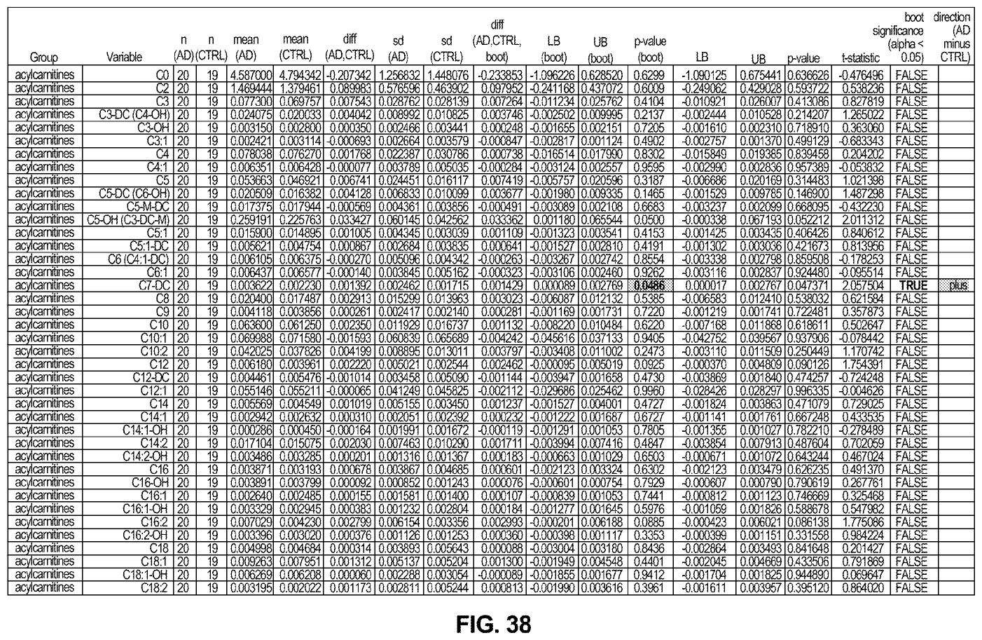

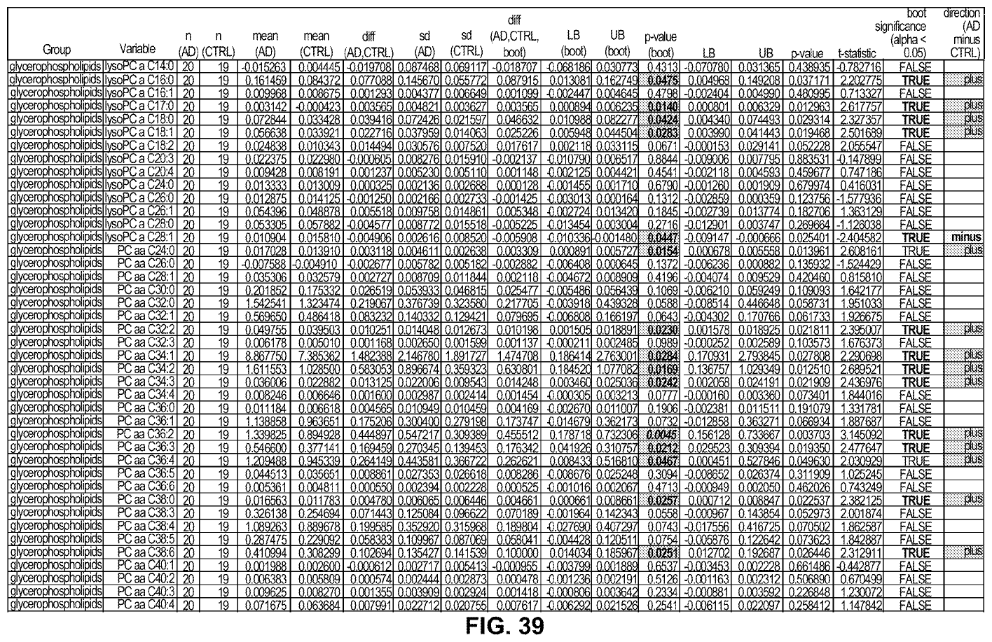

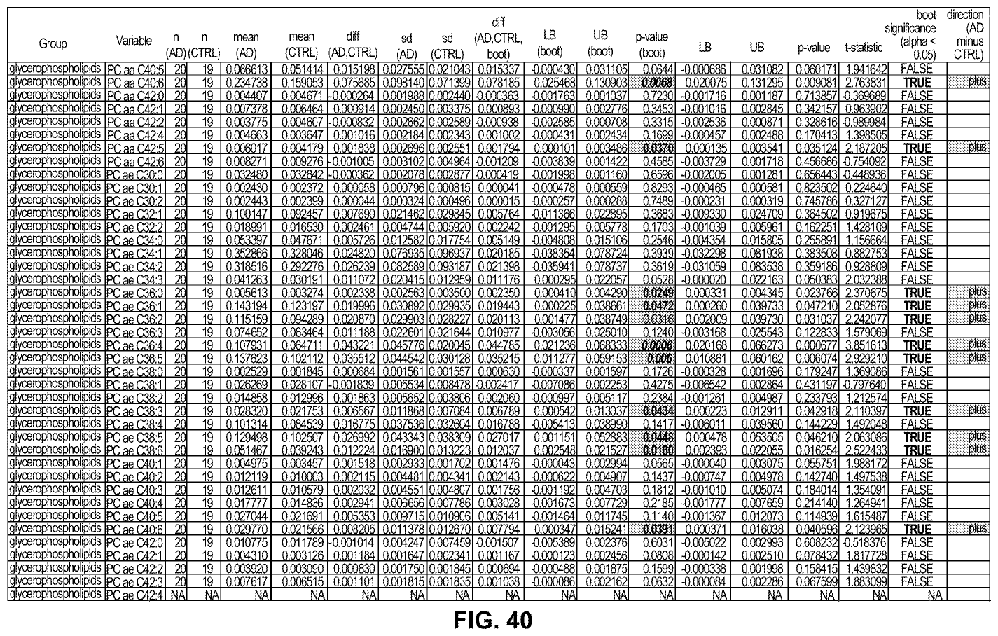

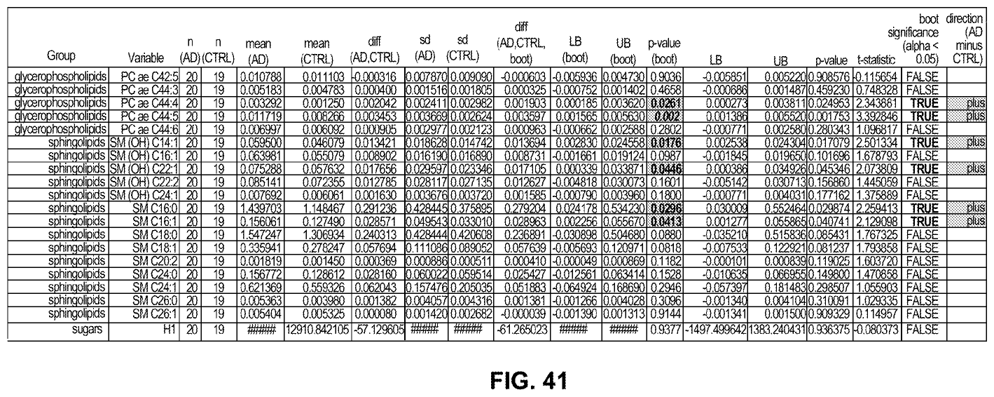

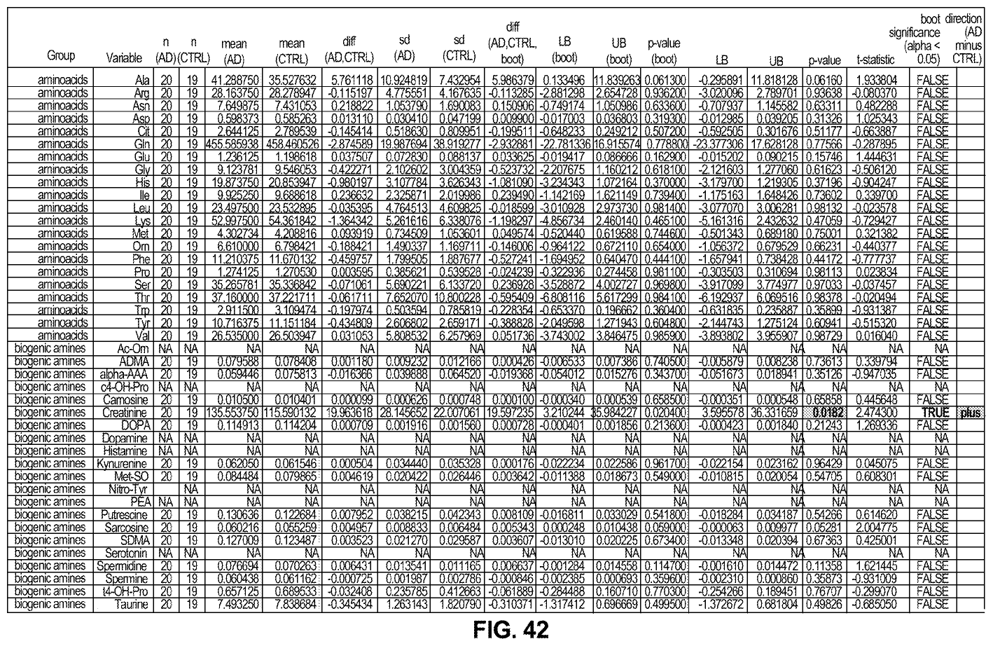

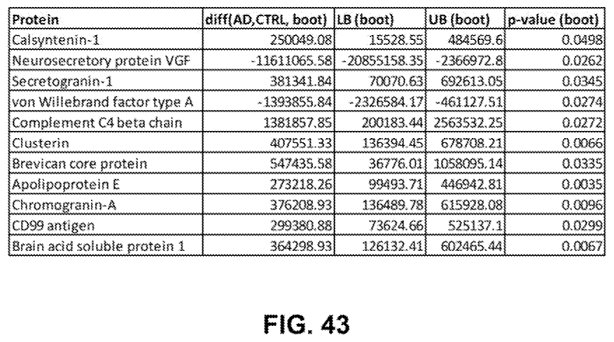

Metabolites, for example amino acids or phospholipids, may also be used as biomarkers to diagnose AD. Metabolites are the intermediate end products of metabolism. Examples of metabolites include but are not limited to alcohols, amino acids, nucleotides, antioxidants, organic acids, polyols, and vitamins. In some embodiments, an AD diagnosis is made at least in part based on measurements of one or more metabolites. Oresic et al., Translational Psychiatry, (2016) Translational Psychiatry, (2011) 1:e57. Exemplary metabolites that may serve as biomarkers for AD include, but are not limited to 2,4-dihydroxybutanoic acid, phospholipids, phosphatidylcholines, sphingomyelins, sterols, arginine, serotonin, spermidine, sphingomyelin, propionylcarnitine. In some embodiments, an AD diagnosis is made at least in part based on measurements of one or more metabolites. In some embodiments, metabolites may comprise one or more of the metabolites listed in FIGS. 38-42.

Methods for measuring metabolite levels are known in the art, and include but are not limited to mass spectrometry and enzyme-linked immunosorbent assays. In some embodiments, the amount of metabolite in a patient is measured by liquid chromatography coupled to mass spectrometry. In some embodiments, the amount of metabolite in a patient is measured by time-of-flight mass spectrometry. In some embodiments, the amount of metabolite in a patient is measured by tandem mass spectrometry. In some embodiments, the amount of metabolite in a patient's blood plasma or CSF is compared to that from a control sample or a threshold.

In some embodiments, the amount of metabolites in a patient's blood plasma or CSF is compared to that from a control sample or a threshold. A control sample can be a negative control sample or a positive control sample, and can be derived from a healthy individual or from a patient known to have Alzheimer's disease. Comparisons may also be made to disease progression, or disease state at different time points or treatment courses. In some embodiments, a control sample is a sample of blood plasma or CSF obtained from an individual that has not been diagnosed with Alzheimer's disease. In some embodiments, a control sample is a sample of blood plasma or CSF obtained from an individual with Alzheimer's disease. In some embodiments, a threshold is determined based on an individual who has not been diagnosed with Alzheimer's disease. In some embodiments, a threshold is determined based on an individual who has Alzheimer's disease.

In some embodiments, a method for diagnosing a patient suffering from AD comprises: (a) detecting the presence and/or amount of one or more metabolites in the patient or a sample from the patient; (b) wherein the presence of one or more metabolites and/or an altered amount relative to an amount in a control sample or threshold indicates Alzheimer's Disease in the subject; and (c) diagnosing the presence or absence of Alzheimer's Disease in the patient based on step (b). In some embodiments, the amount of metabolites in the patient is 1, 2, 3, 4, 5, 6, 7, 8, 9, 10, 15, 20, 25, 50, 100, or more fold different than in the control sample. In some embodiments, the amount of metabolites in the patient is 1, 2, 3, 4, 5, 6, 7, 8, 9, 10, 15, 20, 25, 50, 100, or more fold greater than in the control sample. In some embodiments, the amount of metabolites in the patient is 1, 2, 3, 4, 5, 6, 7, 8, 9, 10, 15, 20, 25, 50, 100, or more fold less than in the control sample. In some embodiments, presence and/or altered amount of at least 2, at least 3, at least 4, at least 5, at least 6, or at least 7 or more of metabolites relative to the control sample or threshold indicates Alzheimer's Disease in the subject.

In some embodiments, a method for diagnosing a patient suffering from AD comprises obtaining a CSF, serum, or blood plasma sample from the patient. In some embodiments, the method further comprises extracting one or more metabolites from the CSF, serum, or blood plasma of the patient. In some embodiments, detection the presence or amount of the one or more metabolites is performed by mass spectrometry.

In some embodiments, the amount of metabolites in the patient is 1, 2, 3, 4, 5, 6, 7, 8, 9, 10, 15, 20, 25, 50, 100, or more fold different than in the control sample. In some embodiments, the amount of metabolites in the patient is 10%, 20%, 30%, 40%, 50%, 60%, 70%, 80%, 90%, 100%, 150%, 200%, 250%, 500%, or 1000% greater than in the control sample. In some embodiments, the amount of metabolites in the patient is 10%, 20%, 30%, 40%, 50%, 60%, 70%, 80%, 90%, or 100% less than in the control sample. In some embodiments, the presence and/or an altered amount of at least 2, at least 3, at least 4, at least 5, at least 6, or at least 7 or more of metabolites relative to the control sample or threshold indicates Alzheimer's Disease in the subject.

In some embodiments, a patient suffering from Alzheimer's disease has metabolite levels at least 1, 2, 3, 4, 5, 6, 7, 8, 9, 10, 15, 20, 25, 50, 100, or more fold higher than in a control sample. In some embodiments, a patient suffering from Alzheimer's disease has metabolite levels at least 1, 2, 3, 4, 5, 6, 7, 8, 9, 10, 15, 20, 25, 50, 100, or more fold lower than in the control sample.

In some embodiments, diagnosis of a patient is carried out in vitro.

Methods of Selecting Patients—Biomarkers

Disclosed herein are biomarkers that may be used to select patients who are suitable for treatment as disclosed herein, as well as methods of treating the selected patients. In some embodiments, biomarkers may be used to differentiate between subpopulation of patients who will gain therapeutic benefit from the treatments disclosed herein. In some embodiments, biomarkers are used to select patients who will benefit from treatments with a therapeutic composition as discussed herein, e.g., one comprising an immunogenic peptide, e.g., one comprising SEQ ID NO: 1 or SEQ ID NO: 2, e.g., AADvac1. In some embodiments, biomarkers are used to select patients who will not benefit from treatments with an immunogenic composition such as AADvac1. In some embodiments, the presence, or altered levels as compared to a control or threshold, of one or more biomarkers in a patient or a sample from the patient indicates that a patient is likely to benefit from treatments with AADvac1. In some embodiments, the presence, or altered levels as compared to a control or threshold, of one or more biomarkers indicates that a patient is unlikely to benefit from treatments with AADvac1.

In some embodiments, biomarkers used to select patients for treatment with a therapeutic composition as discussed herein, e.g., one comprising an immunogenic peptide, e.g., one comprising SEQ ID NO: 1 or SEQ ID NO: 2, e.g., AADvac1, comprise one or more of hsa-let-7a-5p, hsa-miR-15a-5p, hsa-miR191-5p, and hsa-miR-23a-3p. In some embodiments, hsa-let-7a-5p is used to select patients for treatment with AADvac1. In some embodiments, hsa-miR-15a-5p is used to select patients for treatment with AADvac1. In some embodiments, hsa-miR191-5p is used to select patients for treatment with AADvac1. In some embodiments, hsa-miR-23a-3p is used to select patients for treatment with AADvac1. In some embodiments, the presence or altered level of one of more of hsa-let-7a-5p, hsa-miR-15a-5p, hsa-miR191-5p, and hsa-miR-23a-3p indicates that a patient is likely to benefit from treatment. In some embodiment, miRNA biomarker(s) are used alone or in combination with one or more additional biomarkers, e.g., one or more of the other biomarkers discussed herein.

In some embodiments, a method of selecting and/or treating a patient for Alzheimer's Disease comprises: (a) detecting the presence and/or amount of one or more of hsa-let-7a-5p, hsa-miR-15a-5p, hsa-miR191-5p, and hsa-miR-23a-3p in a patient or a sample from the patient (e.g., a CSF, plasma, or serum sample); (b) selecting the patient for treatment when the patient or a sample from the patient has one or more of hsa-let-7a-5p, hsa-miR-15a-5p, hsa-miR191-5p, and hsa-miR-23a-3p or an altered level of one or more of hsa-let-7a-5p, hsa-miR-15a-5p, hsa-miR191-5p, and hsa-miR-23a-3p relative to a control sample or threshold; and (c) treating the patient by administering one or more doses of an immunogenic composition comprising an immunogenic peptide comprising SEQ ID NO: 1 or SEQ ID NO: 2.

In some embodiments, the amount of hsa-let-7a-5p, hsa-miR-15a-5p, hsa-miR191-5p, and/or hsa-miR-23a-3p is detected and/or compared to a control sample or threshold. In some embodiments, the amount of hsa-let-7a-5p, hsa-miR-15a-5p, hsa-miR191-5p, and/or hsa-miR-23a-3p in a patient that is 1, 2, 3, 4, 5, 6, 7, 8, 9, 10, 15, 20, 25, 50, 100, or more fold different than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of hsa-let-7a-5p, hsa-miR-15a-5p, hsa-miR191-5p, and/or hsa-miR-23a-3p in a patient that is 1, 2, 3, 4, 5, 6, 7, 8, 9, 10, 15, 20, 25, 50, 100, or more fold less than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of hsa-let-7a-5p, hsa-miR-15a-5p, hsa-miR191-5p, and/or hsa-miR-23a-3p in a patient that is 1, 2, 3, 4, 5, 6, 7, 8, 9, 10, 15, 20, 25, 50, 100, or more fold greater than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of hsa-let-7a-5p, hsa-miR-15a-5p, hsa-miR191-5p, and/or hsa-miR-23a-3p in the patient that is 10%, 20%, 30%, 40%, 50%, 60%, 70%, 80%, 90%, 100%, 200%, or 500% greater than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of hsa-let-7a-5p, hsa-miR-15a-5p, hsa-miR191-5p, and/or hsa-miR-23a-3p in the patient that is 10%, 20%, 30%, 40%, 50%, 60%, 70%, 80%, 90%, or 100% less than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, the presence and/or altered amount of at least 2, at least 3, or at least 4 of hsa-let-7a-5p, hsa-miR-15a-5p, hsa-miR191-5p, and/or hsa-miR-23a-3p in the patient indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1.

In some embodiments, a method of selecting and/or treating a patient for Alzheimer's Disease comprises: (a) detecting the presence and/or amount of one or more metabolites in a patient or a sample from the patient (e.g., a plasma or serum sample); (b) selecting the patient for treatment when the patient or a sample from the patient has one or more metabolites or an altered level of one or more metabolites relative to a control sample or threshold; and (c) treating the patient by administering one or more doses of an immunogenic composition comprising an immunogenic peptide comprising SEQ ID NO: 1 or SEQ ID NO: 2. In some embodiments, metabolites may comprise one or more of the metabolites listed in FIGS. 38-42.

In some embodiments, the amount of one or more metabolites is detected and/or compared to a control sample or threshold. In some embodiments, the amount of metabolites in the patient that is 1, 2, 3, 4, 5, 6, 7, 8, 9, 10, 15, 20, 25, 50, 100, or more fold different than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, the amount of metabolites in the patient that is 1, 2, 3, 4, 5, 6, 7, 8, 9, 10, 15, 20, 25, 50, 100, or more fold less than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of metabolites in the patient that is 1, 2, 3, 4, 5, 6, 7, 8, 9, 10, 15, 20, 25, 50, 100, or more fold greater than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of metabolites in the patient that is 10%, 20%, 30%, 40%, 50%, 60%, 70%, 80%, 90%, 100%, 200%, or 500% greater than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of metabolites in the patient that is 10%, 20%, 30%, 40%, 50%, 60%, 70%, 80%, 90%, or 100% less than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, the presence and/or altered amount of at least 2, at least 3, or at least 4, at least 5, at least 6, at least 7, or more metabolites in the patient indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1.

Neurofilament light chain (NfL) is a biomarker for neuroaxonal damage that, without being bound by theory, may display increased levels in patients with mild cognitive impairment (MCI) and A D. Mattsson et al., JAMA Neurol., (2017) 74(5):557-566; Bergman et al., Neurol. Neuroimmunol. Neuroinflamm., (2016) 3(5):e271. The presence of NfL may also correlate with other markers of AD progression, such as future atrophy, hypometabolism, and cognitive decline. Mattsson et al., JAMA Neurol., (2019) 76(7):791-799. Without being bound by theory, NfL may serve as a biomarker to select a patient for treatment or to monitor or assess the efficacy of treatment as discussed herein.

In some embodiments, NfL is used as a biomarker to select patients for treatment with a therapeutic composition as discussed herein, e.g., one comprising an immunogenic peptide, e.g., one comprising SEQ ID NO: 1 or SEQ ID NO: 2, e.g., AADvac1. In some embodiments, the presence or altered level of NfL (alone or in combination with one or more additional biomarker) indicates that a patient is likely to benefit from treatment.

Methods for measuring neurofilament light chain levels are known in the art, and include but are not limited to Bradford assays, Western blots, immunoassays, e.g., ELISA and Simoa NF-light® assay, and mass spectrometry. In some embodiments, neurofilament light chain levels are measured by the Simoa NF-light® assay.

In some embodiments, a method of selecting a patient to treat for Alzheimer's Disease comprises: (a) detecting the presence and/or amount of NfL in a patient or a sample from the patient; (b) selecting the patient for treatment when the patient or a sample from the patient has NfL or an altered level of NfL relative to a control sample or threshold; and (c) treating the patient by administering one or more doses of an immunogenic composition comprising an immunogenic peptide comprising SEQ ID NO: 1 or SEQ ID NO: 2.

In some embodiments, the amount of NfL is detected and/or compared to a control sample or threshold. In some embodiments, the amount of neurofilament light chain in the patient that is 1, 2, 3, 4, 5, 6, 7, 8, 9, 10, 15, 20, 25, 50, 100, or more fold different than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of NfL in the patient that is more than a threshold of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 pg/ml, or 100 pg/mL indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, the method further comprises confirming AD in the patient by one or more methods disclosed herein, before selecting the patient for treatment in step (b).

In some embodiments, homocysteine is used to select patients for treatment with a therapeutic composition as discussed herein, e.g., one comprising an immunogenic peptide, e.g., one comprising SEQ ID NO: 1 or SEQ ID NO: 2, e.g., AADvac1. In some embodiments, the presence or altered level of homocysteine (alone or in combination with one or more additional biomarker) indicates that a patient is likely to benefit from treatment.

Methods for measuring homocysteine levels are known in the art, and include but are not limited to immunoassays and ELISA.

In some embodiments, a method of selecting a patient to treat for Alzheimer's Disease comprises: (a) detecting the presence and/or amount of homocysteine in a patient or a sample from the patient; (b) selecting the patient for treatment when the patient or a sample from the patient has homocysteine or an altered level of homocysteine relative to a control sample or threshold; and (c) treating the patient by administering one or more doses of an immunogenic composition comprising an immunogenic peptide comprising SEQ ID NO: 1 or SEQ ID NO: 2.

In some embodiments, the amount of homocysteine is detected and/or compared to a control sample or threshold. In some embodiments, the amount of homocysteine in the patient that is 1, 2, 3, 4, 5, 6, 7, 8, 9, 10, 15, 20, 25, 50, 100, or more fold different than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, the amount of homocysteine in the patient that is greater than a threshold of 1, 2, 3, 4, 5, 6, 7, 8, 9, 10, 15, 20, 25, 50, or 100 pM indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, the amount of homocysteine in the patient that is greater than a threshold of 15 μM indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1.

Neurogranin is a calmodulin-binding protein expressed primarily in the brain, particularly in dendritic spines, and participates in the protein kinase C signaling pathway. Neurogranin is the main postsynaptic protein regulating the availability of calmodulin, binding to it in the absence of calcium. Neurogranin concentration in CSF has been suggested as marker for synaptic dysfunction in age-related neurodegeneration and A D. Casaletto et al., (2017) Neurology, 89(17): 1782-1788; De Vos et al., (2015) Alzheimer's & Dementia, 11(12): 1461-1469; Willemse et al., (2018) Clinical Chemistry, 64(6): 927-937. Without being bound by theory, neurogranin may serve as a biomarker to select a patient for treatment or to monitor treatment as discussed herein.

In some embodiments, a method of selecting a patient to treat for Alzheimer's Disease comprises: (a) detecting the presence and/or amount of neurogranin in a patient or a sample from the patient; (b) selecting the patient for treatment when the patient or a sample from the patient has neurogranin or an altered level of neurogranin relative to a control sample or threshold; and (c) treating the patient by administering one or more doses of an immunogenic composition comprising an immunogenic peptide comprising SEQ ID NO: 1 or SEQ ID NO: 2.

In some embodiments, the amount of neurogranin is detected and/or compared to a control sample or threshold. In some embodiments, the amount of neurogranin in the patient that is 1, 2, 3, 4, 5, 6, 7, 8, 9, 10, 15, 20, 25, 50, 100, or more fold different than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, the amount of neurogranin in the patient that is greater than a threshold of 1, 2, 3, 4, 5, 6, 7, 8, 9, 10, 15, 20, 25, 50, or 100 pg/ml indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of neurogranin in the patient that is 10%, 20%, 30%, 40%, 50%, 60%, 70%, 80%, 90%, 100%, 200%, or 500% greater than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an amount of neurogranin in the patient that is 10%, 20%, 30%, 40%, 50%, 60%, 70%, 80%, 90%, or 100% less than in the control sample indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1.

In some embodiments, the selected patient has AD. In some embodiments, the selected patient is diagnosed as having AD using a method as disclosed herein.

In some embodiments, the selected patient has MCI. In some embodiments, the selected patient is diagnosed as having MCI using a method as disclosed herein.

Methods of Selecting Patients—Genotype

A patient's genotype may provide additional factors in deciding treatment options. In some embodiments, a patient's genotype is used to select patients for treatment with a therapeutic composition as discussed herein, e.g., one comprising an immunogenic peptide, e.g., one comprising SEQ ID NO: 1 or SEQ ID NO: 2, e.g., AADvac1, is homocysteine. In some embodiments, the presence of at least one ε4 allele indicates that a patient is likely to benefit from treatment.

In some embodiments, a method of selecting a patient to treat for Alzheimer's Disease comprises: (a) determining the genotype for each of the two alleles of the ApoE gene present in the patient; (b) selecting the patient for treatment when the patient has at least one ApoE-ε4 allele; and (c) treating the patient by administering one or more doses of an immunogenic composition comprising an immunogenic peptide comprising SEQ ID NO: 1 or SEQ ID NO: 2.

Methods for determining the genotype or allele are known in the art, and include but are not limited to reverse-transcriptase quantitative PCR (RT-qPCR), PCR, high-throughput sequencing, Sanger sequencing, or fluorescence in-situ hybridization (FISH). In some embodiments, the genotype is determined by Sanger sequencing. In some embodiments, the genotype is determined by whole genome sequencing. In some embodiments, the genotype is determined by PCR. In some embodiments, the presence of one ApoE-ε4 allele indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, the presence of two ApoE-ε4 allele indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1.

In some embodiments, the selected patient has AD. In some embodiments, the selected patient is diagnosed as having AD using a method as disclosed herein.

In some embodiments, the selected patient has MCI. In some embodiments, the selected patient is diagnosed as having MCI using a method as disclosed herein.

Methods of Selecting Patients—Patient Characteristics

The selection of patients amenable for, or likely to benefit from treatment with the compositions disclosed herein, may also be determined by patient characteristics. Such characteristics may include, but are not limited to age, gender, severity of disease, or age of disease onset. In some embodiments, severity of disease is used to select patients for treatment with a therapeutic composition as discussed herein, e.g., one comprising an immunogenic peptide, e.g., one comprising SEQ ID NO: 1 or SEQ ID NO: 2, e.g., AADvac1. In some embodiments, patient MMSE score is used to select patients for treatment with a therapeutic composition as discussed herein, e.g., one comprising an immunogenic peptide, e.g., one comprising SEQ ID NO: 1 or SEQ ID NO: 2, e.g., AADvac1.

In some embodiments, a method of selecting a patient to treat for Alzheimer's Disease comprises: (a) determining a Mini-Mental State Examination (MMSE) score for the patient; (b) comparing the score from step a to a threshold score; (c) selecting the patient for treatment wherein the patient has a MMSE score above the threshold; and (d) treating the patient by administering one or more doses of an immunogenic composition comprising a peptide comprising SEQ ID NO: 1 or SEQ ID NO: 2.

In some embodiments, the MMSE score is determined and compared to a control patient or threshold. In some embodiments, an MMSE score that is 1, 2, 3, 4, 5, 6, 7, 8, 9, or 10 points lower than the control or threshold indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an MMSE score that is 1, 2, 3, 4, 5, 6, 7, 8, 9, or 10 points higher than the control or threshold indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an MMSE score that is at least 15, 16, 17, 18, 19, 20, 21, 22, 23, 24, 25, 26, 27, 28, or 29 indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an MMSE score that is at most 15, 16, 17, 18, 19, 20, 21, 22, 23, 24, 25, 26, 27, 28, or 29 indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1. In some embodiments, an MMSE score that is 24-26 indicates that the patient is likely to benefit from treatment with a therapeutic composition as discussed herein, e.g., one comprising an immunogenic peptide, e.g., one comprising SEQ ID NO: 1 or SEQ ID NO: 2, e.g., AADvac1.

The age of a patient may also be used in the selection of patients amenable for, or likely to benefit from treatment with the compositions disclosed herein, e.g., AADvac1. Without being bound by theory, treatment with the compositions disclosed herein, e.g., AADvac1, which target pathological tau, may be more beneficial to patients with early onset AD, e.g., in younger patients, when tau may play a larger role in the pathology of the disease. In some embodiments, patients selected for treatment has early-onset Alzheimer's disease. In some embodiments, patients selected for treatment are less than 80 years, 78 years, 76 years, 74 years, 72 years, 70 years, 68 years, 66 years, 64 years 62 years, 60 years, 58 years, 56 years, 54 years, 54 years or 50 years of age. In some embodiments, patients selected for treated are 50-68 years of age. In some embodiments, patients selected for treated are 50-70 years of age. In some embodiments, patients selected for treated are no more than about 60 years, 62 years, 64 years, 66 years, 68 years, 70 years, 72 years, 74 years or 76 years of age.

In some embodiments, the selected patient has AD. In some embodiments, the selected patient is diagnosed as having AD using a method as disclosed herein.

In some embodiments, the selected patient has MCI. In some embodiments, the selected patient is diagnosed as having MCI using a method as disclosed herein.

Methods of Diagnosis—Brain Imaging

In some embodiments, an AD diagnosis and/or a determination that a patient is suitable for treatment as disclosed herein is made at least in part based on brain imaging techniques. Brain imaging techniques may provide direct structural and functional details of the brain. Techniques like computerized tomography (CT), magnetic resonance imaging (MRI) techniques, and positron emission tomography (PET) may be able to visualize loss of brain volume, neuronal loss, atrophy of medial temporal regions, and/or presence of neurofibrillary tangles in the brains of AD patients. Frisoni G B, J. Neurol. Neurosurgery and Psychiatry, (2001) 70(6):711-718; Scheltens et al., Lancet Neurology, (2002) 1(1):13-21; Barnes et al., Archives of Neurology, (2006) 63(10):1434-1439; Barnes et al., Neurobiology of Aging, (2007) 28(1):20-28; Whitwell et al., Topics in Magnetic Resonance Imaging, (2005) 16(6):409-425; Becker et al., Neurology, (1996) 46(3):692-700; Grady et al., Brain, (2001) 124(4):739-756; Woodard et al., Neuropsychology, (1998) 12(4):491-504.

In some embodiments, the AD diagnosis and/or a determination that a patient is suitable for treatment is made based on brain imaging tests. In some embodiments, the diagnosis and/or determination is made based on magnetic resonance imaging (MRI) scans on the brain. In some embodiments, the diagnosis and/or determination is made based on computerized tomography (CT) scans on the brain. In some embodiments, the diagnosis and/or determination is made based on positron emission tomography (PET) scans on the brain. In some embodiments, a patient has a brain MRI finding consistent with the diagnosis of AD at screening. In some embodiments, a patient has evidence of AD pathophysiological process.

In some embodiments, a radiological classification scale is used to evaluate the brain imaging. For instance, one radiological classification scale based on imaging that may be used in the methods disclosed herein is the Medial Temporal Lobe Atrophy scale (MTA-scale) or Scheltens scale. Scheltens et al., J. Neurol., (1995) 242:557-560; Scheltens et al., J. Neurol. Neurosurg. Psychiatry, (1992) 55:967-972; Dubois et al., Lancet Neurol., (2007) 6:734-746. The analysis is performed on coronal T1-weighted images, e.g., on a slice through the corpus of the hippocampus (level of the anterior pons). The scale may be based on a visual score of the width of the choroid fissure, the width of the temporal horn, and the height of the hippocampal formation. A score of 0 indicates no atrophy; 1 indicates only widening of choroid fissure; 2 includes widening of temporal horn of lateral ventricle; 3 indicates moderate loss of hippocampal volume (a decrease in height); and a score of 4 indicates a severe volume loss of hippocampus. In some embodiments, a patient has a Scheltens score of greater or equal to 2 based on a brain MRI, e.g., a Scheltens score of 2, 3, or 4.

In some embodiments, the AD diagnosis and/or a determination that a patient is suitable for treatment is made based on a clinical scale score. For instance, the Hachinski Ischemia Score (HIS) scale is a clinical tool currently used for differentiating types of dementia (primary degenerative, vascular or multi-infarct, mixed type). Hachinski et al., Arch. Neurol., (1975) 32:632-637. It is a 13-item test where patients with a higher score are more likely to have vascular dementia, and patients with a lower score are more likely to have senile dementia of the Alzheimer's type. The 13 items assess for, e.g., abrupt onset, stepwise deterioration, fluctuating course, nocturnal confusion, relative preservation of personality, depression, somatic symptoms, emotional incontinence, history of hypertension, history of strokes, evidence of associated atherosclerosis, focal neurological symptoms, and focal neurological signs. In some embodiments, a patient treated according to the methods herein has a HIS score of less than or equal to 4, e.g., a score of 4, 3, 2, or 1.

Brain imaging techniques may also assist in predicting, diagnosing, and/or monitoring AD disease progression. In some embodiments, brain imaging is used to monitor AD disease progression. In some embodiments, brain imaging is used to monitor AD disease severity throughout treatment. In some embodiments, AD disease progression is monitored by magnetic resonance imaging (MRI) scans on the brain. In some embodiments, AD disease progression is monitored by computerized tomography (CT) scans on the brain. In some embodiments, AD disease progression is monitored by positron emission tomography (PET) scans on the brain. In some embodiments, AD disease progression throughout treatment is monitored by magnetic resonance imaging (MRI) scans on the brain. In some embodiments, AD disease progression throughout treatment is monitored by computerized tomography (CT) scans on the brain. In some embodiments, AD disease progression throughout treatment is monitored by positron emission tomography (PET) scans on the brain. Any of the other techniques for diagnosis may also be used for monitoring the progression of the disease, e.g., biomarkers.

Methods of Monitoring Disease Progression and Treatment Efficacy—Biomarkers

In some embodiments, biomarkers may be used to monitor the progression of Alzheimer's Disease, and assess the efficacy of treatment as disclosed herein. Biomarkers specific for the pathologies of AD may provide a way of assessing the severity of the disease, and/or distinguish AD from other neurological diseases with similar symptoms.

In some embodiments, the progression of AD is monitored by measuring levels of one or more of hsa-let-7a-5p, hsa-miR-15a-5p, hsa-miR191-5p, and hsa-miR-23a-3p, e.g., in plasma and/or CSF. In some embodiments, efficacy of treatment is assessed by evaluating the presence and/or level of one or more of hsa-let-7a-5p, hsa-miR-15a-5p, hsa-miR191-5p, and hsa-miR-23a-3p, e.g., in plasma, serum, and/or CSF.

In some embodiments, presence and/or amount of one or more of hsa-let-7a-5p, hsa-miR-15a-5p, hsa-miR191-5p, and hsa-miR-23a-3p may be used to monitor efficacy of treatment. In some embodiments, a method of treating a patient suffering from Alzheimer's Disease or mild cognitive impairment comprises: (a) determining the presence and/or amount of one or more of hsa-let-7a-5p, hsa-miR-15a-5p, hsa-miR191-5p, and hsa-miR-23a-3p in the patient (e.g., by measuring level(s) in a CSF, serum, or plasma sample); (b) administering one or more doses of an immunogenic composition comprising a peptide comprising SEQ ID NO: 1 of SEQ ID NO: 2; (c) after administering the one or more doses, determining the presence and/or amount of one or more of hsa-let-7a-5p, hsa-miR-15a-5p, hsa-miR191-5p, and hsa-miR-23a-3p in the patient (e.g., by measuring level(s) in a CSF, serum, or plasma sample); (d) comparing the presence and/or amount from step (c) after treatment to the presence and/or amount from step a before treatment, wherein an altered amount of the one or more of hsa-let-7a-5p, hsa-miR-15a-5p, hsa-miR191-5p, and hsa-miR-23a-3p indicates treatment efficacy; and (e) administering one or more additional doses of the immunogenic composition comprising a peptide comprising SEQ ID NO: 1 or SEQ ID NO: 2 when the patient demonstrates treatment efficacy in step (d). In some embodiments, decreased amounts of hsa-let-7a-5p and/or hsa-miR-15a-5p after treatment indicates efficacy. In some embodiments, about 1, 2, 3, 4, 5, 6, 7, 8, 9, 10, 15, 20, 25, 50, 100, or more fold reduction of hsa-let-7a-5p and/or hsa-miR-15a-5p after treatment indicates efficacy. In some embodiments, about 1, 2, 3, 4, 5, 6, 7, 8, 9, 10, 15, 20, 25, 50, 100, or more fold increase of hsa-miR191-5p and/or hsa-miR-23a-3p after treatment indicates efficacy.

In some embodiments, the patient has been treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of AADvac1 before the presence and/or amount of one or more of hsa-let-7a-5p, hsa-miR-15a-5p, hsa-miR191-5p, and hsa-miR-23a-3p is determined. In some embodiments, at least 2, 3, 4, 5, 6, 7, 8, 9, 10, 11, 12, 13, 14, or 15 additional doses of AADvac1 is administered after the presence and/or amount of one or more of hsa-let-7a-5p, hsa-miR-15a-5p, hsa-miR191-5p, and hsa-miR-23a-3p is determined.

In some embodiments, the progression of AD is monitored by measuring levels of one or more metabolites, e.g., in plasma, serum, and/or CSF. In some embodiments, efficacy of treatment is assessed by evaluating the presence and/or level of one or more metabolites, e.g., in plasma, serum, and/or CSF. In some embodiments, metabolites may comprise one or more of the metabolites listed in FIGS. 38-42.

In some embodiments, presence and/or amount of one or more metabolites may be used to monitor efficacy of treatment. In some embodiments, a method of treating a patient suffering from Alzheimer's Disease or mild cognitive impairment comprises: (a) determining the presence and/or amount of one or more metabolites in the patient (e.g., by measuring level(s) in a CSF, serum, or plasma sample); (b) administering one or more doses of an immunogenic composition comprising a peptide comprising SEQ ID NO: 1 of SEQ ID NO: 2; (c) after administering the one or more doses, determining the presence and/or amount of one or more metabolites in the patient (e.g., by measuring level(s) in a CSF, serum, or plasma sample); (d) comparing the presence and/or amount from step (c) after treatment to the presence and/or amount from step a before treatment, wherein an altered amount of the one or more metabolites indicates treatment efficacy; and (e) administering one or more additional doses of the immunogenic composition comprising a peptide comprising SEQ ID NO: 1 or SEQ ID NO: 2 when the patient demonstrates treatment efficacy in step (d). In some embodiments, decreased amounts of one or more metabolites after treatment indicates efficacy. In some embodiments, about 1, 2, 3, 4, 5, 6, 7, 8, 9, 10, 15, 20, 25, 50, 100, or more fold reduction of metabolites after treatment indicates efficacy. In some embodiments, about 1, 2, 3, 4, 5, 6, 7, 8, 9, 10, 15, 20, 25, 50, 100, or more fold increase of one or more metabolites after treatment indicates efficacy.

In some embodiments, the patient has been treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of AADvac1 before the presence and/or amount of one or more metabolites is determined. In some embodiments, at least 2, 3, 4, 5, 6, 7, 8, 9, 10, 11, 12, 13, 14, or 15 additional doses of AADvac1 is administered after the presence and/or amount of one or more metabolites is determined.

A longitudinal increase in NfL correlates with high pT181 and total tau (tTau) levels, and low Aβ levels in CSF, low hippocampal volumes, low FDG-PET, and poor cognitive performance. A faster increase of NfL correlates with faster rates of atrophy, hypometabolism, and cognitive worsening. In some embodiments, the progression of AD is monitored by measuring levels of NfL in plasma and/or CSF. In some embodiments, efficacy of treatment is assessed by measuring levels of NfL in plasma and/or CSF.

In some embodiments, a method of treating a patient suffering from Alzheimer's Disease or mild cognitive impairment comprises: (a) determining the presence and/or amount of NfL chain in the patient (e.g., in a serum, plasma, or CSF sample); (b) administering one or more doses of an immunogenic peptide comprising SEQ ID NO: 1 or SEQ ID NO: 2; (c) after administering the one or more doses, determining the presence and/or amount of NfL in the patient (e.g., in a serum, plasma, or CSF sample); (d) comparing the presence and/or amount from step (c) after treatment to the presence and/or amount from step a before treatment, wherein an altered amount of NfL indicates treatment efficacy; and (e) administering one or more additional doses of the immunogenic composition comprising a peptide comprising SEQ ID NO: 1 or SEQ ID NO: 2 when the patient demonstrates treatment efficacy in step (d). In some embodiments, a stable or decreased amount of NfL after treatment indicates efficacy. In some embodiments, efficacy is indicated by reduced accumulation (e.g., a lower annual or biannual rate of accumulation) of NfL after treatment as compared to the accumulation (e.g., the rate of accumulation) prior to treatment. In some embodiments, efficacy is indicated by reduced accumulation (e.g., a lower annual or biannual rate of accumulation) of NfL after treatment as compared to the accumulation (e.g., the rate of accumulation) in an average population of Alzheimer's Disease patients. In some embodiments, an amount of NfL that does not increase by more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after treatment indicates efficacy. In some embodiments, an amount of NfL that does not increase by more than about 6 pg/mL, 5 pg/mL 4 pg/mL, 3 pg/mL or 2 pg/mL after treatment indicates efficacy. In some embodiments, an amount of NfL that decreases by more than about 30%, 25%, 20%, 15%, 14%, 13%, 12%, 11%, 10%, 9%, 8%, 7%, 6%, 5%, 4%, 3%, 2%, or 1% after treatment indicates efficacy.

In some embodiments, the patient has been treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of AADvac1 before the presence and/or amount of NfL is determined. In some embodiments, at least 2, 3, 4, 5, 6, 7, 8, 9, 10, 11, 12, 13, 14, or 15 additional doses of AADvac1 is administered after the presence and/or amount of NfL is determined.

In some embodiments, a method of treating a patient suffering from Alzheimer's Disease or mild cognitive impairment comprises: (a) determining the presence and/or amount of neurogranin in the patient (e.g., in a serum, plasma, or CSF sample); (b) administering one or more doses of an immunogenic peptide comprising SEQ ID NO: 1 or SEQ ID NO: 2; (c) after administering the one or more doses, determining the presence and/or amount of neurogranin in the patient (e.g., in a serum, plasma, or CSF sample); (d) comparing the presence and/or amount from step (c) after treatment to the presence and/or amount from step a before treatment, wherein an altered amount of neurogranin indicates treatment efficacy; and (e) administering one or more additional doses of the immunogenic composition comprising a peptide comprising SEQ ID NO: 1 or SEQ ID NO: 2 when the patient demonstrates treatment efficacy in step (d). In some embodiments, a stable or decreased amount of neurogranin after treatment indicates efficacy. In some embodiments, an amount of neurogranin that does not increase by more than about 25%, 24%, 23%, 22%, 21%, 20%, 19%, 18%, 17%, 16%, 15%, 14%, 13%, 12% 11%, 10%, 9%, 8%, 7%, 6%, 5%, 4%, 3%, 2%, or 1% after treatment indicates efficacy. In some embodiments, an amount of neurogranin that does not increase by more than about 20 pg/mL, 18 pg/mL, 16 pg/mL, 14 pg/mL, 12 pg/mL, 10 pg/mL, 8 pg/mL, 6 pg/mL, 5 pg/mL 4 pg/mL, 3 pg/mL, or 2 pg/mL after treatment indicates efficacy. In some embodiments, an amount of neurogranin that decreases by more than about 30%, 25%, 20%, 15%, 14%, 13%, 12%, 11%, 10%, 9%, 8%, 7%, 6%, 5%, 4%, 3%, 2%, or 1% after treatment indicates efficacy.

In some embodiments, the patient has been treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of AADvac1 before the presence and/or amount of neurogranin is determined. In some embodiments, at least 2, 3, 4, 5, 6, 7, 8, 9, 10, 11, 12, 13, 14, or 15 additional doses of AADvac1 is administered after the presence and/or amount of neurogranin is determined.

In some embodiments, behavioral measures, e.g., MMSE, are used alone or in combination with one or more biomarkers discussed herein, before, during, and/or after treatment to confirm efficacy. In some embodiments, a method of treating a patient suffering from Alzheimer's Disease or mild cognitive impairment comprises: (a) determining the total MMSE score of the patient; (b) administering one or more doses of an immunogenic peptide comprising SEQ ID NO: 1 or SEQ ID NO: 2; (c) after administering the one or more doses, determining the total MMSE score of the patient; (d) comparing the total MMSE score from step (c) after treatment to the MMSE score from step (a) before treatment, wherein an altered amount of neurogranin indicates treatment efficacy; and (e) administering one or more additional doses of the immunogenic composition comprising a peptide comprising SEQ ID NO: 1 or SEQ ID NO: 2 when the patient demonstrates treatment efficacy in step (d). In some embodiments, a stable total MMSE score indicates efficacy. In some embodiments, a total MMSE score that does not decrease by more than 20 point, 19 points, 18 points, 17 points, 16 points, 15 points, 14 points, 13 points, 12 points, 11 points, 10 points, 9 points, 8 points, 7 points, 6 points, 5 points, 4 points, 3 points 2 points, or 1 point indicates efficacy.

In some embodiments, the patient has been treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of AADvac1 before the presence and/or amount of neurogranin is determined. In some embodiments, at least 2, 3, 4, 5, 6, 7, 8, 9, 10, 11, 12, 13, 14, or 15 additional doses of AADvac1 is administered after the total MMSE score is determined in step (c).

In some embodiments, a patient is diagnosed with AD using a combination of brain imaging (e.g., MRI imaging of brain atrophy) and biomarker assessment. In some embodiments, the biomarker assessment comprises measuring the presence and/or amount of amyloid in a biological sample from the patient. In some embodiments, the biomarker assessment comprises measuring the presence and/or amount of tau, e.g., pathological tau, in a biological sample from the patient. In some embodiments, the biomarker assessment comprises measuring the presence and/or amount of amyloid and tau (e.g., pathological tau) in a biological sample from the patient in combination with brain imaging. In some embodiments, the biomarker assessment further comprises measuring NfL and/or neurogranin in a biological sample from the patient. In some embodiments, the biomarker assessment comprises measuring amyloid and/or tau (e.g., pathological tau) and further comprises measuring hsa-let-7a-5p, hsa-miR-15a-5p, hsa-miR191-5p, and hsa-miR-23a-3p in a biological sample from the patient.

Assessments of Treatment Efficacy

In some embodiments, treatment is effective in preventing, reducing, alleviating, slowing and/or partially or fully reversing one or more symptom of AD. In some embodiments, treatment is monitored or evaluated before, during, and/or after administration of an immunogenic peptide or pharmaceutical composition disclosed herein. In some embodiments, treatment is monitored or evaluated using one or more of the tests disclosed herein. In some embodiments, a baseline measurement on a test is taken before treatment and compared to a test measurement after treatment to monitor and/or confirm treatment. In some embodiments, the baseline test and/or testing administered during and/or after treatment comprises any of the tests (e.g., behavioral, biomarker, neuroimaging, cognitive, functional, etc.) discussed previously for diagnosing a patient with AD and/or as suitable for treatment.

One example of a test for evaluating treatment effectiveness that may be used is the Washington University Clinical Dementia Rating (CDR) that yields both a global score and a summated score (sum of box (SB) score). Hughes et al., Br. J. Psychiatry, (1982) 140:566-572; Berg et al., Ann Neurol., (1988) 23(5):477-484; Lynch et al., Dement. Geriatr. Cogn. Disord., (2006) 21(1):40-43. In some embodiments, the CDR is obtained through semi-structured interviews of patients and informants, and cognitive functioning is rated in 6 domains of functioning: memory, orientation, judgment and problem solving, community affairs, home and hobbies, and personal care. Each domain is rated on a 5-point scale of functioning as follows: 0, no impairment; 0.5, questionable impairment; 1, mild impairment; 2, moderate impairment; and 3, severe impairment (personal care is scored on a 4-point scale without a 0.5 rating available). The CDR-SB score is obtained by summing each of the domain box scores, with scores ranging from 0 to 18. Berg et al., Arch. Neurol., (1993) 50(4):349-358; Morris et al., Neurology, (1991) 41(4):469-478; Morris et al., Neurology, (1996) 46(3):707-719. In some embodiments, efficacy of treatment is assessed by the CDR-SB after one or more rounds of administration of an immunogenic peptide or pharmaceutical composition disclosed herein. In some embodiments, the patient's domain scores in the standard 6-domain CDR assessment is summed up to obtain a sum-of-boxes score. In some embodiments, the patient's CDR-SB score before and after treatment are compared to determine treatment efficacy. In some embodiments, treatment efficacy is assessed by the mean change in CDR-SB score from baseline to 4 week, 8 week, 12 weeks, 16 weeks, 20 weeks, 24 weeks, 3 months, 6 months, 9 months, 12 months, 15, months, 18 months, 21 months, 24 months, or 104 weeks after the start of treatment. In some embodiments, treatment is efficacious if the patient's CDR-SB score does not increase after treatment compared to baseline before treatment. In some embodiments, treatment is efficacious if the patient's CDR-SB score increases by no more than 1, 2, 3, 4, 5, 6, 7, or 8 after treatment compared to baseline before treatment. In some embodiments, treatment is efficacious if the patient's CDR-SB score increases by no more than 1 in each of the 6 domains after treatment compared to baseline before treatment. In some embodiments, treatment efficacy is assessed by a change in CDR-SB score from baseline to 4 week, 8 week, 12 weeks, 16 weeks, 20 weeks, 24 weeks, 3 months, 6 months, 9 months, 12 months, 15, months, 18 months, 21 months, 24 months, or 104 weeks after the start of treatment, as compared to a reference placebo score. In some embodiments, the difference in CDR-SB score for a patient treated with AADvac1 as described herein compared to a patient administered placebo is about −0.60 at week 104 post-treatment. In some embodiments, the difference in CDR-SB score for a patient treated with AADvac1 as described herein compared to a patient administered placebo is about 0.05 at week 104 post-treatment. In some embodiments, the difference in CDR-SB score for a patient treated with AADvac1 as described herein compared to a patient administered placebo is about −0.97 at week 104 post-treatment. In some embodiments, the difference in CDR-SB score for a patient treated with AADvac1 as described herein compared to a patient administered placebo is about −0.15 at week 104 post-treatment. In some embodiments, the difference in CDR-SB score for a patient treated with AADvac1 as described herein compared to a patient administered placebo is about −0.70 at week 104 post-treatment. In some embodiments, the difference in CDR-SB score for a patient treated with AADvac1 as described herein compared to a patient administered placebo is about 0.80 at week 104 post-treatment. In some embodiments, the difference in CDR-SB score for a patient treated with AADvac1 as described herein compared to a patient administered placebo is about −0.12 at week 104 post-treatment. In some embodiments, the difference in CDR-SB score for a patient treated with AADvac1 as described herein compared to a patient administered placebo is about −0.97 to about 0.80 at week 104 post-treatment. In some embodiments, treatment is efficacious if the patient's CDR-SB score does not increase after treatment compared to baseline before treatment. In some embodiments, treatment is efficacious if the patient's CDR-SB score increases by no more than 1, 2, 3, 4, 5, 6, 7, or 8 after treatment compared to baseline before treatment. In some embodiments, treatment is efficacious if the patient's CDR-SB score increases by no more than 1 in each of the 6 domains after treatment compared to baseline before treatment.

Other cognitive tests that may also be used to assessed treatment efficacy include, but are not limited to, tests of memory, language, executive function. One such test is the Cogstate International Shopping List Task (ISLT), which is a computer-administered and scored auditory/verbal list-learning test. Lim et al., Behav. Res. Methods, (2009) 41(4):1190-1200; Lim et al., Arch. Clin. Neuropsychol., (2012) 27(2):136-147; Ames et al., J. Clin. Exp. Neuropsych., (2012) 34(8):853-863. During the test, patients try to memorize 12 common food items that are read to them in a random order, and are assessed on their ability to recall the words, either immediately (immediate free recall) or 20 min later (delayed free recall). The test is performed three times and the number of items recalled is summed to give a total recall ISLT score and a delayed recall ISLT score. In some embodiments, efficacy of treatment is assessed by the Cogstate ISLT. In some embodiments, the patient's Cogstate ISLT score before and after treatment are compared to determine treatment efficacy. In some embodiments, treatment is efficacious if the ISLT score is higher or unchanged after treatment compared to baseline before treatment. In some embodiments, treatment is efficacious if the total recall ISLT score is no more than 1, 2, 3, 4, 5, 6, 7, 8, or 9 points worse after treatment compared to baseline before treatment. In some embodiments, treatment is efficacious if the delayed recall ISLT score is no more than 1, 2, 3, or 4 points worse after treatment compared to baseline before treatment. In some embodiments, the patient's Cogstate ISLT score after treatment relative to baseline is compared to a reference score obtained from a placebo group, e.g., a group provided with a treatment that is not expected to have any therapeutic value. In some embodiments, treatment is efficacious if the ISLT score after treatment relative to baseline is higher, more improved, or less worsened, compared to a reference score obtained from a placebo group, e.g., a group provided with a treatment that is not expected to have any therapeutic value.

Another cognitive test that may be used is the Cogstate One Card Learning Task, which is a continuous visual recognition learning task that assesses visual recognition memory and attention. During the test, a playing card is presented face-down in the center of a computer screen on a green background. After a randomly varied interval of 2.5-3.5 seconds, the card is turned face up and the patient is required to respond “yes” if the card has appeared in the test before, and “no” if it has not yet appeared. Fredrickson et al., Met. Neuroepidemiology, (2010), 34:66-75. In some embodiments, accuracy of performance is defined as the number of correct responses made expressed as a proportion of the total trials attempted. In some embodiments, accuracy of performance is expressed as the arcsine transformation of the square root of the proportion of correct responses. In some embodiments, efficacy of treatment is assessed by the Cogstate One Card Learning Task. In some embodiments, the patient's Cogstate One Card Learning Task score before and after treatment are compared to determine treatment efficacy. In some embodiments, treatment is efficacious if the Cogstate One Card Learning Task score is higher or unchanged after treatment compared to baseline before treatment. In some embodiments, the patient's Cogstate One Card Learning Task score after treatment relative to baseline is compared to a reference score obtained from a placebo group, e.g., a group provided with a treatment that is not expected to have any therapeutic value. In some embodiments, treatment is efficacious if the One Card Learning score after treatment relative to baseline is higher, more improved, or less worsened, compared to a reference score obtained from a placebo group, e.g., a group provided with a treatment that is not expected to have any therapeutic value.

Another cognitive test that may be used is the Cogstate One Back Task. This test assesses working memory and attention, and uses a similar setup as the One Card Learning Task. Instead of determining if the card has appeared in the test before, a patient is required to respond “yes” if the card is exactly the same as the immediately previous card, or “no” if it is not the same as the previous card. Fredrickson et al., Met. Neuroepidemiology, (2010), 34:66-75. Speed of performance may be measured as the mean of the log 10 transformed reaction time for a correct response. In some embodiments, efficacy of treatment is assessed by the Cogstate One Back Task. In some embodiments, the patient's Cogstate One Back Task score before and after treatment are compared to determine treatment efficacy. In some embodiments, treatment is efficacious if the Cogstate One Back Task score is lower or unchanged after treatment compared to baseline before treatment. In some embodiments, the patient's Cogstate One Back Task score after treatment relative to baseline is compared to a reference score obtained from a placebo group, e.g., a group provided with a treatment that is not expected to have any therapeutic value. In some embodiments, treatment is efficacious if the One Back Task score after treatment relative to baseline is lower, more improved, or less worsened, compared to a reference score obtained from a placebo group, e.g., a group provided with a treatment that is not expected to have any therapeutic value.

Another cognitive test that may be used is the Category Fluency Test and the Letter Fluency Test. These are assessments of executive function and language. Lezak et al., Neuropsychological Assessment. (2012) New York, N.Y.: Oxford University Press. In the Category Fluency Test, patients are asked to produce as many words as possible from a given category, such as animals or fruits, within a given time. In the Letter Fluency Test, patients are asked to produce as many words as possible that start with a given alphabet letter within a given time. In some embodiments, the Category Fluency Test score is defined by the number of acceptable words produced. In some embodiments, the Letter Fluency Test score is defined by the total number of acceptable words produced over three trials. In some embodiments, efficacy of treatment is assessed by a Category Fluency Test. In some embodiments, the patient's Category Fluency Test score before and after treatment are compared to determine treatment efficacy. In some embodiments, efficacy of treatment is assessed by a Letter Fluency Test. In some embodiments, the patient's Letter Fluency Test score before and after treatment are compared to determine treatment efficacy. In some embodiments, treatment is efficacious if the Category Fluency Test and the Letter Fluency Test scores are higher or unchanged after treatment compared to baseline before treatment. In some embodiments, the patient's Category Fluency Test score after treatment relative to baseline is compared to a reference score obtained from a placebo group, e.g., a group provided with a treatment that is not expected to have any therapeutic value. In some embodiments, treatment is efficacious if the Category Fluency Test score after treatment relative to baseline is higher, more improved, or less worsened, compared to a reference score obtained from a placebo group, e.g., a group provided with a treatment that is not expected to have any therapeutic value. In some embodiments, the patient's Letter Fluency Test score after treatment relative to baseline is compared to a reference score obtained from a placebo group, e.g., a group provided with a treatment that is not expected to have any therapeutic value. In some embodiments, treatment is efficacious if the Letter Fluency Test score after treatment relative to baseline is higher, more improved, or less worsened, compared to a reference score obtained from a placebo group, e.g., a group provided with a treatment that is not expected to have any therapeutic value.

Another cognitive test that may be used is the Digit Symbol Coding Test. This is a measure of executive functioning, working memory and processing speed. Boake et al., J. Clin. Exp. Neuropsychol., (2002) 24:383-405; Lezak et al., Neuropsychological Assessment. (2012) New York, N.Y.: Oxford University Press. A patient is asked to match symbols to numbers according to a key located on the top of a paper. The number of correct symbols within the allowed time, usually 120 seconds, constitutes the score. In some embodiments, efficacy of treatment is assessed by the Digit Symbol Coding Test. In some embodiments, the patient's Digit Symbol Coding Test score before and after treatment are compared to determine treatment efficacy. In some embodiments, treatment is efficacious if the Digit Symbol Coding Test score is higher or unchanged after treatment compared to baseline before treatment. In some embodiments, the patient's Digit Symbol Coding Test score after treatment relative to baseline is compared to a reference score obtained from a placebo group. In some embodiments, treatment is efficacious if the Digit Symbol Coding Test score after treatment relative to baseline is higher, more improved, or less worsened, compared to a reference score obtained from a placebo group, e.g., a group provided with a treatment that is not expected to have any therapeutic value.

In some embodiments, treatment efficacy is assessed by a combination of cognitive tests. In some embodiments, a Custom Cognitive Battery includes the Cogstate International Shopping List Task, the Cogstate One Card Learning Task, the Cogstate One Card Back Task, the Category Fluency Test, the Letter Fluency Test, and the Digit Symbol Coding Test. In some embodiments, a z-score is calculated for each test of the Custom Cognitive Battery. In some embodiments, a composite z-score is calculated from the results of the Custom Cognitive Battery of tests. In some embodiments, the z-score is calculated from the patient's actual composite score on the Custom Cognitive Battery at each visit, normalized by the mean baseline score and standard deviation in the full analysis set. In some embodiments, treatment efficacy is assessed by the mean change in composite z-score of the Custom Cognitive Battery from baseline to 4 week, 8 week, 12 weeks, 16 weeks, 20 weeks, 24 weeks, 3 months, 6 months, 9 months, 12 months, 15, months, 18 months, 21 months, 24 months, or 104 weeks after the start of treatment.

In some embodiments, treatment efficacy may also be measured by improvements in a patient's daily living. For instance, the Alzheimer's Disease Cooperative Study Mild Cognitive Impairment activities of Daily Living (ADCS MCI ADL) questionnaire may be used. It is an interview-based informant-reported measure that assesses the competence of patients with AD in basic and instrumental activities of daily living, such as eating, handling finances, shopping, travel, and remembering appointments. Galasko et al., Alzheimer Dis. Assoc. Disord., (1997) 11 Suppl 2:S33-39; Pedrosa et al., J. Nutr. Health Aging, (2010) 14:703-709. The questionnaire rates a patient based on the extent of assistance he/she requires for specific tasks, e.g., 0 if the patient has total independence in performing an activity to 4 for total inability to act independently. An 18-item (ADCS-MCI-ADL-18) version of the test includes questions on shopping, hobbies, and personal appliances, while a 24-item version (ADCS-MCI-ADL-24) of the test has an additional 6 items including driving a car and organizing medication. In some embodiments, efficacy of treatment is assessed by the ADCS-MCI-ADL-18. In some embodiments, the patient's ADCS-MCI-ADL-18 score before and after treatment are compared to determine treatment efficacy. In some embodiments, efficacy of treatment is assessed by the ADCS-MCI-ADL-24. In some embodiments, the patient's ADCS-MCI-ADL-24 score before and after treatment are compared to determine treatment efficacy. In some embodiments, efficacy of treatment is assessed by both the ADCS-MCI-ADL-18 and the ADCS-MCI-ADL-24. In some embodiments, both the patient's ADCS-MCI-ADL-18 and ADCS-MCI-ADL-24 scores before and after treatment are compared to determine treatment efficacy. In some embodiments, treatment efficacy is assessed by the mean change in the ADCS-MCI-ADL-18 score from baseline to 104 weeks after the start of treatment. In some embodiments, treatment efficacy is assessed by the mean change in the ADCS-MCI-ADL-24 score from baseline to 104 weeks after the start of treatment. In some embodiments, treatment efficacy is assessed by the mean change in both the ADCS-MCI-ADL-18 and ADCS-MCI-ADL-24 scores from baseline to 4 week, 8 week, 12 weeks, 16 weeks, 20 weeks, 24 weeks, 3 months, 6 months, 9 months, 12 months, 15, months, 18 months, 21 months, 24 months, or 104 weeks after the start of treatment.

In some embodiments, treatment efficacy may be measured by antibody titre and/or immunogenicity in a patient (alone or in combination with one or more alternative test, e.g., a test discussed herein). Administration of an immunogenic peptide may cause an antibody response in a patient. In some embodiments, efficacy is assessed by whether a patient has developed an IgG antibody response against the immunogenic peptide disclosed herein. In some embodiments, efficacy is assessed by whether a patient has developed an IgM antibody response against the immunogenic peptide disclosed herein. In some embodiments, efficacy is assessed by the geometric mean titer of immunogenic peptide-induced IgG antibodies. In some embodiments, efficacy is assessed by the geometric mean titer of immunogenic peptide-induced IgM antibodies. In some embodiments, IgG or IgM antibody response is measured by an enzyme-linked immunosorbent assay (ELISA). In some embodiments, mean titers of IgG or IgM antibodies is measured by ELISA.

In some embodiments, treatment efficacy may be measured by changes in the functional levels of brain, e.g., brain metabolism. In some embodiments, fluodeoxyglucose-positron emission tomography (FDG-PET). FDG-PET has been useful for detecting functional brain changes in AD, identifying changes in early AD, and in helping to differentiate AD from other causes of dementia. Using FDG as a tracer, resting state cerebral metabolic rates of glucose, which is an indicator of neuronal activity may be measured. Mosconi, Clin. Transl. Imaging, (2013) 1(4): 217-233; Kantarci, Am. J. Neuroradiol., (2014) 35(6 Suppl):S12-177. In some embodiments, the treatment efficacy is assessed by FDG-PET. In some embodiments, treatment efficacy is assessed by measuring brain metabolism using FDG-PET. In some embodiments, FDG-PET assesses brain metabolism by changes in cerebral glucose metabolic rate. In some embodiments, changes in cerebral glucose metabolic rate are expressed as the change in Standardized Uptake Value Ratio (SUVR) in multiple regions of interest. In some embodiments, treatment efficacy is assessed by change in brain metabolism as measured by FDG-PET from baseline to 4 week, 8 week, 12 weeks, 16 weeks, 20 weeks, 24 weeks, 3 months, 6 months, 9 months, 12 months, 15, months, 18 months, 21 months, or 24 months after the start of treatment.

In some embodiments, treatment efficacy may be measured by hippocampal atrophy, which is a feature of AD that may correlate with AD neuropathology. Atrophy can be detected using MRI, and thus anatomic volumetry using MRI may be used as a biomarker for AD progression. Fleisher et al., Neurology, (2008) 70(3): 191-199. In some embodiments, the treatment efficacy is assessed by MRI volumetry. In some embodiments, treatment efficacy is assessed by change in MRI volumetry from baseline to 4 week, 8 week, 12 weeks, 16 weeks, 20 weeks, 24 weeks, 3 months, 6 months, 9 months, 12 months, 15, months, 18 months, 21 months, or 24 months after the start of treatment.

In some embodiments, the treatment efficacy is assessed by measurements of one or more biomarkers. In some embodiments, treatment efficacy is assessed by the levels of tau proteins in plasma after the start of treatment compared to baseline before treatment. In some embodiments, treatment efficacy is assessed by the levels of tau proteins in cerebrospinal fluid (CSF) after the start of treatment compared to baseline before treatment. In some embodiments, treatment efficacy is assessed by the levels of total tau proteins in the CSF after the start of treatment compared to baseline before treatment. In some embodiments, treatment is continued or discontinued based on one or more efficacy measurement. In some embodiments, total tau is measured using an ELISA assay. In some embodiments, the ELISA assay is an Innotest hTAU Ag and phospho-tau (181P) ELISA assay. In some embodiments, pT181 tau is measured using an ELISA assay. In some embodiments, the ELISA assay is an Innotest hTAU Ag and phospho-tau (181P) ELISA assay. In some embodiments, pT217 tau is measured using an ELISA assay. In some embodiments, the ELISA assay is a digital ELISA assay.

In some embodiments, treatment is efficacious if total tau protein concentration in the CSF is reduced or remains unchanged after the start of treatment compared to baseline before treatment. In some embodiments, treatment is efficacious if total tau protein concentration in the CSF increases by no more than 10%, 20%, 30%, 40%, 50%, 60%, 70%, 80% or 90% after the start of treatment compared to baseline before treatment. In some embodiments, treatment is efficacious if total tau (t-tau) protein concentration in the CSF decreases by at least 10%, 20%, 30%, 40%, 50%, 60%, 70%, 80% or 90% after the start of treatment compared to baseline before treatment. In some embodiments, treatment efficacy is assessed by the levels of tau proteins phosphorylated at threonine 181 (pT181 tau) in the CSF after the start of treatment compared to baseline before treatment. In some embodiments, treatment is efficacious if pT181 tau concentration in the CSF is reduced or remains unchanged after the start of treatment compared to baseline before treatment. In some embodiments, treatment is efficacious if pT181 tau concentration in the CSF increases by no more than 10%, 20%, 30%, 40%, 50%, 60%, 70%, 80% or 90% after the start of treatment compared to baseline before treatment. In some embodiments, treatment is efficacious if pT181 tau concentration in the CSF decreases by at least 10%, 20%, 30%, 40%, 50%, 60%, 70%, 80% or 90% after the start of treatment compared to baseline before treatment. In some embodiments, treatment efficacy is assessed by the levels of tau proteins phosphorylated at threonine 217 (pT217 tau) in the CSF after the start of treatment compared to baseline before treatment. In some embodiments, treatment is efficacious if pT217 tau concentration in the CSF is reduced or remains unchanged after the start of treatment compared to baseline before treatment. In some embodiments, treatment is efficacious if pT217 tau concentration in the CSF increases by no more than 10%, 20%, 30%, 40%, 50%, 60%, 70%, 80% or 90% after the start of treatment compared to baseline before treatment. In some embodiments, treatment is efficacious if pT217 tau concentration in the CSF decreases by at least 10%, 20%, 30%, 40%, 50%, 60%, 70%, 80% or 90% after the start of treatment compared to baseline before treatment. In some embodiments, concentration of pT217 tau is measured as described in WO 2019/186276 A2, the contents of which are incorporated by reference in their entirety.

In some embodiments, treatment efficacy is assessed by the levels of Aβ42 peptides in the CSF after the start of treatment compared to baseline before treatment. In some embodiments, treatment is efficacious if Aβ42 concentration in the CSF remains unchanged after the start of treatment compared to baseline before treatment. In some embodiments, treatment is efficacious if Aβ42 concentration in the CSF changes by no more than 10%, 20%, 30%, 40%, or 50% after the start of treatment compared to baseline before treatment. In some embodiments, treatment efficacy is assessed by the ratio of Aβ42:Aβ40 in the CSF after the start of treatment compared to baseline before treatment. In some embodiments, treatment is efficacious if the ratio of Aβ42:Aβ40 in the CSF remains unchanged after the start of treatment compared to baseline before treatment. In some embodiments, treatment is efficacious if the ratio of Aβ42:Aβ40 in the CSF changes by no more than 10%, 20%, 30%, 40%, or 50% after the start of treatment compared to baseline before treatment. In some embodiments, treatment efficacy is assessed by the levels of one or more of total tau proteins, pathological tau proteins, Aβ42 peptides, Aβ40 peptides, and/or ratio of Aβ42:Aβ40 in the CSF at 4 week, 8 week, 12 weeks, 16 weeks, 20 weeks, 24 weeks, 3 months, 6 months, 9 months, 12 months, 15, months, 18 months, 21 months, or 24 months after treatment compared to baseline before treatment. In some embodiments, treatment is continued if the treatment is deemed efficacious by the methods and criteria described herein, e.g., at least one, two, or more additional doses of a composition comprising an immunogenic peptide is administered to a patient. In some embodiments, treatment is continued indefinitely if the treatment is deemed efficacious by the methods and criteria described herein. In some embodiments, Aβ40 is measured using an ELISA assay. In some embodiments, Aβ42 is measured using an ELISA assay.

Patient Selection Criteria

In various embodiments, patients treated with one or more of the methods disclosed herein may be or have previously been receiving treatment with one or more other drugs, e.g., given before, during, and/or after treatment with the pharmaceutical compositions disclosed herein. Treatments targeting cognitive symptoms (e.g., memory loss, confusion, and problems with thinking and reasoning) may include, but are not limited to cholinesterase inhibitors (Aricept, Exelon, Razadyne) and memantine (Namenda). In some embodiments, the patient has received a stable therapy with an acetylcholinesterase inhibitor (e.g., Aricept, Exelon, Razadyne) for at least 1, 2, 3, 4, 5, or 6 months, e.g., at least 3 months, prior to administration of the immunogenic peptide disclosed herein. In some embodiments, the patient has received a stable therapy with memantine (e.g., Namenda) for at least 1, 2, 3, 4, 5, or 6 months, e.g., at least 3 months, prior to administration of the immunogenic peptide disclosed herein. In some embodiments, the patient has received a stable dose regiment of memantine (e.g., Namenda) for at least 3 months prior to administration of the immunogenic peptide disclosed herein.

Methods of selecting patients who will benefit from the treatment disclosed here are also contemplated. In some embodiments, the patient is not contraindicated for MRI imaging, e.g., patient does not have MRI-incompatible metallic endoprosthesis or MRI-incompatible stent implantation. In some embodiments, the patient does not have an infarction, e.g., in the territory of one or more large brain vessels, as detected by a brain MRI. In some embodiments, the patient does not have more than one lacunar infarct defined as a focal lesion of CSF signal intensity with a diameter of less than 1.5 cm in any dimension, as detected by a brain MRI. In some embodiments, the patient does not have any lacunar infarct, e.g., in the thalamus, hippocampus of either hemisphere, head of the left caudate nucleus, as detected by a brain MRI. The Fazekas scale may be used to quantify the amount of white matter lesions in the brain, where a region of the brain is given a grade for the size and confluence of lesions ranging from 0 for no lesions to 3 for an irregular or large confluent area of lesions. In some embodiments, the patient does not have confluent hemispheric deep white matter lesions (Fazekas grade 3) as detected by a brain MRI, e.g., Fazekas grade 0, 1, or 2. In some embodiments, the patient does not have focal lesions that affect cognitive status stemming from a central nervous system disease other than AD, such as current or acute infectious disease, space occupying lesions, normal pressure hydrocephalus or any other abnormalities associated with significant central nervous disease other than Alzheimer's disease, as detected by a brain MRI. In some embodiments, the patient has not undergone surgery (under general anesthesia) within 3 months prior to the treatment disclosed herein.

In some embodiments, the patient does not have an allergy to one or more components of a vaccine disclosed here, currently or in the past. In some embodiments, patients who are female are not pregnant or breastfeeding. In some embodiments, the patient does not have a history and/or is not currently suffering from a systemic autoimmune disease, e.g., systemic lupus erythematosus, rheumatoid arthritis (RA), or Sjögren syndrome, or a disease that requires immunosuppressive or immunomodulatory therapy. In some embodiments, the patient is not receiving or expected to receive immunosuppressive or immunomodulatory treatment at the time or treatment or in the future. In some embodiments, the patient does not have a recent history of cancer. In some embodiments, the patient has not had specific treatment for cancer (except for basal cell carcinoma or intraepithelial cervical neoplasia) less than 1 month to ten years, e.g., five years or less, prior to the treatment disclosed herein. In some embodiments, the patient has not had a myocardial infarction within zero to five years prior, e.g., within two years prior, to the treatment disclosed herein. In some embodiments, the patient has not had Hepatitis B, C, HIV or syphilis confirmed by serology. In some embodiments, the patient does not suffer from an active infectious disease at the time of the treatment disclosed herein. In some embodiments, the patient does not have a history of immunodeficiency. In some embodiments, the patient does not currently have immunodeficiency at the time of the treatment disclosed herein.

In some embodiments, patients suffering from another systemic illness other than AD may receive less therapeutic benefit from the treatments disclosed herein and may be excluded from treatment. For example, patients with congestive heart failure, high body-mass index, diabetes, renal insufficiency, or liver disease. In some embodiments, the patient does not suffer from another clinically important systemic illness that is likely to result in deterioration of the patient's condition. For example, the New York Heart Association Functional Classification (NYHA) is used to classify patient's heart failure according to the severity of their symptoms, from 1 for no limitation of physical activity, 2 for slight limitation of physical activity, 3 for marked limitation of physical activity to 4 for unable to carry on any physical activity without discomfort. In some embodiments, the patient does not have poorly controlled congestive heart failure defined by NYHA to be 2, 3, or 4. In some embodiments, the patient does not have poorly controlled congestive heart failure defined by NYHA of greater than or equal to 3, i.e. 3 or 4. In some embodiments, the patient does not have a body-mass index (BMI) of greater than 48, 46, 44, 42, 40, 38, 36, 34, or 32. In some embodiments, the patient does not have a body-mass index (BMI) of greater than 40. In some embodiments, the patient does not have poorly controlled diabetes as defined by a level of glycated hemoglobin (HbA1c) in blood that is greater than 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, or 14.0%. In some embodiments, the patient does not have poorly controlled diabetes as defined by a level of glycated hemoglobin (HbA1c) in blood that is greater than 7.5%. In some embodiments, the patient does not have severe renal insufficiency as defined by an estimated glomerular filtration rate (eGFR) level of less than 60 mL/min, 50 mL/min, 40 mL/min, 30 mL/min, 20 mL/min, or 10 mL/min. In some embodiments, the patient does not have severe renal insufficiency as defined by an eGFR level of less than 30 mL/min. In some embodiments, the patient does not have chronic liver disease, as defined by alanine aminotransferase (ALT) levels of greater than 50 U/L, 55 U/L, 60 U/L, 65 U/L, 70 U/L, 75 U/L, or 80 U/L in females or greater than 60 U/L, 65 U/L, 70 U/L, 75 U/L, 80 U/L, 85 U/L, 90 U/L, 95 U/L, or 100 U/L in males, and aspartate aminotransferase (AST) levels of greater than 60 U/L, 65 U/L, 70 U/L, 75 U/L, 80 U/L, 85 U/L, 90 U/L, 95 U/L, or 100 U/L. In some embodiments, the patient does not have chronic liver disease, as defined by ALT levels of greater than 66 U/L in females or greater than 80 U/L in males. In some embodiments, the patient does not have chronic liver disease, as defined by AST levels of greater than 82 U/L.

In some embodiments, the patient does not suffer from hypothyroidism, as defined by thyroid-stimulating hormone (TSH) levels of more than 4.0 mIU/L, 4.5 mIU/L, 5.0 mIU/mL, 5.5 mIU/L, 6.0 mIU/L, 7.0 mIU/L, 8.0 mIU/L, 9.0 mIU/L, or 10.0 mIU/L, and/or thyroxine (FT4) levels of less than 1.5 ng/dL, 1.4 ng/dL, 1.3 ng/dL, 1.2 ng/dL, 1.1 ng/dL, 1.0 ng/dL, 0.9 ng/dL, 0.8 ng/dL, 0.7 ng/dL, 0.6 ng/dL, 0.5 ng/dL, or 0.4 ng/dL. In some embodiments, the patient does not suffer from hypothyroidism, as defined by TSH levels of more than 5.0 mIU/mL, and/or FT4 levels of less than 0.7 ng/d L.

In some embodiments, the patient does not have a diagnosis of significant psychiatric illness such as schizophrenia, any type of psychotic disorder or bipolar affective disorder. In some embodiments, the patient does not have a current depressive episode as defined by the Geriatric Depression Scale (GDS) of greater or equal to 6. In some embodiments, the patient has not had a major depressive episode within one year prior to the treatment disclosed herein. In some embodiments, the patient does not have a metabolic or toxic encephalopathy or dementia due to a general medical condition. In some embodiments, the patient does not have a history of alcohol or drug abuse or dependence within two years prior to the treatment disclosed herein. In some embodiments, the patient does not have Wernicke's encephalopathy. In some embodiments, the patient does not have a history or evidence of any CNS disorder other than AD that could be the cause of dementia (e.g., infectious or inflammatory/demyelinating CNS conditions, Creutzfeldt-Jakob disease, Parkinson's disease, Huntington's disease, brain tumor, subdural hematoma). In some embodiments, the patient does not have a history of cerebrovascular disease (ischemic or hemorrhagic stroke), or diagnosis of possible, probable or definite vascular dementia. In some embodiments, the patient does not have a history or present diagnosis of epilepsy. In some embodiments, the patient is not undergoing treatment with experimental immunotherapeutics including intravenous immunoglobulin (IVIg). In some embodiments, the patient has not undergone treatment with experimental immunotherapeutics including IVIg in the 6 months, 5 months, 4 months, 3 months, 2 months, or 1 month prior to the treatment disclosed herein. In some embodiments, the patient has not undergone treatment with experimental immunotherapeutics including IVIg in the 3 months prior to the treatment disclosed herein. In some embodiments, the patient is not undergoing treatment with an experimental therapy for AD aimed at disease modification. In some embodiments, the patient has not undergone treatment with experimental therapies for AD aimed at disease modification in the 3 months prior to the treatment disclosed herein. In some embodiments, the patient is not undergoing treatment with any active vaccines for AD. In some embodiments, the patient has not undergone treatment with any active vaccines for AD. In some embodiments, the patient is not undergoing treatment with immunosuppressive drugs. In some embodiments, the patient does not have a vitamin B12 deficiency as defined by a serum vitamin B12 level of less than 250 pg/mL, 240 pg/mL, 230 pg/mL, 220 pg/mL, 210 pg/mL, 200 pg/mL, 190 pg/mL, 180 pg/mL, 170 pg/mL, 160 pg/mL, or 150 pg/mL. In some embodiments, the patient does not have a vitamin B12 deficiency as defined by a serum vitamin B12 level of less than 191 pg/mL. In some embodiments, the patient has not changed his/her dose of previous or current medications due to concomitant illness in the 90 days, 60 days, 30 days, 15 days, or 10 days prior to the treatment disclosed herein. In some embodiments, the patient has not changed his/her dose of previous or current medications due to concomitant illness in the 30 days prior to the treatment disclosed herein.

In some embodiments, the patient does not have (a) an MRI finding inconsistent with diagnosis of AD, (b) any neurological condition other than AD which can cause or significantly contribute to the symptoms of dementia, and (c) any systematic autoimmune disorders e.g. systematic lupus erythematosus.

Assessment of Treatment Tolerability

In some embodiments, tolerability of a treatment disclosed herein is measured, e.g., to determine whether to continue treatment and/or to select a suitable dosage. Tolerability may refer to the degree to which overt adverse effects of a drug can be tolerated by a patient. The tolerability of the treatment method by the patient may be determined using a variety of tests for cognitive skills before and after treatment, e.g., brain scans by MRI, physical exam, neurological exam. In some embodiments, the tolerability of treatment is assessed by vital signs of the patient, measured before and after treatment. The treatment method may be determined to be tolerated when the absolute or relative change of a test parameter after treatment compared to baseline before treatment does not exceed a predetermined range.

In some embodiments, the tolerability of treatment is assessed by electrocardiogram (ECG) of the patient, measured before and after treatment. In some embodiments, the treatment is determined to be tolerated if the ECG of the patient, e.g., the rate, rhythm, intervals, or wave morphologies, is unchanged after the treatment compared to baseline before treatment. In some embodiments, the treatment is determined to be tolerated if the ECG of the patient does not show signs of ischemia or infarction after the treatment compared to baseline before treatment. In some embodiments, the tolerability of treatment is assessed by laboratory measures of the patient, measured before and after treatment. In some embodiments, the tolerability of treatment is assessed by MRI scans of the patient's brain, measured before and after treatment. In some embodiments, the treatment is determined to be tolerated if the MRI of the patient is unchanged after the treatment compared to baseline before treatment. In some embodiments, tolerability of treatment is indicated by the lack of changes in atrophy in brain regions after treatment compared to baseline before treatment. In some embodiments, tolerability of treatment is indicated by the absence of microbleeds, lacuna, superficial hemosiderin deposition, or white matter lesions or hyperintensities after treatment compared to baseline before treatment.

In some embodiments, the tolerability of treatment is assessed by physical and/or neurological examinations of the patient, measured before and after treatment. The Columbia-Suicide Severity Rating Scale (C-SSRS) is a commonly used assessment of suicidal ideation and behavior, where six questions are asked to determine if a patient has experienced several thoughts or feelings relating to suicide over the past month and behaviors over his/her lifetime and during the past 3 months, e.g., number of suicidal thoughts and intent to act. The number of affirmative responses may correlate with the risk of suicide. In some embodiments, the tolerability of treatment is assessed by the C-SSRS, measured before and after treatment. In some embodiments, the treatment is determined to be tolerated if the number of affirmative responses on the C-SSRS is unchanged after the treatment compared to baseline. In some embodiments, the treatment is determined to be tolerated if the number of affirmative responses on the C-SSRS increases by no more than one, no more than two, no more than three, or no more than four after the treatment compared to baseline. In some embodiments, the treatment is determined to be tolerated if the number of affirmative responses on the C-SSRS after the treatment is less than five, less than four, less than three, less than two, or less than one. In some embodiments, the tolerability of treatment is assessed by reviewing the patient diary of the patient, compared before and after treatment. In some embodiments, treatment is continued if the treatment is deemed tolerable by the methods and criteria described herein. In some embodiments, treatment is continued but dosage administered is reduced if the treatment is deemed not tolerable by the methods and criteria described herein.

The present disclosure is further illustrated by the following examples that should not be construed as limiting. The contents of all references, patents, and published patent applications cited throughout this application, as well as the figures, are incorporated herein by reference in their entirety for all purposes.

### EXAMPLES

### Example 1: Clinical Trial

A 24-month randomized, placebo-controlled, double-blinded, multi-center phase 2 study was conducted to assess the safety and efficacy of 40 pg synthetic peptide derived from amino acids 294 to 305 of the tau sequence coupled to keyhole limpet hemocyanin (AADvac1) after two years of treatment.

Methods: Patient Inclusion Criteria

1. Patient has a diagnosis of probable Alzheimer's disease according to the revised NIA-AA criteria (McKhann 2011).

2. Patient has a MMSE total score 20 and 26 at the screening visit.

3. Patient has a brain MRI finding consistent with the diagnosis of Alzheimer's disease at the screening visit.

4. Patient has evidence of the AD pathophysiological process at the screening visit, defined as one or both of the following:
a. medial temporal lobe atrophy as assessed on brain MRI and according to a Scheltens score of ≥2 (rated on a scale of 0-4 on the more atrophied side)
b. positive AD biomarker signature in the CSF (total tau protein>400 pg/mL AND pT181 tau protein>60 pg/mL and Aβ42<600 pg/mL and Aβ42:Aβ40 ratio<0.089)

5. Patient has completed 6 years of formal elementary education.

6. Patients aged 50-85 years inclusive at the screening visit.

7. Patient is fluent in the local language and possesses sufficient auditory and visual capacities to allow neuropsychological testing.

8. Patient is able to read and understand the informed consent.

9. Patient is on a stable therapy with an acetylcholinesterase inhibitor for at least 3 months prior to the screening visit.

10. If the patient is on memantine treatment, the dose regimen must be stable for at least 3 months prior to the screening visit.

11. Patient has a Hachinski Ischemia Scale score 4 at the screening visit.

12. Availability of a caregiver who sufficiently knows the patient and will be able to accompany the patient on the study visits and to participate in study assessments of the patient where required.

13. Female patients are only eligible for the study if they are either surgically sterile or at least 2 years postmenopausal.

14. Male patients must either be surgically sterile, or he and his female spouse/partner who is of childbearing potential must be using highly effective contraception consisting of 2 forms of birth control (1 of which must be a barrier method) starting at the screening visit and continuing throughout the study period.

15. Patient provides written informed consent.

Methods: Patient Exclusion Criteria

1. Female patient who is pregnant or breastfeeding.

2. Patient has been participating in another clinical study within 3 months prior to the screening visit.

3. Patient is not expected to complete the clinical study.

4. Patient has known allergy to components of the vaccine currently or in the past, if considered relevant by the investigator.

5. Patient has known contraindication for MRI imaging such as MRI-incompatible metallic endoprosthesis or MRI-incompatible stent implantation or other as judged by the Investigator.

6. Any of the following detected by brain MRI:

a) Infarction in the territory of large vessels;

b) More than one lacunar infarct defined as a focal lesion of CSF signal intensity with a diameter of less than 1.5 cm in any dimension;

c) Any lacunar infarct in a strategically important location such as the thalamus, hippocampus of either hemisphere, head of the left caudate nucleus.

d) Confluent hemispheric deep white matter lesions (Fazekas grade 3); or e) Other focal lesions which may be responsible for the cognitive status of the patient such as infectious disease, space-occupying lesions, normal pressure hydrocephalus or any other abnormalities associated with significant central nervous disease other than Alzheimer's disease.

7. Patient underwent surgery (under general anaesthesia) within 3 months prior to screening and/or has scheduled surgery (under general anaesthesia) during the whole study period.

8. Patient has a history and/or currently suffers from a clinically significant autoimmune disease, or is expected to receive immunosuppressive or immunomodulatory treatment at the present or in the future.

9. Patient has a recent history of cancer (last specific treatment 5 years prior to Screening) (Exceptions: basal cell carcinoma, intraepithelial cervical neoplasia).

10. Patient had myocardial infarction within the last 2 years prior to the screening visit.

11. Patient has Hepatitis B, C, HIV or Syphilis confirmed by serology.

12. Patient suffers from an active infectious disease.

13. Presence and/or history of immunodeficiency.

14. Patient currently suffering from a clinically important systemic illness that is likely to result in deterioration of the patient's condition or affect the patient's safety during the study:
poorly controlled congestive heart failure (NYHA≥3)
BMI>40
poorly controlled diabetes (HbA1c>7.5%)
severe renal insufficiency (eGFR<30 mL/min)
chronic liver disease—ALT (alanine aminotransferase)>66 U/L in females or >80 U/L in males, AST (aspartate aminotransferase)>82 U/L
other clinically significant systemic illness, if considered relevant by the investigator

15. Patient suffers from hypothyroidism, defined as TSH (thyroid-stimulating hormone) elevation>5.0 mIU/mL, and/or FT4 levels<0.7 ng/dL. Patients with corrected hypothyroidism are eligible for the study provided that treatment has been stable for 3 months before study entry.

16. Patient has valid diagnosis of a significant psychiatric illness such as schizophrenia, any type of psychotic disorder or bipolar affective disorder.

17. Patient has a current depressive episode (Geriatric Depression Scale GDS≥6 at Visit 01) or had a major depressive episode within the last 1 year.

18. Patient has a metabolic or toxic encephalopathy or dementia due to a general medical condition.

19. Patient has a history of alcohol or drug abuse or dependence within the past 2 years.

20. Patient has Wernicke's encephalopathy.

21. Patient has a history or evidence of any CNS disorder other than AD that could be the cause of dementia (e.g., infectious or inflammatory/demyelinating CNS conditions, Creutzfeldt-Jakob disease, Parkinson's disease, Huntington's disease, brain tumour, subdural haematoma).

22. Patient has a history with evidence of cerebrovascular disease (ischemic or haemorrhagic stroke), or diagnosis of possible, probable or definite vascular dementia.

23. Patient has a history or presence of diagnosis of epilepsy.

24. Patient is currently treated and/or was treated with experimental immunotherapeutics including IVIG within 3 months prior to the screening visit.

25. Patient is currently being treated and/or was treated with experimental therapies for AD aiming at disease-modification within 3 months prior to the screening visit.

26. Patient is currently being treated or was treated in the past with any active vaccines for AD.

27. Patient is currently being treated with immunosuppressive drugs.

28. Change in dose of previous and current medications which the patient is taking because of concomitant illnesses according to the medical history within the last 30 days prior to the screening visit, if considered clinically relevant.

29. Patient has vitamin B12 deficiency (serum vitamin B12<191 pg/mL).

Methods: Protocol

The clinical trial was performed according to the schedule as shown in Table 5.

TABLE 5
Study Schedule

| | Study Visit Number | | | | | | | | | | |
|---|---|---|---|---|---|---|---|---|---|---|---|
| | V01 | V02 | | | | | | | | | |
| | (Screening) | (Baseline) | V03 | V04 | V05 | V06 | V07 | V08 | V08a | V09 | V10 |
| | Week of the Study Visit | | | | | | | | | | |
| | | Week +0 | Week +4 | Week +8 | Week +12 | Week +16 | Week +20 | Week +24 | Week +34 | Week +38 | Week +48 |
| Cognitive | X | X | | | X | | | X | | X | |
| Testing | | | | | | | | | | | |
| MRI | X | | | | | | | X | | | |
| FDG PET | | X | | | | | | | | | |
| CSF Sampling | | X | | | | | | | | | |
| Blood | | X | X | X | X | X | X | X | X | X | X |
| Collection | | | | | | | | | | | |
| (Safety, | | | | | | | | | | | |
| antibody | | | | | | | | | | | |
| response, etc.) | | | | | | | | | | | |
| Randomization | | X | | | | | | | | | |
| Administration | | X | X | X | X | X | X | | X | | X |
| of AADvac1 or | | | | | | | | | | | |
| placebo | | | | | | | | | | | |

| | Study Visit Number | | | | | | | | |
|---|---|---|---|---|---|---|---|---|---|
| | | | | | | | | V16 | V17 |
| | | | | | | | | (End of | (Follow- |
| | V11 | V11a | V12 | V13 | V14 | V14a | V15 | study) | up) |
| | Week of the Study Visit | | | | | | | | |
| | Week +52 | Week +62 | Week +66 | Week +76 | Week +80 | Week +90 | Week +94 | Week +104 | Week +108 |
| Cognitive | X | | X | | X | | X | X | |
| Testing | | | | | | | | | |
| MRI | X | | | | X | | | X | |
| FDG PET | X | | | | | | | X | |
| CSF Sampling | X | | | | | | | X | |
| Blood | X | X | X | X | X | X | X | X | |
| Collection | | | | | | | | | |
| (Safety, | | | | | | | | | |
| antibody | | | | | | | | | |
| response, etc.) | | | | | | | | | |
| Randomization | | | | | | | | | |
| Administration | | X | | X | | X | | | |
| of AADvac1 or | | | | | | | | | |
| placebo | | | | | | | | | |

Results:

Patients were selected at the screening visit and baseline tests were conducted. The patient selected were between 50 and 85 years of age, who were on a stable dose of an acetylcholinesterase inhibitor (and eventually on memantine). The patients had an MMSE score between 20 and 26 at the time of the screening visit, with a diagnosis of Alzheimer's disease as defined by the NIAA-AA diagnostic criteria laid out in McKhann et al., Alzheimer's & Dementia, (2011) 7:263-269.

In total, 117 patients were selected for treatment with AADvac1 while 79 patients were selected for treatment with placebo. Of these, 100 patients treated with AADvac1 and 63 patients treated with placebo completed the clinical study. The patient demographic was as shown in Table 6.

TABLE 6
Patient profile

| Demographics | AADvac 1 | Placebo | Total |
|---|---|---|---|
| Safety set | (N = 117) | (N = 79) | (N =1 96) |
| Age (years) | | | |
| Mean (SD) | 70.6 (8.37) | 72.7 (6.83) | 71.4 (7.84) |
| Min, Max | 53, 84 | 51,84 | 51,84 |
| Sex n (%) | | | |
| Male | 55 (47.0) | 32 (40.5) | 87 (44.4) |
| Female | 62 (53.0) | 47 (59.5) | 109 (55.6) |
| Race n (%) | | | |
| White | 117 (100.0) | 79 (100.0) | 196 (100.0) |
| BMI(kg/m²) | | | |
| Mean (SD) | 25.9 (3.95) | 26.3 (3.89) | 26.0 (3.92) |
| Min, Max | 17, 36 | 20, 38 | 17, 38 |
| Level of | | | |
| Education (years) | | | |
| Mean (SD) | 12.9 (3.48) | 12.2 (3.32) | 12.6 (3.42) |
| Min, Max | 7, 21 | 6, 19 | 6, 21 |

At baseline, the patient population had the characteristics as shown in Tables 7-12.

TABLE 7
Baseline demographic characteristics of patient population

| Demographics | AADvac1 | Placebo | Total |
|---|---|---|---|
| Safety set | (N = 117) | (N = 79) | (N = 196) |
| Time since AD | 117 | 79 | 196 |
| diagnosis (months) n | | | |
| Mean (SD) | 24.4 (25.46) | 24.3 (16.91) | 24.4 (22.36) |
| Min, Max | 0, 181 | 0, 68 | 0, 181 |
| Duration of previous | 117 | 79 | 196 |
| AChEI treatment | | | |
| (months) n | | | |
| Mean (SD) | 19.1 (23.70) | 20.0 (15.84) | 19.5 (20.85) |
| Min, Max | 4, 184 | 4, 60 | 4, 184 |
| Duration of previous | 18 | 12 | 30 |
| memantine | | | |
| treatment (months) n | | | |
| Mean (SD) | 12.8 (8.99) | 15.5 (10.53) | 13.9 (9.55) |
| Min, Max | 5, 39 | 4, 38 | 4, 39 |

TABLE 8
Baseline disease characteristics of patient population

| Baseline Disease Characteristics | AAD vac 1 | Placebo | Total |
|---|---|---|---|
| Safety Set | (N = 117) | (N = 79) | (N =196) |
| MMSE Mean (SD) | 23.0 (1.89) | 23.2 (1.94) | 23.1 (1.91) |
| Min, Max | 20, 26 | 20, 26 | 20, 26 |
| Hachinski Ischemic | 1.2 (0.86) | 1.2 (0.97) | 1.2 (0.91) |
| Scale Mean (SD) | | | |
| Min, Max | 0, 4 | 0, 4 | 0, 4 |
| Geriatric Depression | 1.4 (1.37) | 1.5 (1.26) | 1.4 (1.33) |
| Scale Mean (SD) | | | |
| Min, Max | 0, 5 | 0, 5 | 0, 5 |
| Fazekas Score Mean (SD) | 1.1 (0.61) | 1.1 (0.61) | 1.1 (0.61) |
| Min, Max | 0, 2 | 0, 2 | 0, 2 |
| Scheltens Score Mean (SD) | 2.5 (0.69) | 2.5 (0.66) | 2.5 (0.67) |
| Min, Max | 1,4 | 2, 4 | 1,4 |

TABLE 9
Patients profiles

| | Prior and Concomitant AD Therapy | | | | | | | | | | | |
|---|---|---|---|---|---|---|---|---|---|---|---|---|
| | AADvac1 | | Placebo | | Total | | AADvac1 | | Placebo | | Total | |
| | NUMBER OF SUBJECTS | | | | | | NUMBER OF SUBJECTS | | | | | |
| | WITH AT LEAST ONE | | | | | | WITH AT LEAST ONE | | | | | |
| Active Ingredient | PRIOR AD THERAPY | | | | | | CONCOMITANT AD THERAPY | | | | | |
| Anticholinesterase | 117 | (100.0) | 79 | (100.0) | 196 | (100.0) | 117 | (100) | 79 | (100) | 196 | (100) |
| Inhibitors | | | | | | | | | | | | |
| DONEPEZIL | 81 | (69.2) | 54 | (68.4) | 135 | (68.9) | 82 | (70.1) | 51 | (64.6) | 133 | (67.9) |
| RIVASTIGMINE | 25 | (21.4) | 22 | (27.8) | 47 | (24.0) | 27 | (23.1) | 21 | (26.6) | 48 | (24.5) |
| GALANTAMINE | 11 | (9.4) | 7 | (8.9) | 18 | (9.2) | 11 | (9.4) | 7 | (8.9) | 18 | (9.2) |
| Other anti-dementia | 18 | (15.4) | 12 | (15.2) | 30 | (15.3) | 29 | (24.8) | 15 | (19.0) | 44 | (22.4) |
| drugs | | | | | | | | | | | | |
| Memantine | 18 | (15.4) | 12 | (15.2) | 30 | (15.3) | 29 | (24.8) | 15 | (9.0) | 44 | (22.4) |

TABLE 10
ApoE4 status of patient population (FAS)

| | AADvac1 | | Placebo | | Total | |
|---|---|---|---|---|---|---|
| ApoE Genotype | (N = 116) | | (N = 77) | | (N = 193) | |
| E2-2 | 0 | | 0 | | 0 | |
| E2-3 | 6 | (5.2) | 4 | (5.2) | 10 | (5.2) |
| E2-4 | 2 | (1.7) | 0 | | 2 | (1.0) |
| E3-3 | 41 | (35.3) | 22 | (28.6) | 63 | (32.6) |
| E3-4 | 54 | (46.6) | 39 | (50.6) | 93 | (48.2) |
| E4-4 | 13 | (11.2) | 12 | (15.6) | 25 | (13.0) |
| ApoE4 Negative | 47 | (40.5) | 26 | (33.8) | 73 | (37.8) |
| ApoE4 Positive | 69 | (59.5) | 51 | (66.2) | 120 | (62.2) |
| ApoE4 Positive | 56 | (48.3) | 39 | (50.6) | 95 | (49.2) |
| (Heterozygous) | | | | | | |
| ApoE4 Positive | 13 | (11.2) | 12 | (15.6) | 25 | (13.0) |
| Homozygous) | | | | | | |

Tables 11A and 11B: Baseline demographic characteristics of patient population by age subgroup (50-67 and 68-85) (FAS)

TABLE 11A

| | Age 50-67 | | Age 68-85 | |
|---|---|---|---|---|
| | n | % | n | % |
| Total | 51 | | 142 | |
| Female | 25 | 49.0 | 81 | 57.0 |
| Male | 26 | 51.0 | 61 | 43.0 |
| AADvac1 | 36 | 70.6 | 80 | 56.3 |
| Placebo | 15 | 29.4 | 62 | 43.7 |
| 0 microhaemorrhages (%) | 43 | 84.3 | 116 | 81.7 |
| ApoE4−/− | 22 | 43.1 | 51 | 35.9 |
| ApoE4−/+ | 23 | 45.1 | 71 | 50.0 |
| ApoE4+/+ | 5 | 9.8 | 20 | 14.1 |
| Memantine | 14 | 27.5 | 29 | 20.4 |

| | | |
|---|---|---|
| Mean Age (years) | 60.5 | 75.3 |
| MMSE (V01) | 23 | 23.13 |
| CDR-SB | 4.23 | 4.51 |
| HIS | 1.02 | 1.25 |
| Fazekas 0/1/2 (%) | 27/59/14 | 10/63/27 |
| CCB z-score | −0.034 | 0.012 |
| NfL (pg/mL) | 18.64 | 23.93 |

TABLE 11B

| | |
|---|---|
| Age 50-67 | Age 58-85 |

| | LHVN* | RHVN* | LHVN | RHVN |
|---|---|---|---|---|
| mean (mm³⁾ | 4295 | 4447 | 4016 | 4206 |
| SD (mm³⁾ | 568.2 | 659.9 | 651.1 | 686 |

*LHVN: Left hippocampus volume normalized
*RHVN: Right hippocampus volume normalised

Tables 12A and 12B: Baseline demographic characteristics of patient population by age subgroup (50-70 and 71-85) (FAS)

TABLE 12A

| | Age 50-70 | | Age 71-85 | |
|---|---|---|---|---|
| | n | % | n | % |
| Total | 74 | | 119 | |
| Female | 39 | 52.7 | 67 | 56.3 |
| Male | 35 | 47.3 | 52 | 43.7 |
| AADvac1 | 52 | 70.3 | 64 | 53.8 |
| Placebo | 22 | 29.7 | 55 | 46.2 |
| ApoE4−/− | 31 | 41.9 | 42 | 35.3 |
| ApoE4−/+ | 32 | 43.2 | 63 | 52.9 |
| ApoE4+/+ | 11 | 14.9 | 14 | 11.8 |
| Memantine | 18 | 24.3 | 26 | 21.8 |

| | | |
|---|---|---|
| Mean Age (years) | 63.2 | 76.5 |
| MMSE (V01) | 22.74 | 23.3 |
| CDR-SB | 4.41 | 4.45 |
| HIS | 1.04 | 1.29 |
| Fazekas 0/1/2 (%) | 24/61/15 | 8/63/29 |
| 0 microhaemorrhages (%) | 86.5 | 70.1 |
| CSF pT181+/− (%) | 30.1/13.7 | 16.7/6.7 |
| CCB z-score | −0.068 | 0.042 |
| NfL (pg/mL) | 18.57 | 23.93 |

TABLE 12B

| | |
|---|---|
| Age 50-70 | Age 71-85 |

| | LHVN | RHVN | LHVN | RHVN |
|---|---|---|---|---|
| mean (mm³⁾ | 4289 | 4451 | 3966 | 4158 |
| SD (mm³⁾ | 615.4 | 673.1 | 627.3 | 672.1 |

### Example 2: Clinical Trial—Cognitive Testing

Cognition testing was performed according to the study schedule shown in Table 5. The tests for cognition included the Clinical Dementia Rating Scale Sum of Boxes (CDR-SB) test, the Alzheimer's Disease Cooperative Study Mild Cognitive Impairment activities of Daily Living (ADCS MCI ADL) questionnaire, the Mini-Mental State Examination (MMSE), and a custom cognitive battery comprising the Cogstate International Shopping List Task, the Cogstate One Card Learning and One Card Back Tasks, the Letter Fluency Test and Category Fluency Test, and the Digit Symbol Coding test.

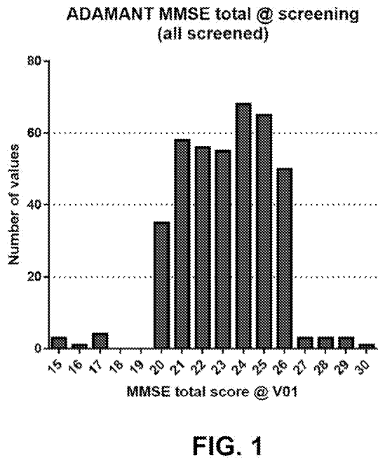

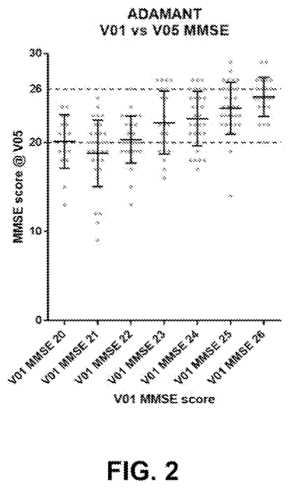

The distribution of total MMSE scores in the patient population measured at the screening visit is shown in FIG. 1. The total score for each patient was tracked over the course of the treatment. For example, FIG. 2 shows the total MMSE for each patient over a 12-week timespan from V01 to V05.

Efficacy of treatment was assessed by the mean change in CDR-SB score, ADCS-MCI-ADL score, or MMSE score from baseline to Week 104. Efficacy of treatment was also assessed by the mean change in standard (composite z-score) of the custom cognitive battery from baseline to Week 104.

Baseline CDR-SB score, ADCS-MCI-ADL score, and MMSE score, as well as other patient demographics information are provided in Table 13A.

TABLE 13A
Patient demographics and baseline data

| | Age 50-67 | | | | Age 68-85 | | | | | |
|---|---|---|---|---|---|---|---|---|---|---|
| | (N=51) | | | | (N = 142) | | | | | |
| | AADvac1 | | Placebo | | AADvac1 | | Placebo | | Total | |
| | (N = 36) | | (N = 15) | | (N = 80) | | (N = 62) | | (N = 193) | |
| AGE (years) | | | | | | | | | | |
| Mean | 59.9 | | 61.7 | | 75.3 | | 75.4 | | 71.4 | |
| Min, Max | 53, 67 | | 51, 67 | | 68, 84 | | 68, 84 | | 51, 84 | |
| SEX n (%) | | | | | | | | | | |
| Male | 18 | (50.0) | 8 | (53.3) | 36 | (45.0) | 24 | (38.7) | 87 | (44.4) |
| Female | 18 | (50.0) | 7 | (46.7) | 44 | (55.0) | 38 | (61.3) | 109 | (55.6) |
| LEVEL OF | | | | | | | | | | |
| EDUCATION (years) | | | | | | | | | | |
| Mean (SD) | 13.54 | (3.00) | 11.87 | (2.85) | 12.64 | (3.67) | 12.31 | (3.45) | 12.6 | (3.42) |
| Min, Max | 8, 19 | | 6, 17 | | 7, 21 | | 6, 19 | | 6, 21 | |
| APOE4 | | | | | | | | | | |
| APOE4 Negative | 17 | (48.6) | 5 | (33.3) | 30 | (37.5) | 21 | (33.9) | 73 | (37.8) |
| APOE4 Positive | 15 | (42.9) | 8 | (53.3) | 40 | (50.0) | 31 | (50.0) | 95 | (49.2) |
| (Heterozygous) | | | | | | | | | | |
| APOE4 Positive | 3 | (8.6) | 2 | (13.3) | 10 | (12.5) | 10 | (16.1) | 25 | (13.0) |
| (Homozygous) | | | | | | | | | | |
| MMSE at baseline | | | | | | | | | | |
| Mean (SD) | 23.0 | (1.91) | 22.9 | (2.26) | 23.0 | (1.90) | 23.27 | (1.87) | 23.1 | (1.91) |
| Min, Max | 20, 26 | | 20, 26 | | 20, 26 | | 20, 26 | | 20, 26 | |
| CDR-SB at baseline | | | | | | | | | | |
| Mean (SD) | 4.28 | (1.91) | 4.1 | (1.43) | 4.4 | (2.15) | 4.6 | (2.61) | 4.4 | (2.21) |
| Min, Max | 1.5, 11 | | 1, 6 | | 1, 11 | | 1, 12 | | 1, 12 | |
| NfL at baseline | | | | | | | | | | |
| Mean (SD) | 19.2 | (11.63) | 17.3 | (4.53) | 22.5 | (8.92) | 22.0 | (9.21) | 21.3 | (9.42) |

| | | | | | |
|---|---|---|---|---|---|
| Min, Max | 6, 65.88 | 6.9, 23.25 | 8.4, 50.9 | 10.4, 64.4 | 6, 65.9 |

The results of cognition testing in patients at Week 104 compared to baseline are shown in Tables 13B and 13C.

TABLE 13B
Mixed model repeated measures (MMRM) analysis of change from baseline at Week 104

| | | | AADvac1 | | Placebo | | AADvac1 - | |
|---|---|---|---|---|---|---|---|---|
| | Timepoint | Statistic | (n = 116) | | (n = 77) | | Placebo | |
| CDR-SB | Week | n | 96 | | 61 | | | |
| | 104 | LS mean (SE) | 3.35 | (0.314) | 3.23 | (0.393) | 0.12 | (0.504) |
| | | 95% confidence | 2.73, 3.97 | | 2.45, 4.01 | | −0.87, 1.12 | |
| | | interval | | | | | | |
| | | p-value | | | | | 0.806 | |
| ADCS- | Week | n | 99 | | 61 | | | |
| MCI-ADL | 104 | LS mean (SE) | −8.2 | (0.95) | −7.8 | (1.20) | −0.4 | (1.53) |
| | | 95% confidence | −10.1, −6.3 | | −10.1, −5.4 | | −3.4, 2.6 | |
| | interval | | | | | | | |
| | | p-value | | | | | 0.794 | |
| MMSE | Week | n | 96 | | 60 | | | |
| | 104 | LS mean (SE) | −5.6 | (0.52) | −4.9 | (0.65) | −0.6 | (0.83) |
| | | 95% confidence | −6.6, −4.5 | | −6.2, −3.7 | | −2.3, 1.0 | |
| | interval | | | | | | | |
| | | p-value | | | | | 0.459 | |
| Cognitive | Week | n | 91 | | 56 | | | |
| Battery | 104 | LS mean (SE) | −0.3537 | (0.05533) | −0.3017 | (0.06975) | −0.0519 | (0.08919) |

| | | | |
|---|---|---|---|
| 95% confidence | −0.4630, −0.2443 | −0.4396, −0.1639 | −0.2282, 0.1244 |
| interval | | | |
| p-value | | | 0.561 |

ANCOVA was also used to assess the difference in mean change from baseline to Week 104 between the two treatment groups. ANCOVA modelled the change from baseline to Week 104, including terms for treatment group, pooled country, sex, age, years of education, ApoE4 status, baseline MRI hippocampal volume, and baseline CDR-SB. For endpoints other than CDR-SB, the baseline value of the given assessment was included as an ANCOVA term. From this ANCOVA model, the LS mean treatment difference (AADvac1—Placebo) was presented along with the 95% CI and the two-sided p-value for treatment effect. LS means were estimated at the mean level of continuous covariates (age, years of education, baseline MRI hippocampal volume, baseline CDR-SB) with the classification covariates (sex, pooled country, ApoE4) weighted to be proportional to the number of subjects at each level in the FAS at baseline. These covariates adjust the means at each level of the interaction of time (visits) and treatment. The results are shown in Table 13C.

TABLE 13C
ANCOVA_analysis of change from baseline at Week 104

| | | 95% CI of | |
|---|---|---|---|
| Assessment | Difference | difference | p-value |
| CDR-SB | −0.36 | −1.306, 0.589 | 0.456 |
| CCB8 | 0.0008 | −0.16999, 0.17155 | 0.993 |
| ADCS-MCI-ADL18 | 1.1 | −1.76, 3.90 | 0.457 |
| ADCS-MCI-ADL24 | 0.5 | −3.00, 3.91 | 0.795 |

The results of patients on the CDR-SB scores may be further analyzed by subgroups as shown in Table 14.

TABLE 14
CDR-SB scores at Week 104 by subgroups

| Sub-group | Timepoint | | AADvac1-Placebo |
|---|---|---|---|
| ≥50 and <65 | Week | Difference | −0.5976 |
| age-group | 104 | 95% confidence | −3.8953, 2.7002 |
| | | interval | |
| ≥65 and ≤85 | Week | Difference | 0.0502 |
| age-group | 104 | 95% confidence | −0.9670, 1.0674 |
| | | interval | |
| | | Difference | −0.9700 |
| ≥50 and <71 | Week | 95% confidence | −2.9250, 0.9850 |
| age-group | 104 | interval | |
| ≥71 and ≤85 | Week | Difference | 0.15 |
| age-group | 104 | 95% confidence | −0.94, 1.24 |
| | | interval | |
| Male | Week | Difference | −0.6976 |
| | 104 | 95% confidence | −2.1845, 0.7893 |
| | | interval | |
| Female | Week | Difference | 0.7992 |
| | 104 | 95% confidence | −0.5985, 2.1970 |
| | | interval | |
| Overall | Week | Difference | 0.1241 |
| | 104 | 95% confidence | −0.8720, 1.1202 |
| | | interval | |

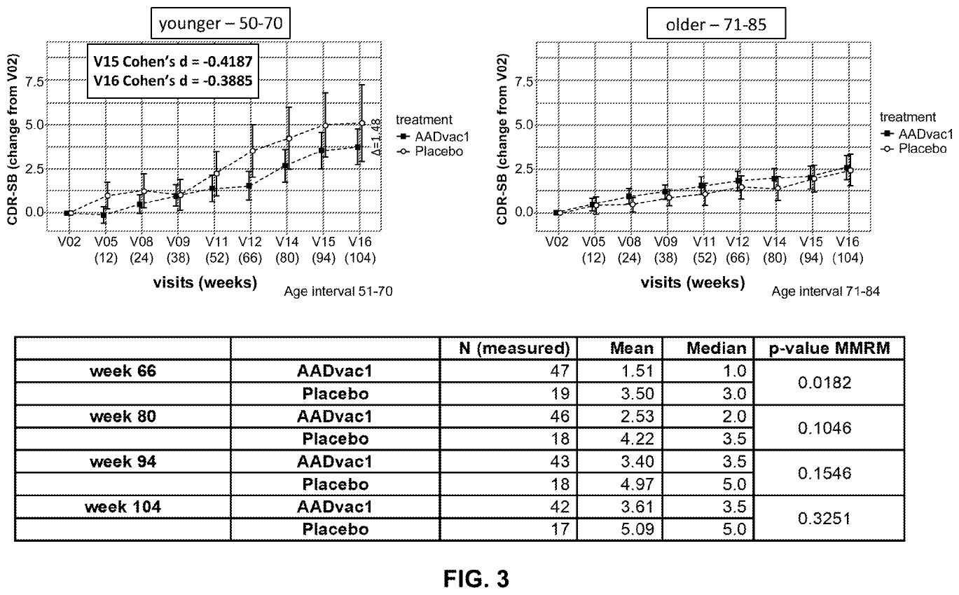

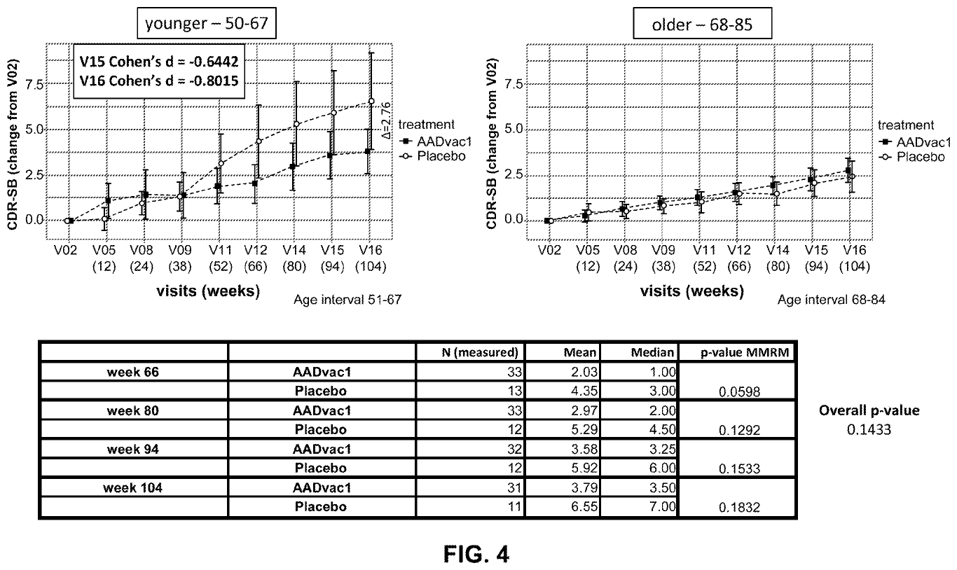

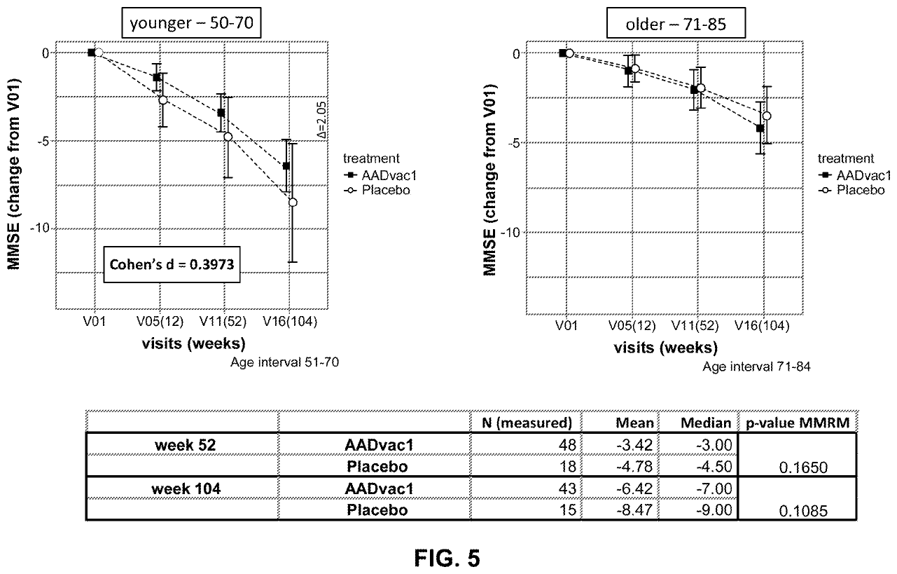

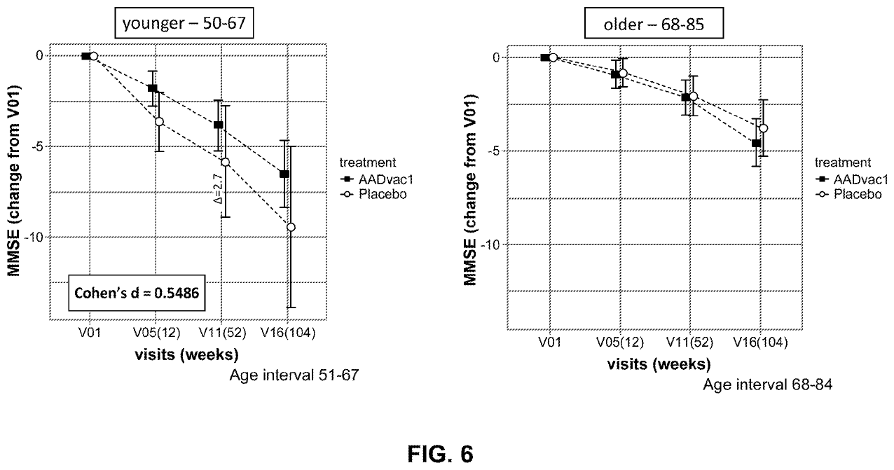

Similarly, FIGS. 3-6 show the change in CDR-SB scores of patients from different age subgroups as treatment progresses.

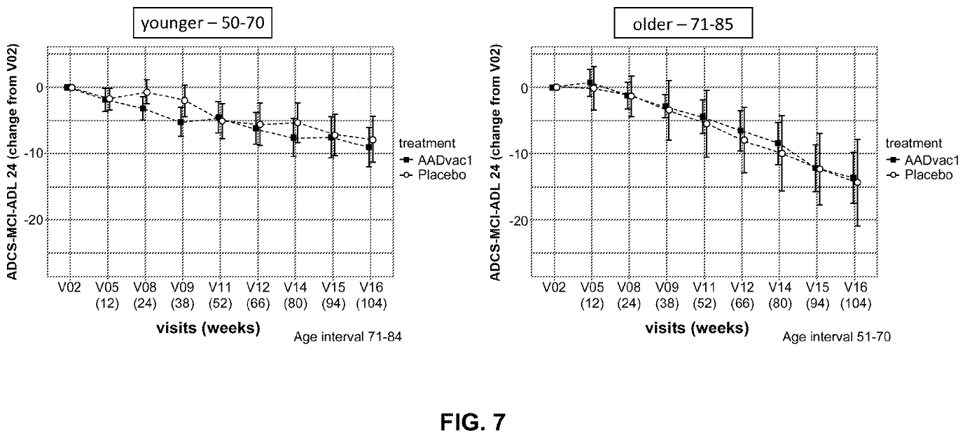

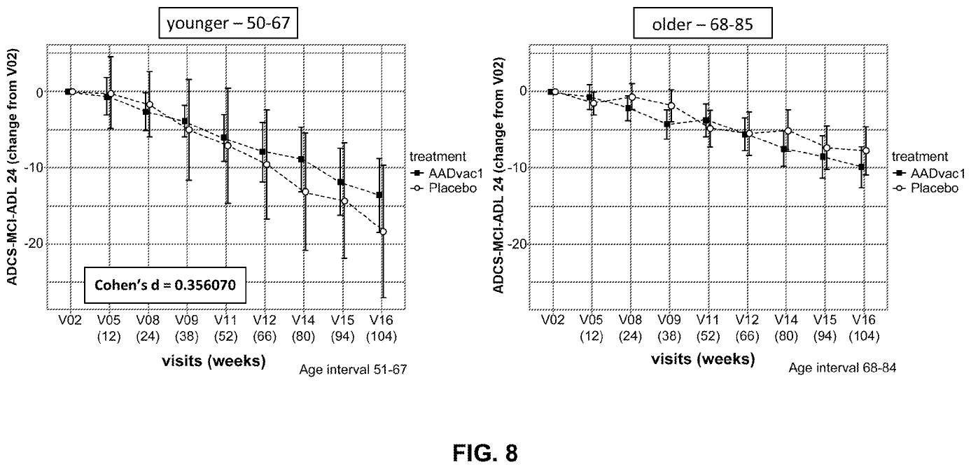

The ADCS-MCI-ADL scores were also assessed over the course of the study, as shown in FIGS. 7-8.

### Example 3: Antibody Response

Blood collection for testing of antibody response was performed according to the study schedule as shown in Table 5. The titers of antibodies in the patient plasma were analyzed by indirect and quantitative ELISA.

Method: Determination of Human Antibody Titres by Indirect ELISA

The titers of vaccine-induced antibodies were determined using serially diluted plasma samples with indirect ELISA. AADvac1 was immobilized on microtiter plates (High Binding strip plates, Greiner Bio One, Frickenhausen, Germany) at a final concentration of 5 pg/ml, KLH was immobilized at a final concentration of 2.5 pg/ml and incubated at 37° C. for 2 hrs. After the blocking with PBS-0.05% Tween 20 for 1 hr at 25° C., the plated samples were incubated overnight at 4° C. with serially diluted serum samples of the patients. After washing, bound antibodies were detected by anti-human immunoglobulins (IgG and IgM) conjugated to horseradish peroxidase (Thermo Scientific, Pierce, Rockford, Ill., USA). The amount of bound secondary antibodies was detected with the chromogenic substrate TMB One (KEM-EN-TEC Diagnostic, Taastrup, Denmark). The resulting signal was compared with that obtained for the patient's serum collected at baseline. The titer of the antibodies in the serum is determined by measuring the highest dilution at which the absorbance at 450 nm was defined at least twice the absorbance of equally diluted preimmunization serum samples. To ensure the assay consistency and quality, quality control samples were used with two concentrations of the humanized version of monoclonal antibody DC8E8 (AX004) on all plates.

Results: AADvac1 Stimulates Production of IgG and IgM

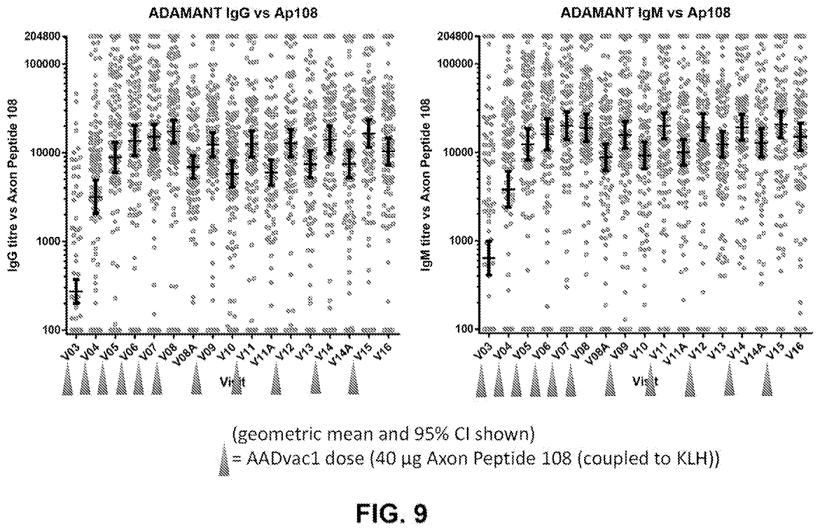

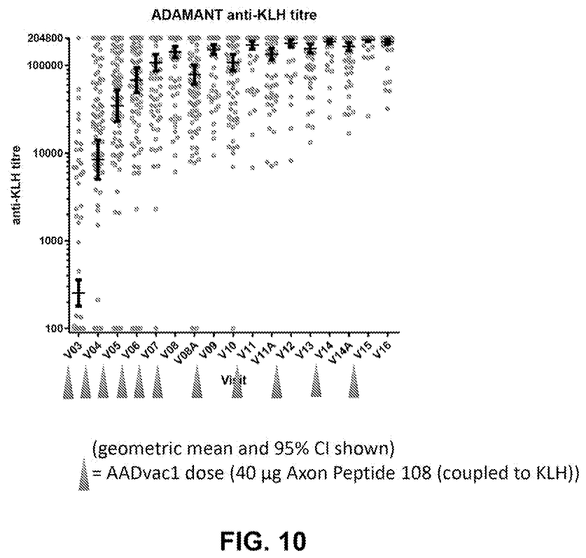

The area-under-the-curve reflects cumulative amount of antibody produced by the patient over a given time span. As shown in FIGS. 9A and 9B, vaccination of patients with 6 initial doses and 5 booster doses of 40 μg AADvac1 stimulated production of both IgG and IgM against the peptide. Similarly, anti-KLH antibodies were also detected at high titers in the plasma of AADvac1-treated patients (FIG. 10).

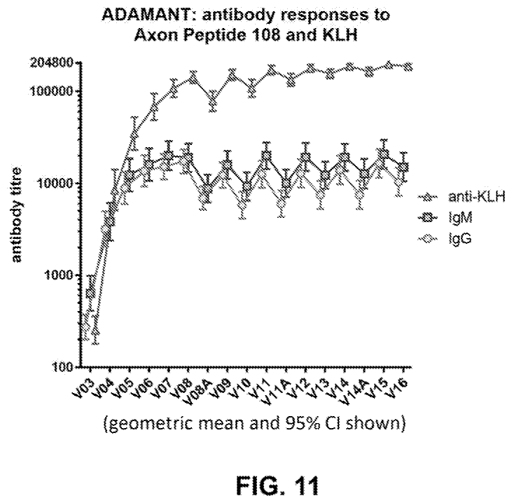

Furthermore, as shown in FIG. 11, anti-AADvac1 IgG and IgM and anti-KLH antibody titers increased with individual doses (initial dosing regimen of six doses, once per month) and responded to booster doses (five boosters, once per three months). Up to 96.5% of treated patients develop an IgG response after the initial 6-dose vaccination regimen, as shown in Table 15. Overall, about 98.2% of treated patients developed an IgG response.

TABLE 15
Response to vaccination regimen over time

| | V03 | V04 | V05 | V06 | V07 | V08 |
|---|---|---|---|---|---|---|
| Number of values | 116 | 115 | 115 | 114 | 114 | 114 |
| Minimum | 100 | 100 | 100 | 100 | 100 | 100 |
| 25% Percentile | 100 | 338 | 4582 | 5383 | 7152 | 8796 |
| Median | 100 | 6041 | 11362 | 17616 | 14303 | 15776 |
| 75% Percentile | 673 | 13822 | 40020 | 62708 | 56271 | 49202 |
| Maximum | 46138 | 204800 | 204800 | 204800 | 204800 | 204800 |
| Mean | 2034 | 22344 | 34763 | 48389 | 41123 | 43077 |
| Std. Deviation | 6271 | 47630 | 51826 | 64615 | 56907 | 56242 |
| Std. Error of Mean | 582.3 | 4442 | 4833 | 6052 | 5330 | 5268 |
| Lower 95% CI of mean | 880.6 | 13545 | 25189 | 36400 | 30564 | 32641 |
| Upper 95% CI of mean | 3187 | 31142 | 44337 | 60379 | 51683 | 53513 |
| Geometric mean | 272.8 | 3182 | 8868 | 13676 | 15014 | 17350 |
| Lower 95% CI of geo. | 200.3 | 2065 | 5968 | 9293 | 10960 | 12809 |
| mean | | | | | | |
| Upper 95% CI of geo. | 371.6 | 4902 | 13176 | 20126 | 20569 | 23500 |
| mean | | | | | | |
| IgG responder | 34.5 | 77.4 | 88.7 | 91.2 | 96.5 | 96.5 |
| percentage | | | | | | |

Method: Quantitative ELISA for Determination of Antibodies Specific to Peptide 108 in Human Serum

The amounts of human antibodies specific to immunogenic peptide 108 was determined using quantitative ELISA. Immunogenic peptide 108 was immobilized on microtiter plates (High Binding strip plates, Greiner Bio One, Frickenhausen, Germany) at a final concentration of 5 μg/ml and incubated at 37° C. for 2 hrs. A humanized version of monoclonal antibody DC8E8 (AX004) standard was diluted in standard diluent (normal human serum (IPLA-SER, Innovative Research, Inc.), diluted 2000× in PBS-0.05% Tween 20, 5% BSA and 1% casein) in serial 2,3-fold dilutions starting from 50 pg/ml, following by 21.74; 9.452; 4.109; 1.787; 0.777; 0.338 and 0.147 ng/ml. Then, after the blocking with PBS-0.05% Tween 20 for 1 hr at 25° C., 50 μl/well of each dilution of standard, serum samples, diluted 2000× in sample diluent (PBS-0.05% Tween 20, 5% BSA and 1% casein), negative control (standard diluent), and blank (sample diluent) were added into the peptide immobilized plate and incubated overnight at 4° C. After washing, bound antibodies were detected with anti-human immunoglobulins (IgG, IgM) conjugated to horseradish peroxidase (Thermo Scientific, Pierce, Rockford, Ill., USA) using chromogenic substrate TMB One (KEM-EN-TEC Diagnostic, Taastrup, Denmark). Then, analysis of results was done by Graphpad Prism software.

Method: Quantitative Assay for Determination of Antibodies Specific to Tau 151-391 in Human Serum

The amounts of antibodies specific to tau 151-391/4R was determined using the fluorescent tau-bead method. A standard was diluted in standard diluent (normal human serum, (IPLA-SER, Innovative Research, Inc.) diluted 500× in PBS-0.05% Tween 20, 5% BSA and 1% casein) in serial 3-fold dilutions starting from 80 ng/ml, followed by 26.6; 8.88; 2.96; 0.98; 0.32; 0.109 and 0.036 ng/ml. Next, 50 μl/well of each dilution of standard, serum samples 1000× diluted in sample diluent (PBS-0.05% Tween 20, 5% BSA and 1% casein), negative control (standard diluent), and blank (sample diluent) were applied to the Bio-Plex plate. Then, 0.5 μl/well of tau coupled magnetic beads (MagPlex-c-Microspheres 026, Bio-Rad, MC10026-01) were added into each sample of the Bio-Plex plate and incubated with mixing on the shaker (Titramax 100, overnight/4° C., 950 rpm) overnight at 4° C. After washing, the plate was incubated with secondary antibody IgG/PE (diluted 2000× in PBS-T, Sigma-Aldrich) for 1 hour at room temperature with mixing on a shaker (Thermo Labsystem, max speed). Then, analysis of results was done on a BioPlex 200 reader using Bio-Plex Manager 6.1 software.

Results: Quantitative ELISA Confirms the Amount of Specific Antibodies

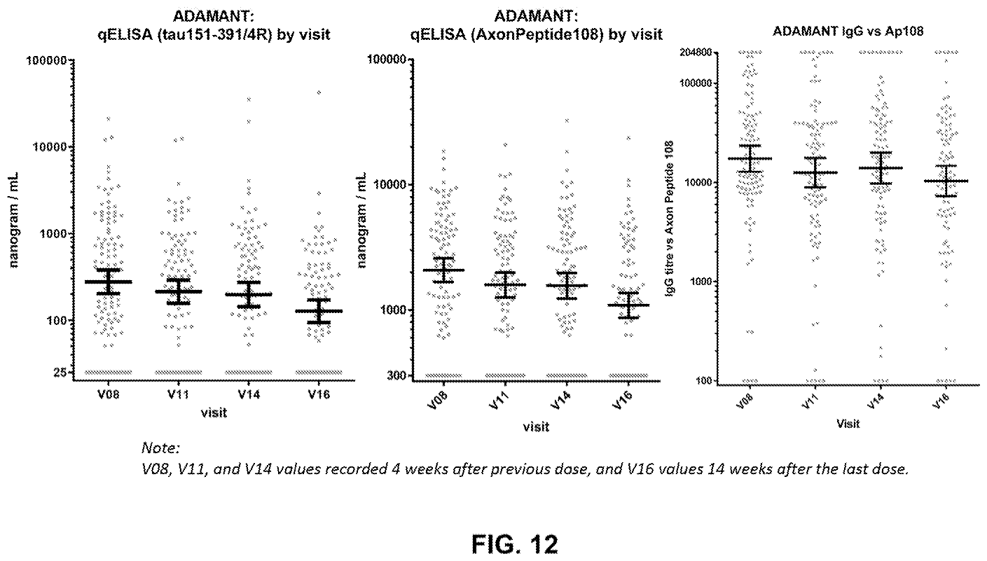

As shown in FIG. 12, quantitative ELISA confirmed that 40 μg AADvac1 stimulated production of antibodies against tau151-394, and AADvac1.

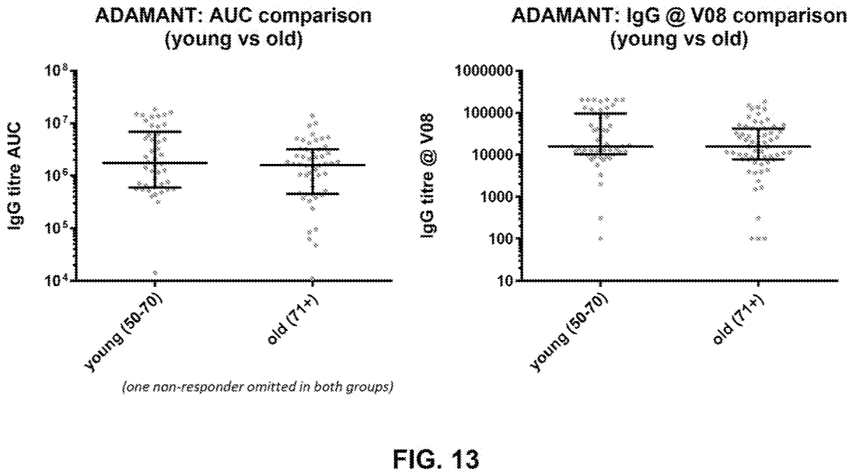

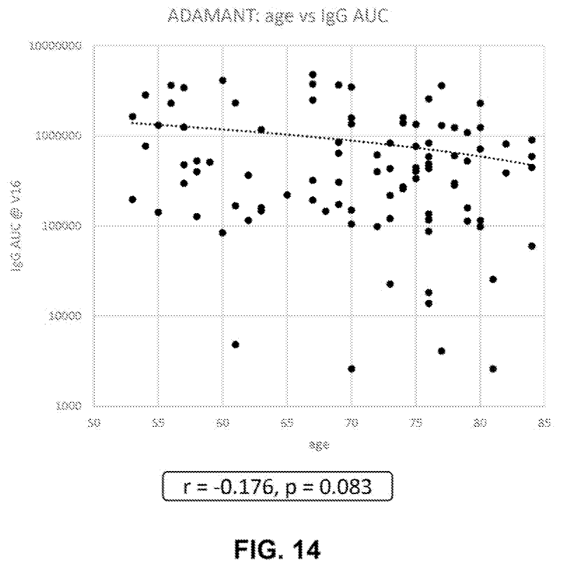

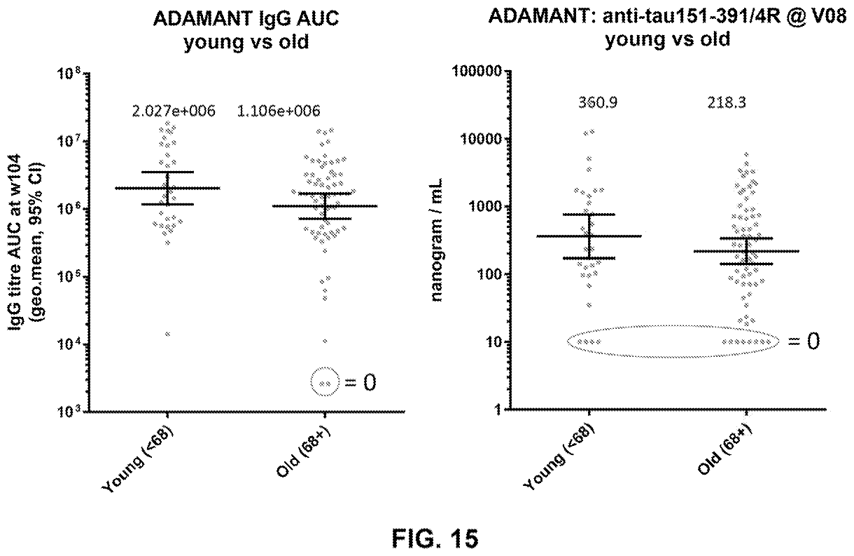

Additionally, as shown in FIGS. 13 and 14, when results were analyzed by age-group, the younger subgroup (50-70 years old) showed a higher antibody response than the older subgroup. This effect was more pronounced when younger patients (67 years old) were compared to older patients (68 years old), as shown in FIG. 15. Specifically, IgG Titre Area Under the Curve (AUC) values were observed to be twice as high in the younger subgroup compared to the older subgroup. Calculation of the AUC was performed as follows: For each subject, the titre Area under the Curve (AUC) from baseline was calculated for each visit. The AUC of titres was calculated using the trapezoidal rule as follows: Let A0, A4, A8, . . . , A104 denote the titre measurements at each of the timepoints mentioned above (i.e., each visit; A0 is the value measured at baseline, A4 is the value measured at week 4, A8 is the values measured at week 8, . . . , A104 is the value measured at week 104) and T0, T4, T8, . . . , T104 are the corresponding timepoints (actual time measured in study days); the AUC0-104 is calculated as: AUC0-104=[(A0+A4)×(T4−T0)+(A4+A8)×(T8-T4)+ . . . +(A92+A104)×(T104−T92)]/2, i.e., AUC between baseline and a visit denoted as j (where i denotes the previous visit) was calculated as follows: AUC0-j=[(A0+A4)×(T4−T0)+ . . . +(Ai+Aj)×(Tj−Ti)]/2. All values at available timepoints up to the relevant visit were used to calculate the AUC for that visit. If a value was missing at a timepoint (between 0 and j), then the next available value and timepoint was used, as long as this timepoint occurred prior to the visit for which the AUC was being calculated (i.e. prior to visit j). An AUC was not derived if the baseline sample was missing. If a value was missing at timepoint j then AUC for that timepoint was not calculated.

### Example 4: Biomarkers for Alzheimer's Disease

Blood and cerebrospinal fluid for testing of biomarkers were collected according to the study schedule as shown in Table 5. The levels of pT217, pT181, neurofilament light (NfL) and total tau, were measured according to the methods described below.

In total, 60 patients provided CSF samples during at least one of the study visits; 27 of those provided both a baseline sample and an end-of-study sample.

Method: Determination of Total Tau and pT181 Tau in Cerebrospinal Fluid

Measurements of total tau and pT181 tau in cerebrospinal fluid were performed using an Innotest hTAU Ag and phospho-tau (181P) ELISA assay kits, respectively, according to the instructions of the manufacturer.

Method: Determination of pT217 Tau in Cerebrospinal Fluid by pT217 Digital ELISA Assay

A high-sensitive format digital ELISA was set up using a Simoa HD1 analyzer (Quanterix). Reagents for digital ELISA were prepared according to the Quanterix Homebrew Assay Development Guide with the following details. DC2E7 antibody was used as a capture antibody and DC2E2 antibody was used as a detector antibody. DC2E7 was coupled to magnetic beads (Quanterix) at a concentration of 0.5 mg/ml. Detector antibody was prepared by biotinylation of DC2E2, whereby a 120-fold excess of Biotin, EZ-Link NHS-PEG4-Biotin (Thermo Scientific, #21329) over antibody concentration was used. The DC2E7 calibrator (synthetic peptide carrying epitopes of DC2E7 and DC2E2) was diluted in calibrator diluent (20 mM sodium phosphate pH 7.4, 137 mM NaCl, 2.7 mM KCl, 2% BSA) in serial 1.6-fold dilutions starting from 20 pg/ml, to yield calibrants at 2000, 1250, 781.25, 488.28, 305.18, 190.73, 119.21, 74.5 and 0 pg/ml. Prepared calibrator concentrations were mixed in a 3:1 ratio with sample diluent (80 mM sodium phosphate pH 7.4, 548 mM NaCl, 10.8 mM KCl, 0.04% casein and 0.4% Tween 20) containing heterophilic blocker. CSF samples from control individuals were diluted with sample diluent in the same way as described. Spike recovery of CSF sample was performed using spikes of CSF with 1200, 700, 200 and 0 pg/ml of DC2E7 calibrator. Diluted calibrators and samples were pipetted into a 96-well plate and inserted into the Simoa HD1 analyzer. Specifically, capture antibody DC2E7 beads diluted in bead diluent, detector antibody DC2E2 diluted in detector diluent to 4 μg/ml, Steptavidin beta-galactosidase (SbG) diluted in SbG diluent to 150 nM and substrate resorufin-β-D-galactopyranoside (RGP) were all inserted into the analyzer (all buffers, SBG and RGP were obtained from Quanterix). The assay was programmed into the Simoa 1.5 software and analysis was performed. Post-analysis evaluation was done by Graphpad Prism and/or by software included in Simoa 1.5 software.

Results: pT217 and pT181 Immunoassays were Specific for Alzheimer's Disease

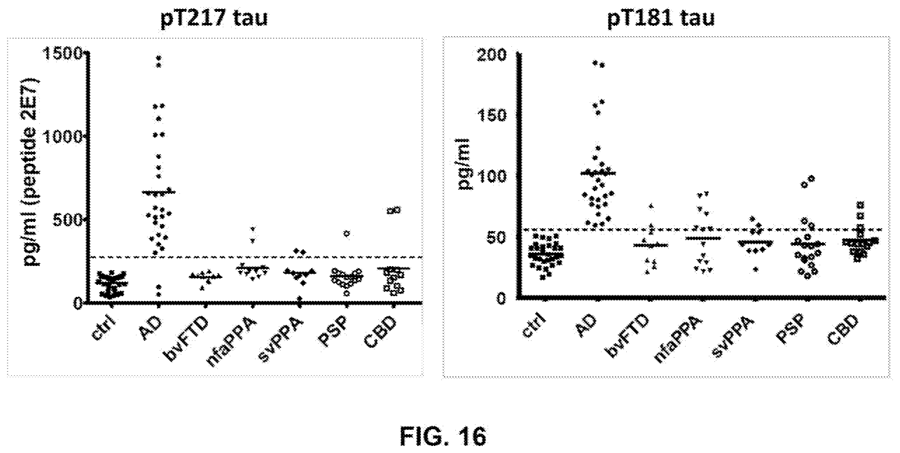

As shown in FIG. 16, the levels of pT217 and pT181 tau in cerebrospinal fluid were elevated in patients with Alzheimer's Disease, compared to control patients, and patients diagnosed with other neurological diseases such as behavioral variant frontotemporal dementia (bvFTD), nonfluent agrammatic variant of primary progressive aphasia (nfaPPA), semantic variant of primary progressive aphasia (svPPA), progressive supranuclear palsy (PSP), and corticobasal degeneration (CBD).

Results: pT217 and pT181 Show Disease Progression

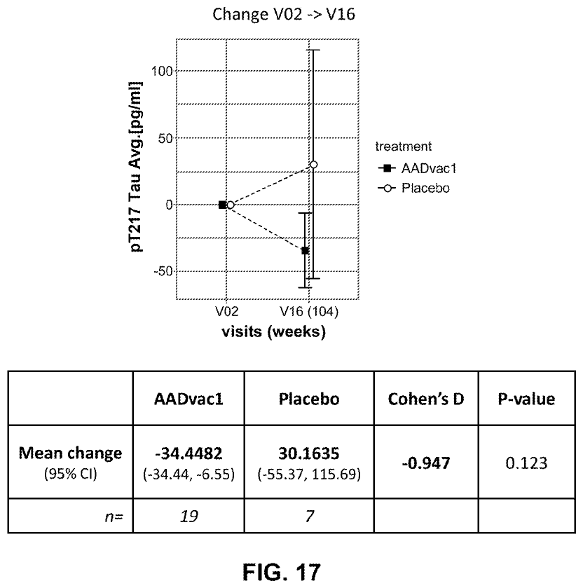

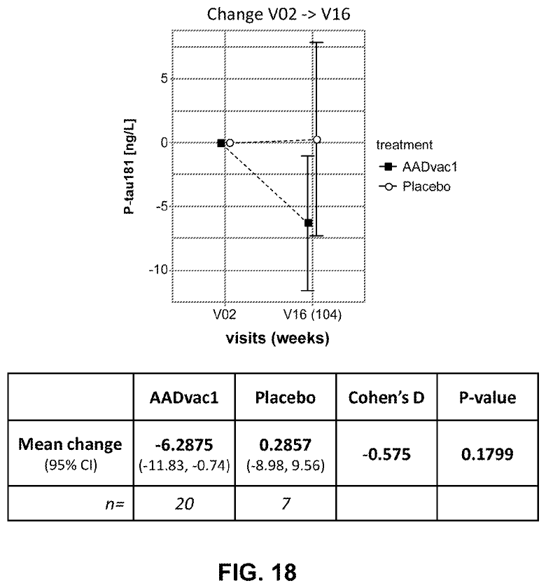

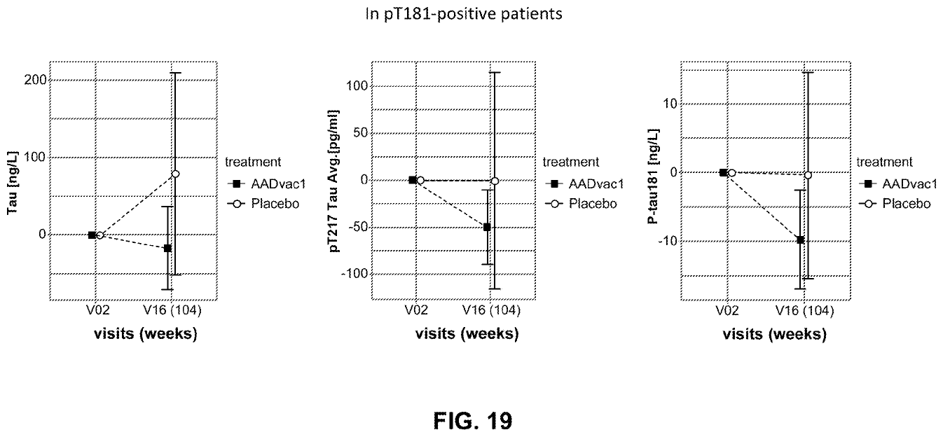

As shown in FIGS. 17 and 18, levels of pT217 and pT181 tau (ng/L) were reduced in patients that received AADvac1 treatment, compared to patients that received the placebo treatment. In patients that were positive for pT181 tau, levels of tau, pT217 and pT181 tau were reduced in AADvac1-treated patients, as shown in FIG. 19.

Method: Determination of Neurofilament Light (NfL) in the Blood Plasma

The Simoa NF-light® assay—a digital immunoassay (Quanterix corporation, MA, USA), was used for the measurement of neurofilament light in the blood samples. Sample measurements were carried out according to the manufacturer's protocol. The lower limit of detection of the assay is 0.038 μg/mL while the lower limit of quantification is 0.174 μg/mL.

Results: Neurofilament Light (NfL) is a Biomarker of Ongoing Neurodegeneration

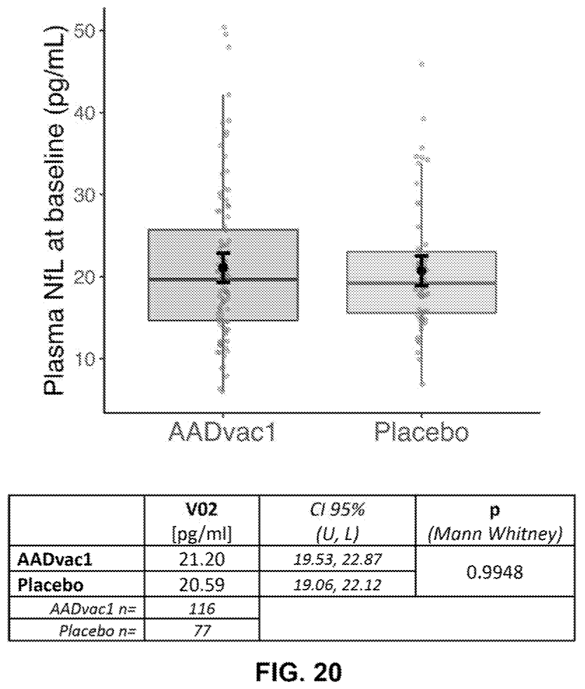

The distribution of NfL in the tested patient population is shown in FIG. 20. At baseline, the levels of plasma NfL were not different between patients who were subsequently treated with AADvac1 or with placebo. The change in levels of NfL over baseline in plasma collected from patients treated with AADvac1 correlated with disease progression. As shown in Table 16, the change in levels of NfL from baseline in the study population also correlated with changes in brain volume, brain metabolism, and cognitive performance.

TABLE 16
Change in levels of NfL from baseline correlates with changes
in brain volume, brain metabolism, and cognitive performance

| | | | r (Spearman |
|---|---|---|---|
| | Change over study course | p-value | coefficient) |
| Brain Volume | Cortical volume | 0.0142 | −0.1985 |
| | Temporal lobe volume | 0.0010 | −0.2653 |
| | Frontal lobe volume | 0.0266 | −0.1793 |
| | Ventricular volume | 0.0020 | 0.2517 |
| Brain | FDG-PET composite | 0.0229 | −0.3591 |
| metabolism | | | |
| Cognitive | CDR-SB | 0.0091 | 0.2076 |
| performance | ADL24 | 0.0050 | −0.2250 |
| | MMSE (from V05 to V16) | 0.0059 | −0.2210 |

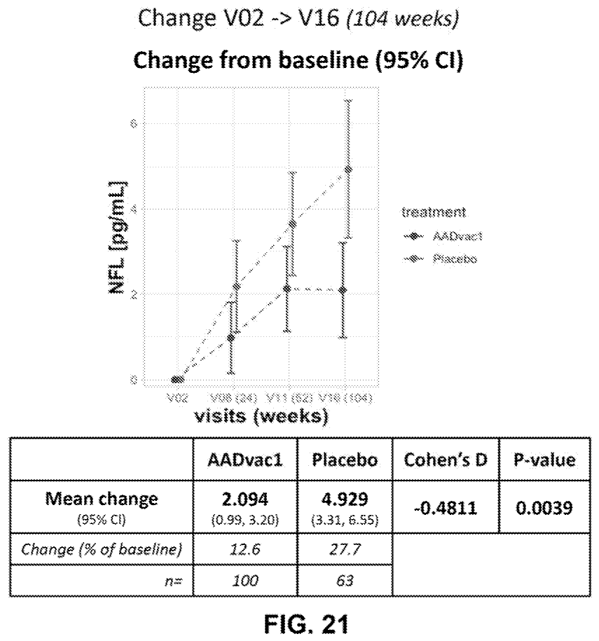

As shown in FIG. 21 and Table 17, treatment with AADvac1 reduced accumulation of NfL in the plasma over the course of the treatment, i.e., reduced accumulation of NfL in the plasma by 58% compared to placebo, indicating that it slowed the progression of neurodegeneration. Furthermore, as shown in Table 18, AADvac1 reduced the accumulation of plasma NfL across all patient subgroups. For comparison to the results obtained with AADvac1 and placebo, respectively, the expected accumulation of plasma NfL is about 14% for healthy controls and about 24% for AD patients. See Mattsson et al., 2019.

TABLE 17
Differences in the change in levels
of NfL due to the AADvac1 treatment

| | AADvac1 | Placebo | Cohen's D | p-value |
|---|---|---|---|---|
| Mean change (pg/mL) | +2.094 | +4.929 | −0.4811 | 0.0039 |
| Change (% of | 12.6 | 27.7 | | |
| baseline) | | | | |
| n | 100 | 63 | | |

TABLE 18
Changes from baseline of plasma NfL levels across all patient subgroups

| | | | | | | Cohen's | Bootstrap |
|---|---|---|---|---|---|---|---|
| | | AADvac1 | | Placebo | | D | p-value |
| Total | Mean change (n) | 2.094 | (100) | 4.929 | (63) | −0.4811 | 0.0039 |
| population | 95% confidence | 0.99, 3.2 | | 3.32, 6.53 | | | |
| | interval | | | | | | |
| Males | Mean change (n) | 1.392 | (49) | 5.302 | (25) | −0.6711 | 0.0149 |
| | 95% confidence | −0.148, 2.931 | | 2.565, 8.039 | | | |
| | interval | | | | | | |
| Females | Mean change (n) | 2.772 | (51) | 4.676 | (38) | −0.3204 | 0.1458 |
| | 95% confidence | 1.154, 4.391 | | 2.629, 6.722 | | | |
| | interval | | | | | | |
| 50-70 | Mean change (n) | 2.116 | (46) | 6.319 | (18) | −0.8010 | 0.0198 |
| years old | 95% confidence | 0.670, 3.562 | | 3.269, 9.369 | | | |
| | interval | | | | | | |
| 71-85 | Mean change (n) | 2.079 | (54) | 4.366 | (45) | −0.3649 | 0.0760 |
| years old | 95% confidence | 0.409, 3.748 | | 2.429, 6.304 | | | |
| | interval | | | | | | |
| Scheltens | Mean change (n) | 1.453 | (62) | 5.667 | (40) | −0.7952 | 0.0003 |
| score 0-2 | 95% confidence | 0.254, 2.652 | | 3.700, 7.635 | | | |
| | interval | | | | | | |
| Scheltens | Mean change (n) | 3.145 | (38) | 3.632 | (23) | −0.0730 | 0.79 |

| | | | |
|---|---|---|---|
| score >2 | 95% confidence | 0.951, 5.338 | 0.777, 6.487 |
| | interval | | |

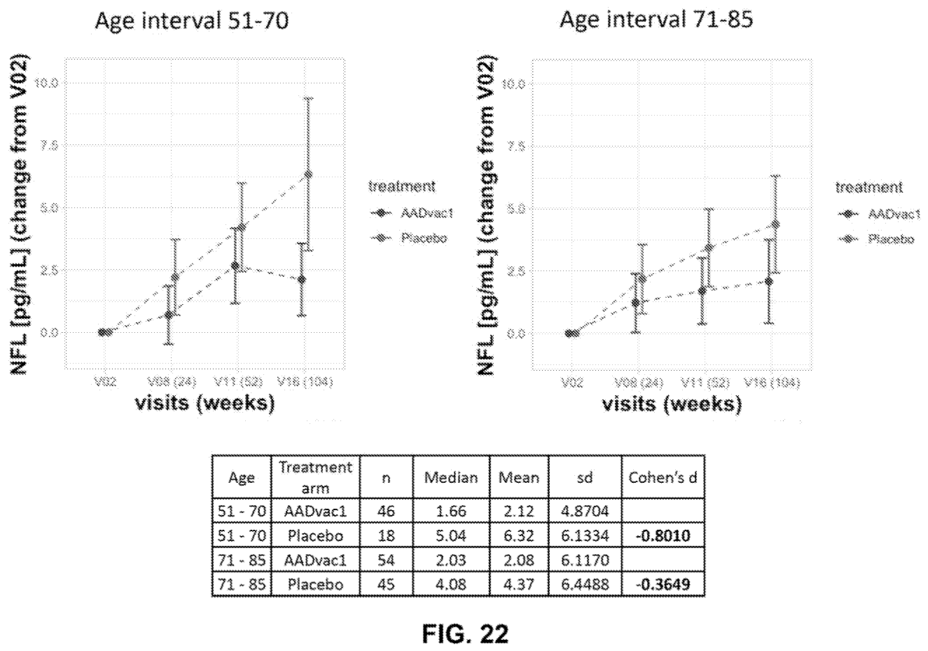

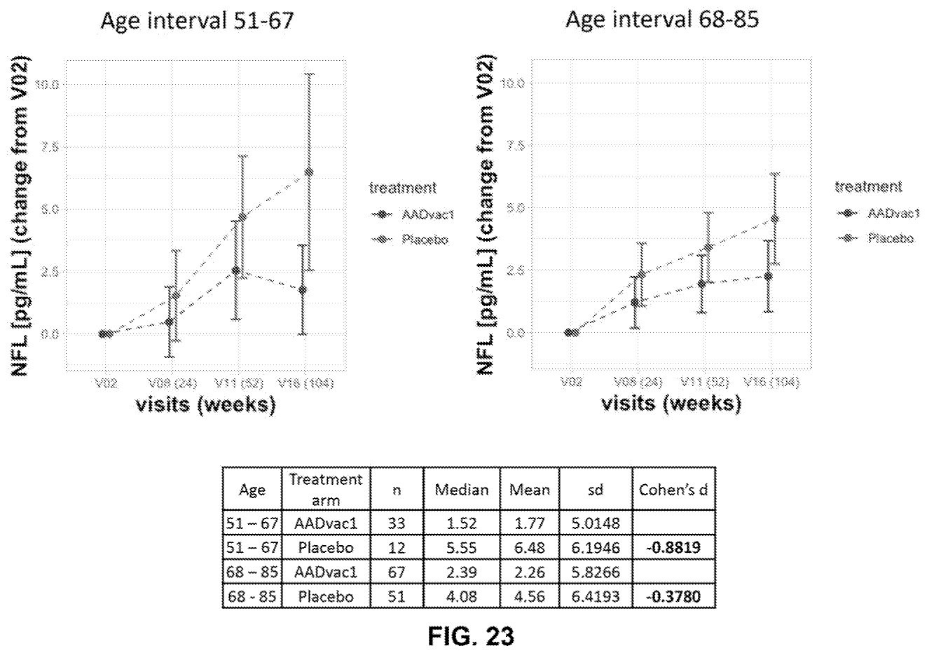

The effect of treatment on the levels of NfL are shown in Tables 19-20 and FIGS. 22-23. Levels of NfL after treatment showed differences across different age groups.

TABLE 19
Changes in the levels of NfL in 51-70 and 71-85 age groups

| | Treatment | | Median | Mean | | |
|---|---|---|---|---|---|---|
| Age | Arm | n | change | change | SD | Cohen's D |
| 51-70 | AADvac1 | 46 | 1.66 | 2.12 | 4.8704 | −0.8010 |
| 51-70 | Placebo | 18 | 5.04 | 6.32 | 6.1334 | |
| 71-85 | AADvac1 | 54 | 2.03 | 2.08 | 6.1170 | −0.3649 |
| 71-85 | Placebo | 45 | 4.08 | 4.37 | 6.4488 | |

TABLE 20
Change in the levels of NfL from baseline
in 51-67 and 68-85 age groups

| | Treatment | | | | | |
|---|---|---|---|---|---|---|
| Age | Arm | n | Median | Mean | SD | Cohen's D |
| 51-67 | AADvac1 | 33 | 1.52 | 1.77 | 5.0148 | −0.8819 |
| 51-67 | Placebo | 12 | 5.55 | 6.48 | 6.1946 | |
| 68-85 | AADvac1 | 67 | 2.39 | 2.26 | 5.8266 | −0.3780 |
| 68-85 | Placebo | 51 | 4.08 | 4.56 | 6.4193 | |

Method: Determination of Human Total Tau in CSF

Measurement of total tau in cerebrospinal fluid was performed using an Innotest hTAU Ag ELISA assay kit according to the instructions of the manufacturer.

Results: Total Tau in CSF is a Biomarker that Correlates with AD

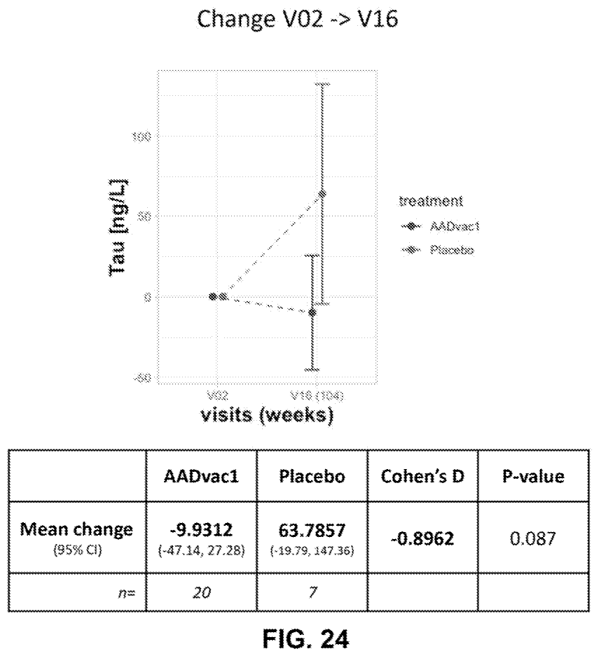

As shown in FIG. 24 and Table 21, levels of total tau (ng/L) in CSF stabilized in patients that received AADvac1 treatment, and increased in patients that received the placebo treatment.

TABLE 21
Change in CSF levels of total tau, pT181, and pT217 from V16 to V02

| | | | | | Difference | | | | | | |
|---|---|---|---|---|---|---|---|---|---|---|---|
| | n | | Mean | | (AADvac1 − | SD | | | | | Cohen's |
| (V16 = V02) | AADvac1 | Placebo | AADvac1 | Placebo | Placebo) | AADvac1 | Placebo | LB | UB | p-value | D |
| Tau | 20 | 7 | −9.93125 | 63.78571 | −73.71696 | 79.51190 | 90.37589 | −160.17322 | 12.73929 | 0.087 | 0.896 |
| pT181 | 20 | 7 | −6.28750 | 0.28471 | −6.57321 | 11.84300 | 10.02853 | −16.61557 | 3.46914 | 0.1799 | 0.575 |
| pT217 | 19 | 7 | −34.44828 | 30.16351 | −64.61179 | 57.87938 | 92.48061 | −151.21821 | 21.99462 | 0.1232 | 0.947 |

Method: Determination of Neurogranin in the CSF

The detection and measurement of neurogranin in CSF was performed by sandwich ELISA according to the methods as detailed in Portelius et al., (2018) Acta Neuropathologica, 136(3):363-376. Briefly, anti-neurogranin antibodies were obtained and coated onto plates. The plates were washed and remaining binding sites were blocked with bovine serum albumin (BSA) before CSF samples were introduced. Recombinant full-length neurogranin protein with a GST-tag was used for calibration. The detection antibody was biotin-labeled anti-neurogranin antibodies, which could be detected by streptavidin-labeled horse radish peroxidase. The binding was measured by a plate reader via absorbance at 450 nm.

Results: Neurogranin is a Biomarker that May Indicate Synaptic Degeneration

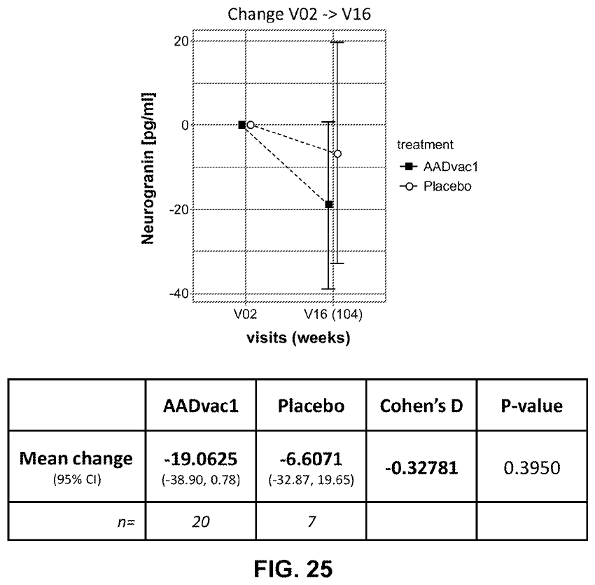

As shown in FIG. 25, levels of neurogranin in CSF were similar at V02 in patients treated with AADvac1 or placebo. However, at V16, levels of neurogranin showed stronger decrease in patients treated with AADvac1 compared to that treated with placebo. The effect size was modest (Cohen's D=−0.327) and not statistically significant due to the small sample size.

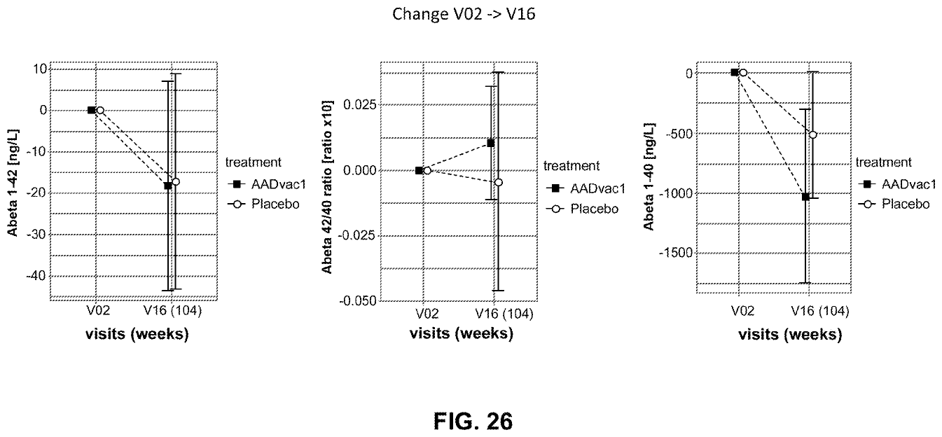

No significant difference was found for levels of Aβ1-42, Aβ1-40, and the ratio of Aβ42/Aβ40 between treatment and placebo groups after treatment (FIG. 26).

### Example 5: Bioimaging for Monitoring Disease Progression

MRI imaging was performed to assess safety of treatment by volumetric analysis of the brain. MRI scans were performed according to the schedule as shown in Table 5.

The following regions of interest were subjected to volumetric analysis: total brain, hippocampus (left and right), and lateral ventricles.

Whole brain volume at different time points throughout the study and the percentage change in whole brain volume compared to baseline were calculated with SIENAX 2.6. Lobe volumes and regional volumes at any time point before and after longitudinal registration were calculated with FREESURFER 6.0.

Diffusion tensor imaging (DTI) is a magnetic resonance imaging technique that allows quantifying microstructural tissue alterations, which can be invisible on conventional MRI. Anisotropic diffusion of water in the brain is imaged by MRI, allowing the calculation of fractional anisotropy (FA), which quantifies how strongly directional the local white matter tract structures are. Baykara et al., (2016) Annals of Neurology, 80(4):581-592. MRI and a standardized DTI scan with 42 diffusion directions was performed. Pre-processing of data including eddy current correction and brain extraction was performed. Then, pixel-wise calculation of FA maps was performed from pre-processed diffusion weighted and reference scans and skeletonized. The maps were registered and the transformed FA values for the fornix, the genu, body and splenium of the corpus callosum were read out.

Results:

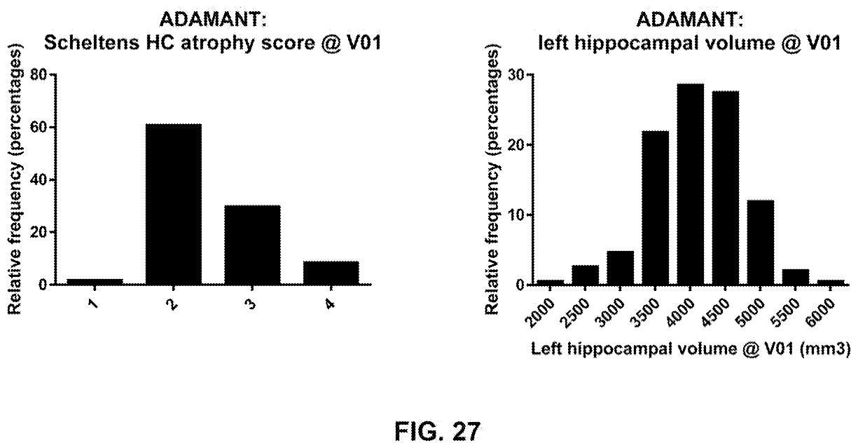

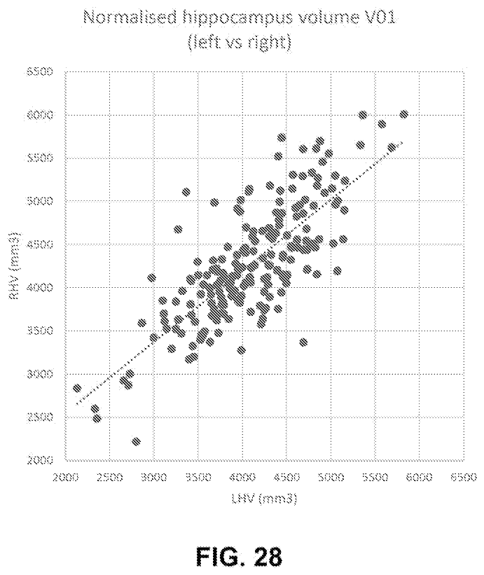

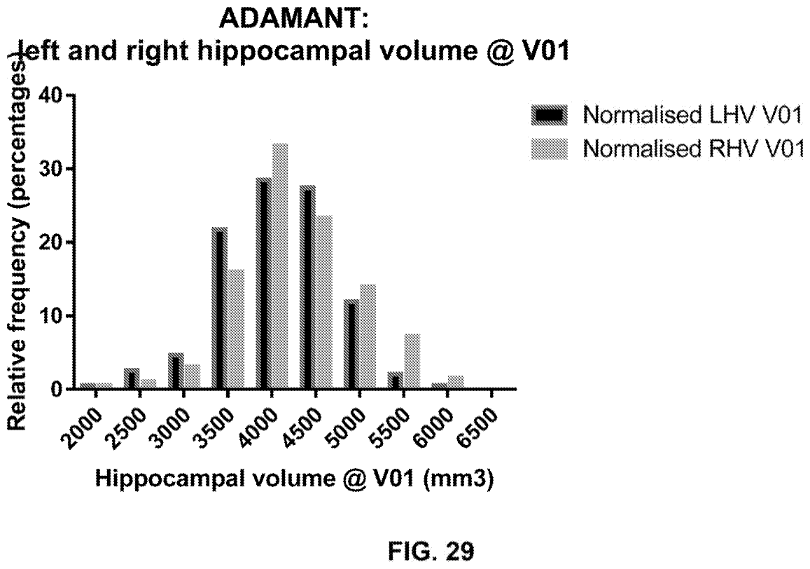

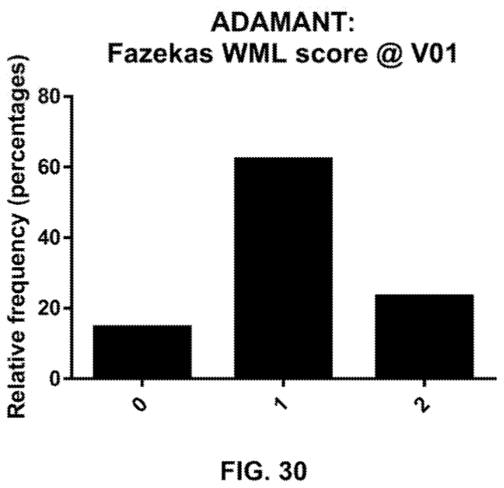

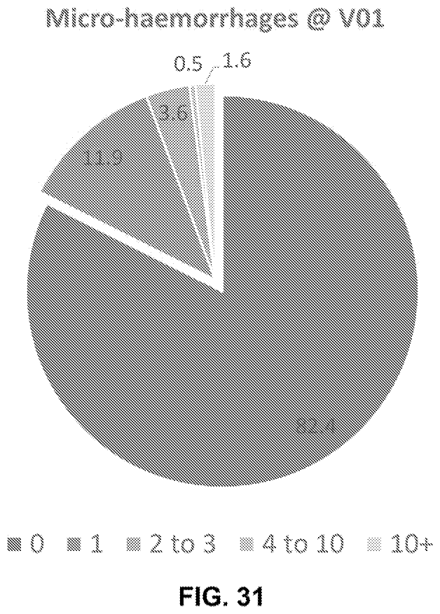

Patients displayed a wide range in the degree of brain atrophy at baseline, as can be seen in the range of Scheltens score and left hippocampal volume at Visit 01 (FIGS. 27A and 27B). While hippocampal atrophy appears to be symmetrical (FIG. 28), the left hippocampal volume appears smaller (FIG. 29). Patients also displayed an age- and disease-appropriate number of white matter lesions (WML) and micro-hemorrhages at baseline, as can be seen in FIGS. 30 and 31 respectively.

At the group level, there was no difference between the AADvac1-treated groups and the placebo treated groups at baseline as visualized by MRI volumetry. In the older subgroup, patients displayed lower hippocampal volumes, more white matter lesions, and lower atrophy rates in most regions of the brain over the duration of the study. In the subgroup of patients who are ApoE4 carriers, patients displayed lower hippocampal volumes at baseline (p=0.0003) and slightly higher hippocampal atrophy rates (p=0.01).

Change in brain volumes after treatment with AADvac1 is as shown in Table 22. Volumetry results for the entire patient cohort showed high heterogeneity.

TABLE 22

| | LS mean of % volume | | | | | | |
|---|---|---|---|---|---|---|---|
| | change (SE) | | | | Group | | |
| Region of interest | AADvac1 | | Placebo | | difference | | P-value |
| Left hippocampus | −8.54 | (0.443) | −8.26 | (0.553) | −0.28 | (0.711) | 0.696 |
| Right hippocampus | −8.50 | (0.432) | −7.79 | (0.539) | −0.71 | (0.694) | 0.310 |
| Left entorhinal cortex | −10.36 | (0.793) | −10.58 | (1.004) | 0.22 | (1.289) | 0.862 |
| Right entorhinal cortex | −9.98 | (0.857) | −10.05 | (1.090) | 0.07 | (1.395) | 0.960 |
| Lateral ventricles | 18.06 | (0.915) | 17.73 | (1.142) | 0.33 | (1.465) | 0.821 |
| Entire cortex | −3.16 | (0.346) | −3.07 | (0.432) | −0.09 | (0.556) | 0.876 |
| Parietal cortex | −2.84 | (0.450) | −2.94 | (0.563) | 0.10 | (0.725) | 0.890 |
| Temporal cortex | −5.76 | (0.418) | −5.76 | (0.418) | 0.03 | (0.672) | 0.963 |
| Occipital cortex | −2.11 | (0.371) | −2.79 | (0.463) | 0.68 | (0.596) | 0.256 |
| Frontal cortex | −2.37 | (0.355) | −1.91 | (0.443) | −0.46 | (0.572) | 0.423 |
| Total brain volume | −3.97 | (0.195) | −3.96 | (0.244) | 0.00 | (0.313) | 0.991 |

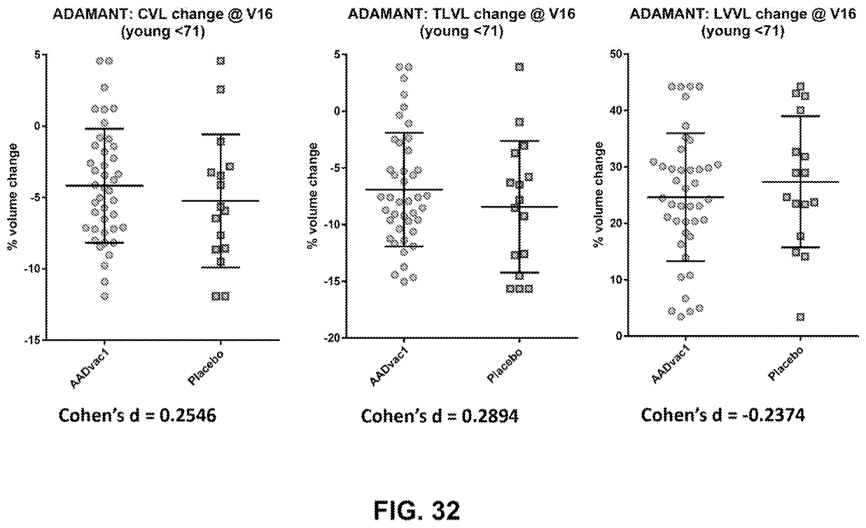

In a subgroup of young patients (<71 years), small differences were observed in the percentage volume change in AADvac1-treated patients compared to placebo-treated patients at V16, as shown in FIG. 32.

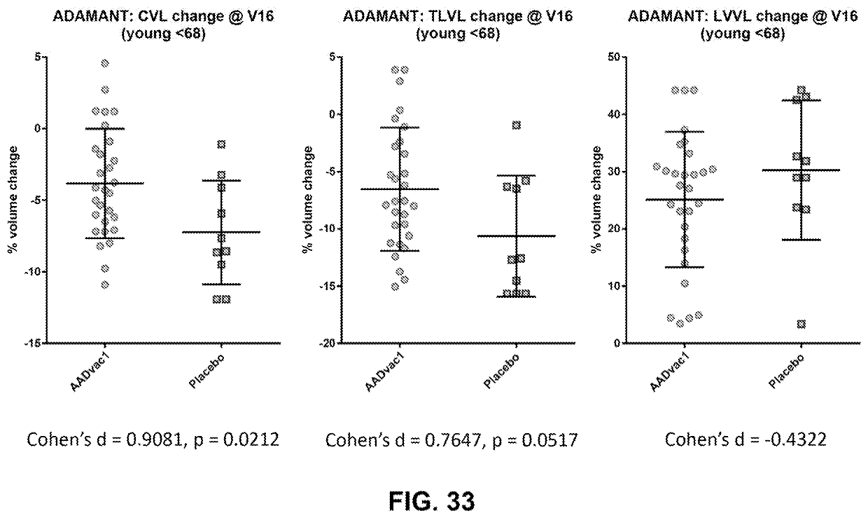

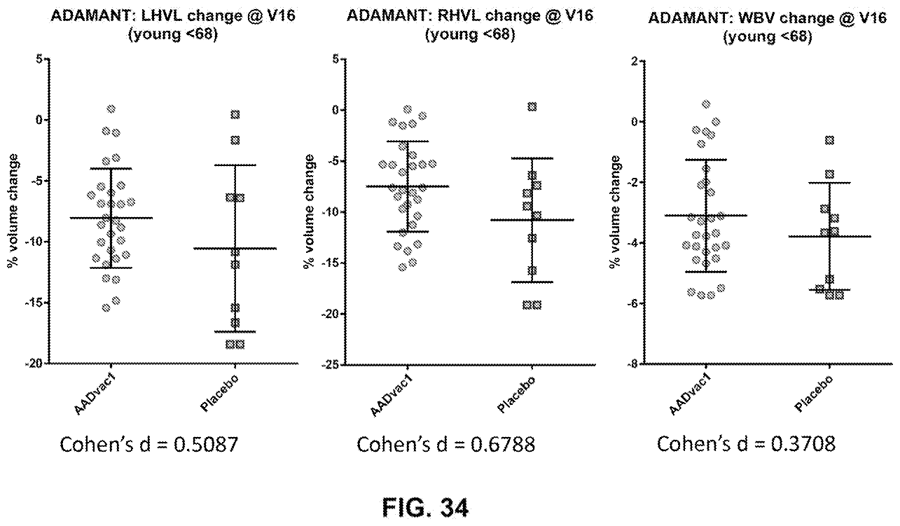

This difference is more visible when comparing amongst the younger age groups. For example, in the subgroup of young patients (<68 years), moderate to large differences were observed in multiple regions of interest (temporal lobe, ventricles and whole cortex volume) in AADvac1-treated patients compared to placebo-treated patients at V16, as shown in FIGS. 33-34.

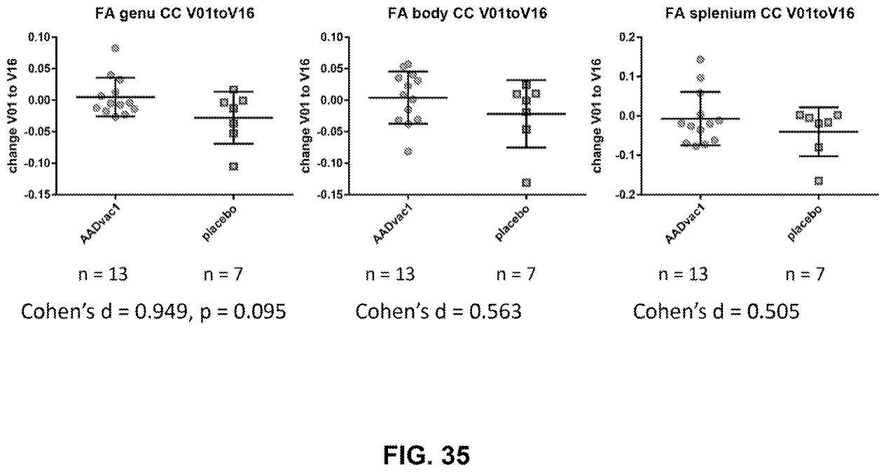

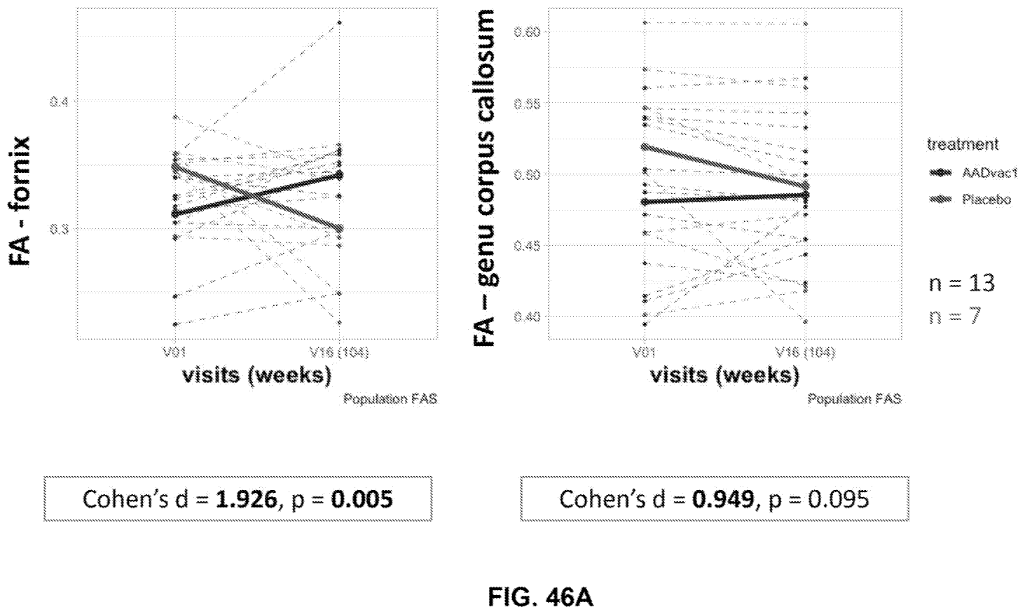

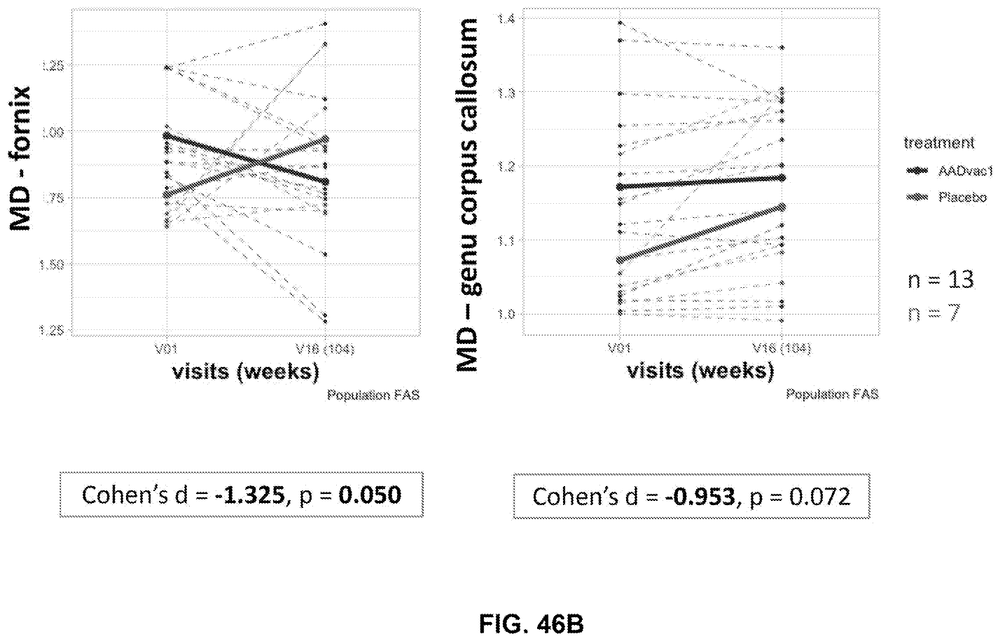

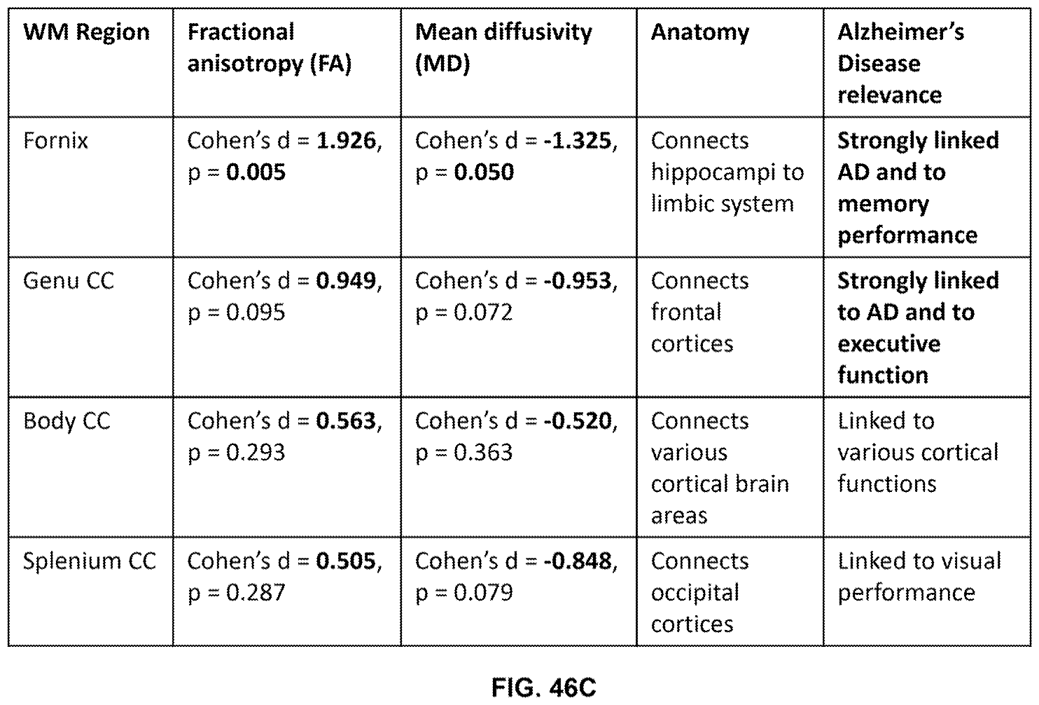

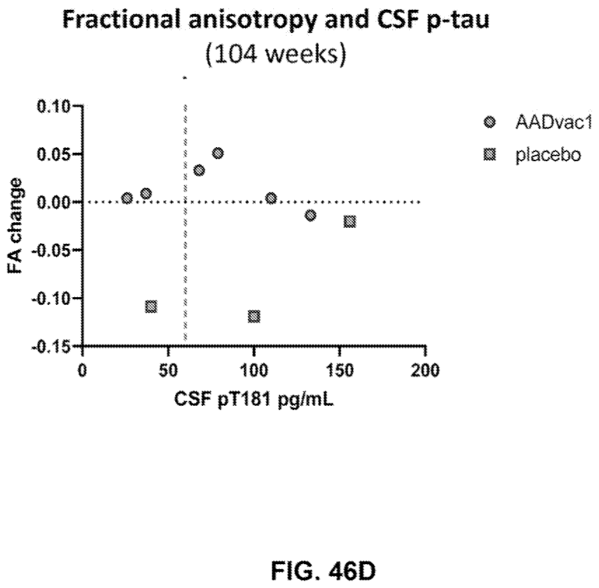

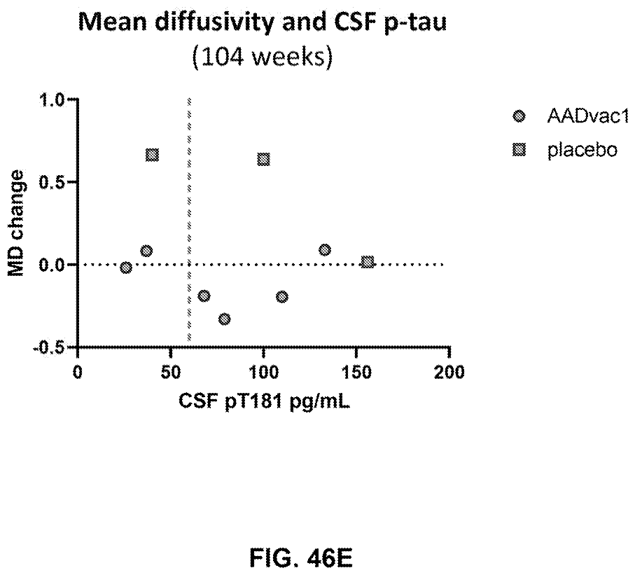

Diffusion Tensor Imaging (DTI) provides mapping of the amount of white matter in the brains of patients. As shown in FIG. 35 and FIG. 46, the change in fractional anisotropy from V01 to V16 as measured by DTI in four different regions of the brain (fornix, and genu, body/corpus and splenium of the corpus callosum) showed differences between AADvac1- and placebo-treated patients, with the most pronounced difference being observed on the fornix, a pathway originating in the hippocampus. This indicates that white matter integrity in the brains of AADvac1-treated patients was preserved, when compared to that in placebo-treated patients.

### Example 6: Determination of MicroRNA in Human Plasma by Quantitative Real-Time PCR

Human plasma samples (400 μL) were thawed on ice and centrifuged for 3000×g for 5 min at 4° C. to pellet any precipitates that might have formed. The resulting supernatant (300 μL) was aspirated to into a clean 1.5 mL RNAse-free tube (Ambion, Cat. AM12450). RNA extraction was performed using miRNeasy Serum/plasma Advanced kit (Qiagen, Cat. 217204) according to manufacturer's recommendation using 2 μg of RNAse-free glycogen as carrier (Thermo Scientific, Cat.R0551). Purified RNA was eluted with 20 μL of RNase-free water into a clean 1.5 mL RNAse-free tube and stored at −80° C.

Reverse transcription of miRNA into cDNA was performed using miScript II RT kit (Qiagen, Cat. No. 218161) following the manufacturer's protocol, and 1.6 μL of RNA eluate as used as the template. cDNA synthesis protocol is as follows: 60 min at 37° C., 5 min at 95° C. and 1 min at 4° C. The final reaction of 20 μL was diluted by addition of 80 μL of RNase-free water (Qiagen, Cat. No. 129112) and the diluted cDNA was stored at −80° C. until use.

Transcriptomic profiling of miRNA levels was performed using quantitative real-time PCR (qPCR) method together with miScript SYBR Green PCR kit (Qiagen, Cat. No. 218076). The composition of each 20 μL PCR reaction was as follows: 10 μL of 2×QuantiTect SYBR Green PCR Master Mix, 2 μL 10×miScript universal primer, 4 μL of 5×miRNA specific primer, 2 μL RNase-free water and 2 μL of diluted cDNA as template. All components were pipetted into an optical 96-wellplate (Applied Biosystems, Cat. No. 4306737) and sealed with optical adhesive film (Applied Biosystems, Cat. No. 4311971). The plate was mixed by pulse vortexing and centrifuged at 1000×g for 1 min at room temperature. qPCR cycling protocol was as follows: initial denaturation 15 min at 95° C., 42 cycles of 15 sec at 94° C., 30 sec at 55° C. and 35 sec at 70° C. The cycling protocol included a melt curve (60° C. to 90° C., Ramp rate (1%)=0.35° C./30 sec.

The miRNAs profiled in the study by qPCR were:
hsa-let-7a-5p
hsa-miR-10a-5p
hsa-miR-145-5p
hsa-miR-103a-3p
hsa-miR-191-5p
hsa-miR-374a-5p
hsa-miR-26a-5p
hsa-miR-107
hsa-miR-15a-5p
hsa-miR-126-3p
hsa-miR-224-5p
hsa-miR-18a-5p
hsa-miR-23a-3p
hsa-miR-26b-5p
hsa-miR-21-5p

Evaluation of miRNA expression levels was performed using the relative quantification method, where dCT and 2\hat{ }(-dCT) values were calculated using the average cycle threshold (CT) mean of all miRNAs of individual sample as an internal calibration control. The resulting values represent relative expression of the target miRNA in the plasma sample.

Results:

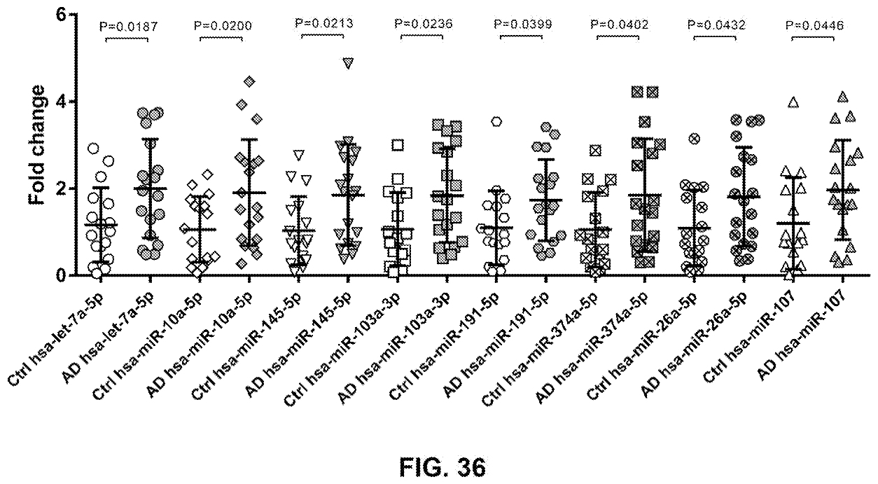

The levels of specific miRNA in patients with AD were evaluated and compared to those of control patients who have depressive symptoms but have low pTau levels. As shown in FIG. 36, levels of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p were different in patients with AD compared to control.

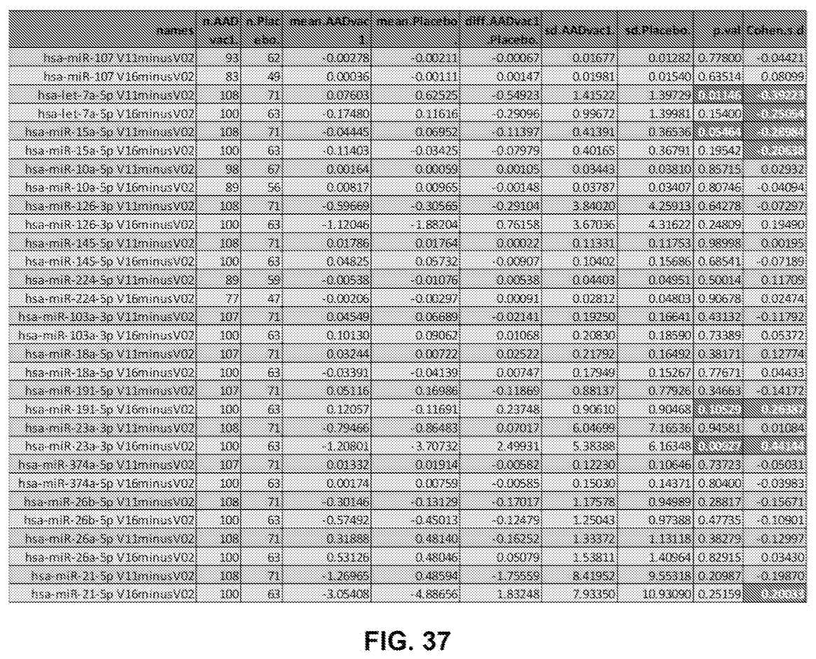

The levels of specific miRNA in patients with AD were also measured throughout the course of the study, and the levels in patients who received treatment with AADvac1 were compared to those in patients who received the placebo treatment. As shown in FIG. 37, the levels of hsa-let-7a-5p and hsa-miR-15a-5p were decreased in the treatment group compared to the placebo group, while the levels of hsa-miR-23a-3p and hsa-miR-191-5p increased in the treatment group compared to the placebo group.

### Example 7: Determination of Metabolites in the Blood Plasma and Cerebrospinal Fluid

Quantitative liquid chromatography-mass spectrometry (LC-MS/MS) metabolomic analysis is performed by combining direct injection mass spectrometry with a reverse-phase LC-MS/MS (AbsoluteIDQ p180Kit; Biocrates Life Sciences, Austria). Sample preparation and measurements are carried out according to the manufacturer's protocol and have been described in detail in the literature, e.g., Wang-Sattler et al., PLoS ONE, (2008) 3(12): e3863.

### Example 8: Determination of Thiols in the Blood Plasma

Quantitative LC-MS/MS analysis of cysteine, homocysteine, cysteinyl-glycine and glutathione is performed according to Forgacsova et al., (2019) Journal of Pharmaceutical and Biomedical Analysis 164 (2019) 442-451. Briefly, samples are prepared by mixing plasma with Millipore Water, solution of internal standards, and 10 μl of TCEP (10 g/l). The resulting mixtures are kept for 30 min at the room temperature. Proteins are precipitated with the addition of 25 μL of trichloroacetic acid (2 mol/l). The capped tubes with the precipitate are immediately vortexed and centrifuged at 30000×g for 10 min. Then, potassium phosphate dibasic (0.5 mol/l) is added to the supernatant and the samples are derivatized at room temperature with N-phenylmaleimide (20 mmol/l in ACN). After 10 min the samples are diluted and loaded onto a pre-conditioned solid phase extraction (SPE) column (OASIS HLB 30 mg, Waters, Prag, Czech Republic). The eluate is dried for 2 hours in Savant SpeedVac (Thermo Fischer Scientific, Waltham, USA). The dried samples are reconstituted in 80% acetonitrile. The tubes are vortexed, centrifuged, the supernatants are transferred into sample vials and analysed by UHPLC-MS/MS.

### Example 9: Determination of Peptides in the Cerebrospinal Fluid

Acetonitrile is added to 500 μL of human CSF (20% final v/v) and samples are centrifuged at 4° C. for 10 min (20,000×g). Then the CSF samples are centrifuged through the MWCO filters (10 kDa) according to the manufacturer's instructions. All steps are performed on ice without addition of protease inhibitors. The filtrate containing the peptide fraction is dried using the SpeedVac concentrator (Pragolab, Bratislava, Slovakia) and then stored at −80° C. until analyzed. Peptides are separated using Acquity M-Class UHPLC (Waters) using the nanoEase HSS T3 C18 analytical column (250 mm length, 75 μm diameter, 1.8 μm particle size). For the thorough separation, a 120 min gradient of 5-90% acetonitrile with 0.1% formic acid is applied at a flow rate of 300 nL/min. The column outlet is connected to the PicoTip emitter (360 μm outer diameter, 20 μm inner diameter, 10 μm tip diameter) and samples are nanosprayed (3.1 kV capillary voltage) into the quadrupole time-of-flight mass spectrometer Synapt G2-Si with ion mobility (Waters). Spectra are recorded in a data-dependent manner in high definition DDA mode. Ions with 50-2000 m/z are detected, with a 0.2 s in MS and 0.1 s in MS/MS spectral acquisition scan rate. The external mass calibrant Glu1-Fibrinopeptide B (100 fmol/mL) is infused through the reference line at a flow rate of 500 nL/min and used for mass correction. The data are processed in Progenesis QI (Waters).

### Example 10: Determination of Affinity of Human Serum Antibodies to Tau 151-391 4R by Surface Plasmon Resonance (SPR)

Isolation of serum antibodies reactive with mis-disordered tau:

Tosyl-activated magnetic beads (Dynabeads) are coated with highly purified recombinant human disordered tau protein (tau151-391/4R) by an overnight incubation according to the manufacturer instructions. The prepared tau beads are incubated on ice with an aliquot of patient serum for one hour, washed on ice and the anti-tau antibodies of all immunoglobulin classes bound to the beads are washed out by elution at low pH. The eluted pool of affinity-isolated antibodies is immediately neutralized and stored at −20° C.

Preparation of SPR Biochip:

A CM-5 sensorchip and BIACORE3000 instrument (GE Healthcare) is used for analysis. The sensorchip is washed and coated with affinity-purified anti-human immunoglobulins IgG Fc fragment polyclonal antibody (Merck) using amino-reactive immobilization chemistry, according to the manufacturer instructions.

SPR Kinetic Analysis:

In the initial step, a 10-20 fold diluted affinity-isolated antibody pool is applied on the biosensor surface pre-coated with an anti-IgG polyclonal antibody. Therefore, only IgG class antibodies are captured for further affinity analysis. Subsequently, a 50 nM solution of recombinant human mis-disordered tau protein (tau151-391/4R) is introduced and the binding and dissociation are monitored in real time. To record a background signal in the assay, buffer was injected instead of tau under otherwise identical conditions. All analyses are performed in duplicates. Regeneration of the biosensor surface is performed by a 10-second injection of 100 mM HCl. As a quality control of the system and the whole assay, the injection of humanized DC8E8 antibody is performed and the shape and level of tau protein binding sensorgram are registered. Injection-to-injection and run-to-run repeatability were evaluated.

Evaluation of Kinetic Sensorgrams:

The binding kinetics of tau is corrected by a double-referencing method as described in Myszka, (1999) J. Mol. Recognit., 12:279-284. The corrected curve is fitted to a heterogeneous ligand (parallel reactions) binding model, as implemented in the BIAevaluation software v. 4.1.1 (GE Healthcare), which assumes two antibody pools present in the patient serum. Fitting parameters are set as follows: ka1 fitted globally, initial value=5×10⁶ M⁻¹s⁻¹; kd1 fitted globally, initial value=0.001 s⁻¹; ka2 fitted globally, initial value=1×10⁶ M⁻¹s⁻¹; kd2 fitted globally, initial value=0.01 s⁻¹; RI=0 RU constant value; Rmax1, Rmax2 fitted globally, initial value as proposed by the software. Equilibrium association constants (affinities, KA) for both antibody pools are calculated from kinetic rate constants directly in the Biaevaluation software. The fractions p1, p2 of the individual antibody pools are calculated from Rmax values:

p1=Rmax1/(Rmax1+Rmax2)

p2=Rmax2/(Rmax1+Rmax2)=1-p1

Calculation of Functional Affinities:

Dimensionless values of functional antibody affinities in serum are calculated using antibody concentrations in serum determined by quantitative ELISA in μg/ml, c:

functional affinity=c*(p1*KA1+p2*KA2)/1000000

The obtained values are plotted using GraphPad Prism v. 6.

### Example 11: Further Results and Analysis from Phase 2 Clinical Trial

This Example provides further results and analysis from the phase 2 clinical trial described, e.g., in Example 1.

Low Drop-Out Rate Over Two-Year Study:

The clinical study had a low drop-out rate over 24 months, particularly in the AADvac1 group. Specifically, the drop-out rate was 14.5% for the AADvac1 group and 20.3% for the placebo group.

Heterogeneous Study Sample:

CSF biomarkers for Alzheimer's Disease were analyzed in 60 patients. Almost 30% of these patients did not fulfill biomarker criteria for Alzheimer's Disease, according to Jack et al., NIA-AA Research Framework: Toward a biological definition of Alzheimer's disease. Alzheimer's Dement. 2018 14(4):535-562. This finding suggests that approximately one third of the study population are tau negative and do not suffer from Alzheimer's Disease.

Safety of AADvac1:

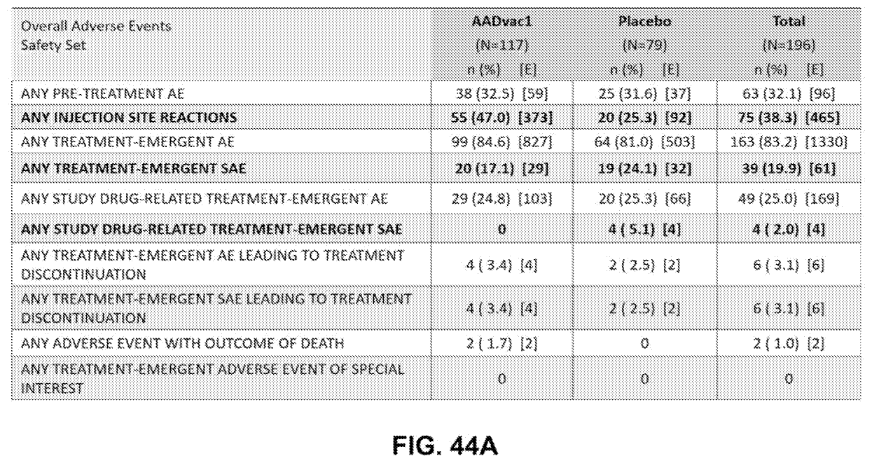

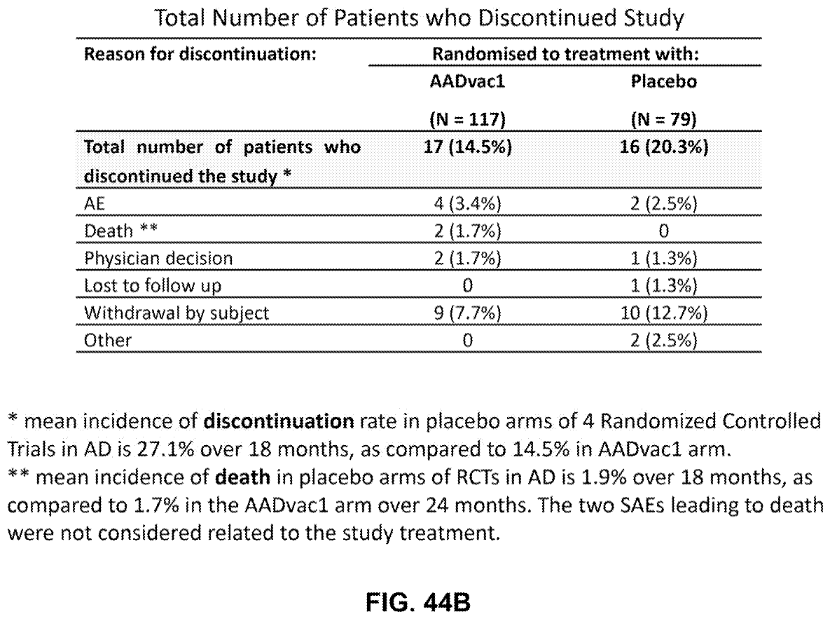

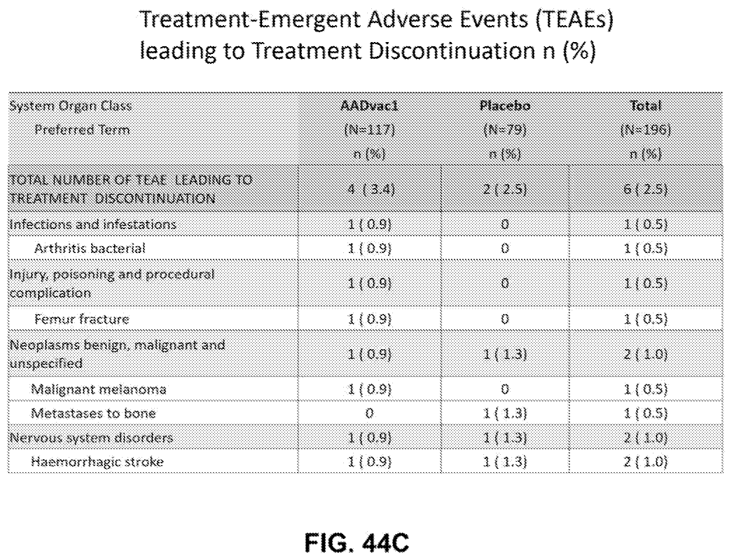

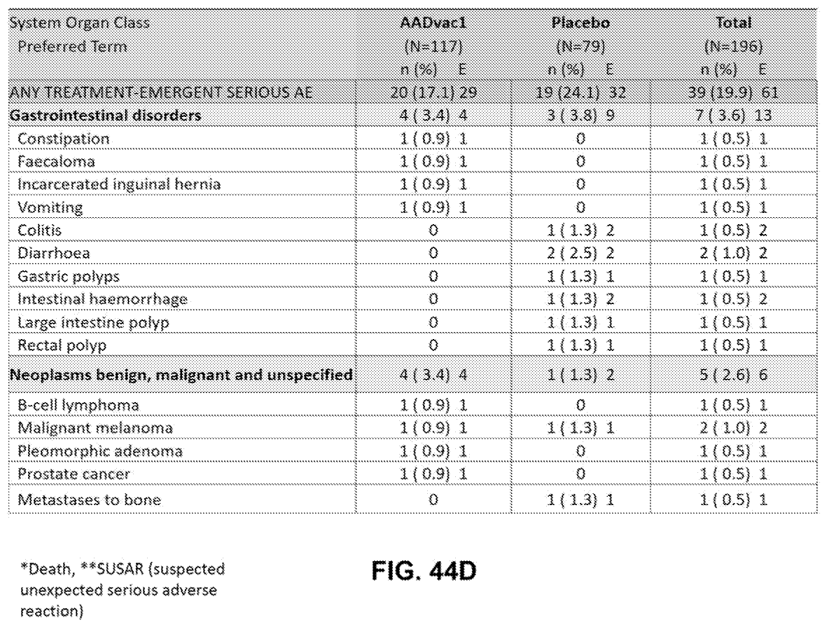

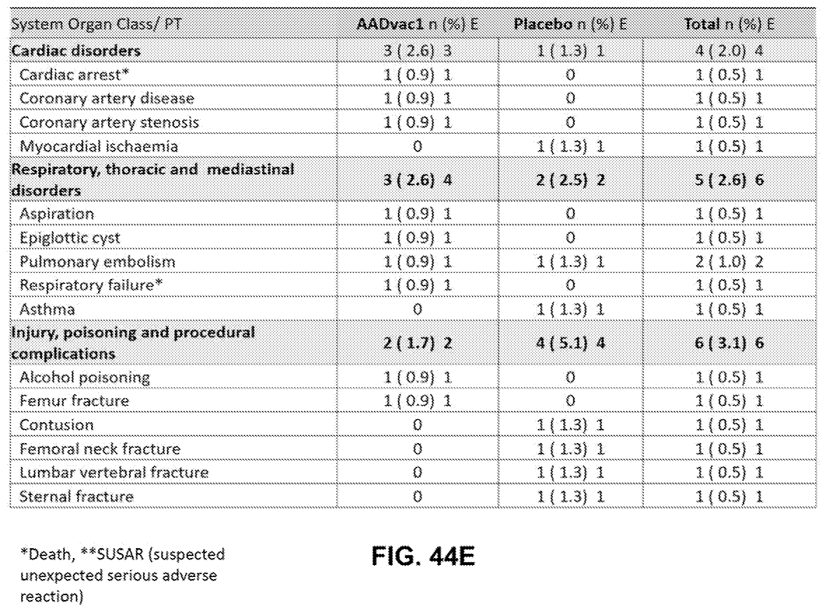

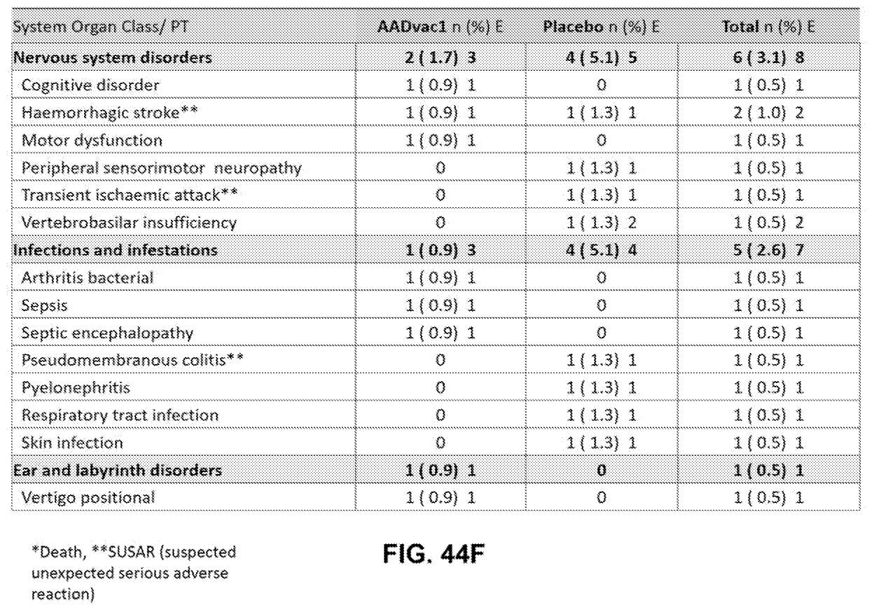

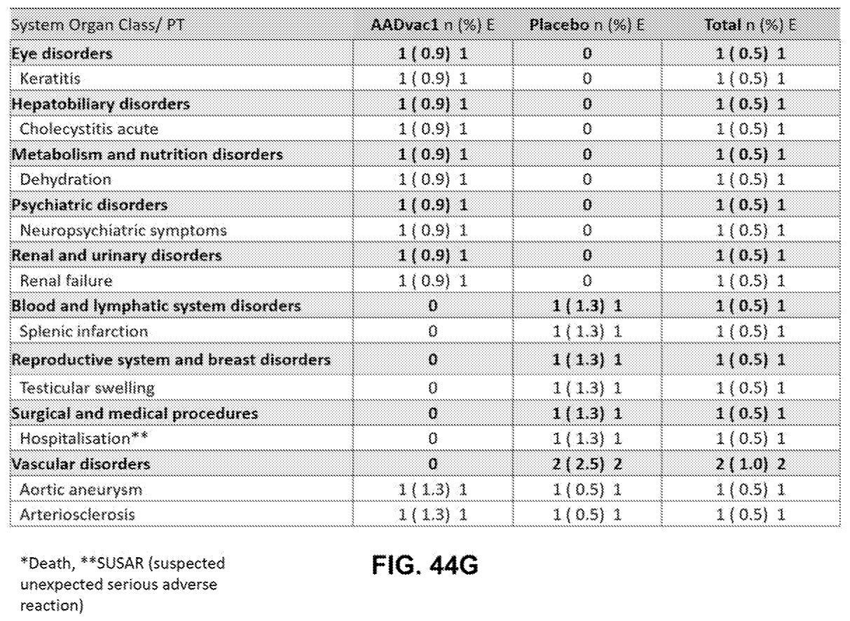

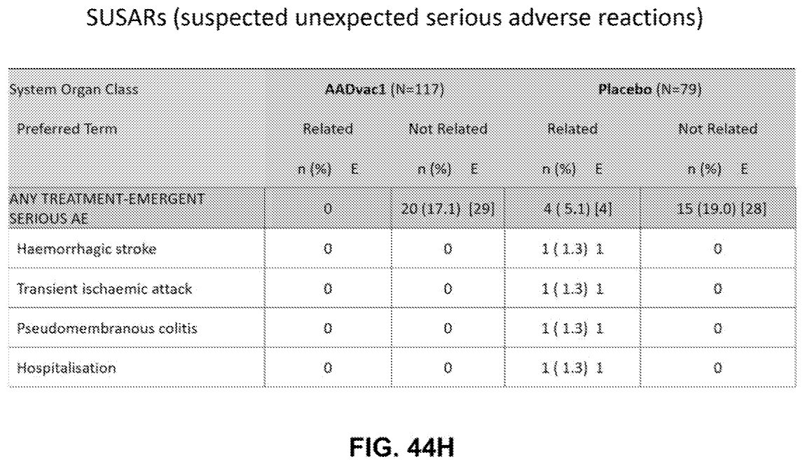

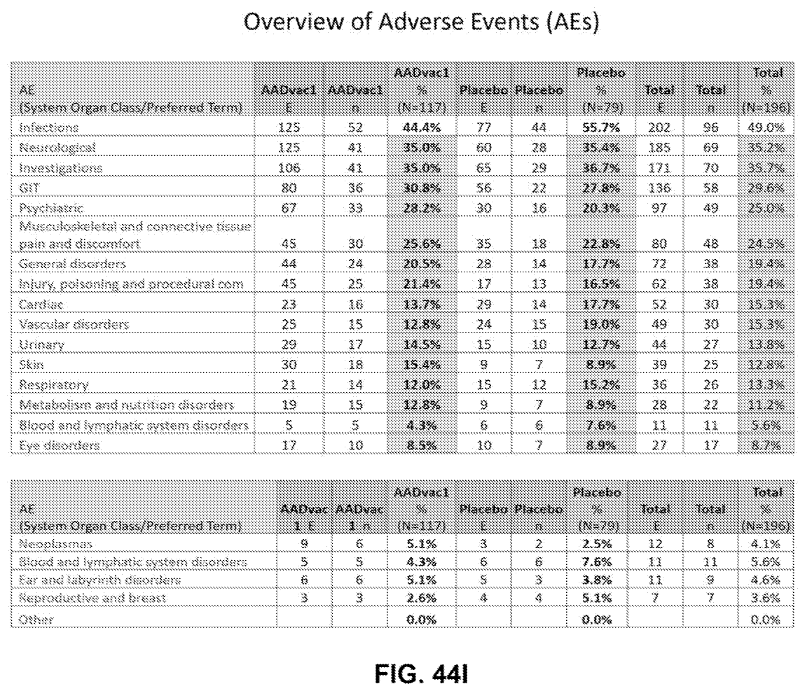

AADvac1 was found to be safe and well tolerated. Safety results from the phase 2 clinical study are summarized in FIGS. 44A-441. Local injection site reactions were observed with higher incidence in the AADvac1 group as compared to placebo. These were observed in both arms as expected and do not prevent blinding of the studies in the future.

A borderline significant difference in the incidence of confusion was observed between the treatment arms (AADvac1 n=6, placebo n=0, p<0.05, not corrected for multiplicity). Confusion commonly occurs as Alzheimer's disease progresses, and patients' memory, orientation, and judgment become progressively impaired. The adverse event of “Confusion” is usually a caregiver-reported outcome, with the caregiver informing the investigator that the patient was more confused than usual. It can also be ascertained by the investigator during the examination at a study visit. If the confusion is judged to be clinically significant, it is reported as an adverse event. For the purpose of statistical analysis, the number of patients who experienced one or more instances of confusion is compared between study arms, using tests that evaluate the difference between the expected frequencies and the observed frequencies, for example Fischer's exact test, or Chi square test. As noted above, the phase 2 clinical study found zero cases of confusion in the placebo arm (less than expected), and six cases of confusion in the AADvac1 arm (more than expected, and marginally statistically significant). As confusion is a natural part of the AD disease process, a causal relationship between AADvac1 treatment and confusion is not certain. Confusion is presently being considered a ‘potential risk’, which will be confirmed or refuted by a future larger study.

No other significant difference in the incidence and nature of adverse events (AEs) were observed between the AADvac1 and placebo groups. The observed AEs were in line with what is expected in untreated patients with AD (i.e., background incidence) and no safety signal emerged from any other safety and medical assessments. For example, there was no difference in ARIA-H (micro-haemorrhages), no ARIA-E (vascular oedema), no meningoencephalitis, and no anaphylaxis.

CSF Biomarker Results:

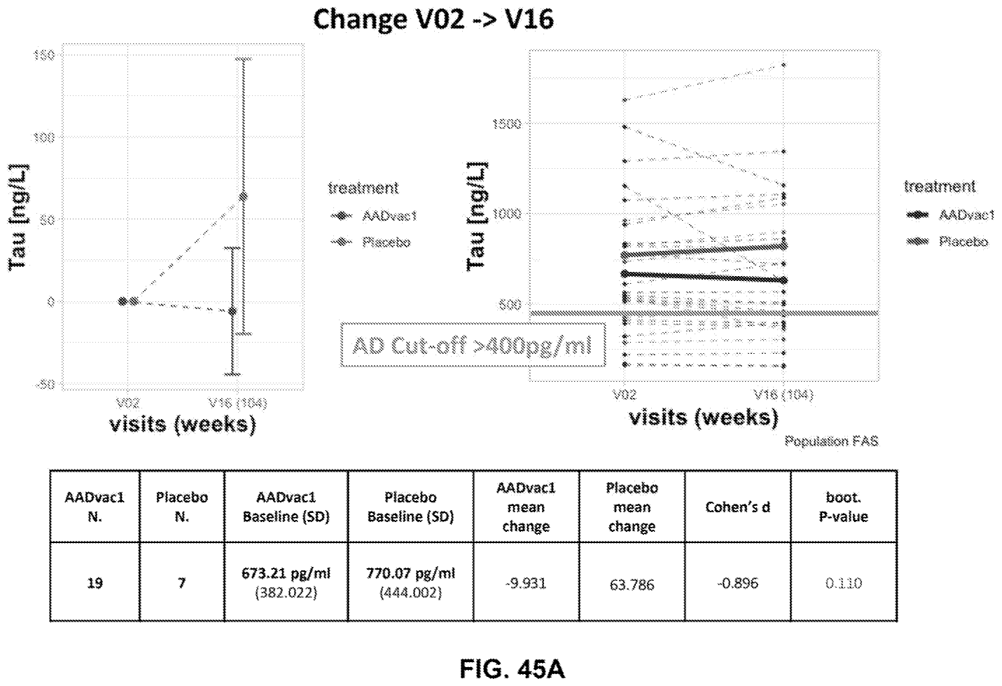

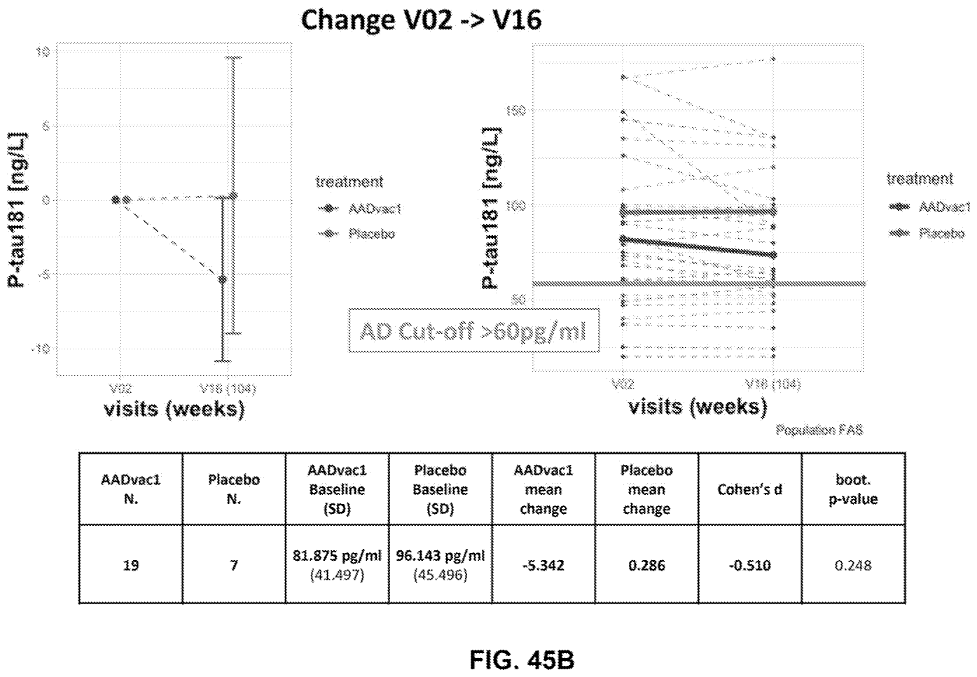

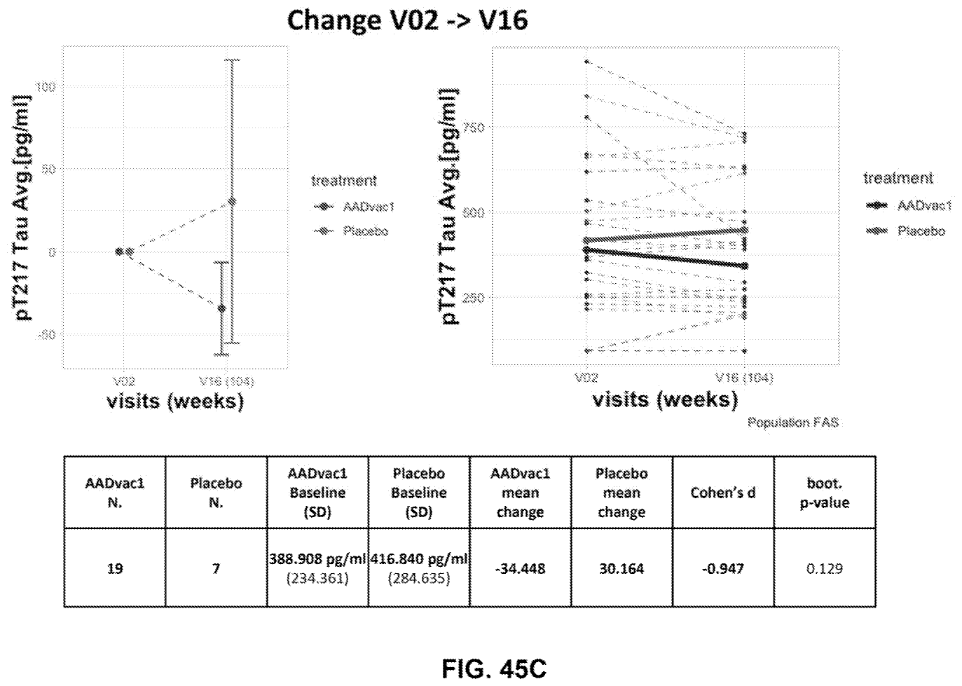

Further analyses or plotting of tau (e.g., total tau, phosphorylated tau) biomarker results in CSF are summarized in FIGS. 45A-45C. The tau biomarkers support the positive signal from plasma NfL described above. For example, FIG. 45A shows that total tau levels stabilized due to AADvac1 treatment, indicating a reduction in neurodegenerative processes; FIG. 45B shows that pT181 tau levels were reduced after AADvac1 treatment, indicating a reduction in the accumulation of tau pathology; and FIG. 45C shows that pT217 tau levels were reduced after AADvac1 treatment, further indicating a reduction in the accumulation of tau pathology.

Diffusion Tensor Imaging (DTI) MRI Results:

Data demonstrating that white matter integrity was preserved (white matter degeneration was halted) by AADvac1 treatment are shown in FIGS. 46A-46E. The MRI measurements looked at the fornix and corpus callosum. Without being bound by theory, the fornix is considered to be an imaging marker for episodic memory deficits. FIG. 46A shows results obtained using fractional anisotropy (FA), while FIG. 46B shows results obtained using mean diffusivity (MD). Results are summarized further in FIG. 46C. FIGS. 46D-46E show fractional anisotropy or mean diffusivity data plotted against CSF p-tau levels.

Summary of Plasma, CSF, DTI Biomarker Results:

Plasma NfL levels show reduced accumulation in the patients treated with AADvac1. These results indicate that AADvac1 slows the progression of the neurodegenerative process to levels that are more typically seen in healthy elderly individuals. The results observed with plasma NfL levels are supported by the data on CSF tau biomarkers (total Tau and phospho-Tau pT181 & pT217), suggesting target engagement with tau, a decline in the progress of tau pathology, and halting of neurodegenerative processes. There was also a positive correlation between plasma NfL levels and CSF NfL levels.

DTI MRI results (e.g., assessing white matter degeneration in the fornix and corpus callosum) also suggest that AADvac1 inhibits tau spreading and halts axonal degeneration. MRI volumetry results demonstrate that AADvac1 slows brain atrophy, for example as demonstrated in the cortex and hippocampus. Overall, biomarkers of Alzheimer's Disease consistently supported the disease modifying effect (e.g., halting neurodegeneration, reducing the rate of neuronal loss, halting white matter degeneration, etc.) of AADvac1.

Clinical Outcomes—Age Group Effect:

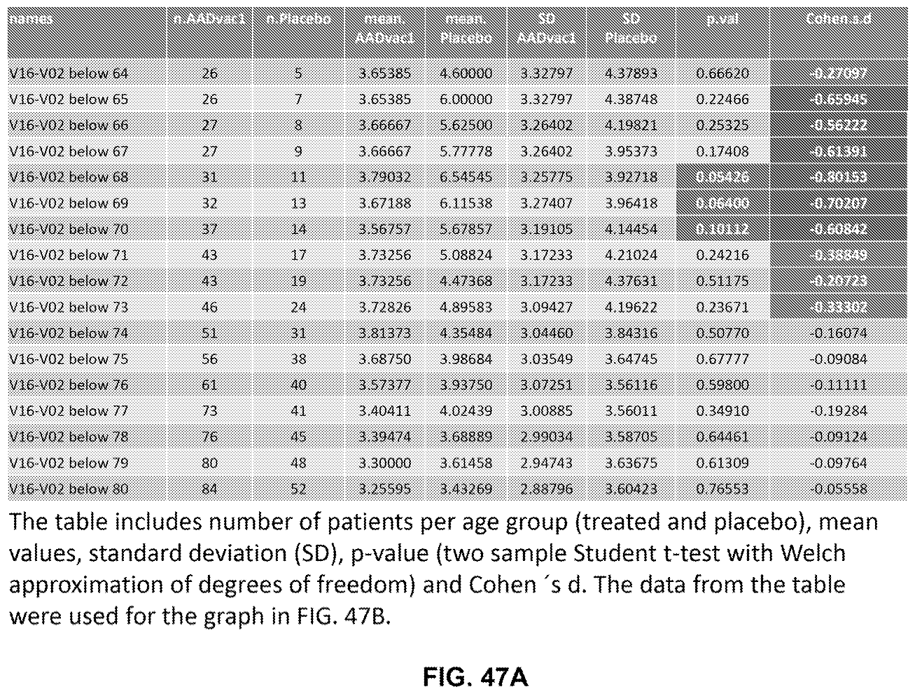

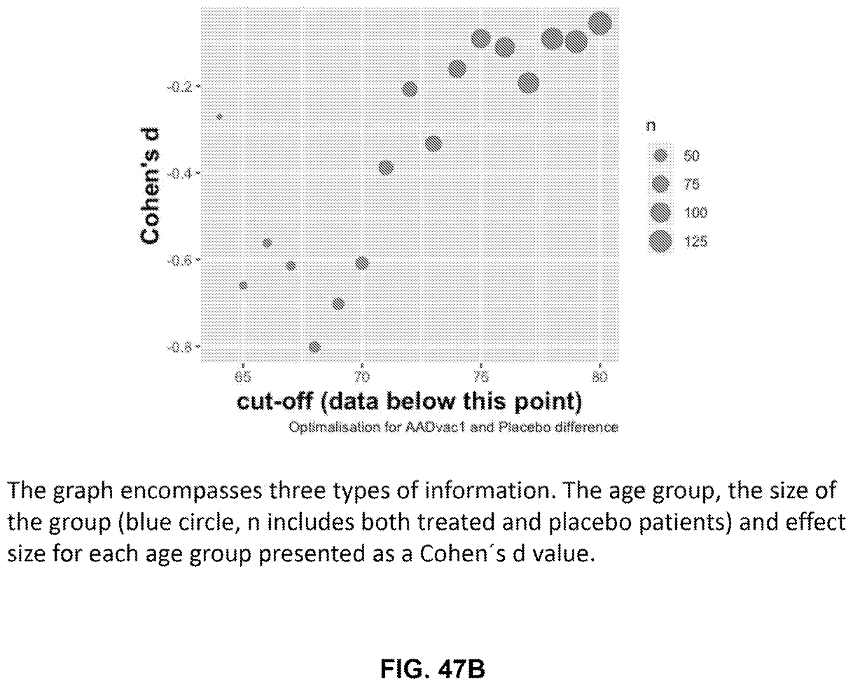

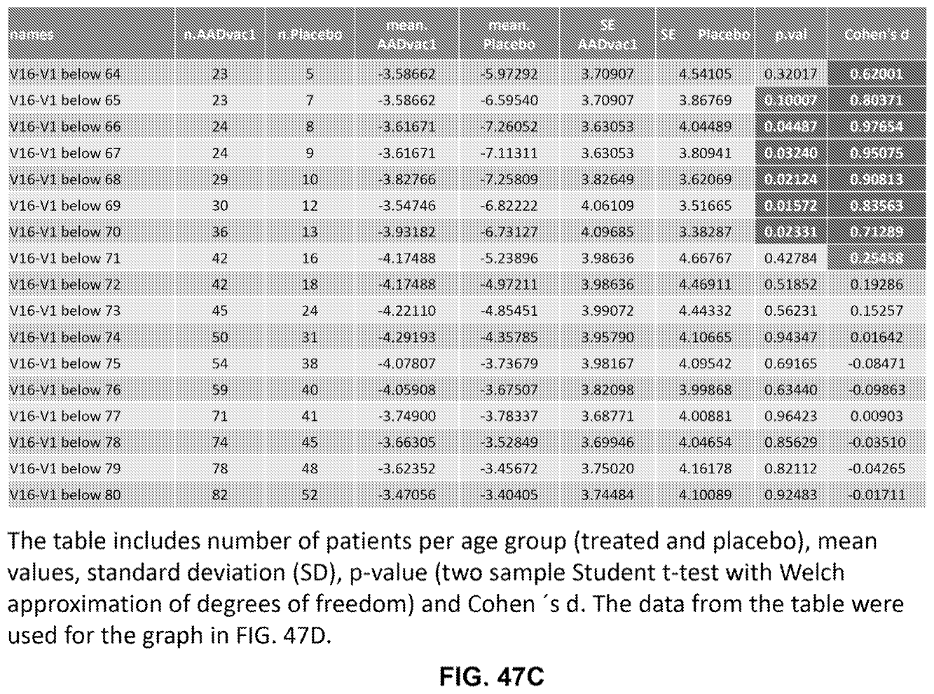

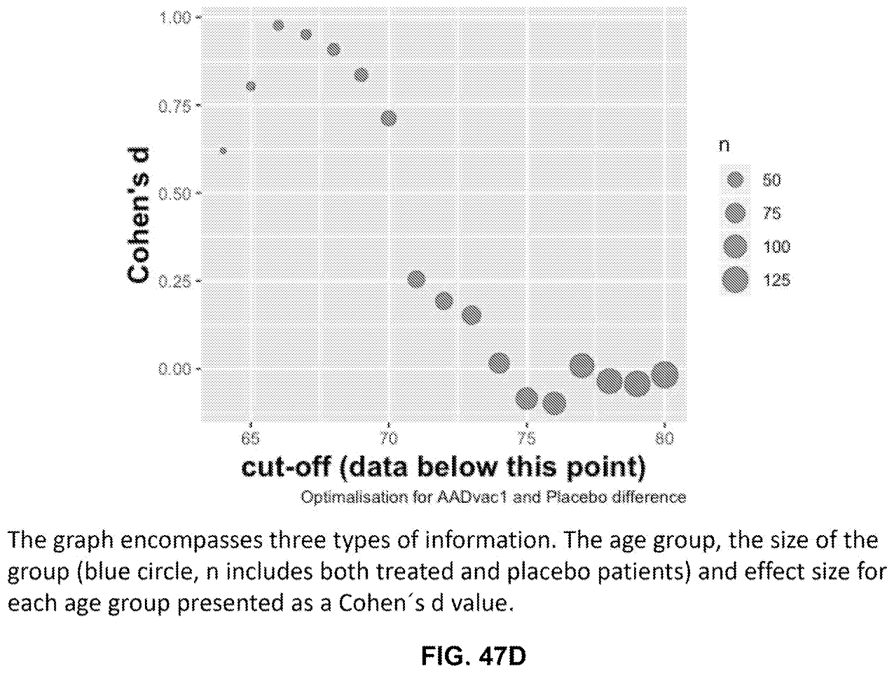

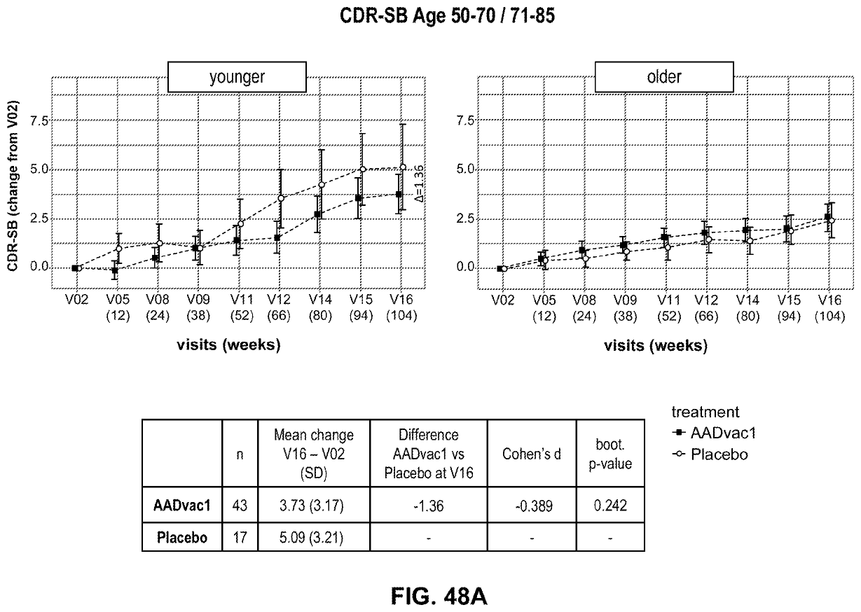

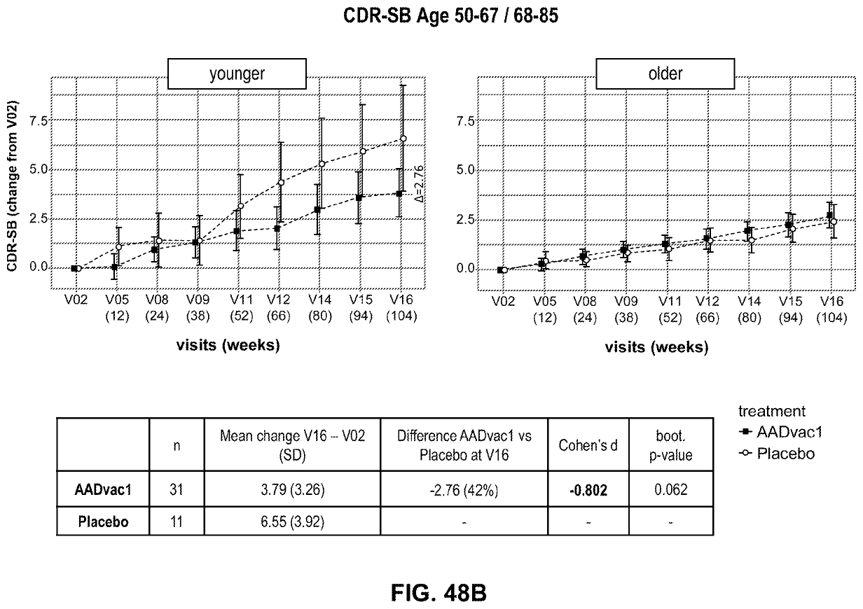

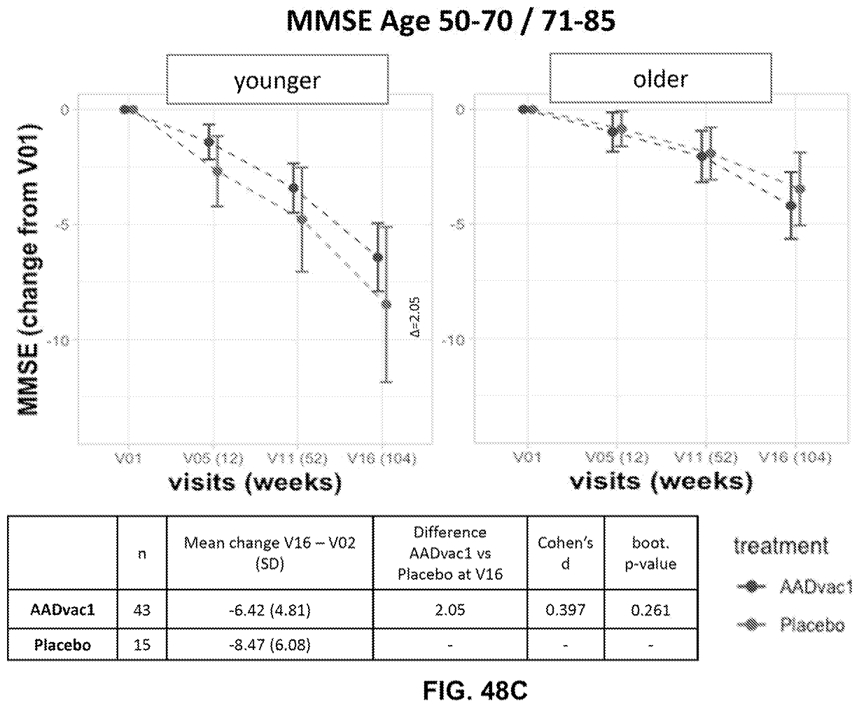

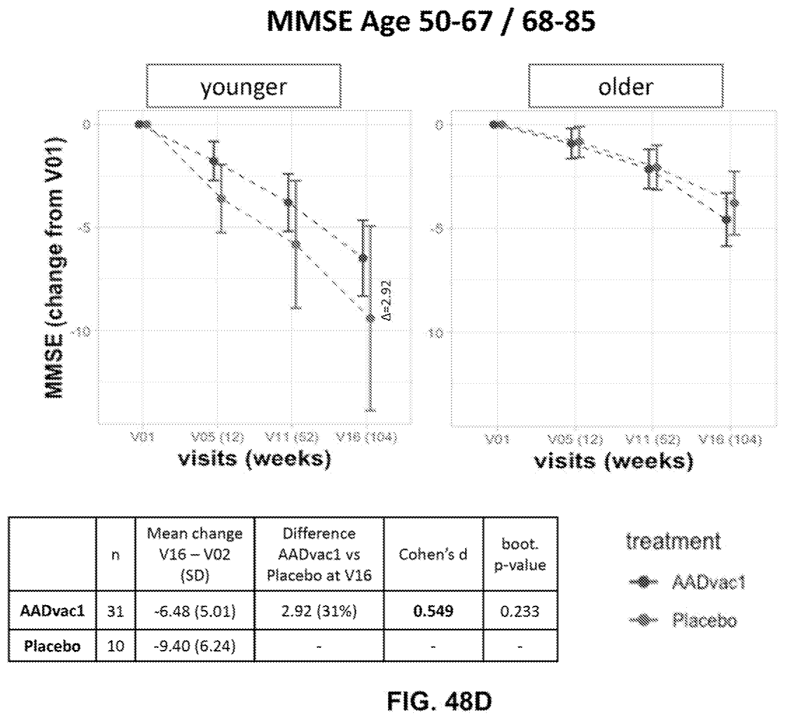

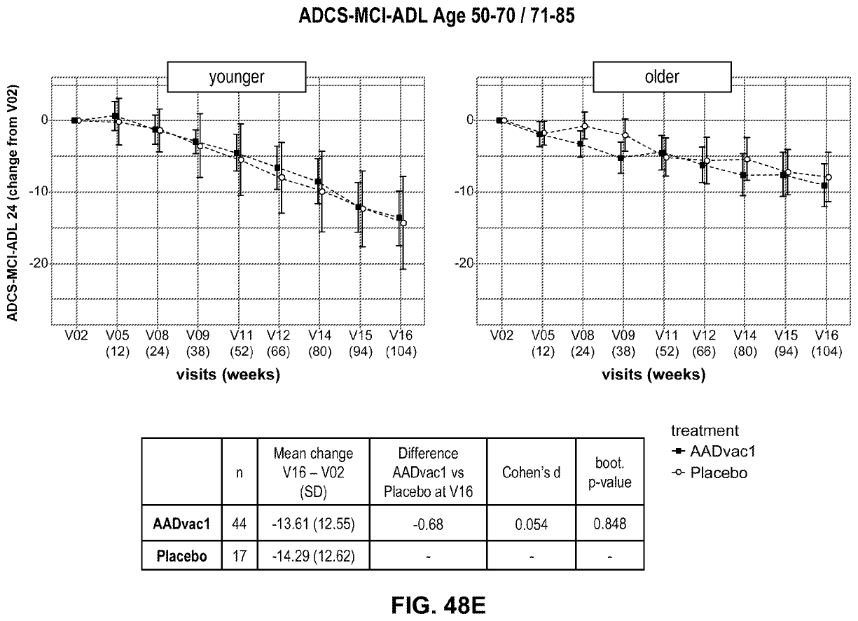

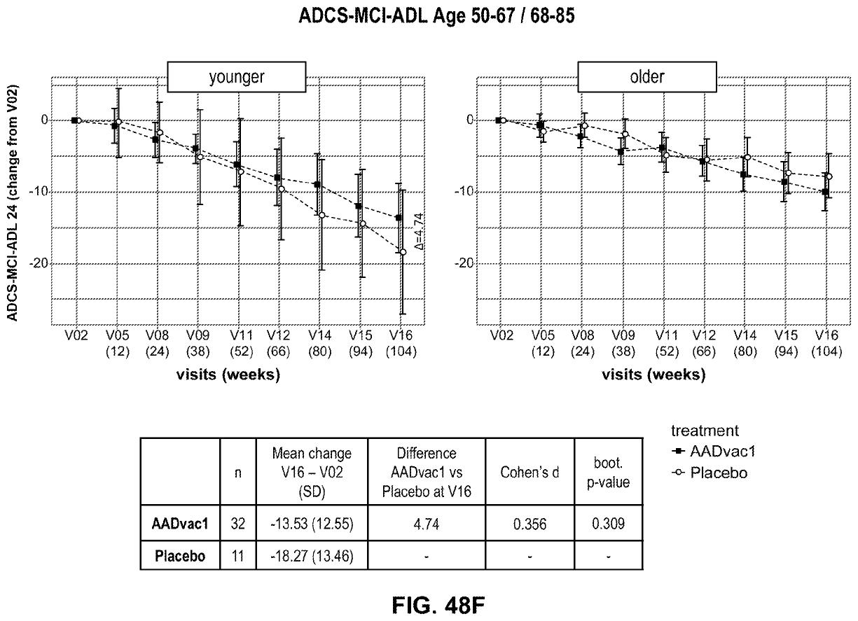

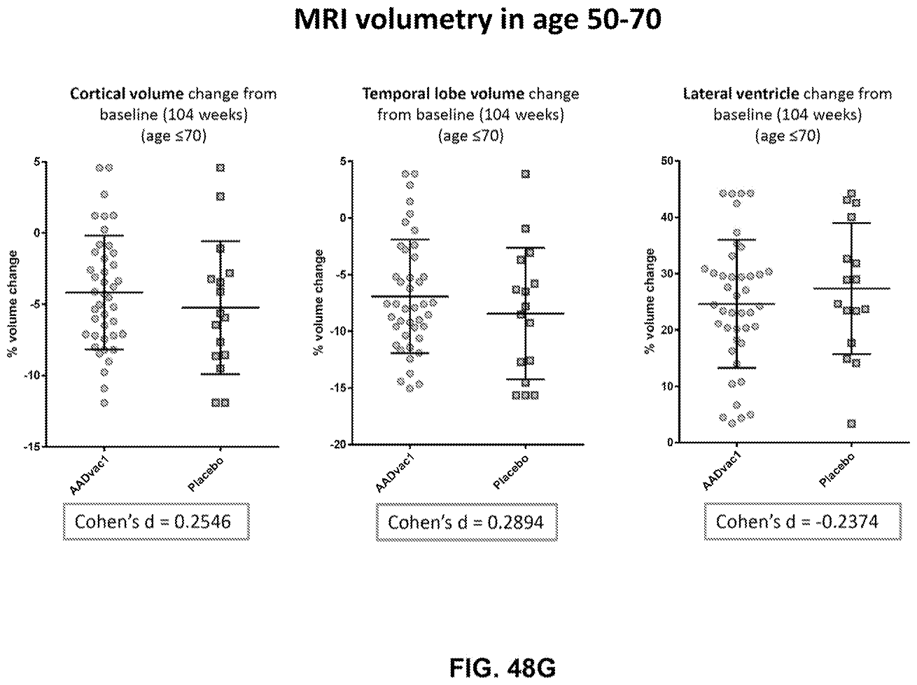

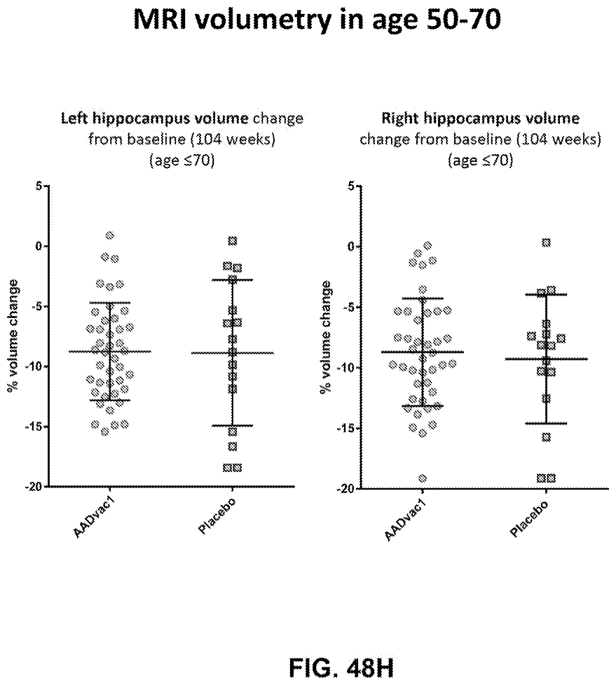

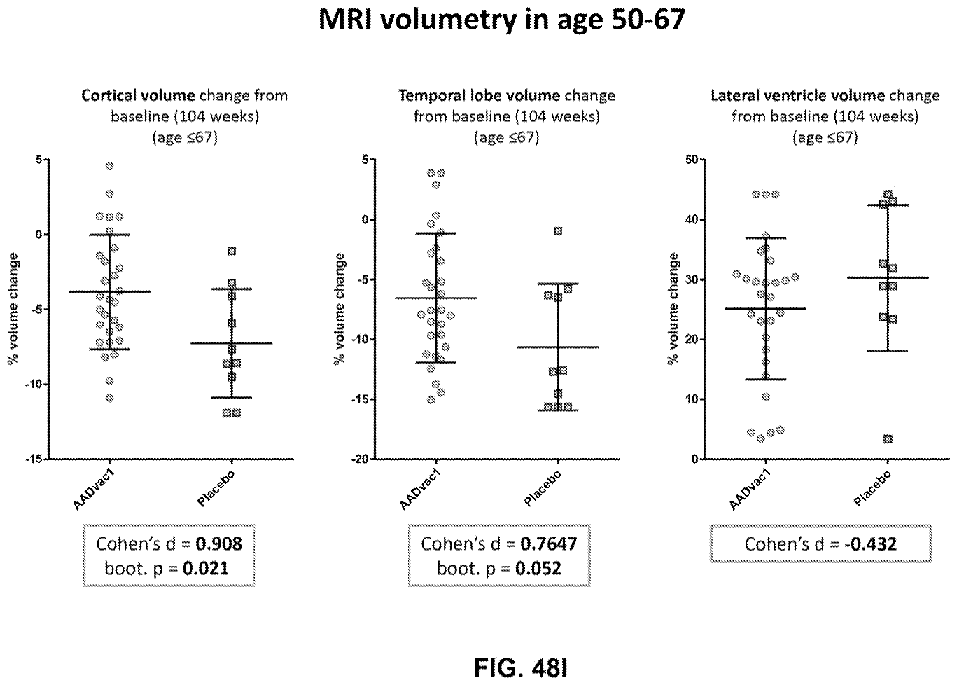

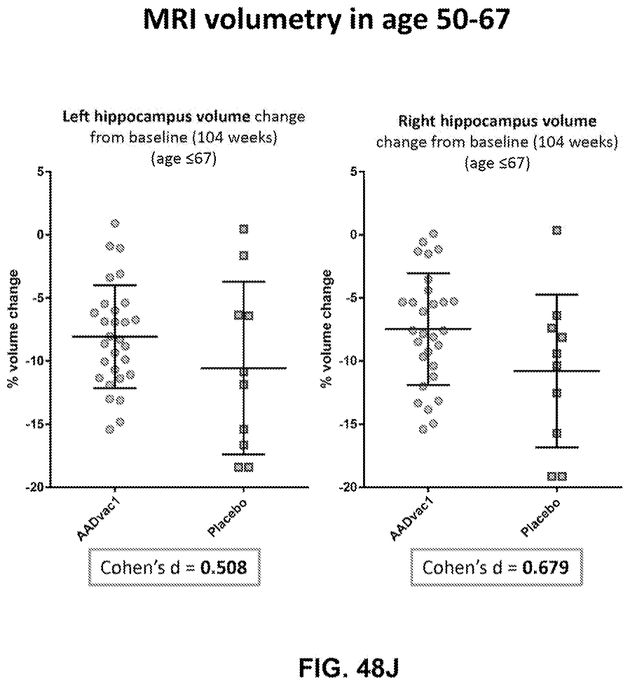

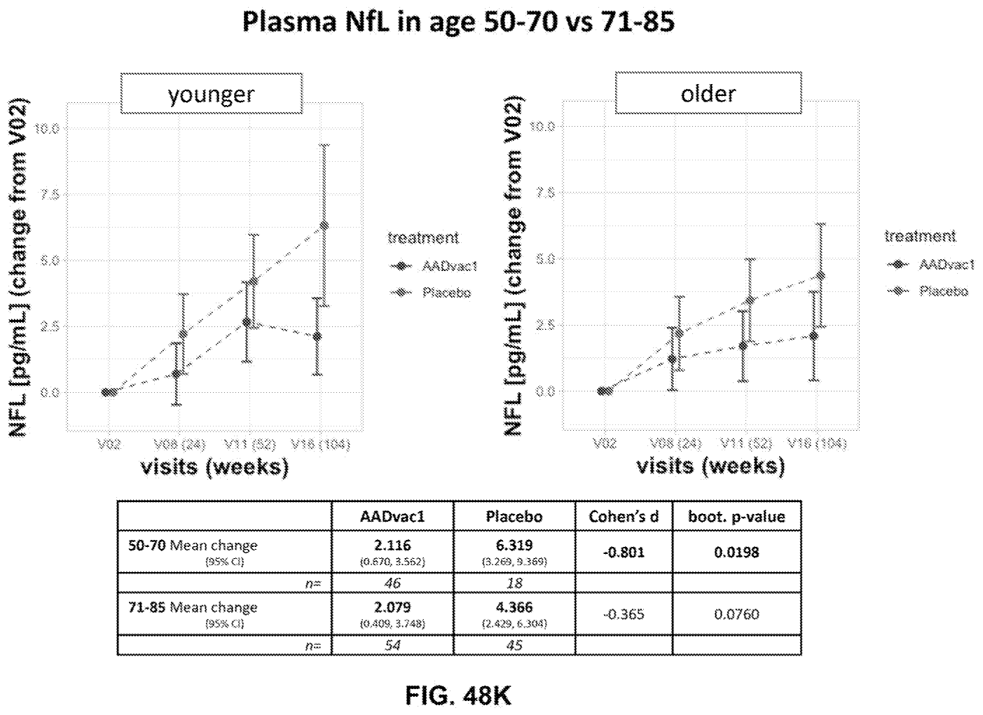

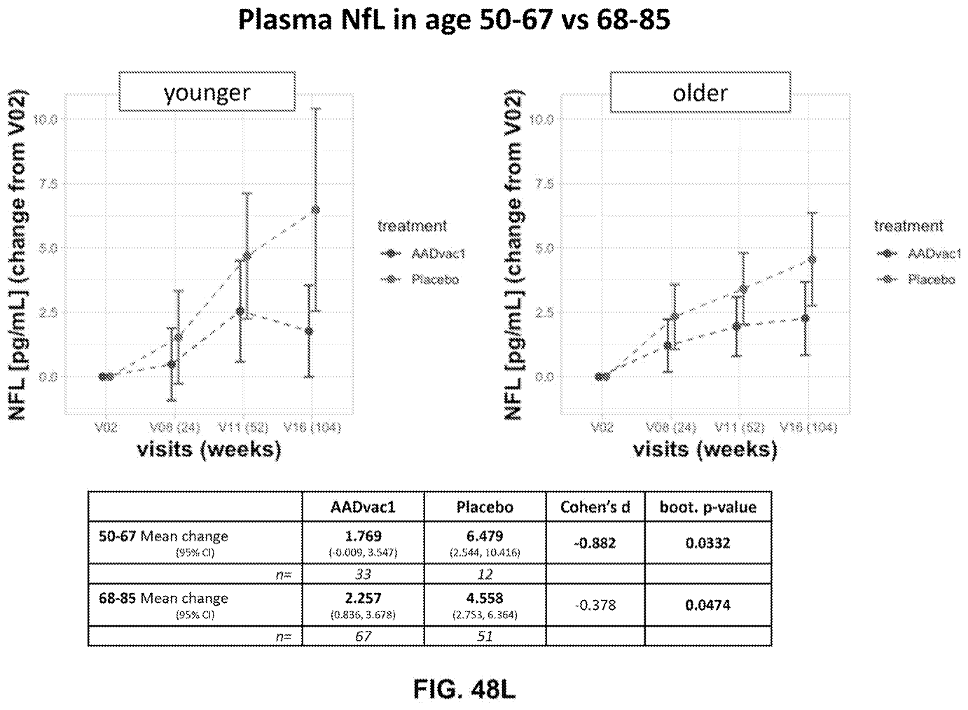

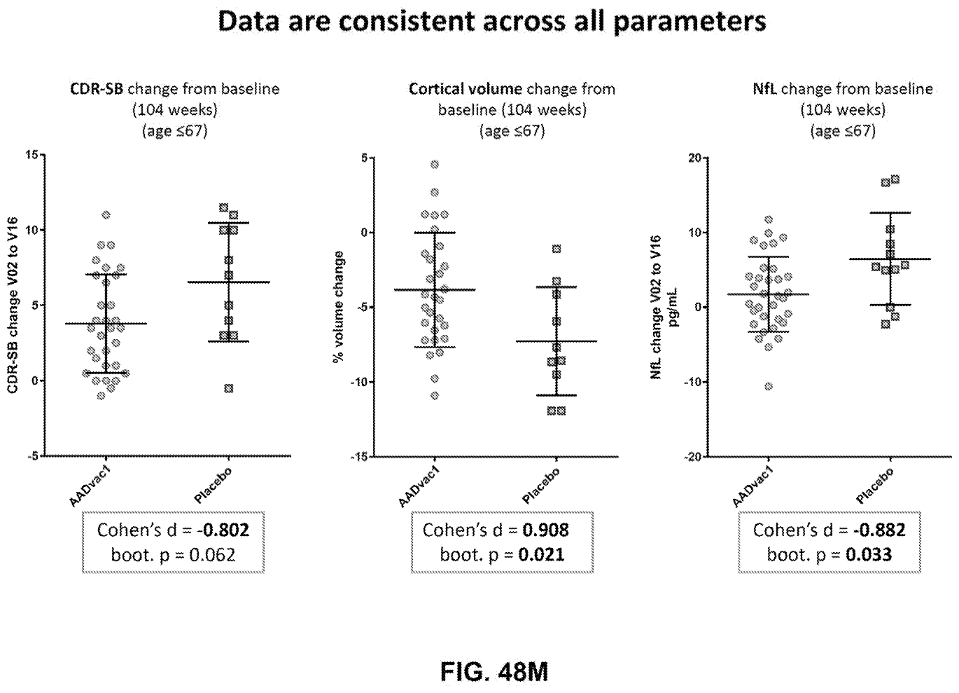

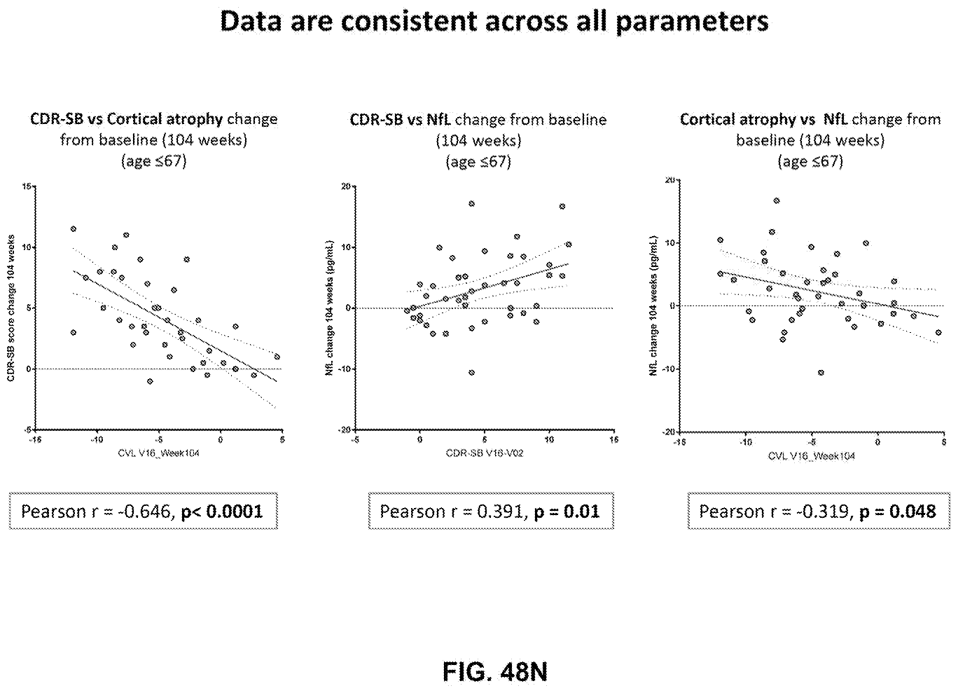

CDR-SB data (FIGS. 47A-47B) and MRI cortical atrophy data (FIGS. 47C-47D) are provided and plotted to demonstrate age group effect size of AADvac1. Further data (CDR-SB, MMSE, ADCS-MCI-ADL, MRI volumetry, plasma NfL) organized by patient age are provided above and in FIGS. 48A-48N.

These data are further summarized in Tables 23, 24A, and 24B, demonstrating particularly pronounced improvements for young onset patients treated with AADvac1 compared to those administered placebo. In addition, the A+/T+ subgroup of patients (patients having biomarkers evidencing the presence of both Aβ and pathologic tau), as defined by the updated NIA-AA (2018) research framework and summarized in Table 3, also exhibited particularly pronounced improvements following treatment with AADvac1 compared to those administered placebo, as analyzed using ANCOVA.

TABLE 23
AADvac1 reduces clinical decline in patients with young onset of
Alzheimer's Disease (Patients with age ≥50 and ≤67)

| Change from | | | % | | |
|---|---|---|---|---|---|
| baseline | AADvac1 | Placebo | Reduction | Cohen's | boot, |
| (104w) | (n) | (n) | vs. Placebo | d | p-value |
| CDR-SB | 31 | 11 | 42 | −0.802 | 0.062 |
| MMSE | 31 | 10 | 31 | 0.549 | 0.233 |
| ADCS-MCI-ADL | 32 | 10 | 26 | 0.356 | 0.309 |

TABLE 24A
AADvac1 significantly reduces levels of NfL and
brain atrophy in patients with young onset of Alzheimer's
Disease (Patients with age ≥50 and ≤67)

| Change from | | | % | | |
|---|---|---|---|---|---|
| baseline | AADvac1 | Placebo | Reduction | Cohen's | boot. |
| (104w) | (n) | (n) | vs. Placebo | d | p-value |
| Plasma NfL | 33 | 12 | 73 | −0.882 | 0.033 |
| Cortical | 29 | 10 | 47 | 0.908 | 0.021 |
| Volume | | | | | |
| Temporal Lobe | 29 | 10 | 39 | 0.765 | 0.052 |
| Right | 29 | 10 | 31 | 0.679 | 0.076 |
| Hippocampus | | | | | |

TABLE 24B
Slower brain atrophy and less clinical decline was observed in AADvac1-treated patients with young onset of
Alzheimer's Disease (≥50 and ≤67); less cognitive decline was also observed in AADvac1-treated A+/T+ patients

| | | | AADvac1 | Placebo | Mean | | | |
|---|---|---|---|---|---|---|---|---|
| Change from | AADvac1 | placebo | (mean | (mean | adjusted | | | |
| baseline (week 104) | (n) | (n) | change) | change) | difference* | 95% CI* | p-value* | Cohen's d |
| Young subgroup | | | | | | | | |
| (≥50 and ≤67 | | | | | | | | |
| CDR-Sum of Boxes | 31 | 11 | 3.79 | 6.55 | −2.73 | (−5.17, −0.29) | 0.02963 | −0.802 |
| MMSE score | 31 | 10 | −6.48 | −9.40 | 3.35 | (−0.40, 7.10) | 0.07869 | 0.549 |
| ADCS-MCI-ADL 24 | 32 | 11 | −13.53 | −18.27 | 7.89 | (−0.92, 16.69) | 0.07769 | 0.356 |
| Algorithm-defined | | | | | | | | |
| A+T+ group | | | | | | | | |
| CDR-Sum of Boxes | 53 | 38 | 3.24 | 4.08 | −1.17 | (−2.32, −0.01) | 0.04809 | −0.273 |
| MMSE score | 53 | 37 | −5.68 | −6.38 | 1.35 | (−0.78, 3.48) | 0.21000 | 0.129 |
| ADCS-MCI-ADL 24 | 52 | 37 | −10.92 | −13.24 | 4.25 | (0.23, 8.28) | 0.03874 | 0.216 |

*= ANCOVA, adjusted for baseline value of the given assessment, age, and baseline plasma NfL.

AADvac1 was able to slow down cognitive decline, particularly in younger patients. The effect on cognition was supported by the brain atrophy measured by MRI and NfL. The effect of AADvac1 treatment on NfL is twice as large in younger patients as in older patients. Results on cognitive and functional assessments significantly correlated with biomarker outcomes.

Antibody Response:

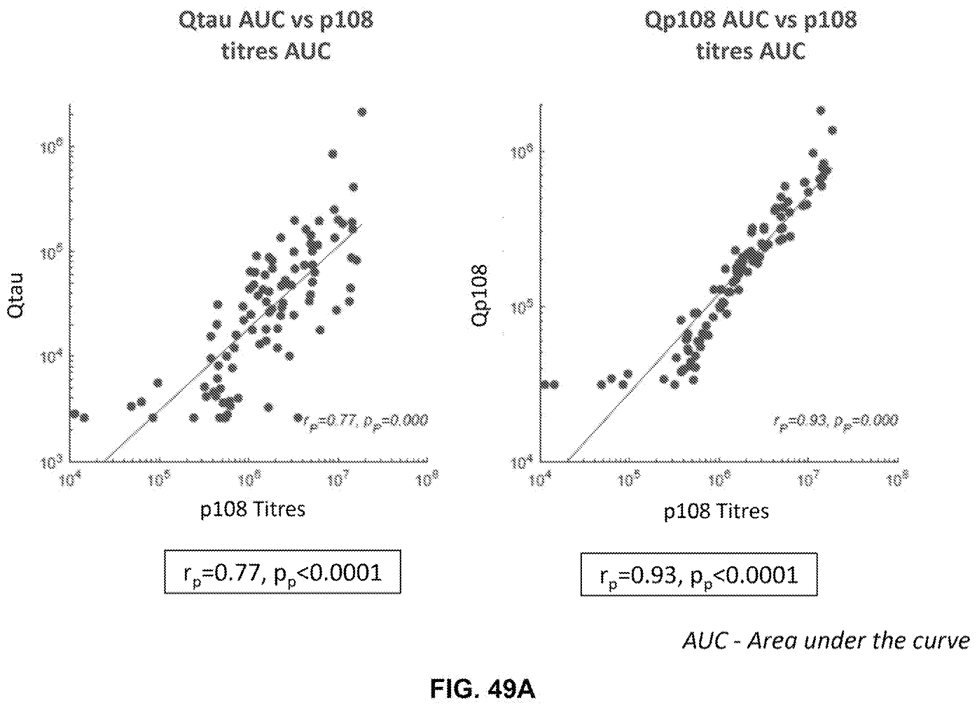

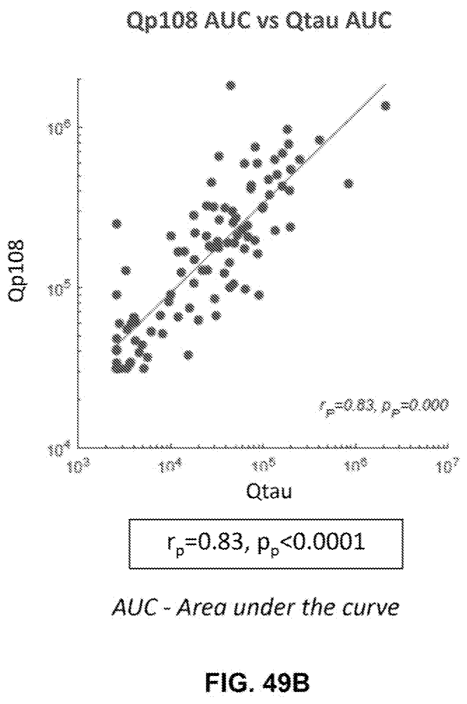

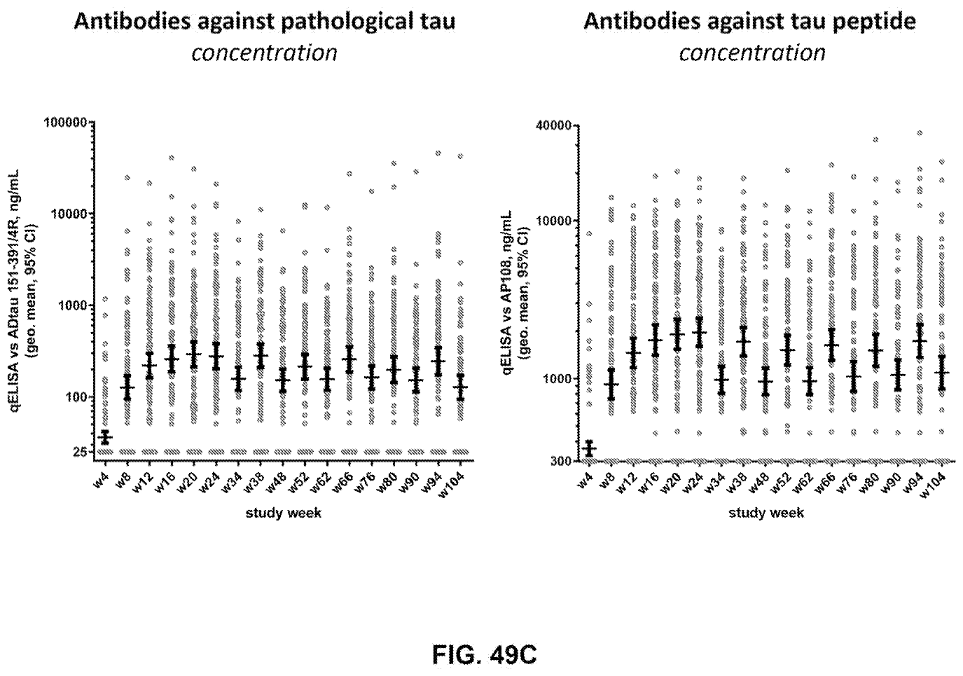

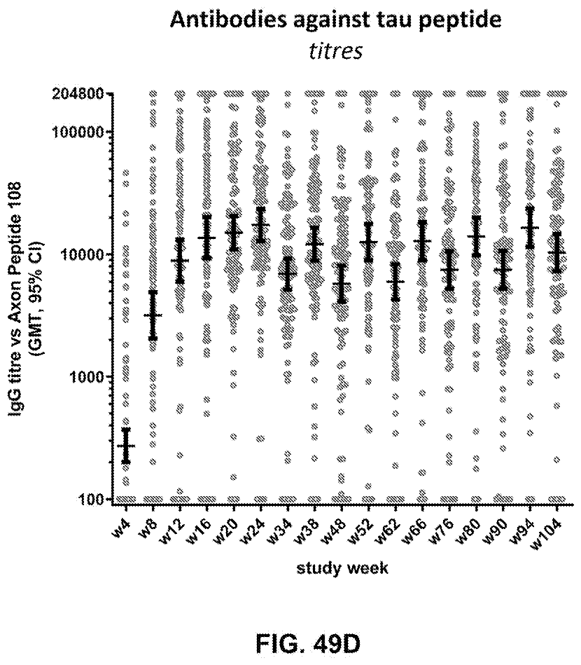

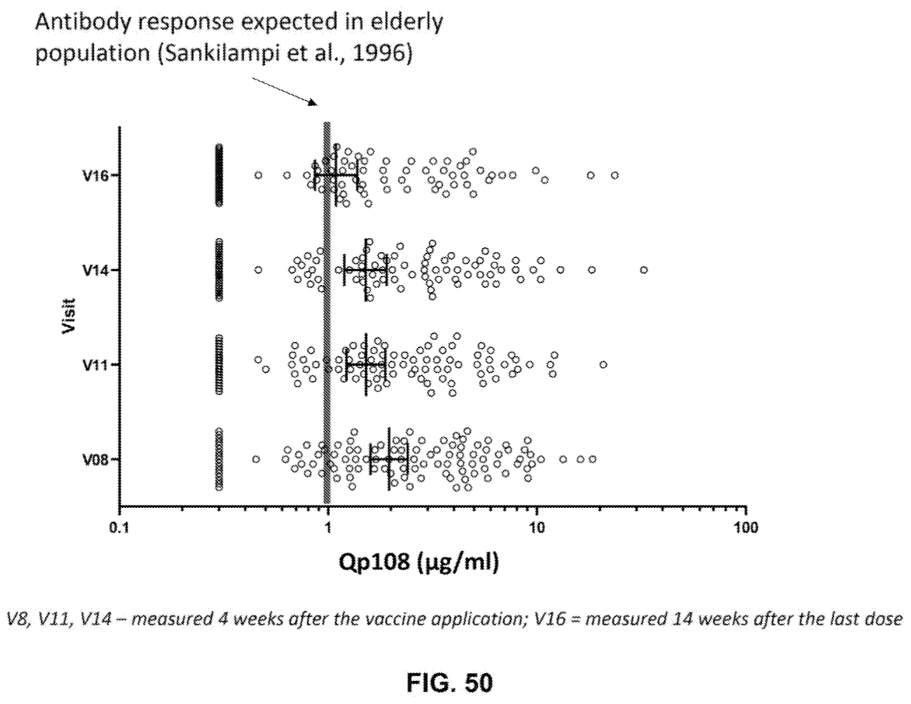

As shown in FIGS. 49A-49D, antibody response against tau peptide and pathological tau strongly correlate and show the same profile. Antibody response was excellent in the elderly population (FIG. 50). The strength of the antibody response in the majority of patients was observed to be in a higher range than expected for the elderly population. See Sankilampi et al., Antibody response in the elderly. J Infect Dis. 1996. AADvac1 yielded a high responder rate, with 98% of patients able to develop antibody titres. Boosters were effective at maintaining antibody level after an initial (e.g., six dose) dosing regimen. The data in FIG. 50 for V08, V11, and V14 demonstrate antibody response following the initial six dose regimen, the second booster, and the fourth booster, respectively.

Antibody Affinity:

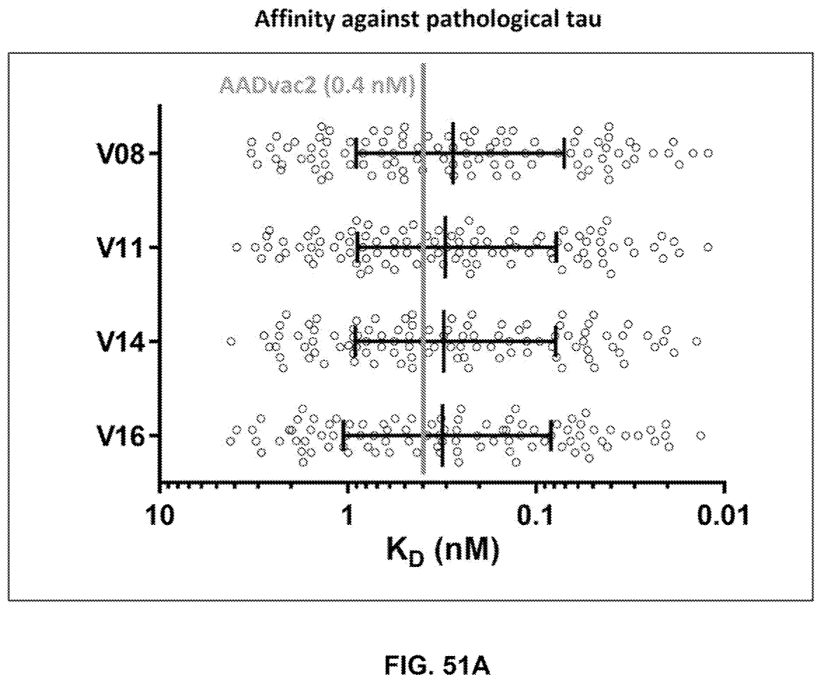

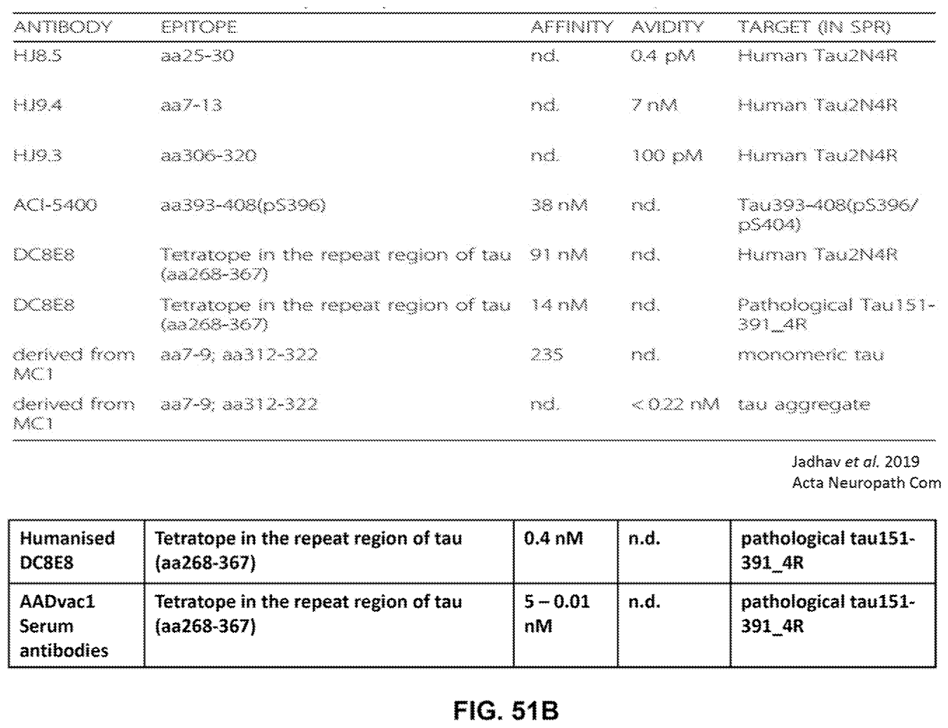

The affinity of AADvac1 serum antibodies was measured and is shown in FIG. 51A. Affinities ranged from about 4.2 nM to about 0.01 nM (FIG. 51A). Almost 80% of patients developed affinity better than 1 nM (e.g., in the range of 1-0.01 nM). This affinity is in the range of affinities of monoclonal antibodies reported in the literature. See Jadhav S. et al., A walk through tau therapeutic strategies. Acta Neuropathol Commun. 2019.

The affinity of AADvac1 serum antibodies was comparable to a humanized DC8E8 antibody as described in WO 2016/079597 (herein incorporated by reference in its entirely) and similar or better than other tested competitive tau monoclonal antibodies (FIG. 51B).

Immune Response and Efficacy (Alzheimer's Disease Biomarker (CSF) Positive Subgroup):

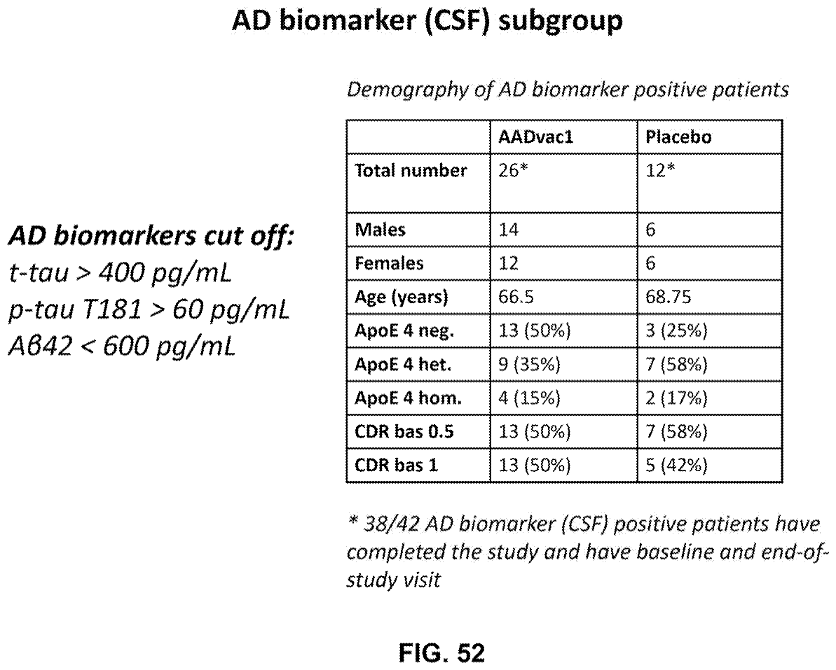

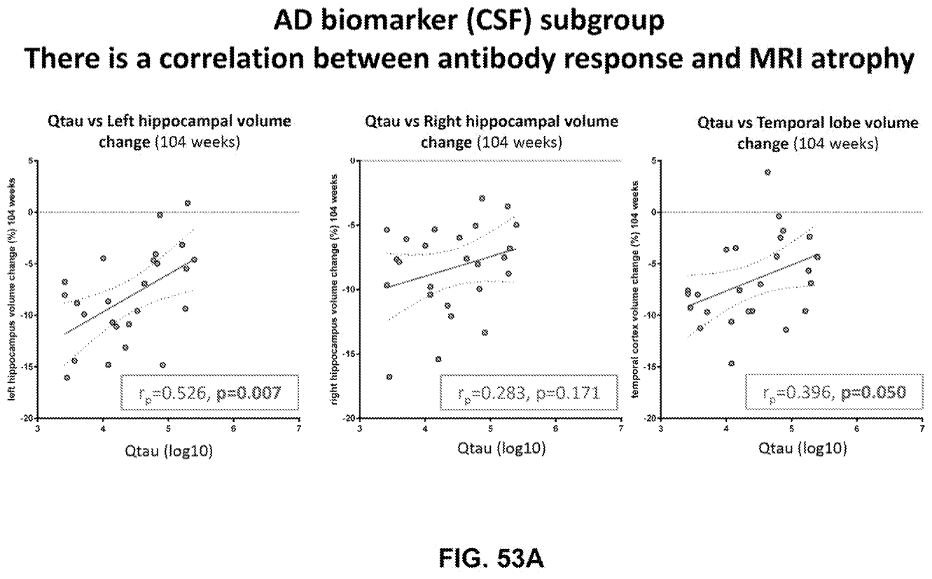

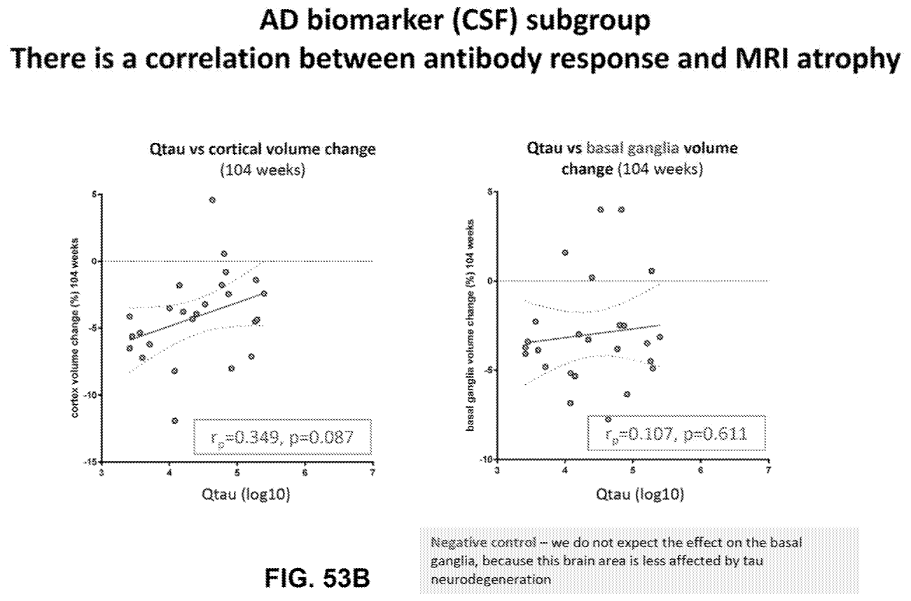

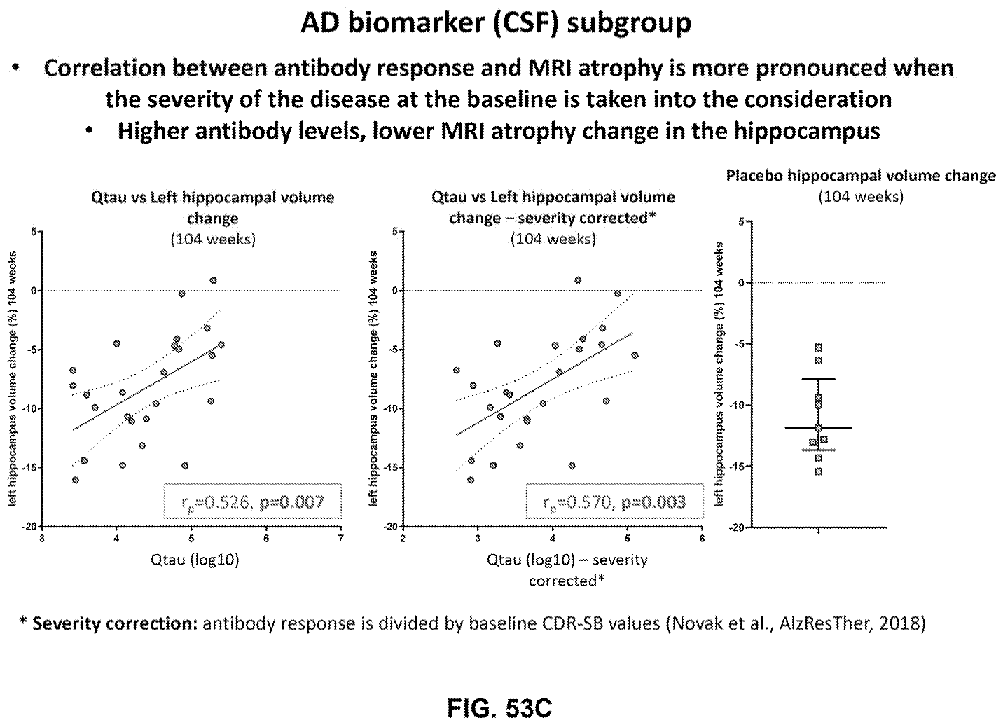

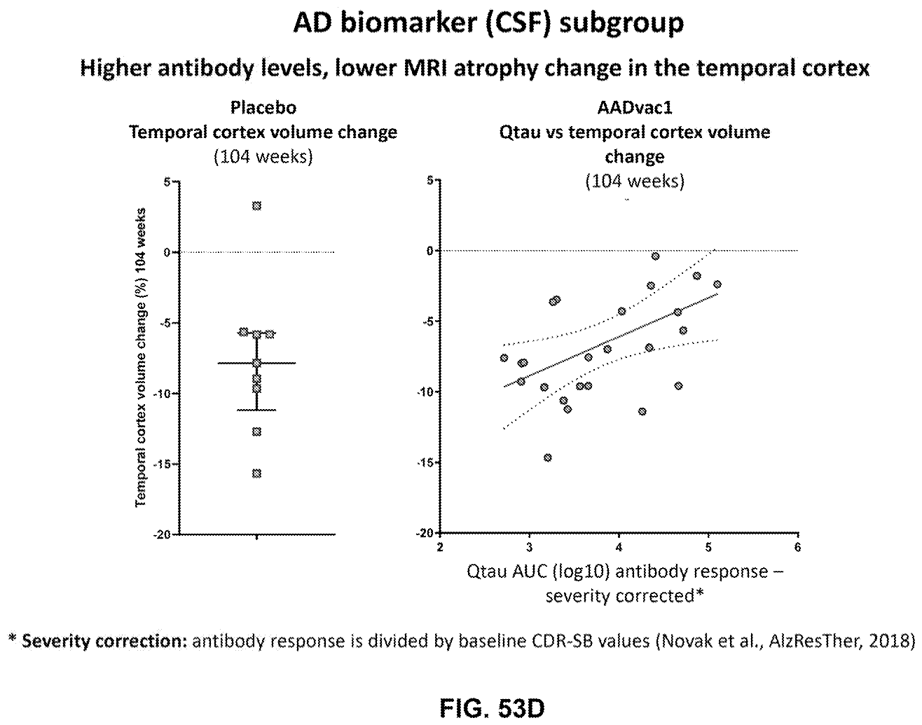

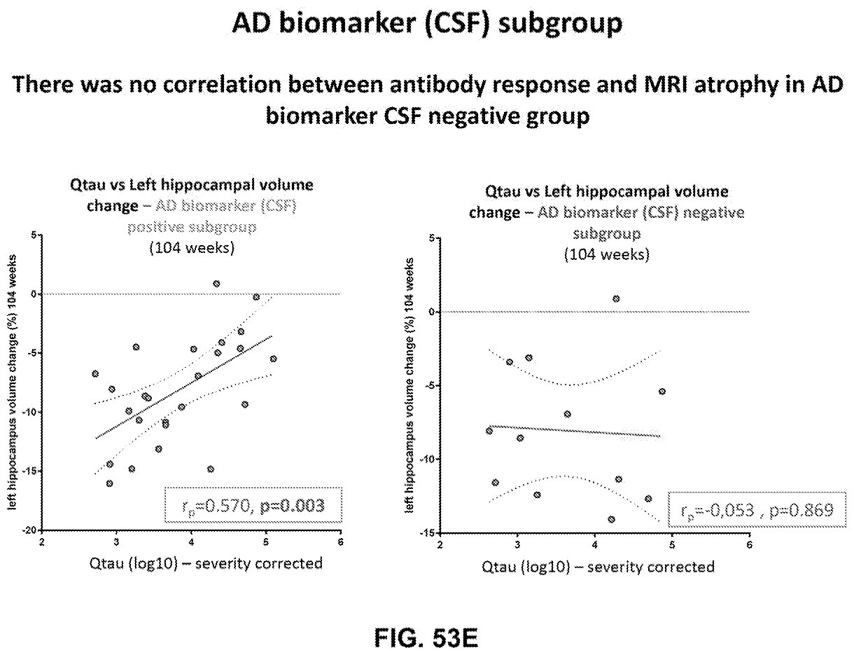

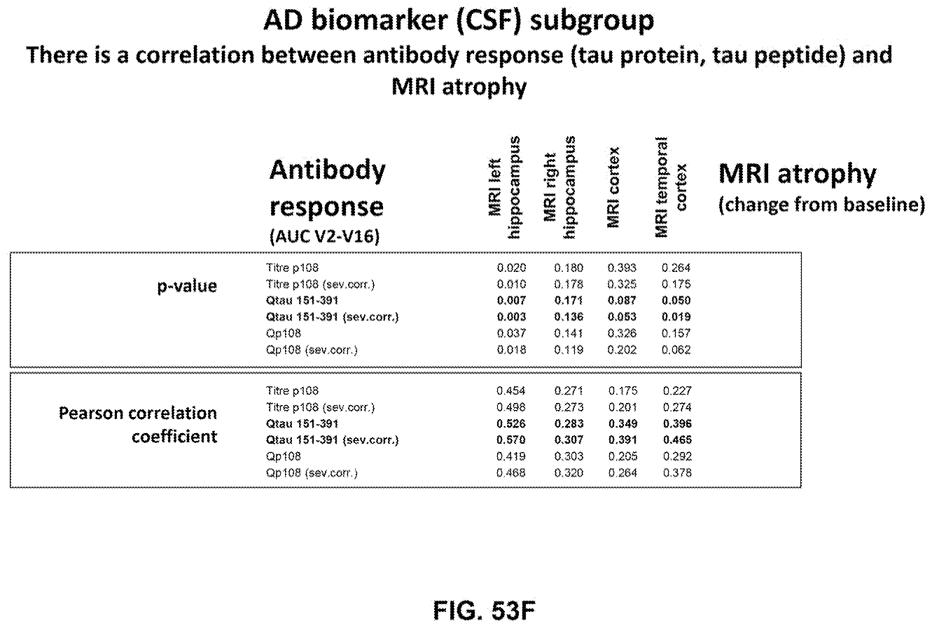

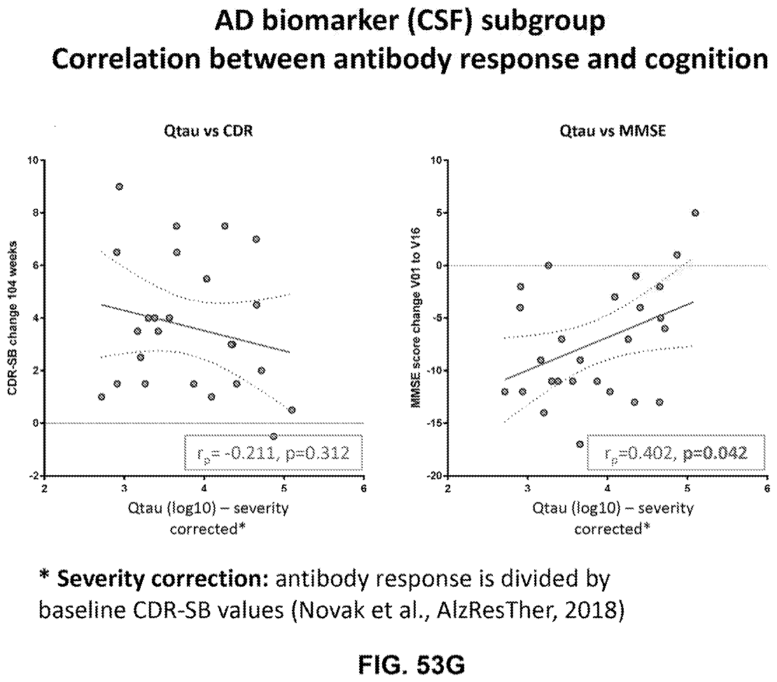

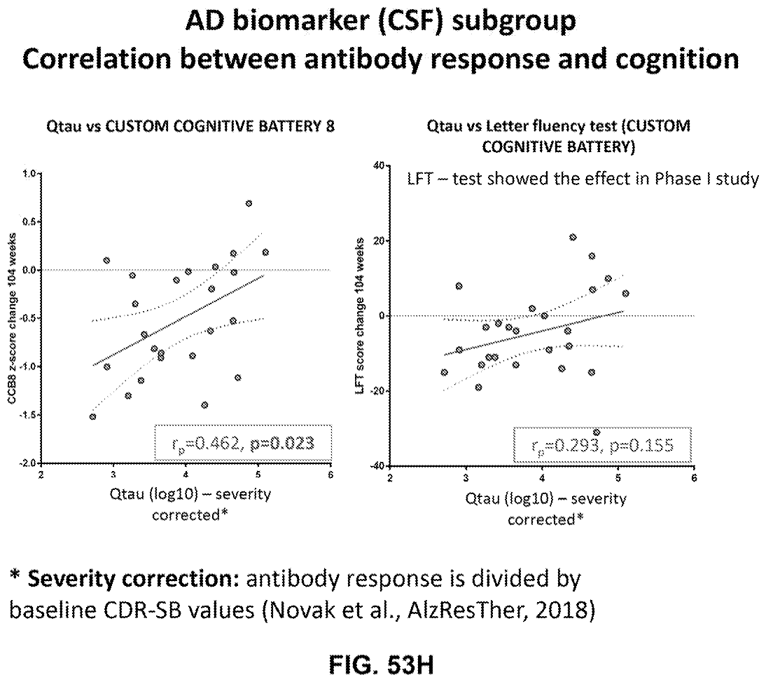

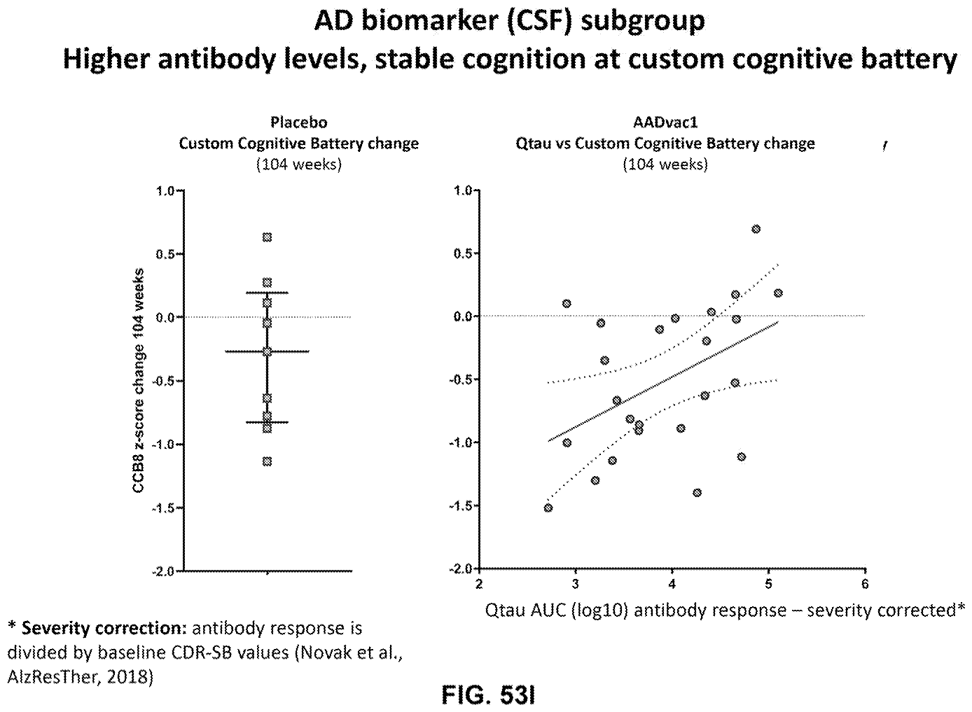

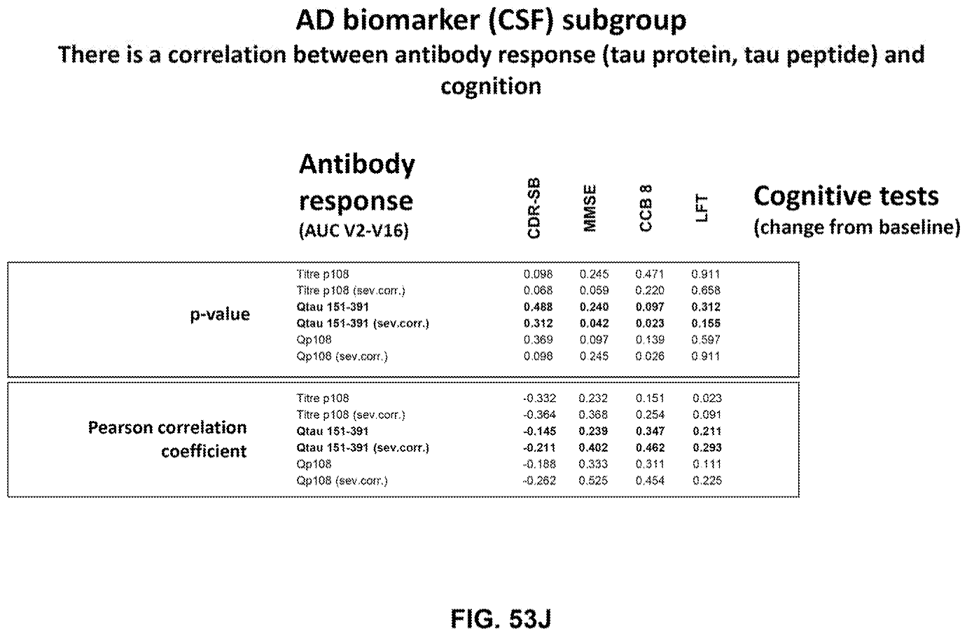

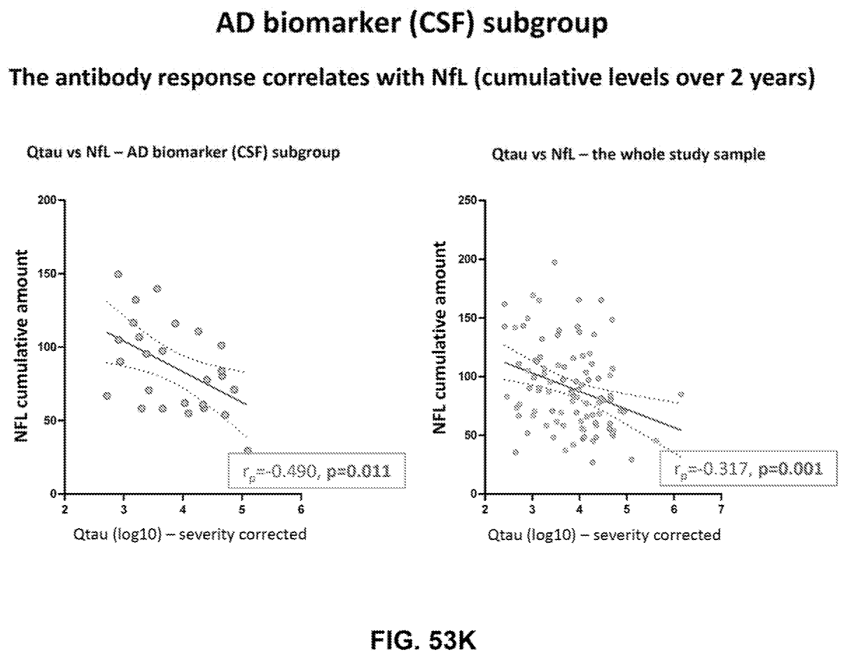

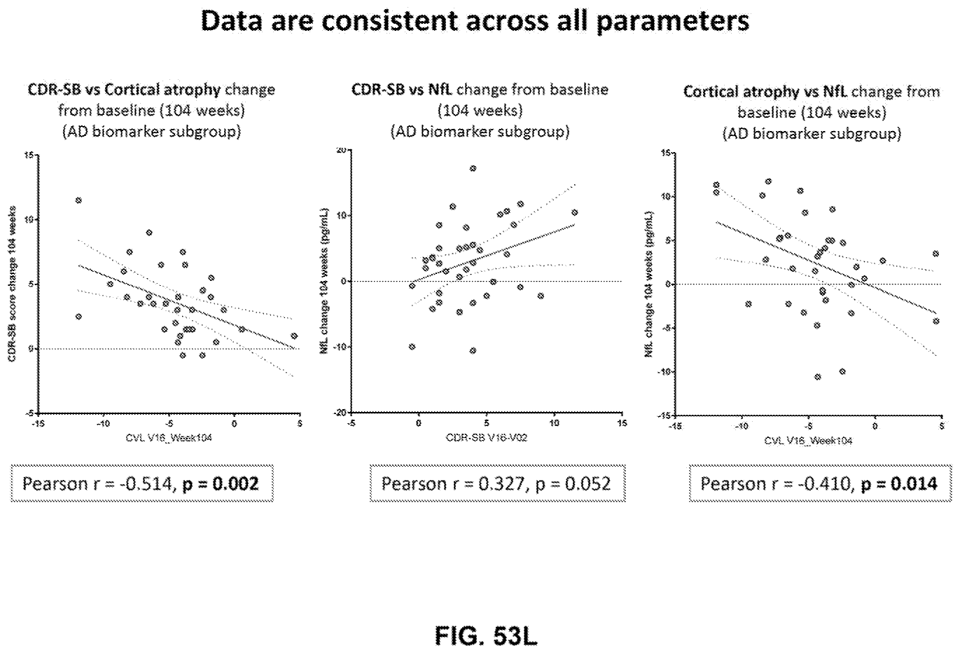

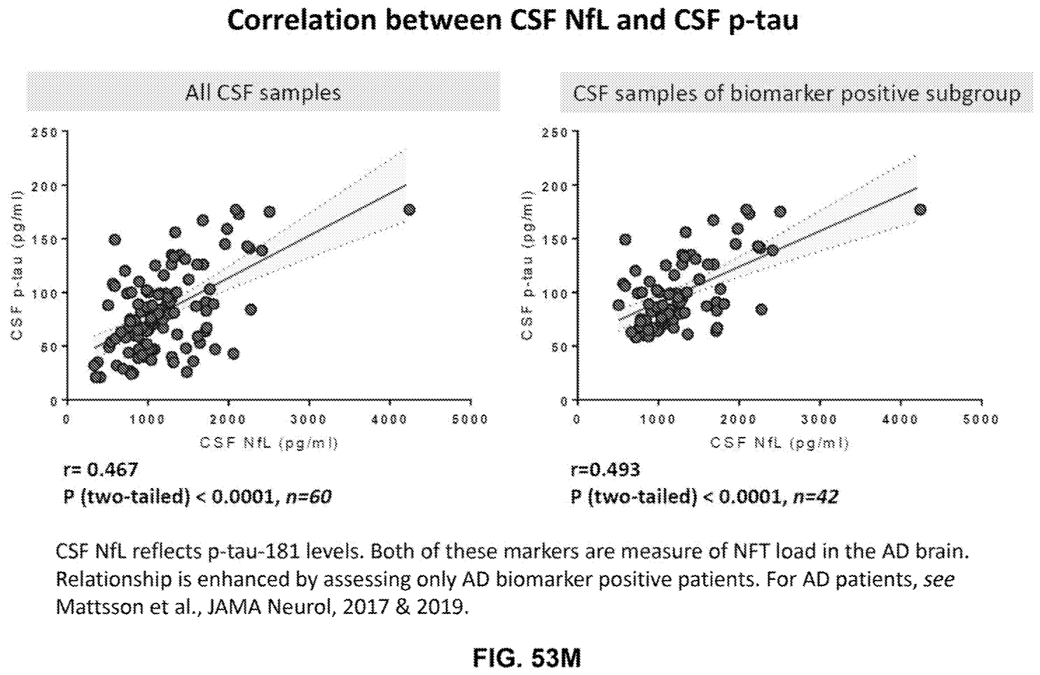

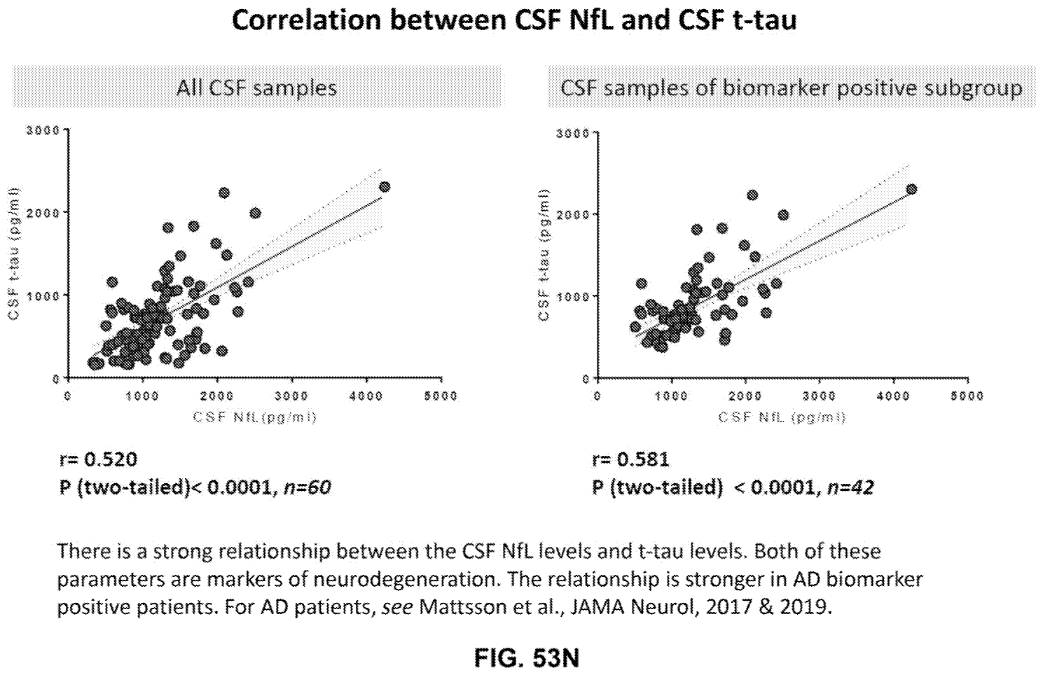

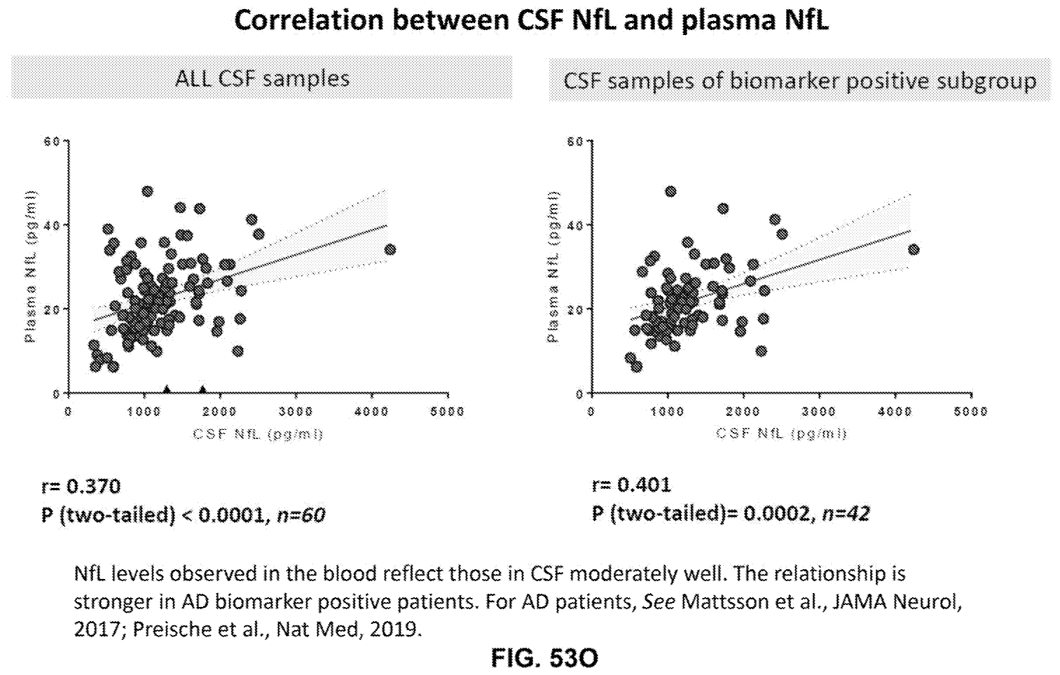

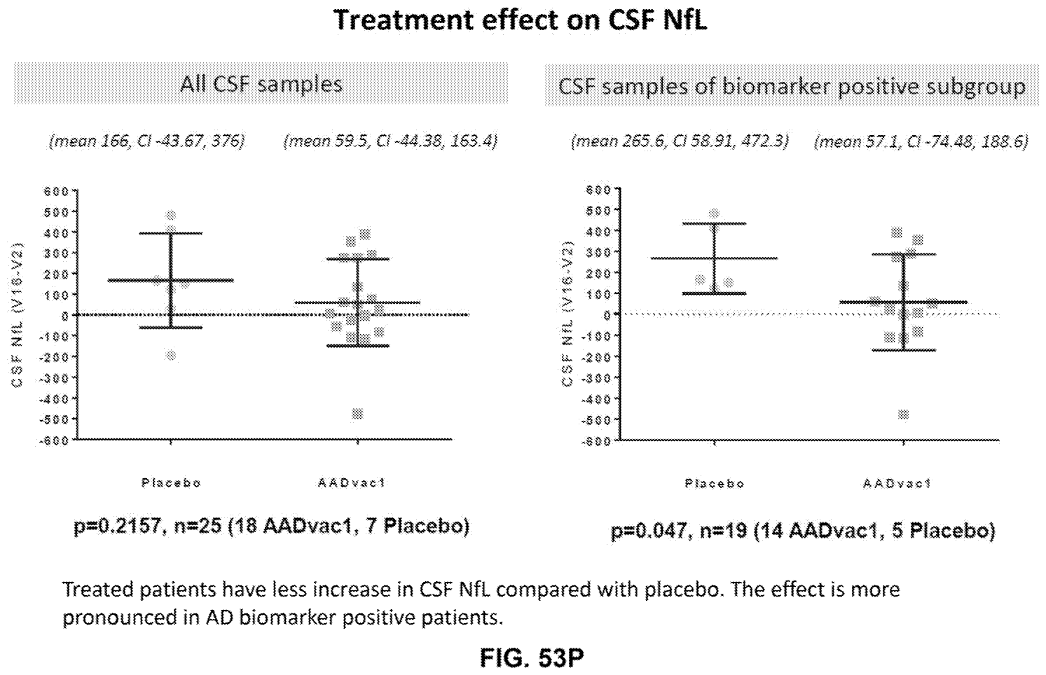

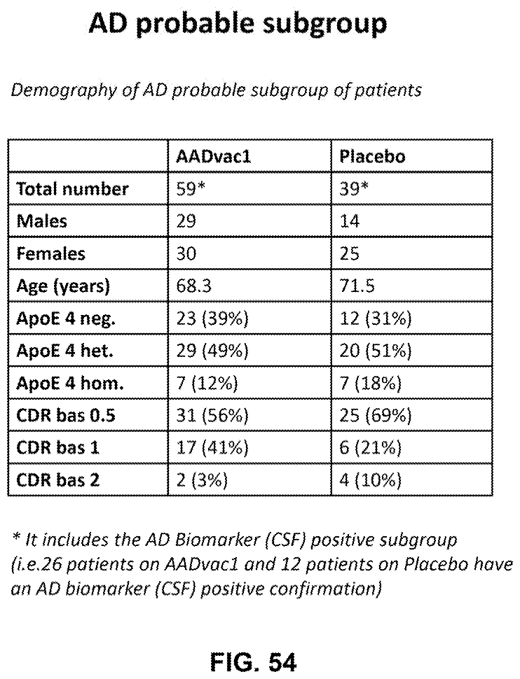

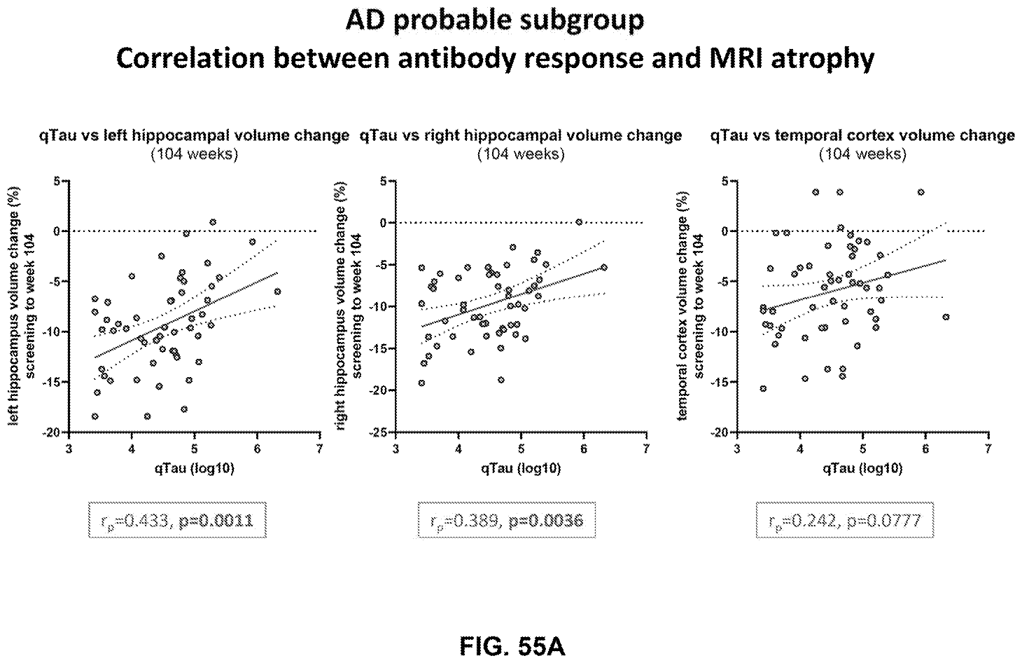

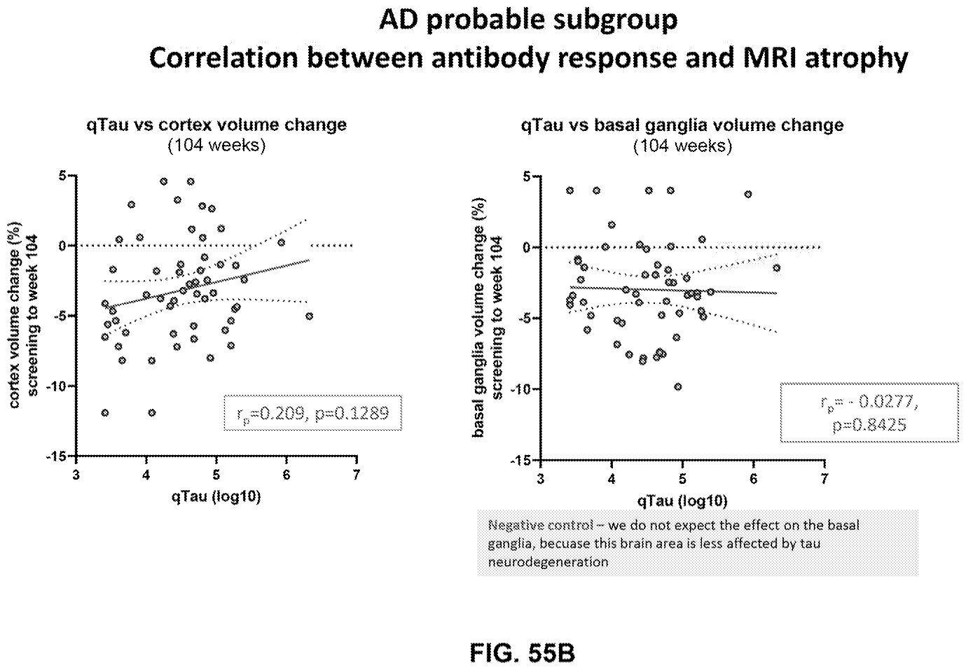

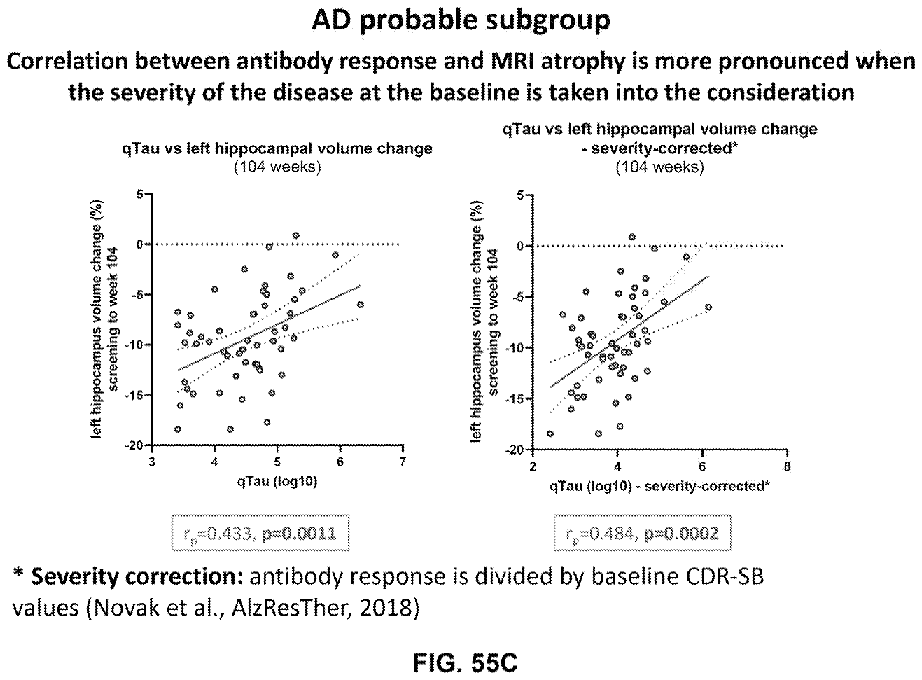

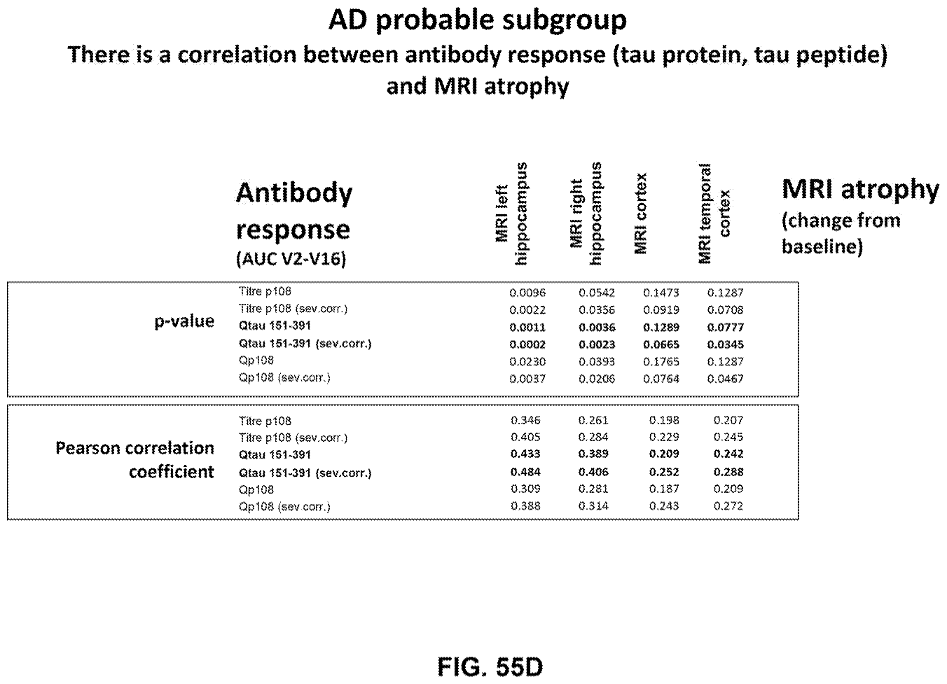

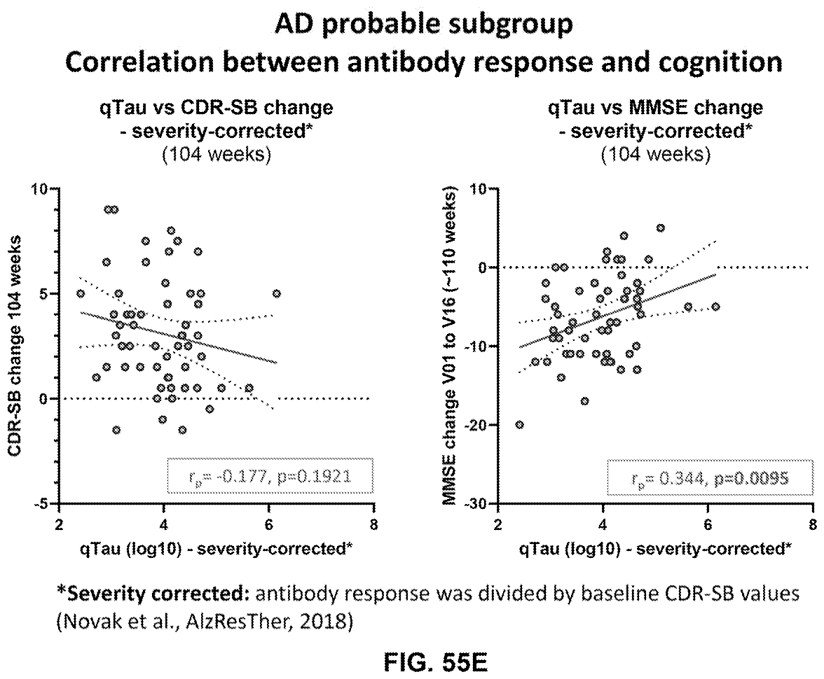

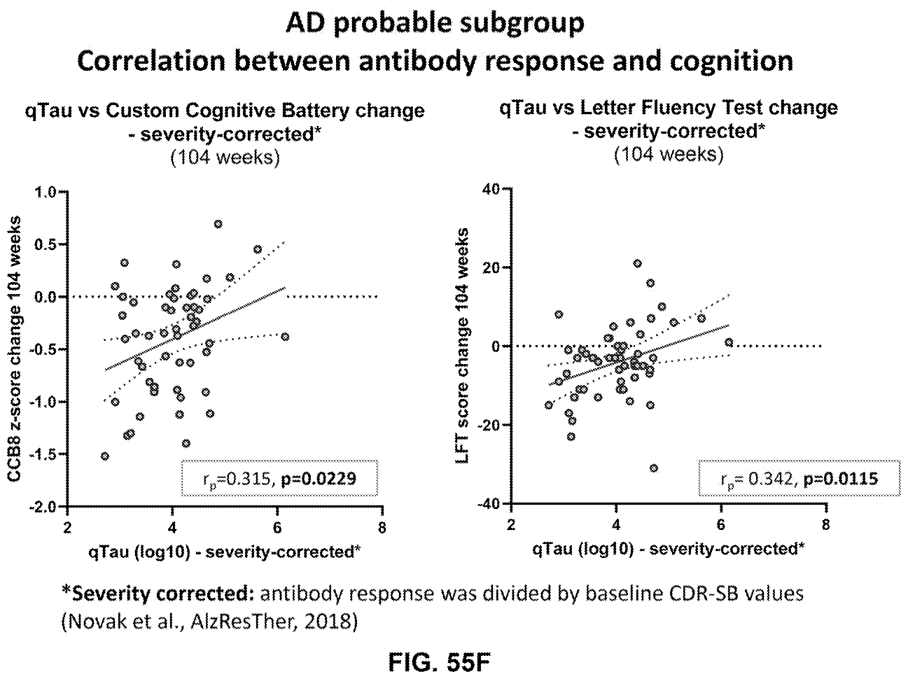

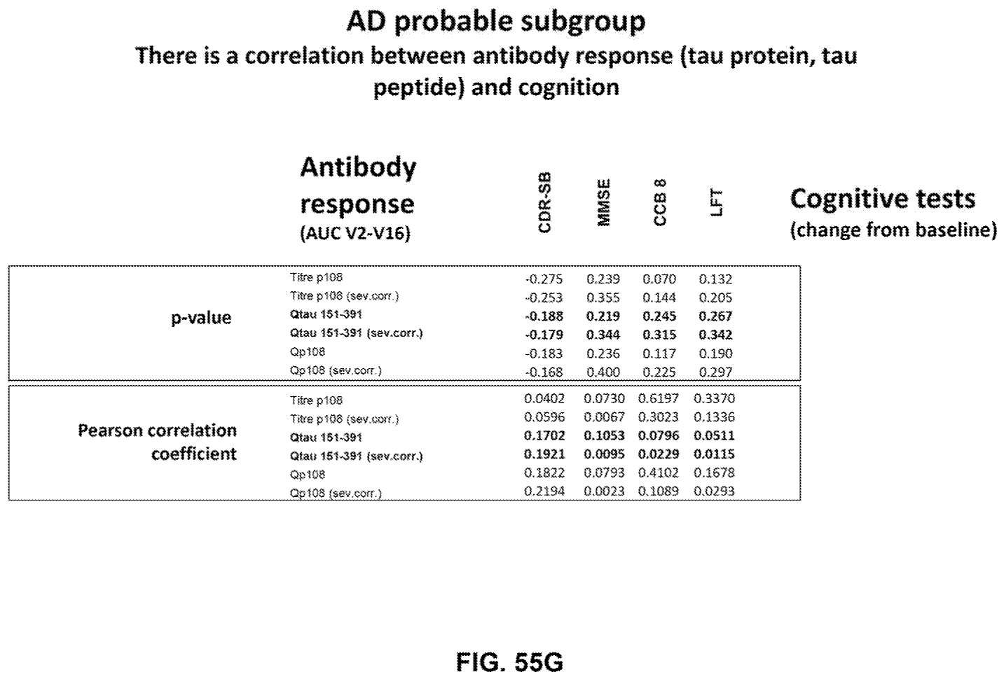

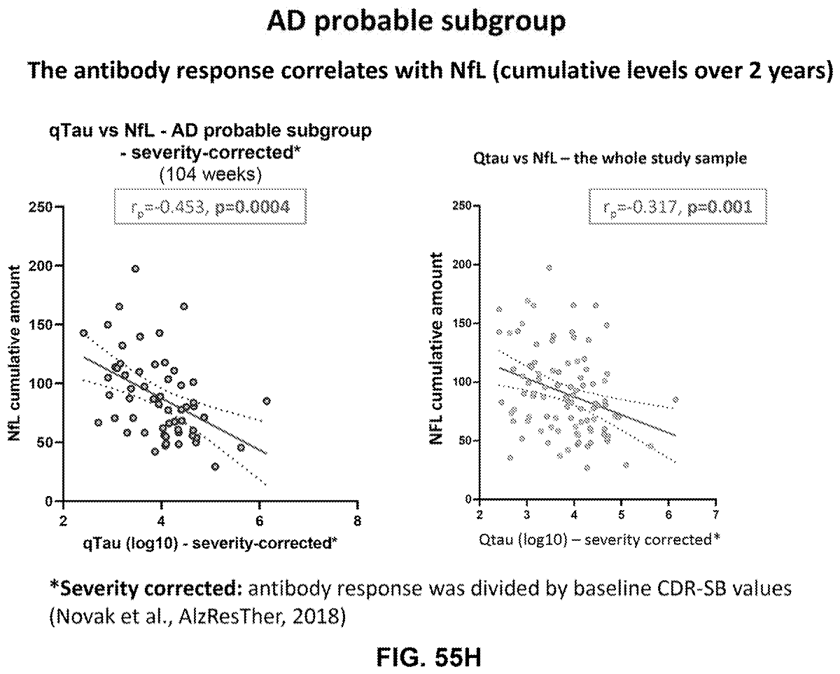

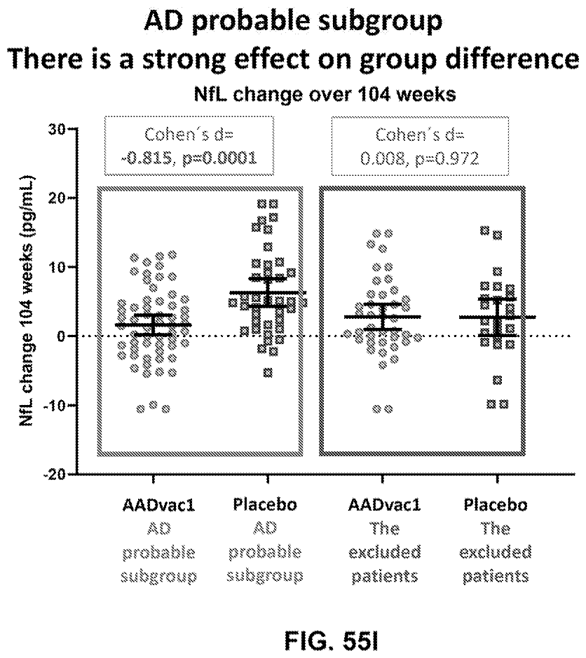

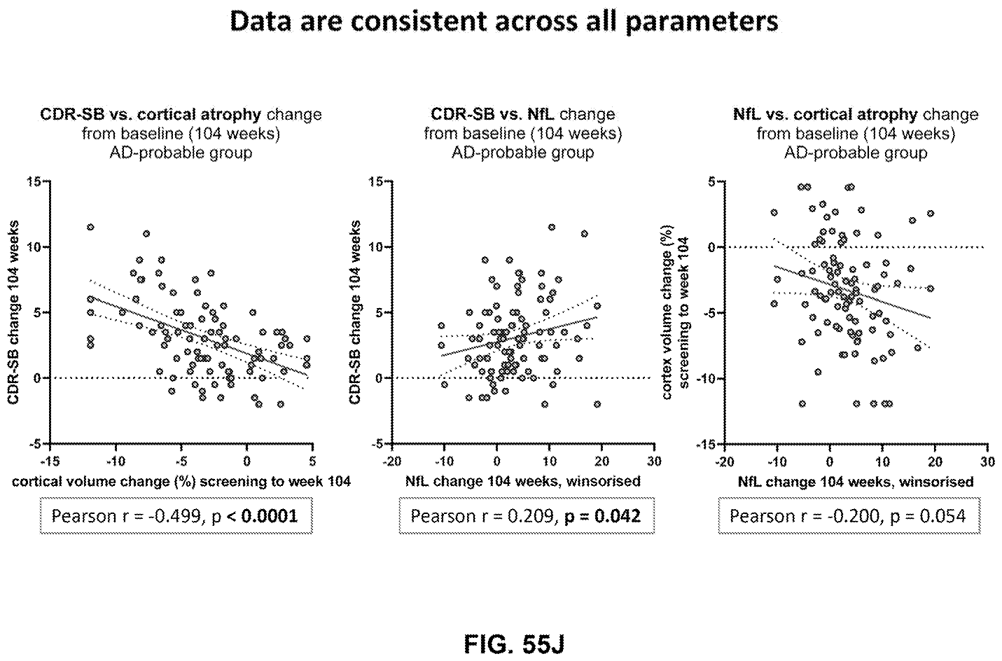

FIGS. 52, 53A-53L describe the AD biomarker (CSF) positive patient subgroup and demonstrate correlations between immune response and treatment effect in this subgroup. In the AD biomarker (CSF) positive patient subgroup, antibody response (e.g., titre) strongly correlated with MRI atrophy in the cortex and hippocampus and with cognitive battery testing. The correlation was even stronger when severity of the disease at the baseline was taken into consideration. On the contrary, in the AD biomarker negative subgroup there was no correlation observed between antibody response and MRI volumetry or between antibody response and cognition.

Compared with placebo, patients with higher antibody response displayed less atrophy in the hippocampus and temporal cortex. Patients with higher antibody response also demonstrated stable cognition during the two year study.

There was a correlation between antibody response and NfL (cumulative levels over 2 years) (FIG. 53K), showing lower NfL accumulation with higher titre, particularly when the data were corrected for disease severity at baseline.

The positive correlations between CSF NfL and CSF t-tau and CSF p-tau and between CSF NfL and plasma NfL observed for the treated population overall were even stronger in the AD biomarker (CSF) positive group (FIGS. 53M-53O). A treatment effect was observed on CSF NfL in the AD biomarker (CSF) positive group (FIG. 53P).

Immune Response and Efficacy (Exploratory Alzheimer's Disease-Probable Subgroup):

For patients where CSF samples were available, the presence of AD pathology (amyloid+, tau+) was assessed using the biomarkers (amyloid, tau) in the CSF. All patients who were tau and/or amyloid negative were excluded from the AD probable group.

From the remaining patients, those with lower likelihood of AD and/or with indicators of mixed pathology were excluded:
Patients with hallucinations are likely to have Lewy body pathology (α-synuclein), which is not expected to respond to AADvac1 treatment;
Patients with indicators of poor cerebrovascular health (Fazekas score 2, micro-bleeds) have a pronounced vascular dementia component, and a non-negligible portion of their decline is not driven by AD pathology;
Patients with low NfL levels either do not suffer from neurodegeneration, or only from a very mild case, and are not likely to progress even in the absence of treatment, confounding results;
Patients with excessive hippocampal atrophy (Scheltens 4) have a higher likelihood of hippocampal sclerosis (TDP-43 pathology), and a lower likelihood of AD pathology;
The oldest (80 and older) typically have slower-progressing AD, and very often mixed CNS pathology where only a fraction of decline is being driven by AD pathology, thus confounding results.

Without being bound by theory, these exclusion criteria facilitate identification of patients with Alzheimer's Disease from those having other diseases, disorders, or conditions, such as but not limited to TDP-43, vascular dementia, hippocampal sclerosis, or dementia with Lewy Bodies (DLB). Similar to the AD biomarker (CSF) positive subgroup, in the post-hoc defined AD probable subgroup antibody response strongly correlated with MRI atrophy in the cortex and hippocampus and with changes in cognitive battery. The correlation was even stronger when severity of the disease at the baseline was taken into the consideration. There was a correlation between antibody response and NfL (cumulative levels over 2 years). There was also a strong effect on group difference, as similarly observed in the AD biomarker (CSF) positive subgroup.

### Example 12: Further Studies

Early AD patients, e.g., patients 50-70 years of age with mild cognitive impairment (MCI) or mild AD, are administered AADvac1 or placebo. The patients are confirmed with diagnosis of Alzheimer's Disease according to the 2018 NIA-AA criteria, for example: the patients are screened for CSF biomarkers, total tau protein>400 pg/mL, pT181 tau protein>60 pg/mL, and Aβ42<600 pg/mL. Patients are assessed for at least 24-30 months. The dosing regimen comprises six initial monthly doses followed by booster doses every three months (e.g., for a study duration of 30 months, seven boosters are administered). The primary efficacy endpoint is CDR-SB score. Secondary endpoints include other cognitive and function tests, MRI, and plasma NfL. Further endpoints may include biomarker assessment in CSF, plasma, and/or using DTI; PK assessment in CSF and/or plasma; tau PET, and safety.

### Exemplary Embodiments

Disclosed embodiments may include, but are not limited to, any of the following:
1. A method for diagnosing a patient suffering from Alzheimer's Disease (AD) or mild cognitive impairment, the method comprising:
   (a) detecting the presence and/or amount of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p in the patient or a sample from the patient;
   (b) wherein the presence of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p and/or an altered amount relative to an amount in a control sample or threshold indicates Alzheimer's Disease in the subject; and
   (c) diagnosing the presence or absence of Alzheimer's Disease in the patient based on step (b).
2. The method of embodiment 1, wherein step (a) comprises:
   (a) obtaining a cerebrospinal fluid (CSF), serum, or blood plasma sample from the patient; and
   (b) optionally extracting one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p from the CSF, serum, or blood plasma of the patient; and
   (c) optionally performing cDNA synthesis; and
   (d) detecting the presence and/or amount of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p, e.g., using quantitative polymerase chain reaction (qPCR).
3. The method of embodiment 1 or embodiment 2, wherein the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, or hsa-miR-21-5p in the patient is 1, 2, 3, 4, 5, 6, 7, 8, 9, 10, 15, 20, 25, 50, 100, or more fold different than in the control sample.
4. The method of any one of embodiments 1-3, wherein the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, or hsa-miR-21-5p in the patient is 10%, 20%, 30%, 40%, 50%, 60%, 70%, 80%, 90%, or 100% less than in the control sample.
5. The method of any one of embodiments 1-4, wherein the amount of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, or hsa-miR-21-5p in the patient is 10%, 20%, 30%, 40%, 50%, 60%, 70%, 80%, 90%, 100%, 200%, or 500% greater than in the control sample.
6. The method of any one of embodiments 1-5, wherein the presence and/or an altered amount of at least 2, at least 3, at least 4, at least 5, at least 6, or at least 7 or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p relative to the control sample or threshold indicates Alzheimer's Disease in the subject.
7. The method of any one of embodiments 1-6, wherein the presence and/or amount of one or more of hsa-let-7a-5p, hsa-miR-15a-5p, hsa-miR191-5p, and hsa-miR-23a-3p is detected.
8. A method for diagnosing a patient suffering from Alzheimer's Disease (AD) or mild cognitive impairment, the method comprising:
   (a) detecting the presence and/or amount of one or more metabolites, e.g., 2,4-dihydroxybutanoic acid, a phospholipid, phosphatidylcholine, sphingomyelin, or sterol, in blood plasma, serum, or cerebrospinal fluid (CSF) of the patient;
   (b) wherein the presence of one or more metabolites and/or an altered amount relative to an amount in a control sample or threshold indicates Alzheimer's Disease in the subject; and
   (c) diagnosing the presence or absence of Alzheimer's Disease in the patient based on step (b).
9. The method of embodiment 8, wherein step (a) further comprises
   (a) obtaining a cerebrospinal fluid (CSF), serum, or blood plasma sample from the patient;
   (b) extracting one or more metabolites from the CSF, serum, or blood plasma of the patient; and
   (c) determining the amount of one or more metabolites, e.g., using mass spectrometry.
10. The method of embodiment 9, wherein the mass spectrometry is time-of-flight mass spectrometry or tandem mass spectrometry/mass spectrometry.
11. The method of any one of embodiments 1-10, wherein the control sample is from a healthy individual or a patient with Alzheimer's Disease, wherein optionally the control sample is blood plasma or CSF.
12. The method of any one of embodiments 8-11, wherein the amount of metabolites in the patient is 1, 2, 3, 4, 5, 6, 7, 8, 9, 10, 15, 20, 25, 50, 100, or more fold different than in the control sample.
13. The method of any one of embodiments 8-12, wherein the amount of metabolites in the patient is 10%, 20%, 30%, 40%, 50%, 60%, 70%, 80%, 90%, 100%, 200%, or 500% greater than in the control sample.
14. The method of any one of embodiments 8-12, wherein the amount of metabolites in the patient is 10%, 20%, 30%, 40%, 50%, 60%, 70%, 80%, 90%, or 100%, less than in the control sample.
15. The method of any one of embodiments 8-14, wherein the presence and/or an altered amount of at least 2, at least 3, at least 4, at least 5, at least 6, or at least 7 or more metabolites relative to the control sample or threshold indicates Alzheimer's Disease in the subject.
16. A method of treating Alzheimer's Disease (AD) or mild cognitive impairment, comprising:
   a. detecting the presence and/or amount of one or more of hsa-let-7a-5p, hsa-miR-15a-5p, hsa-miR191-5p, and hsa-miR-23a-3p in a sample from a patient;
   b. selecting the patient for treatment when the sample from the patient has one or more of hsa-let-7a-5p, hsa-miR-15a-5p, hsa-miR191-5p, and hsa-miR-23a-3p or an altered level of one or more of hsa-let-7a-5p, hsa-miR-15a-5p, hsa-miR191-5p, and hsa-miR-23a-3p relative to a control sample or threshold; and
   c. administering to the selected patient one or more doses of an immunogenic composition comprising an immunogenic peptide comprising SEQ ID NO: 1 or SEQ ID NO: 2.
17. The method of embodiment 16, wherein the amount of hsa-let-7a-5p, hsa-miR-15a-5p, hsa-miR191-5p, and/or hsa-miR-23a-3p in the sample from the patient is 1, 2, 3, 4, 5, 6, 7, 8, 9, 10, 15, 20, 25, 50, 100, or more fold different than in the control sample.
18. The method of embodiment 16 or embodiment 17, wherein the amount of hsa-let-7a-5p and/or hsa-miR-15a-5p in the sample from the patient is 1, 2, 3, 4, 5, 6, 7, 8, 9, 10, 15, 20, 25, 50, 100, or more fold less than in the control sample.
19. The method of any one of embodiments 16-18, wherein the amount of hsa-miR-15a-5p in the sample from the patient is 1, 2, 3, 4, 5, 6, 7, 8, 9, 10, 15, 20, 25, 50, 100, or more fold less than in the control sample.
20. The method of any one of embodiments 16-19, wherein the amount of hsa-miR191-5p and/or hsa-miR-23a-3p in the sample from the patient is 1, 2, 3, 4, 5, 6, 7, 8, 9, 10, 15, 20, 25, 50, 100, or more fold greater than in the control sample.
21. The method of any one of embodiments 16-20, wherein the amount of hsa-miR-23a-3p in the sample from the patient is 1, 2, 3, 4, 5, 6, 7, 8, 9, 10, 15, 20, 25, 50, 100, or more fold greater than in the control sample.
22. The method of any one of embodiments 16-21, wherein the presence and/or an altered amount of at least 2, at least 3, or at least 4 of hsa-let-7a-5p, hsa-miR-15a-5p, hsa-miR191-5p, and hsa-miR-23a-3p in the sample from the patient relative to the control sample or threshold indicates Alzheimer's Disease in the patient.
23. The method of any one of embodiments 16-22, wherein the presence and/or an altered amount of at least 2, at least 3, or at least 4 of hsa-let-7a-5p, hsa-miR-15a-5p, hsa-miR191-5p, and hsa-miR-23a-3p in the sample from the patient relative to the control sample or threshold indicates that the patient is selected for treatment with one or more doses of an immunogenic composition comprising an immunogenic peptide comprising SEQ ID NO: 1 or SEQ ID NO: 2.
24. A method of treating Alzheimer's Disease (AD) or mild cognitive impairment, comprising administering one or more doses of an immunogenic composition comprising an immunogenic peptide comprising SEQ ID NO: 1 or SEQ ID NO: 2 to a patient expressing one or more of hsa-let-7a-5p, hsa-miR-15a-5p, hsa-miR191-5p, and hsa-miR-23a-3p in a sample, wherein optionally expression of one or more of hsa-let-7a-5p, hsa-miR-15a-5p, hsa-miR191-5p, and hsa-miR-23a-3p is compared to expression in a control sample or to a threshold.
25. The method of embodiment 24, wherein the amount of hsa-let-7a-5p, hsa-miR-15a-5p, hsa-miR191-5p, and/or hsa-miR-23a-3p in the sample from the patient is about 1, 2, 3, 4, 5, 6, 7, 8, 9, 10, 15, 20, 25, 50, 100, or more fold different relative to a control sample or threshold.
26. The method of embodiment 25, wherein the amount of hsa-miR191-5p and/or hsa-miR-23a-3p in the sample from the patient is about 1, 2, 3, 4, 5, 6, 7, 8, 9, 10, 15, 20, 25, 50, 100, or more fold greater than in a control sample or threshold.
27. The method of embodiment 25, wherein the amount of hsa-let-7a-5p and/or hsa-miR-15a-5p in the sample from the patient is about 1, 2, 3, 4, 5, 6, 7, 8, 9, 10, 15, 20, 25, 50, 100, or more fold less than in a control sample or threshold.
28. A method of treating Alzheimer's Disease (AD) or mild cognitive impairment, comprising:
   (a) determining the genotype for each of the two alleles of the ApoE gene present in the patient;
   (b) selecting the patient for treatment when the patient has at least one ApoE-ε4 allele; and
   (c) administering to the selected patient one or more doses of an immunogenic composition comprising an immunogenic peptide comprising SEQ ID NO: 1 or SEQ ID NO: 2.
29. The method of embodiment 28, wherein the patient has two c4 alleles.
30. A method of treating Alzheimer's Disease (AD) or mild cognitive impairment, comprising administering one or more doses of an immunogenic composition comprising an immunogenic peptide comprising SEQ ID NO: 1 or SEQ ID NO: 2 to a patient having at least one c4 allele of the ApoE gene.
31. A method of treating Alzheimer's Disease (AD) or mild cognitive impairment, comprising:
   (a) detecting the presence and/or amount of neurofilament light chain (NfL) in a sample from a patient;
   (b) selecting the patient for treatment when the sample from the patient contains NfL and/or an altered amount of NfL relative to a control sample or threshold; and
   (c) administering to the selected patient one or more doses of an immunogenic composition comprising an immunogenic peptide comprising SEQ ID NO: 1 or SEQ ID NO: 2.
32. The method of embodiment 31, wherein the amount of NfL in the sample from the patient is 1, 2, 3, 4, 5, 6, 7, 8, 9, 10, 15, 20, 25, 50, 100, or more fold higher than in the control sample.
33. The method of embodiment 31 or embodiment 32, wherein the amount of NfL in the sample from the patient is more than a threshold of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg/ml.
34. The method of any one of embodiments 31-33, wherein the amount of NfL in the sample from the patient is 10%, 20%, 30%, 40%, 50%, 60%, 70%, 80%, 90%, or 100% greater than in the control sample.
35. A method of treating Alzheimer's Disease (AD) or mild cognitive impairment, comprising administering one or more doses of an immunogenic composition comprising an immunogenic peptide comprising SEQ ID NO: 1 or SEQ ID NO: 2 to a patient having NfL in a sample.
36. The method of embodiment 35, wherein the amount of NfL in the sample from the patient is less than 10, 20, 30, 40, 50, 60, 70, 80, 90, or 100 μg/ml.
37. The method of embodiment 35 wherein the amount of NfL in the sample from the patient is greater than 20 μg/ml.
38. A method of treating Alzheimer's Disease (AD) or mild cognitive impairment, comprising:
   (a) detecting the presence and/or amount of neurogranin in a sample from a patient;
   (b) selecting the patient for treatment when the sample from the patient contains neurogranin and/or an increased amount relative to a control sample or threshold; and
   (c) administering to the selected patient one or more doses of an immunogenic composition comprising an immunogenic peptide comprising SEQ ID NO: 1 or SEQ ID NO: 2.
39. The method of embodiment 38, wherein the amount of neurogranin in the sample from the patient is 1, 2, 3, 4, 5, 6, 7, 8, 9, 10, 15, 20, 25, 50, 100, or more fold different than in the control sample.
40. The method of embodiment 38 or claim 39, wherein the amount of neurogranin in the sample from the patient is greater than a threshold of 1, 2, 3, 4, 5, 6, 7, 8, 9, 10, 15, 20, 25, 50, or 100 pg/ml.
41. The method of any one of embodiments 38-40, wherein the amount of neurogranin in the sample from the patient is 10%, 20%, 30%, 40%, 50%, 60%, 70%, 80%, 90%, or 100% less than in the control sample.
42. The method of any one of embodiments 38-40, wherein the amount of neurogranin in the sample from the patient is 10%, 20%, 30%, 40%, 50%, 60%, 70%, 80%, 90%, or 100% greater than in the control sample.
43. The method of any one of embodiments 16-42, wherein the immunogenic peptide further comprises a carrier protein or peptide.
44. The method of embodiment 43, wherein the carrier is keyhole limpet hemocyanin (KLH) or a fragment thereof.
45. The method of embodiment 44, wherein the KLH or fragment thereof is coupled to SEQ ID NO: 1 or SEQ ID NO: 2 via a maleimide linker, wherein optionally the maleimide linker is N-γ-maleimidobutyryl-oxysulfosuccinimide ester (Sulfo-GMBS).
46. The method of any one of embodiments 16-45, wherein a dose comprises about 20-50 μg, e.g., at least about 20 μg, at least about 30 μg, at least about 40 μg, or at least about 50 μg of the immunogenic peptide.
47. The method of any one of embodiments 16-46, wherein each dose comprises about 40 μg of the immunogenic peptide.
48. The method of any one of embodiments 16-47, wherein the immunogenic composition further comprises an adjuvant.
49. The method of embodiment 48, wherein the adjuvant comprises an aluminum compound.
50. The method of embodiment 49, wherein the aluminum compound comprises aluminum hydroxide (Al(OH)₃).
51. The method of any one of embodiments 48-50, wherein the adjuvant comprises about 0.1 mg to about 10 mg aluminum (Al³⁺), optionally, about 0.5 mg aluminum (Al³⁺).
52. The method of any one of embodiments 16-51, wherein the immunogenic composition further comprises a phosphate buffer, optionally in a volume of about 0.3 mL.
53. The method of any one of embodiments 16-52, wherein the immunogenic composition comprises an immunogenic peptide comprising SEQ ID NO: 2 coupled via a maleimide linker (optionally Sulfo-GMBS) to KLH, about 0.5 mg aluminum (Al³⁺), and a phosphate buffer, optionally in a volume of about 0.3 mL.
54. The method of any one of embodiments 16-53, wherein treating the patient comprises administering 5 doses, 6 doses, 7 doses, 8 doses, 9 doses, 10 doses, 11 doses, 12 doses, 13 doses, 14 doses, 15 doses, 20 doses, 25 doses, 50 doses or 100 doses of the immunogenic peptide.
55. The method of any one of embodiments 16-54, wherein treating the patient comprises administering doses at 2-week intervals, 3-week intervals, 4-week intervals, 5-week intervals, 6-week intervals, 7-week intervals, 8-week intervals, 9-week intervals, 10-week intervals, 11-week intervals, 12-week intervals, 13-week intervals, 14-week intervals, 15-week intervals, 20-week intervals, 25-week intervals, 30-week intervals, 35-week intervals, or 40-week intervals.
56. The method of any one of embodiments 16-55, wherein the method comprises administering one or more (e.g., 6) doses of the immunogenic composition at 4-week intervals followed by one or more (e.g., 5, 6) doses of the immunogenic composition at 20-week intervals.
57. The method of any one of embodiments 16-56, wherein the control sample is from a healthy individual or a patient with Alzheimer's Disease, wherein optionally the control sample is blood plasma or CSF.
58. The method of any one of embodiments 16-57, wherein the patient has a diagnosis of probable Alzheimer's Disease according to the revised 2011 NIA-AA criteria or the 2018 NIA-AA criteria.
59. The method of any one of embodiments 16-58, wherein the patient has a total Mini-Mental State Examination (MMSE) score of 20 and 26.
60. The method of any one of embodiments 16-59, wherein the patient has a brain MRI finding consistent with a diagnosis of Alzheimer's Disease.
61. The method of any one of embodiments 16-60, wherein the patient has medial temporal lobe atrophy as assessed according to a brain MRI and a Scheltens score of ≥2.
62. The method of any one of embodiments 16-61, wherein the patient has a positive AD biomarker signature in the CSF (e.g., one or more of total tau protein>400 pg/mL; pT181 tau protein>60 pg/mL; Aβ42<600 pg/mL; and Aβ42:Aβ40 ratio<0.089).
63. The method of any one of embodiments 16-62, wherein, prior to treatment, the patient is less than 80 years old, has a plasma neurofilament light chain concentration of greater than 10 μg/mL, has detectable tau protein in CSF, and lacks micro-hemorrhages, beginning or large confluent hemispheric deep white matter lesions (Fazekas grade 2 or 3), severe hippocampal atrophy (Scheltens score of 4), and hallucinations.
64. The method of any one of embodiments 16-63, wherein, prior to treatment, the patient is 50-85 years of age, e.g., 50-67 years of age, 50-70 years of age, 68-85 years of age, or 71-85 years of age.
65. The method of embodiment 64 wherein, prior to treatment, the patient is 50-70 years of age.
66. The method of embodiment 65, wherein, prior to treatment, the patient is 50-67 years of age.
67. The method of any one of embodiments 16-66, wherein the patient is on a stable therapy with an acetylcholinesterase inhibitor for at least 3 months prior to treatment.
68. The method of any one of embodiments 16-67, wherein the patient is on a stable dose of memantine treatment for at least 3 months prior to treatment.
69. The method of any one of embodiments 16-68, wherein the patient is male.
70. The method of any one of embodiments 16-69, wherein the immunogenic composition is administered in an amount effective to yield
   (a) an antibody titre of at least 100 ng/mL of antibodies against pathological tau;
   (b) an antibody titre of at least 100 ng/mL of antibodies against p108 tau peptide; and/or
   (c) a calculated area under the curve (AUC) of titre for IgG antibodies that bind p108 tau peptide of at least 100,000.
71. The method of embodiment 70, wherein the immunogenic peptide induces antibodies characterized by a Kd against pathological tau of about 10 nM or less, optionally about 4.2 nM or less (e.g., about 4.2 nM to about 0.01 nM).
72. The method of embodiment 71, wherein the immunogenic peptide induces antibodies characterized by a Kd against pathological tau of about 1 nM or less (e.g., about 1 nM to about 0.01 nM).
73. The method of any one of embodiments 70-72, wherein pathological tau comprises tau151-391/4R.
74. A method of treating Alzheimer's Disease (AD) or mild cognitive impairment, comprising:
   (a) determining a Mini-Mental State Examination (MMSE) score for the patient;
   (b) comparing the score from step (a) to a threshold score, wherein optionally the threshold score is 20-26;
   (c) selecting the patient for treatment wherein the patient has a MMSE score above the threshold; and
   (d) administering to the selected patient one or more doses of an immunogenic composition comprising an immunogenic peptide comprising SEQ ID NO: 1 or SEQ ID NO: 2.
75. The method of embodiment 74, wherein the MMSE score of the patient is at least 29, at least 28, at least 27, at least 26, at least 25, at least 24, at least 23, at least 22, at least 21, at least 20, at least 19, at least 18, at least 17, at least 16, at least 15, at least 14, at least 13, or at least 12.
76. The method of embodiment 74 or claim 75, wherein the MMSE score of the patient is at or greater than the threshold of 29, 28, 27, 26, 25, 24, 23, 22, 21, 20, 19, 18, 17, 16, 15, 14, 13, or 12.
77. The method of any one of embodiments 74-76, wherein the MMSE score of the patient is 24-26.
78. A method of treating Alzheimer's Disease (AD) or mild cognitive impairment, comprising administering one or more doses of an immunogenic composition comprising an immunogenic peptide comprising SEQ ID NO: 1 or SEQ ID NO: 2 to a patient having an MMSE score of greater than 23.
79. The method of embodiment 78, wherein the MMSE score of the patient is 26, 27, 28, 29, or 30.
80. The method of any one of embodiments 74-79, wherein the immunogenic peptide further comprises a carrier protein or peptide.
81. The method of embodiment 80, wherein the carrier is keyhole limpet hemocyanin (KLH) or a fragment thereof.
82. The method of embodiment 81, wherein the KLH or fragment thereof is coupled to SEQ ID NO: 1 or SEQ ID NO: 2 via a maleimide linker, wherein optionally the maleimide linker is N-γ-maleimidobutyryl-oxysulfosuccinimide ester (Sulfo-GMBS).
83. The method of any one of embodiments 74-82, wherein a dose comprises about 20-50 μg, e.g., at least about 20 μg, at least about 30 μg, at least about 40 μg, or at least about 50 μg of the immunogenic peptide.
84. The method of any one of embodiments 74-83, wherein each dose comprises about 40 μg of the immunogenic peptide.
85. The method of any one of embodiments 74-84, wherein the immunogenic composition further comprises an adjuvant.
86. The method of embodiment 85, wherein the adjuvant comprises an aluminum compound.
87. The method of embodiment 86, wherein the aluminum compound comprises aluminum hydroxide (Al(OH)₃).
88. The method of any one of embodiments 85-87, wherein the adjuvant comprises about 0.1 mg to about 10 mg aluminum (Al³⁺), optionally, about 0.5 mg aluminum (Al³⁺).
89. The method of any one of embodiments 74-88, wherein the immunogenic composition further comprises a phosphate buffer, optionally in a volume of about 0.3 mL.
90. The method of any one of embodiments 74-89, wherein the immunogenic composition comprises an immunogenic peptide comprising SEQ ID NO: 2 coupled via a maleimide linker (optionally Sulfo-GMBS) to KLH, about 0.5 mg aluminum (Al³⁺), and a phosphate buffer, optionally in a volume of about 0.3 mL.
91. The method of any one of embodiments 74-90, wherein treating the patient comprises administering 5 doses, 6 doses, 7 doses, 8 doses, 9 doses, 10 doses, 11 doses, 12 doses, 13 doses, 14 doses, 15 doses, 20 doses, 25 doses, 50 doses or 100 doses of the immunogenic peptide.
92. The method of any one of embodiments 74-91, wherein treating the patient comprises administering doses at 2-week intervals, 3-week intervals, 4-week intervals, 5-week intervals, 6-week intervals, 7-week intervals, 8-week intervals, 9-week intervals, 10-week intervals, 11-week intervals, 12-week intervals, 13-week intervals, 14-week intervals, 15-week intervals, 20-week intervals, 25-week intervals, 30-week intervals, 35-week intervals, or 40-week intervals.
93. The method of any one of embodiments 74-92, wherein treating the patient comprises administering one or more (e.g., 6) doses of the immunogenic composition at 4-week intervals followed by one or more (e.g., 5, 6, 7) doses of the immunogenic composition at 20-week intervals.
94. The method of any one of embodiments 74-93, wherein the control sample is from a healthy individual or a patient with Alzheimer's Disease, wherein optionally the control sample is blood plasma or CSF.
95. The method of any one of embodiments 74-94, wherein the patient has a diagnosis of probable Alzheimer's Disease according to the revised 2011 NIA-AA criteria or the 2018 NIA-AA criteria.
96. The method of any one of embodiments 74-95, wherein the patient has a brain MRI finding consistent with a diagnosis of Alzheimer's Disease.
97. The method of any one of embodiments 74-96, wherein the patient has medial temporal lobe atrophy as assessed according to a brain MRI and a Scheltens score of ≥2.
98. The method of any one of embodiments 74-97, wherein the patient has a positive AD biomarker signature in the CSF (e.g., one or more of total tau protein>400 pg/mL; pT181 tau protein>60 pg/mL; Aβ42<600 pg/mL; and Aβ42:Aβ40 ratio<0.089).
99. The method of any one of embodiments 74-98, wherein, prior to treatment, the patient is less than 80 years old, has a plasma neurofilament light chain concentration of greater than 10 μg/mL, has detectable tau protein in CSF, and lacks micro-hemorrhages, beginning or large confluent hemispheric deep white matter lesions (Fazekas grade 2 or 3), severe hippocampal atrophy (Scheltens score of 4), and hallucinations.
100. The method of any one of embodiments 74-99, wherein, prior to treatment, the patient is 50-85 years of age, e.g., 50-67 years of age, 50-70 years of age, 68-85 years of age, or 71-85 years of age.
101. The method of claim 100, wherein, prior to treatment, the patient is 50-70 years of age.
102. The method of embodiment 101, wherein, prior to treatment, the patient is 50-67 years of age.
103. The method of any one of embodiments 74-102, wherein the patient is on a stable therapy with an acetylcholinesterase inhibitor for at least 3 months prior to treatment.
104. The method of any one of embodiments 74-103, wherein the patient is on a stable dose of memantine treatment for at least 3 months prior to treatment.
105. The method of any one of embodiments 74-104, wherein the patient is male.
106. The method of any one of embodiments 74-105, wherein the immunogenic composition is administered in an amount effective to yield
   (a) an antibody titre of at least 100 ng/mL of antibodies against pathological tau;
   (b) an antibody titre of at least 100 ng/mL of antibodies against p108 tau peptide; and/or
   (c) a calculated area under the curve (AUC) of titre for IgG antibodies that bind p108 tau peptide of at least 100,000.
107. The method of embodiment 106, wherein the immunogenic peptide induces antibodies characterized by a Kd against pathological tau of about 10 nM or less, optionally about 4.2 nM or less (e.g., about 4.2 nM to about 0.01 nM).
108. The method of embodiment 107, wherein the immunogenic peptide induces antibodies characterized by a Kd against pathological tau of about 1 nM or less (e.g., about 1 nM to about 0.01 nM).
109. The method of any one of embodiments 106-108, wherein pathological tau comprises tau151-391/4R.
110. A method of treating a patient suffering from Alzheimer's Disease (AD) or mild cognitive impairment, the method comprising:
   (a) determining the presence and/or amount of one or more of hsa-let-7a-5p, hsa-miR-15a-5p, hsa-miR191-5p, and hsa-miR-23a-3p in the patient;
   (b) administering one or more doses of an immunogenic composition comprising an immunogenic peptide comprising SEQ ID NO: 1 or SEQ ID NO: 2;
   (c) after administering the one or more doses, determining the presence and/or amount of one or more of hsa-let-7a-5p, hsa-miR-15a-5p, hsa-miR191-5p, and hsa-miR-23a-3p in the patient;
   (d) comparing the presence and/or amount from step (c) after treatment to the presence and/or amount from step a before treatment, wherein an altered amount of the one or more of hsa-let-7a-5p, hsa-miR-15a-5p, hsa-miR191-5p, and hsa-miR-23a-3p indicates treatment efficacy; and
      (e) administering one or more additional doses of the immunogenic composition when the patient demonstrates treatment efficacy in step (d).
111. The method of embodiment 110, wherein decreased amounts of hsa-let-7a-5p and/or hsa-miR-15a-5p after treatment indicates efficacy.
112. The method of embodiment 110, wherein about 1, 2, 3, 4, 5, 6, 7, 8, 9, 10, 15, 20, 25, 50, 100, or more fold reduction of hsa-let-7a-5p and/or hsa-miR-15a-5p after treatment indicates efficacy.
113. The method of embodiment 110, wherein increased amounts of hsa-miR191-5p and/or hsa-miR-23a-3p after treatment indicates efficacy.
114. The method of embodiment 110, wherein about 1, 2, 3, 4, 5, 6, 7, 8, 9, 10, 15, 20, 25, 50, 100, or more fold increase of hsa-miR191-5p and/or hsa-miR-23 after treatment indicates efficacy.
115. A method of treating a patient suffering from Alzheimer's Disease (AD) or mild cognitive impairment, the method comprising:
   (a) determining the presence and/or amount of NfL in the patient;
   (b) administering one or more doses of an immunogenic composition comprising an immunogenic peptide comprising SEQ ID NO: 1 or SEQ ID NO: 2;
   (c) after administering the one or more doses, determining the presence and/or amount of NfL in the patient;
   (d) comparing the presence and/or amount from step (c) after treatment to the presence and/or amount from step a before treatment, wherein an altered amount of NfL indicates treatment efficacy; and
      (e) administering one or more additional doses of the immunogenic composition when the patient demonstrates treatment efficacy in step (d).
116. The method of embodiment 115, wherein a stable or decreased amount of NfL after treatment indicates efficacy.
117. The method of embodiment 115, wherein an amount of NfL that does not increase by more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after treatment indicates efficacy.
118. The method of embodiment 115, wherein an amount of NfL that decreases by more than about 30%, 25%, 20%, 15%, 14%, 13%, 12%, 11%, 10%, 9%, 8%, 7%, 6%, 5%, 4%, 3%, 2%, or 1% after treatment indicates efficacy.
119. The method of embodiment 115, wherein an amount of NfL that does not increase by more than about 6 pg/mL, 5 pg/mL 4 pg/mL, 3 pg/mL or 2 pg/mL after treatment indicates efficacy.
120. A method of treating a patient suffering from Alzheimer's Disease (AD) or mild cognitive impairment, the method comprising:
   (a) determining the presence and/or amount of neurogranin in the patient;
   (b) administering one or more doses of an immunogenic composition comprising an immunogenic peptide comprising SEQ ID NO: 1 or SEQ ID NO: 2;
   (c) after administering the one or more doses, determining the presence and/or amount of neurogranin in the patient;
   (d) comparing the presence and/or amount from step (c) after treatment to the presence and/or amount from step a before treatment, wherein an altered amount of neurogranin indicates treatment efficacy; and
   (e) administering one or more additional doses of the immunogenic composition when the patient demonstrates treatment efficacy in step (d).
121. The method of embodiment 120, wherein a stable or decreased amount of neurogranin after treatment indicates efficacy.
122. The method of embodiment 120, wherein an amount of neurogranin that does not increase by more than about 25%, 24%, 23%, 22%, 21%, 20%, 19%, 18%, 17%, 16%, 15%, 14%, 13%, 12%, 11%, 10%, 9%, 8%, 7%, 5%, 4%, 3%, 2%, or 1% after treatment indicates efficacy.
123. The method of embodiment 120, wherein an amount of neurogranin that decreases by more than about 30%, 25%, 20%, 15%, 14%, 13%, 12%, 11%, 10%, 9%, 8%, 7%, 6%, 5%, 4%, 3%, 2%, or 1% after treatment indicates efficacy.
124. The method of embodiment 120, wherein an amount of neurogranin that does not increase by more than about 20 pg/mL, 18 pg/mL, 16 pg/mL, 14 pg/mL, 12 pg/mL, 10 pg/mL, 8 pg/mL, 6 pg/mL, 5 pg/mL 4 pg/mL, 3 pg/mL or 2 pg/mL after treatment indicates efficacy.
125. A method of treating a patient suffering from Alzheimer's Disease (AD) or mild cognitive impairment, the method comprising:
   (a) determining the presence and/or amount of one or more metabolites in the patient;
   (b) administering one or more doses of an immunogenic composition comprising an immunogenic peptide comprising SEQ ID NO: 1 or SEQ ID NO: 2;
   (c) after administering the one or more doses, determining the presence and/or amount of one or more metabolites in the patient;
   (d) comparing the presence and/or amount from step (c) after treatment to the presence and/or amount from step a before treatment, wherein an altered amount of neurogranin indicates treatment efficacy; and
   (e) administering one or more additional doses of the immunogenic composition when the patient demonstrates treatment efficacy in step (d).
126. The method of embodiment 125, wherein an altered amount of one or more metabolites after treatment indicates efficacy.
127. The method of embodiment 125, wherein an amount of the one or more metabolites that increases by more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after treatment indicates efficacy.
128. The method of embodiment 125, wherein an amount of the one or more metabolites that decreases by more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after treatment indicates efficacy.
129. The method of embodiment 125, wherein an altered amount of at least 2, at least 3, at least 4, at least 5, at least 6 or at least 7 metabolites after treatment indicates efficacy.
130. The method of any one of embodiments 110-129, wherein the immunogenic peptide further comprises a carrier protein or peptide.
131. The method of embodiment 130, wherein the carrier is keyhole limpet hemocyanin (KLH) or a fragment thereof.
132. The method of embodiment 131, wherein the KLH or fragment thereof is coupled to SEQ ID NO: 1 or SEQ ID NO: 2 via a maleimide linker, wherein optionally the maleimide linker is N-γ-maleimidobutyryl-oxysulfosuccinimide ester (Sulfo-GMBS).
133. The method of any one of embodiments 110-132, wherein a dose comprises about 20-50 μg, e.g., at least about 20 μg, at least about 30 μg, at least about 40 μg, or at least about 50 μg of the immunogenic peptide.
134. The method of any one of embodiments 110-133, wherein each dose comprises about 40 μg of the immunogenic peptide.
135. The method of any one of embodiments 110-134, wherein the immunogenic composition further comprises an adjuvant.
136. The method of embodiment 135, wherein the adjuvant comprises an aluminum compound.
137. The method of embodiment 136, wherein the aluminum compound comprises aluminum hydroxide (Al(OH)₃).
138. The method of any one of embodiments 135-137, wherein the adjuvant comprises about 0.1 mg to about 10 mg aluminum (Al³⁺), optionally, about 0.5 mg aluminum (Al³⁺).
139. The method of any one of embodiments 110-138, wherein the immunogenic composition further comprises a phosphate buffer, optionally in a volume of about 0.3 mL.
140. The method of any one of embodiments 110-139, wherein the immunogenic composition comprises an immunogenic peptide comprising SEQ ID NO: 2 coupled via a maleimide linker (optionally Sulfo-GMBS) to KLH, about 0.5 mg aluminum (Al³⁺), and a phosphate buffer, optionally in a volume of about 0.3 mL.
141. The method of any one of embodiments 110-140, wherein the patient is treated with at least 2 doses, 3 doses, 4 doses, 5 doses, 6 doses, 7 doses, 8 doses, 9 doses, 10 doses, 11 doses, 12 doses, 13 doses, 14 doses, or 15 doses of the immunogenic peptide in step (b).
142. The method of any one of embodiments 110-141, wherein treating the patient comprises administering doses at 2-week intervals, 3-week intervals, 4-week intervals, 5-week intervals, 6-week intervals, 7-week intervals, 8-week intervals, 9-week intervals, 10-week intervals, 11-week intervals, 12-week intervals, 13-week intervals, 14-week intervals, 15-week intervals, 20-week intervals, 25-week intervals, 30-week intervals, 35-week intervals, or 40-week intervals.
143. The method of any one of embodiments 110-142, wherein the patient is treated with at least 2, 3, 4, 5, 6, 7, 8, 9, 10, 11, 12, 13, 14, or 15 additional doses of the immunogenic peptide in step (e).
144. The method of any one of embodiments 110-143, wherein the control sample is from a healthy individual or a patient with Alzheimer's Disease, wherein optionally the control sample is blood plasma or CSF.
145. The method of any one of embodiments 110-144, wherein the patient has a diagnosis of probable Alzheimer's Disease according to the revised 2011 NIA-AA criteria or the 2018 NIA-AA criteria.
146. The method of any one of embodiments 110-145, wherein the patient has a brain MRI finding consistent with a diagnosis of Alzheimer's Disease.
147. The method of any one of embodiments 110-146, wherein the patient has medial temporal lobe atrophy as assessed according to a brain MRI and a Scheltens score of ≥2.
148. The method of any one of embodiments 110-147, wherein the patient has a positive AD biomarker signature in the CSF (e.g., one or more of total tau protein>400 pg/mL; pT181 tau protein>60 pg/mL; Aβ42<600 pg/mL; and Aβ42:Aβ40 ratio<0.089).
149. The method of any one of embodiments 110-148, wherein, prior to treatment, the patient is less than 80 years old, has a plasma neurofilament light chain concentration of greater than 10 pg/mL, has detectable tau protein in CSF, and lacks micro-hemorrhages, beginning or large confluent hemispheric deep white matter lesions (Fazekas grade 2 or 3), severe hippocampal atrophy (Scheltens score of 4), and hallucinations.
150. The method of any one of embodiments 110-149, wherein, prior to treatment, the patient is 50-85 years of age, e.g., 50-67 years of age, 50-70 years of age, 68-85 years of age, or 71-85 years of age.
151. The method of embodiment 150, wherein, prior to treatment, the patient is 50-70 years of age.
152. The method of embodiment 151, wherein, prior to treatment, the patient is 50-67 years of age.
153. The method of any one of embodiments 110-152, wherein the patient is on a stable therapy with an acetylcholinesterase inhibitor for at least 3 months prior to treatment.
154. The method of any one of embodiments 110-153, wherein the patient is on a stable dose of memantine treatment for at least 3 months prior to treatment.
155. The method of any one of embodiments 110-154, wherein the patient is male.
156. The method of any one of embodiments 110-155, wherein the immunogenic composition is administered in an amount effective to yield
   (a) an antibody titre of at least 100 ng/mL of antibodies against pathological tau;
   (b) an antibody titre of at least 100 ng/mL of antibodies against p108 tau peptide; and/or
   (c) a calculated area under the curve (AUC) of titre for IgG antibodies that bind p108 tau peptide of at least 100,000.
157. The method of embodiment 156, wherein the immunogenic peptide induces antibodies characterized by a Kd against pathological tau of about 10 nM or less, optionally about 4.2 nM or less (e.g., about 4.2 nM to about 0.01 nM).
158. The method of embodiment 157, wherein the immunogenic peptide induces antibodies characterized by a Kd against pathological tau of about 1 nM or less (e.g., about 1 nM to about 0.01 nM).
159. The method of any one of embodiments 156-158, wherein pathological tau comprises tau151-391/4R.
160. A method of treating a patient suffering from Alzheimer's Disease or mild cognitive impairment, the method comprising administering to the patient one or more doses of an immunogenic composition comprising an immunogenic peptide comprising SEQ ID NO: 1 or SEQ ID NO: 2, wherein the immunogenic composition is administered in an amount effective to yield
   (a) an antibody titre of at least 100 ng/mL of antibodies against pathological tau;
   (b) an antibody titre of at least 100 ng/mL of antibodies against p108 tau peptide; and/or
   (c) a calculated area under the curve (AUC) of titre for IgG antibodies that bind p108 tau peptide of at least 100,000.
161. The method of embodiment 160 wherein the immunogenic peptide induces antibodies characterized by a Kd against pathological tau of about 10 nM or less, optionally about 4.2 nM or less (e.g., about 4.2 nM to about 0.01 nM).
162. The method of embodiment 161, wherein the immunogenic peptide induces antibodies characterized by a Kd against pathological tau of about 1 nM or less (e.g., about 1 nM to about 0.01 nM).
163. The method of any one of embodiments 160-162, wherein pathological tau comprises tau151-391/4R.
164. The method of any one of embodiments 160-163, wherein the method comprises an initial dosing regimen comprising administering a dose of the immunogenic composition once per month for e.g., six months.
165. The method of embodiment 164, wherein the method further comprises administering one or more boosters following the initial dosing regimen.
166. The method of embodiment 164, wherein, if the titre of antibodies against pathological tau falls below 100 ng/mL, the method further comprises administering one or more boosters following the initial dosing regimen in an amount effective to restore the titre of antibodies against pathological tau to at least 100 ng/mL, wherein optionally, following administration of the one or more boosters, the average titre of antibodies against pathological tau is at least 100 ng/mL for at least two years measured from the initial dosing regimen.
167. The method of embodiment 165 or embodiment 166, wherein the one or more boosters are administered once every three months.
168. The method of any one of embodiments 165-167, wherein the method comprises administering at least five boosters.
169. The method of any one of embodiments 160-168, wherein the patient suffers from Alzheimer's Disease.
170. The method of any one of embodiments 160-169, wherein the patient has a diagnosis of probable Alzheimer's Disease according to the revised 2011 NIA-AA criteria or the 2018 NIA-AA criteria.
171. The method of any one of embodiments 160-170, wherein the patient has a total Mini-Mental State Examination score of ≥20 and ≤26.
172. The method of any one of embodiments 160-171, wherein the patient has a brain MRI finding consistent with a diagnosis of Alzheimer's Disease.
173. The method of any one of embodiments 160-172, wherein the patient has a medial temporal lobe atrophy as assessed on brain MRI and according to a Scheltens score of ≥2.
174. The method of any one of embodiments 160-173, wherein the patient has a positive Alzheimer's Disease biomarker signature in cerebrospinal fluid (CSF) (e.g., one or more of total tau protein>400 pg/mL; pT181 tau protein>60 pg/mL; Aβ42<600 pg/mL; and Aβ42:Aβ40 ratio<0.089).
175. The method of any one of embodiments 160-174, wherein the patient is 50-85 years of age, e.g., 50-67 years of age, 50-70 years of age, 68-85 years of age, or 71-85 years of age.
176. The method of embodiment 175, wherein the patient is 50-70 years of age. 177. The method of embodiment 176, wherein the patient is 50-67 years of age. 178. The method of embodiment 175, wherein the patient is 68-85 years of age.
179. The method of any one of embodiments 160-178, wherein the patient is on a stable therapy with an acetylcholinesterase inhibitor for at least 3 months before the start of treatment.
180. The method of any one of embodiments 160-179, wherein the patient is on a stable dose of memantine treatment for at least 3 months before the start of treatment.
181. The method of any one of embodiments 160-180, wherein the patient is male.
182. The method of any one of embodiments 160-181, wherein, before administration of the immunogenic peptide, the patient has an Aβ42 concentration of less 600 pg/mL in cerebrospinal fluid (CSF), a phosphorylated tau (p-tau) T181 concentration of greater than 60 pg/mL in CSF, and a total tau (t-tau) concentration of greater than 400 pg/mL in CSF.
183. The method of any one of embodiments 160-182, wherein, prior to treatment, the patient is less than 80 years old, has a plasma neurofilament light chain concentration of greater than 10 pg/mL, has detectable tau protein in CSF, and lacks micro-hemorrhages, beginning or large confluent hemispheric deep white matter lesions (Fazekas grade 2 or 3), severe hippocampal atrophy (Scheltens score of 4), and hallucinations.
184. The method of any one of embodiments 160-183, wherein the immunogenic composition is administered in an amount effective to stabilize or reduce accumulation of neurofilament light chain in plasma, relative to baseline accumulation of neurofilament light chain in plasma.
185. The method of embodiment 184, wherein the patient has stable neurofilament light chain concentration (e.g., no more than a 1%, 5%, 10%, 15%, 20%, or 25% increase in neurofilament light chain concentration) in plasma, relative to baseline accumulation, following the initial dosing regimen.
186. The method of embodiment 184, wherein the patient has reduced accumulation of neurofilament light chain in plasma, relative to baseline accumulation, following the initial dosing regimen.
187. The method of embodiment 185 or embodiment 186, wherein the method further comprises administering one or more boosters following the initial dosing regimen.
188. The method of embodiment 185 or embodiment 186, wherein, if the patient has an increased accumulation (e.g., more than about a 25% increase) of neurofilament light chain in plasma relative to baseline accumulation following the initial dosing regimen, the method further comprises administering one or more boosters following the initial dosing regimen in an amount effective to stabilize or reduce accumulation of neurofilament light chain in plasma, wherein optionally, following administration of the one or more boosters, the patient has stable neurofilament light chain concentration or reduced accumulation of neurofilament light chain in plasma for at least two years measured from the initial dosing regimen.
189. The method of embodiment 187 or embodiment 188, wherein the one or more boosters are administered once every three months.
190. The method of any one of embodiments 187-189, wherein the method comprises administering at least five boosters.
191. The method of any one of embodiments 160-190, wherein, following administration of the immunogenic composition (e.g., following administration of the initial dosing regimen):

(a) if the patient is 50-67 years of age, the concentration of neurofilament light chain in plasma increases by no more than 2 pg/mL, preferably by no more than 1.8 pg/mL, for at least two years; or
(b) if the patient is 68-85 years of age, the concentration of neurofilament light chain in plasma increases by no more than 2.3 pg/mL for at least two years.
192. The method of embodiment 191, wherein, the method further comprises administering one or more boosters following the initial dosing regimen if:
   during the two years following the initial dosing regimen, the average concentration of neurofilament light chain in plasma increases by more than 2 pg/mL (e.g., if the patient is 50-67 years of age), or by more than 2.3 pg/mL (e.g., if the patient is 58-85 years of age);
   wherein the one or more boosters are administered in an amount effective to maintain an average increase in neurofilament light chain in plasma of no more than 2 pg/mL (e.g., if the patient is 50-67 years of age) or no more than 2.3 pg/mL (e.g., if the patient is 58-85 years of age).
193. The method of embodiment 192, wherein the one or more boosters are administered once every three months.
194. The method of embodiment 192 or claim 193, wherein the method comprises administering at least five boosters.
195. The method of any one of embodiments 160-194, wherein the immunogenic composition is administered in an amount effective to stabilize or reduce total tau (t-tau) levels in CSF, relative to t-tau levels in CSF before administration of the immunogenic composition.
196. The method of embodiment 195, wherein the patient has stable t-tau levels in CSF following the initial dosing regimen, relative to t-tau levels before administration of the initial dosing regimen.
197. The method of embodiment 195, wherein the patient has reduced t-tau levels in CSF following the initial dosing regimen, relative to t-tau levels before administration of the initial dosing regimen.
198. The method of embodiment 197, wherein, by two years following administration of the immunogenic composition (e.g., by two years following administration of the initial dosing regimen), the concentration of t-tau in CSF is reduced by at least 2 ng/L, optionally by at least 2.5 ng/L, and further optionally by at least 2.7 ng/L.
199. The method of any one of embodiments 195-198, wherein the method further comprises administering one or more boosters following the initial dosing regimen.
200. The method of any one of embodiments 195-198, wherein, if the concentration of t-tau in CSF has not decreased by at least 2 ng/L, optionally by at least 2.5 ng/L, or further optionally by at least 2.7 ng/mL after the initial dosing regimen, e.g., by two years after the initial dosing regimen, the method further comprises administering one or more boosters following the initial dosing regimen in an amount effective to stabilize or reduce t-tau levels in CSF, wherein optionally, following administration of the one or more boosters, the patient has stable or reduced t-tau levels for at least two years measured from the initial dosing regimen or from the date of booster administration, wherein, further optionally, by two years after the initial dosing regimen or two years after booster administration, the concentration of t-tau in CSF is decreased by at least 2 ng/L, optionally by at least 2.5 ng/L, and further optionally by at least 2.7 ng/L.
201. The method of embodiment 199 or embodiment 200, wherein the one or more boosters are administered once every three months.
202. The method of any one of embodiments 199-201, wherein the method comprises administering at least five boosters.
203. The method of any one of embodiments 160-202, wherein the immunogenic composition is administered in an amount effective to inhibit spreading of pathological tau, wherein optionally pathological tau comprises tau151-391/4R.
204. The method of embodiment 203, wherein the immunogenic composition is effective to inhibit spreading of pathological tau following the initial dosing regimen.
205. The method of embodiment 204, wherein the method further comprises administering one or more boosters following the initial dosing regimen.
206. The method of embodiment 204, wherein, if spreading of pathological tau increases, the method further comprises administering one or more boosters following the initial dosing regimen in an amount effective to inhibit spreading of pathological tau, wherein optionally, after administration of the one or more boosters, spreading of pathological tau is inhibited for at least two years measured from the initial dosing regimen.
207. The method of embodiment 205 or embodiment 206, wherein the one or more boosters are administered once every three months.
208. The method of any one of embodiments 205-207, wherein the method comprises administering at least five boosters.
209. The method of any one of embodiments 160-208, wherein the immunogenic composition is administered in an amount effective to
   (a) reduce tau protein phosphorylated at threonine-181 (pT181 tau) levels in CSF, relative to pT181 tau levels in CSF before administration of the immunogenic composition; and/or
   (b) reduce tau protein phosphorylated at threonine-217 (pT217 tau) levels in CSF, relative to pT217 tau levels in CSF before administration of the immunogenic composition.
210. The method of embodiment 209, wherein the patient has reduced pT181 tau levels and/or reduced pT217 levels in CSF following the initial dosing regimen.
211. The method of embodiment 210, wherein, by two years following administration of the immunogenic composition,
   (a) the concentration of pT181 tau in CSF is decreased by at least 5 ng/L, optionally by at least 5.3 ng/L over the concentration before administration of the immunogenic composition; and/or
   (b) the concentration of pT217 tau in CSF is decreased by at least 30 ng/L, optionally by at least 34 ng/Lover the concentration before administration of the immunogenic composition.
212. The method of any one of embodiments 209-211, wherein the method further comprises administering one or more boosters following the initial dosing regimen.
213. The method of embodiment 212, wherein, if the patient has increased pT181 tau levels and/or increased pT217 levels in CSF following the initial dosing regimen, the method further comprises administering one or more boosters following the initial dosing regimen in an amount effective to reduce pT181 tau levels and/or reduce pT217 levels in CSF, wherein optionally, following administration of the one or more boosters, the patient has reduced pT181 tau levels and/or reduced pT217 levels in CSF for at least two years measured from the initial dosing regimen.
214. The method of embodiment 212, wherein if by two years following the initial dosing regimen, the concentration of pT181 tau in CSF is not decreased by at least 5 ng/L (optionally by at least 5.3 ng/L) and/or the concentration of pT217 tau in CSF is not decreased by at least 30 ng/L (optionally by at least 34 ng/L), the method further comprises administering one or more boosters following the initial dosing regimen in an amount effective to decrease the concentration of pT181 tau in CSF by at least 5 ng/L (optionally by at least 5.3 ng/L) and/or decrease the concentration of pT217 tau in CSF by at least 30 ng/L (optionally by at least 34 ng/L).
215. The method of any one of embodiments 212-214, wherein the one or more boosters are administered once every three months.
216. The method of any one of embodiments 212-215, wherein the method comprises administering at least five boosters.
217. The method of any one of embodiments 160-216, wherein the immunogenic composition is administered in an amount effective to halt white matter degradation in the patient's fornix and/or genu corpus callosum, relative to white matter degradation before administration of the immunogenic composition.
218. The method of embodiment 217, wherein white matter degradation is measured by fractional anisotropy or mean diffusivity.
219. The method of embodiment 217 or embodiment 218, wherein the immunogenic composition is administered in an amount effective to halt axonal degeneration, relative to axonal degradation before administration of the immunogenic composition.
220. The method of embodiment 219, wherein axonal degeneration is measured by fractional anisotropy or mean diffusivity.
221. The method of embodiment 219 or embodiment 220, wherein white matter degradation is halted and/or axonal degradation is halted after administration of the initial dosing regimen.
222. The method of embodiment 221, wherein the method further comprises administering one or more boosters following the initial dosing regimen.
223. The method of embodiment 221, wherein, if the patient has increased white matter degradation and/or increased axonal degradation following the initial dosing regimen, the method further comprises administering one or more boosters following the initial dosing regimen in an amount effective to halt white matter degeneration and/or halt axonal degeneration, wherein optionally, following administration of the one or more boosters, white matter degradation is halted and/or axonal degradation is halted for at least two years measured from the initial dosing regimen.
224. The method of embodiment 222 or embodiment 223, wherein the one or more boosters are administered once every three months.
225. The method of any one of embodiments 222-224, wherein the method comprises administering at least five boosters.
226. The method of any one of embodiments 160-225, wherein the immunogenic composition is administered in an amount effective to prevent hippocampal atrophy (e.g., effective to prevent a decrease in hippocampal volume relative to hippocampal volume before administration of the immunogenic composition).
227. The method of embodiment 226, wherein the method comprises administering an initial dosing regimen comprising administering the immunogenic composition once per month for six months, optionally followed by one or more boosters.
228. The method of embodiment 226, wherein if the patient's hippocampal volume is reduced following an initial dosing regimen comprising administering the immunogenic composition once per month for six months, relative to the patient's hippocampal volume before administration of the initial dosing regimen, the method further comprises administering one or more boosters in an amount effective to prevent further hippocampal atrophy, wherein optionally the one or more boosters are administered in an amount effective to prevent further hippocampal atrophy for at least two years.
229. The method of embodiment 226, wherein the patient is 50-67 years of age and wherein, by two years following administration of the immunogenic composition, the patient's hippocampal volume is reduced by no more than 10%, preferably by no more than 8%, relative to the patient's hippocampal volume before administration of the immunogenic composition.
230. The method of embodiment 229, wherein if the patient's hippocampal volume is reduced by more than 10% following an initial dosing regimen comprising administering the immunogenic composition once per month for six months, relative to the patient's hippocampal volume before administration of the initial dosing regimen, the method further comprises administering one or more boosters in an amount effective to prevent further hippocampal atrophy (e.g., to prevent further loss of hippocampal volume of more than 10%), optionally for at least two years.
231. The method of embodiment 227, 228, or 230, wherein the one or more boosters are administered once every three months.
232. The method of embodiment 227, 228, 230, or 231, wherein the method comprises administering at least five boosters.
233. The method of any one of embodiments 160-232, wherein the immunogenic composition is administered in an amount effective to prevent cortical atrophy (e.g., effective to prevent a decrease in cortical volume relative to cortical volume before administration of the immunogenic composition).
234. The method of embodiment 233, wherein the method comprises administering an initial dosing regimen comprising administering the immunogenic composition once per month for six months, optionally followed by one or more boosters.
235. The method of embodiment 233, wherein, if the patient's cortical volume is reduced following an initial dosing regimen comprising administering the immunogenic composition once per month for six months, relative to the patient's cortical volume before administration of the initial dosing regimen, the method further comprises administering one or more boosters in an amount effective to prevent further cortical atrophy, wherein optionally the one or more boosters are administered in an amount effective to prevent further cortical atrophy for at least two years.
236. The method of embodiment 233, wherein the patient is 50-67 years of age and wherein, by two years following administration of the immunogenic composition, the patient's cortical volume is reduced by no more than 5%, preferably by no more than 4%, relative to the patient's cortical volume before administration of the immunogenic composition.
237. The method of embodiment 236, wherein if the patient's cortical volume is reduced by more than 5% following an initial dosing regimen comprising administering the immunogenic composition once per month for six months, relative to the patient's cortical volume before administration of the initial dosing regimen, the method further comprises administering one or more boosters in an amount effective to prevent further cortical atrophy (e.g., to prevent further loss of cortical volume of more than 5%), optionally for at least two years.
238. The method of embodiment 234, 235, or 237, wherein the one or more boosters are administered once every three months.
239. The method of embodiment 234, 235, 237, or 238, wherein the method comprises administering at least five boosters.
240. The method of any one of embodiments 226-239, wherein atrophy (e.g., loss of volume) is measured by MRI volumetry.
241. The method of any one of embodiments 160-240, wherein the patient is 50-67 years of age and the immunogenic composition is administered in an amount effective to slow the patient's rate of cognitive decline.
242. The method of embodiment 241, wherein the method comprises administering an initial dosing regimen comprising administering the immunogenic composition once per month for six months, optionally followed by one or more boosters.
243. The method of embodiment 241 or embodiment 242, wherein the patient's rate of cognitive decline is measured using the Clinical Dementia Rating Scale Sum of Boxes (CDR-SB) test, the Alzheimer's Disease Cooperative Study Mild Cognitive Impairment activities of Daily Living (ADCS-MCI-ADL) questionnaire, the Mini-Mental State Examination (MMSE), and/or a cognitive battery comprising the Cogstate International Shopping List Task, the Cogstate One Card Learning and One Card Back Tasks, the Letter Fluency Test and Category Fluency Test, and/or the Digit Symbol Coding test.
244. The method of embodiment 243, wherein, by two years following administration of the immunogenic composition (e.g., following administration of the initial dosing regimen), the patient's CDR-SB test score is increased by no more than 4, relative to the patient's CDR-SB test score before administration of the immunogenic composition.
245. The method of embodiment 243 or embodiment 244, wherein by two years following administration of the immunogenic composition (e.g., following administration of the initial dosing regimen), the patient's MMSE score is reduced by no more than 7 points, preferably by no more than 6.5 points, relative to the patient's MMSE score before administration of the immunogenic composition.
246. The method of any one of embodiments 243-245, wherein by two years following administration of the immunogenic composition (e.g., following administration of the initial dosing regimen), the patient's ADCS MCI ADL questionnaire score is reduced by no more than 14 points, preferably by no more than 13.5 points, relative to the patient's ADCS MCI ADL questionnaire score before administration of the immunogenic composition.
247. The method of any one of embodiments 243-246, wherein the method further comprises administering one or more boosters if:
   (a) the patient's CDR-SB score increases by more than 4 relative to the patient's CDR-SB test score before administration of the immunogenic composition (e.g., the initial dosing regimen);
   (b) the patient's MMSE score reduces by more than 6.5 points or by more than 7 points relative to the patient's MMSE score before administration of the immunogenic composition (e.g., the initial dosing regimen); and/or
   (c) the patient's ADCS-MCI-ADL questionnaire score reduces by more than 13.5 points or by more than 14 points relative to the patient's ADCS-MCI-ADL questionnaire score before administration of the immunogenic composition (e.g., the initial dosing regimen).
248. The method of embodiment 243 or embodiment 247, wherein the one or more boosters are administered in an amount effective to prevent:
   (a) an increase in the patient's CDR-SB score of more than 4 relative to the patient's CDR-SB test score before administration of the immunogenic composition (e.g., the initial dosing regimen);
   (b) a reduction in the patient's MMSE score of more than 6.5 points or more than 7 points relative to the patient's MMSE score before administration of the immunogenic composition (e.g., the initial dosing regimen); and/or
   (c) a reduction in the patient's ADCS-MCI-ADL questionnaire score of more than 13.5 points or more than 14 points relative to the patient's ADCS-MCI-ADL questionnaire score before administration of the immunogenic composition (e.g., the initial dosing regimen).
249. The method of embodiment 247 or embodiment 248, wherein the one or more boosters are administered once every three months.
250. The method of any one of embodiments 247-249, wherein the method comprises administering at least five boosters.
251. The method of any one of embodiments 160-250, wherein the immunogenic peptide of the immunogenic composition further comprises a carrier protein or peptide.
252. The method of embodiment 251, wherein the carrier is keyhole limpet hemocyanin (KLH) or a fragment thereof.
253. The method of embodiment 252, wherein the KLH or fragment thereof is coupled to SEQ ID NO: 1 or SEQ ID NO: 2 via a maleimide linker, wherein optionally the maleimide linker is N-γ-maleimidobutyryl-oxysulfosuccinimide ester (Sulfo-GMBS).
254. The method of any one of embodiments 251-253, wherein the immunogenic composition further comprises an adjuvant.
255. The method of embodiment 254, wherein the adjuvant comprises an aluminum compound.
256. The method of embodiment 255, wherein the aluminum compound comprises aluminum hydroxide (Al(OH)₃).
257. The method of any one of embodiments 254-256, wherein the adjuvant comprises about 0.1 mg to about 10 mg aluminum (Al³⁺), optionally, about 0.5 mg aluminum (Al³⁺).
258. The method of any one of embodiments 251-257, wherein the immunogenic composition further comprises a phosphate buffer, optionally in a volume of about 0.3 mL.
259. The method of any one of embodiments 251-258, wherein the immunogenic composition comprises an immunogenic peptide comprising SEQ ID NO: 2 coupled via a maleimide linker (e.g., Sulfo-GMBS) to KLH, about 0.5 mg aluminum (Al³⁺), and a phosphate buffer, optionally in a volume of about 0.3 mL.
260. The method of any one of embodiments 251-259, wherein a dose of the immunogenic composition comprises at least about 20 μg, at least about 30 μg, at least about 40 μg, or at least about 50 μg of the immunogenic peptide, optionally about 20 to 50 μg of the immunogenic peptide.
261. The method of any one of embodiments 251-260, wherein a dose of the immunogenic composition comprises about 40 μg of the immunogenic peptide.
262. The method of any one of embodiments 165-168, 187-190, 192-194, 199-202, 205-208, 212-216, 222-225, 227, 228, 230-232, 234, 235, 237-239, 242, or 247-250, wherein the booster comprises an immunogenic peptide comprising SEQ ID NO: 1 or SEQ ID NO: 2.
263. The method of embodiment 262, wherein the immunogenic peptide further comprises a carrier protein or peptide.
264. The method of embodiment 263, wherein the carrier is keyhole limpet hemocyanin (KLH) or a fragment thereof.
265. The method of embodiment 264, wherein the KLH or fragment thereof is coupled to SEQ ID NO: 1 or SEQ ID NO: 2 via a maleimide linker, wherein optionally the maleimide linker is N-γ-maleimidobutyryl-oxysulfosuccinimide ester (Sulfo-GMBS).
266. The method of any one of embodiments 262-265, wherein the booster further comprises an adjuvant.
267. The method of embodiment 266, wherein the adjuvant comprises an aluminum compound.
268. The method of embodiment 267, wherein the aluminum compound comprises aluminum hydroxide (Al(OH)₃).
269. The method of any one of embodiments 266-268, wherein the adjuvant comprises about 0.1 mg to about 10 mg aluminum (Al³⁺), optionally, about 0.5 mg aluminum (Al³⁺).
270. The method of any one of embodiments 262-269, wherein the booster further comprises a phosphate buffer, optionally in a volume of about 0.3 mL.
271. The method of any one of embodiments 262-270, wherein the booster comprises an immunogenic peptide comprising SEQ ID NO: 2 coupled via a maleimide linker (e.g., Sulfo-GMBS) to KLH, about 0.5 mg aluminum (Al³⁺), and a phosphate buffer, optionally in a volume of about 0.3 mL.
272. The method of any one of embodiments 262-271, wherein a dose of the booster comprises at least about 20 μg, at least about 30 μg, at least about 40 μg, or at least about 50 μg of the immunogenic peptide, optionally about 20 to 50 μg of the immunogenic peptide.
273. The method of any one of embodiments 262-272, wherein a dose of the booster comprises about 40 μg of the immunogenic peptide.
274. The method of any one of embodiments 262-273, wherein the booster is identical in composition to the immunogenic composition administered as part of an initial dosing regimen.
275. The method of embodiment 274, wherein the booster is administered at the same dose as the immunogenic composition administered as part of an initial dosing regimen.

We claim:

1. An immunogenic composition for use in treating Alzheimer's Disease or mild cognitive impairment in a patient in need thereof, wherein the immunogenic composition comprises an immunogenic peptide comprising SEQ ID NO: 1 or SEQ ID NO: 2, and wherein, prior to treatment, the patient is 50-85 years of age.

2. The immunogenic composition for use of claim 1, wherein, prior to treatment, the patient is 50-70 years of age.

3. The immunogenic composition for use of claim 1, wherein, prior to treatment, the patient is 50-67 years of age.

4. The immunogenic composition for use of any one of claims 1-3, wherein, prior to treatment, the patient has received a stable therapy with an acetylcholinesterase inhibitor for at least three months.

5. The immunogenic composition for use of any one of claims 1-4, wherein, prior to treatment, the patient has received a stable dose of memantine treatment for at least three months.

6. The immunogenic composition for use of any one of claims 1-5, wherein the patient has a diagnosis of probable Alzheimer's Disease according to the revised 2011 NIA-AA criteria or the 2018 NIA-AA criteria.

7. The immunogenic composition for use of any one of claims 1-6, wherein the patient has a brain MRI finding consistent with a diagnosis of Alzheimer's Disease.

8. The immunogenic composition for use of any one of claims 1-7, wherein the patient has medial temporal lobe atrophy as assessed according to a brain MRI and a Scheltens score of ≥2.

9. The immunogenic composition for use of any one of claims 1-8, wherein the patient has one or more of: total tau protein>400 pg/mL in cerebrospinal fluid (CSF), pT181 tau protein>60 pg/mL in CSF, Aβ42<600 pg/mL in CSF, and/or Aβ42:Aβ40 ratio<0.089 in CSF.

10. The immunogenic composition for use of any one of claims 1-9, wherein, prior to treatment, the patient is less than 80 years old, has a plasma neurofilament light chain concentration of greater than 10 pg/mL, has detectable tau protein in CSF, and lacks: micro-hemorrhages, beginning or large confluent hemispheric deep white matter lesions (Fazekas grade 2 or 3), severe hippocampal atrophy (Scheltens score of 4), and/or hallucinations.

11. The immunogenic composition for use of any one of claims 1-10, wherein the patient has Alzheimer's Disease.

12. The immunogenic composition for use of any one of claims 1-11, wherein the patient is male.

13. The immunogenic composition for use of any one of claims 1-12, wherein the immunogenic composition is formulated for administration to the patient in an amount and dosing regimen effective to yield
(a) an antibody titre of at least 100 ng/mL of antibodies against pathological tau; and/or
(b) an antibody titre of at least 100 ng/mL of antibodies against p108 tau peptide.

14. The immunogenic composition for use of any one of claims 1-12, wherein the immunogenic composition is formulated for administration to the patient in an amount and dosing regimen effective to yield
(a) an average antibody titre of at least 100 ng/mL of antibodies against pathological tau for at least two years; and/or
(b) an average antibody titre of at least 100 ng/mL of antibodies against p108 tau peptide for at least two years.

15. The immunogenic composition for use of any one of claims 1-14, wherein the immunogenic peptide induces at least one antibody characterized by a Kd against pathological tau of about 10 nM or less, optionally about 4.2 nM or less, further optionally about 4.2 nM to about 0.01 nM.

16. The immunogenic composition for use of claim 15, wherein the immunogenic peptide induces at least one antibody characterized by a Kd against pathological tau of about 1 nM or less, optionally about 1 nM to about 0.01 nM.

17. The immunogenic composition for use of any one of claims 13-16, wherein pathological tau comprises tau151-391/4R.

18. The immunogenic composition for use of any one of claims 1-17, wherein the immunogenic composition is formulated for administration to the patient in an amount and dosing regimen effective to stabilize or reduce accumulation of neurofilament light chain in plasma, relative to baseline accumulation of neurofilament light chain in plasma.

19. The immunogenic composition for use of claim 18, wherein administration of the immunogenic composition results in no more than a 1%, 5%, 10%, 15%, 20%, or 25% increase in neurofilament light chain concentration in plasma in the patient relative to baseline.

20. The immunogenic composition for use of claim 18, wherein the immunogenic composition is formulated for administration to the patient in an amount and dosing regimen effective to reduce accumulation of neurofilament light chain in plasma, relative to baseline accumulation of neurofilament light chain in plasma.

21. The immunogenic composition for use of claim 18, wherein the immunogenic composition is formulated for administration to the patient in an amount and dosing regimen effective to stabilize or reduce accumulation of neurofilament light chain in plasma, relative to baseline accumulation of neurofilament light chain in plasma, for at least two years.

22. The immunogenic composition for use of any one of claims 18-21, wherein the immunogenic composition is formulated for administration to the patient in an amount and dosing regimen effective to yield
(a) no more than a 2 pg/mL increase (preferably no more than a 1.8 pg/mL increase) in the concentration of neurofilament light chain in plasma for at least two years in a patient treated at 50-67 years of age; or
(b) no more than a 2.3 pg/mL increase in the concentration of neurofilament light chain in plasma for at least two years in a patient treated at 68-85 years of age.

23. The immunogenic composition for use of any one of claims 1-22, wherein the immunogenic composition is formulated for administration to the patient in an amount and dosing regimen effective to stabilize total tau (t-tau) levels in CSF, relative to baseline t-tau levels in CSF.

24. The immunogenic composition for use of any one of claims 1-22, wherein the immunogenic composition is formulated for administration to the patient in an amount and dosing regimen effective to reduce t-tau levels in CSF, relative to baseline t-tau levels in CSF.

25. The immunogenic composition for use of claim 24, wherein the immunogenic composition is formulated for administration to the patient in an amount and dosing regimen effective to reduce t-tau levels in CSF by at least 2 ng/L (optionally at least 2.5 ng/L, further optionally at least 2.7 ng/L), relative to baseline t-tau levels in CSF, wherein optionally the reduction lasts for two years.

26. The immunogenic composition for use of any one of claims 11-25, wherein the immunogenic composition is formulated for administration to the patient in an amount and dosing regimen effective to inhibit spreading of pathological tau, wherein optionally pathological tau comprises tau151-391/4R.

27. The immunogenic composition for use of claim 26, wherein the immunogenic composition is formulated for administration to the patient in an amount and dosing regimen effective to inhibit spreading of pathological tau for at least two years.

28. The immunogenic composition for use of claim 26 or claim 27, wherein pathological tau comprises tau151-391/4R.

29. The immunogenic composition for use of any one of claims 1-28, wherein the immunogenic composition is formulated for administration to the patient in an amount and dosing regimen effective to
(a) reduce tau protein phosphorylated at threonine-181 (pT181 tau) levels in CSF, relative to baseline pT181 tau levels in CSF, optionally for at least two years; and/or
(b) reduce tau protein phosphorylated at threonine-217 (pT217 tau) levels in CSF, relative to baseline pT217 tau levels in CSF, optionally for at least two years.

30. The immunogenic composition for use of claim 29, wherein the immunogenic composition is formulated for administration to the patient in an amount and dosing regimen effective to
(a) reduce the concentration of pT181 tau in CSF by at least 5 ng/L, optionally by at least 5.3 ng/L, relative to the baseline concentration of pT181 tau in CSF, for at least two years; and/or
(b) reduce the concentration of pT217 tau in CSF by at least 30 ng/L, optionally by at least 34 ng/L, relative to the baseline concentration of pT217 tau in CSF, for at least two years.

31. The immunogenic composition for use of any one of claims 1-30, wherein the immunogenic composition is formulated for administration to the patient in an amount and dosing regimen effective to halt white matter degradation in the patient's fornix and/or genu corpus callosum, relative to baseline white matter degradation, optionally for at least two years.

32. The immunogenic composition for use of claim 31, wherein white matter degradation is measured by fractional anisotropy or mean diffusivity.

33. The immunogenic composition for use of any one of claims 1-32, wherein the immunogenic composition is formulated for administration to the patient in an amount and dosing regimen effective to halt axonal degeneration, relative to baseline axonal degradation, optionally for at least two years.

34. The immunogenic composition for use of claim 33, wherein axonal degeneration is measured by fractional anisotropy or mean diffusivity.

35. The immunogenic composition for use of any one of claims 1-34, wherein the immunogenic composition is formulated for administration to the patient in an amount and dosing regimen effective to prevent hippocampal atrophy (e.g., effective to prevent a decrease in hippocampal volume relative to baseline hippocampal volume), optionally for at least two years.

36. The immunogenic composition for use of claim 35, wherein the immunogenic composition is formulated for administration to the patient in an amount and dosing regimen effective to prevent hippocampal atrophy in a patient treated at 50-67 years of age, wherein hippocampal atrophy is indicated by a decrease in the patient's hippocampal volume relative to baseline of more than 10%.

37. The immunogenic composition for use of claim 35 or claim 36, wherein the immunogenic composition is formulated for administration to the patient in an amount and dosing regimen effective to prevent hippocampal atrophy for at least two years.

38. The immunogenic composition for use of any one of claims 1-37, wherein the immunogenic composition is formulated for administration to the patient in an amount and dosing regimen effective to prevent cortical atrophy (e.g., effective to prevent a decrease in cortical volume relative to baseline cortical volume), optionally for at least two years.

39. The immunogenic composition for use of claim 38, wherein the immunogenic composition is formulated for administration to the patient in an amount and dosing regimen effective to prevent cortical atrophy in a patient treated at 50-67 years of age, wherein cortical atrophy is indicated by a decrease in the patient's cortical volume relative to baseline of more than 5%.

40. The immunogenic composition for use of claim 38 or claim 39, wherein the immunogenic composition is formulated for administration to the patient in an amount and dosing regimen effective to prevent cortical atrophy for at least two years.

41. The immunogenic composition for use of any one of claims 35-40, wherein atrophy (e.g., decrease in volume) is measured by MRI volumetry.

42. The immunogenic composition for use of any one of claims 1-41, wherein the immunogenic composition is formulated for administration to the patient in an amount and dosing regimen effective to slow the patient's rate of cognitive decline in a patient treated at 50-67 years of age.

43. The immunogenic composition for use of claim 42, wherein the patient's rate of cognitive decline is measured using the Clinical Dementia Rating Scale Sum of Boxes (CDR-SB) test, the Alzheimer's Disease Cooperative Study Mild Cognitive Impairment activities of Daily Living (ADCS-MCI-ADL) questionnaire, the Mini-Mental State Examination (MMSE), and/or a cognitive battery comprising the Cogstate International Shopping List Task, the Cogstate One Card Learning and One Card Back Tasks, the Letter Fluency Test and Category Fluency Test, and/or the Digit Symbol Coding test.

44. The immunogenic composition for use of claim 43, wherein the immunogenic composition is formulated for administration to the patient in an amount and dosing regimen effective to prevent an increase in the patient's CDR-SB test score of more than 4 relative to the patient's baseline CDR-SB test score, optionally for at least two years.

45. The immunogenic composition for use of claim 43 or claim 44, wherein the immunogenic composition is formulated for administration to the patient in an amount and dosing regimen effective to prevent a decrease in the patient's MMSE score of more than 7 points, preferably effective to prevent a decrease of more than 6.5 points, relative to the patient's baseline MMSE score, optionally for at least two years.

46. The immunogenic composition for use of any one of claims 43-45, wherein the immunogenic composition is formulated for administration to the patient in an amount and dosing regimen effective to prevent a decrease in the patient's ADCS MCI ADL questionnaire score of more than 14 points, preferably effective to prevent a decrease of more than 13.5 points, relative to the patient's baseline ADCS MCI ADL questionnaire score, optionally for at least two years.

47. The immunogenic composition for use of any one of claims 1-46, wherein the patient has at least one ApoE-ε4 allele.

48. The immunogenic composition for use of any one of claims 1-47, wherein, prior to treatment, the patient has an MMSE score of 20-26, optionally 24-26.

49. The immunogenic composition for use of any one of claims 1-48, wherein the immunogenic composition is in a dosing regimen comprising monthly doses, optionally for six months.

50. The immunogenic composition for use of claim 49, wherein the immunogenic composition further comprises one or more boosters for administration following an initial monthly dosing regimen.

51. The immunogenic composition for use of claim 50, wherein the immunogenic composition comprises at least five boosters.

52. The immunogenic composition for use of claim 50 or claim 51, wherein the boosters are administered once every three months.

53. The immunogenic composition for use of any one of claims 1-52, wherein the immunogenic composition comprises one or more doses comprising at least about 20 μg of the immunogenic peptide (optionally at least about 30 μg, at least about 40 μg, or at least about 50 μg of the immunogenic peptide).

54. The immunogenic composition for use of claim 53, wherein the immunogenic composition comprises one or more doses comprising about 20-50 μg of the immunogenic peptide.

55. The immunogenic composition for use of claim 54, wherein the immunogenic composition comprises one or more doses comprising about 40 μg of the immunogenic peptide.

56. The immunogenic composition for use of claim 55, wherein each dose comprises about 40 μg of the immunogenic peptide.

57. The immunogenic composition for use of any one of claims 1-56, wherein the immunogenic composition is administered in 5 doses, 6 doses, 7 doses, 8 doses, 9 doses, 10 doses, 11 doses, 12 doses, 13 doses, 14 doses, 15 doses, 20 doses, 25 doses, 50 doses, or 100 doses.

58. The immunogenic composition for use of any one of claims 1-57, wherein the immunogenic composition is administered as one or more doses (optionally six doses) at monthly (optionally four-week) intervals followed by one or more doses (optionally, five, six, or seven doses) at 20-week intervals.

59. The immunogenic composition for use of any one of claims 1-58, wherein the immunogenic composition further comprises a carrier, and wherein the carrier comprises a serum albumin; keyhole limpet hemocyanin (KLH) or a fragment thereof; an immunoglobulin molecule; thyroglobulin; ovalbumin; tetanus toxoid; a toxoid from another pathogenic bacterium; an attenuated toxin derivative; a cytokine such as IL-1, IL-1α, and IL-1β; IL-2; IFNγ; IL-10; GM-CSF; or a chemokine such as MIP1α, MIP1β, and RANTES.

60. The immunogenic composition for use of claim 59, wherein the carrier is KLH or a fragment thereof.

61. The immunogenic composition for use of claim 60, wherein the KLH or fragment thereof is coupled to the immunogenic peptide via a maleimide linker, wherein optionally the maleimide linker is N-γ-maleimidobutyryl-oxysulfosuccinimide ester (Sulfo-GMBS).

62. The immunogenic composition for use of any one of claims 1-61, wherein the immunogenic composition further comprises an adjuvant.

63. The immunogenic composition for use of claim 62, wherein the adjuvant comprises an aluminum compound.

64. The immunogenic composition for use of claim 63, wherein the aluminum compound comprises aluminum hydroxide (Al(OH)₃).

65. The immunogenic composition for use of any one of claims 1-64, wherein the immunogenic composition further comprises a phosphate buffer, optionally in a volume of about 0.3 mL.

66. The immunogenic composition for use of any one of claims 1-65, wherein the immunogenic peptide comprises SEQ ID NO: 2.

67. The immunogenic composition for use of any one of claims 1-66, wherein the immunogenic composition comprises an immunogenic peptide comprising SEQ ID NO: 2 coupled via a maleimide linker (optionally Sulfo-GMBS) to KLH, about 0.5 mg aluminum (Al³⁺), and a phosphate buffer, in a volume of about 0.3 mL.

68. The immunogenic composition for use of any one of claims 1-65, wherein the immunogenic peptide comprises SEQ ID NO: 1.

69. The immunogenic composition for use of any one of claims 1-65 and 68, wherein the immunogenic peptide consists of SEQ ID NO: 1 and up to 5 additional amino acids at the C-terminus or N-terminus of the sequence from the adjacent amino acids in tau isoform 2N4R.

70. The immunogenic composition for use of any one of claims 1-67, wherein the immunogenic peptide consists of SEQ ID NO: 2 and up to 5 additional amino acids at the C-terminus or N-terminus of the sequence from the adjacent amino acids in tau isoform 2N4R.

71. The immunogenic composition for use of any one of claims 1-65, wherein the immunogenic peptide consists of SEQ ID NO: 1.

72. The immunogenic composition for use of any one of claims 1-65, wherein the immunogenic peptide consists of SEQ ID NO: 2.

73. An immunogenic composition comprising an immunogenic peptide comprising SEQ ID NO: 1 or SEQ ID NO: 2, for use in treating Alzheimer's Disease (AD) or mild cognitive impairment in a patient in need thereof, comprising:
(a) determining the amount of neurofilament light chain (NfL) in the patient;
(b) administering one or more doses of the immunogenic composition;
(c) after administering the one or more doses, determining the amount of NfL in the patient;
(d) comparing the amount from step (c) after treatment to the amount from step (a) before treatment, wherein an altered amount of NfL indicates treatment efficacy; and
(e) administering one or more additional doses of the immunogenic composition when the patient demonstrates treatment efficacy in step (d).

74. The immunogenic composition for use of claim 73, wherein the amount of NfL two years following administration of the one or more doses of the immunogenic composition has not increased more than about 3.2 pg/mL compared to baseline.

75. An immunogenic composition comprising an immunogenic peptide comprising SEQ ID NO: 1 or SEQ ID NO: 2, for use in treating Alzheimer's Disease (AD) or mild cognitive impairment in a patient in need thereof, comprising:
(a) determining the presence and/or amount of neurogranin in the patient;
(b) administering one or more doses of the immunogenic composition;
(c) after administering the one or more doses, determining the amount of neurogranin in the patient;
(d) comparing the amount from step (c) after treatment to the amount from step (a) before treatment, wherein an altered amount of neurogranin indicates treatment efficacy; and
(e) administering one or more additional doses of the immunogenic composition when the patient demonstrates treatment efficacy in step (d).

76. An immunogenic composition comprising an immunogenic peptide comprising SEQ ID NO: 1 or SEQ ID NO: 2, for use in treating Alzheimer's Disease (AD) or mild cognitive impairment in a patient in need thereof, comprising:
(a) determining a Mini-Mental State Examination (MMSE) score for the patient;
(b) comparing the score from step (a) to a threshold score, wherein optionally the threshold score is 20-26;
(c) selecting the patient for treatment wherein the patient has a MMSE score above the threshold;
(d) administering one or more doses of the immunogenic composition;
(e) after administering the one or more doses, determining the MMSE score of the patient;
(f) comparing the MMSE score from step (e) after treatment to the MMSE score from step (a) before treatment, wherein an altered MMSE score indicates treatment efficacy; and
(g) administering one or more additional doses of the immunogenic composition when the patient demonstrates treatment efficacy in step (f).

77. An immunogenic composition comprising an immunogenic peptide comprising SEQ ID NO: 1 or SEQ ID NO: 2, for use in treating Alzheimer's Disease (AD) or mild cognitive impairment in a patient in need thereof, comprising:
(a) determining an AD biomarker signature (optionally, one or more of total tau protein>400 pg/mL; pT181 tau protein>60 pg/mL; Aβ42<600 pg/mL; and Aβ42:Aβ40 ratio<0.089) in the patient;
(b) administering one or more doses of the immunogenic composition;
(c) after administering the one or more doses, determining an AD biomarker signature in the patient;
(d) comparing the biomarker signature from step (c) after treatment to the biomarker signature from step (a) before treatment, wherein an altered biomarker signature indicates treatment efficacy; and
(e) administering one or more additional doses of the immunogenic composition when the patient demonstrates treatment efficacy in step (d).

78. The immunogenic composition for use of claim 77, wherein, two years following administration of the immunogenic composition, pT181 decreased by at least 8.1 pg/mL compared to baseline and/or pT217 decreased by at least 69.2 pg/mL compared to baseline and/or total tau decreased by at least 71.8 pg/mL compared to baseline, and/or Aβ40 decreased at least 888 pg/mL compared to baseline.

79. An immunogenic composition comprising an immunogenic peptide comprising SEQ ID NO: 1 or SEQ ID NO: 2, for use in treating Alzheimer's Disease (AD) or mild cognitive impairment in a patient in need thereof, comprising:
(a) determining a volume of medial temporal lobe atrophy as assessed according to a brain MRI in the patient;
(b) administering one or more doses of the immunogenic composition if medial temporal lobe atrophy is above a threshold (optionally, a Scheltens score of ≥2);
(c) after administering the one or more doses, determining a volume of temporal lobe atrophy in the patient;
(d) comparing temporal lobe atrophy assessed in step (c) after treatment to temporal lobe atrophy assessed in step (a) before treatment, wherein an altered temporal lobe atrophy indicates treatment efficacy; and
(e) administering one or more additional doses of the immunogenic composition when the patient demonstrates treatment efficacy in step (d).

80. The immunogenic composition for use of any one of claims 73-79, wherein the immunogenic composition is formulated for administration to the patient in an amount and dosing regimen effective to yield
(a) an antibody titre of at least 100 ng/mL of antibodies against pathological tau; and/or
(b) an antibody titre of at least 100 ng/mL of antibodies against p108 tau peptide.

81. The immunogenic composition for use of any one of claims 73-79, wherein the immunogenic composition is formulated for administration to the patient in an amount and dosing regimen effective to yield
(a) an average antibody titre of at least 100 ng/mL of antibodies against pathological tau for at least two years; and/or
(b) an average antibody titre of at least 100 ng/mL of antibodies against p108 tau peptide for at least two years.

82. The immunogenic composition for use of any one of claims 73-81, wherein the immunogenic peptide induces at least one antibody characterized by a Kd against pathological tau of about 10 nM or less, optionally about 4.2 nM or less, further optionally, about 4.2 nM to about 0.01 nM.

83. The immunogenic composition for use of claim 82, wherein the immunogenic peptide induces at least one antibody characterized by a Kd against pathological tau of about 1 nM or less, optionally about 1 nM to about 0.01 nM.

84. The immunogenic composition for use of any one of claims 80-83, wherein pathological tau comprises tau151-391/4R.

85. The immunogenic composition for use of any one of claims 73-84, wherein, prior to treatment, the patient is 50-85 years of age.

86. The immunogenic composition for use of any one of claims 73-85, wherein the immunogenic composition comprises one or more doses comprising at least about 20 μg of the immunogenic peptide (optionally, at least about 30 μg, at least about 40 μg, or at least about 50 μg of the immunogenic peptide).

87. The immunogenic composition for use of claim 86, wherein the immunogenic composition comprises one or more doses comprising about 20-50 μg of the immunogenic peptide.

88. The immunogenic composition for use of claim 87, wherein the immunogenic composition comprises one or more doses comprising about 40 μg of the immunogenic peptide.

89. The immunogenic composition for use of claim 88, wherein each dose comprises about 40 μg of the immunogenic peptide.

90. The immunogenic composition for use of any one of claims 73-89, wherein the immunogenic composition further comprises a carrier, and wherein the carrier comprises a serum albumin; keyhole limpet hemocyanin (KLH) or a fragment thereof; an immunoglobulin molecule; thyroglobulin; ovalbumin; tetanus toxoid; a toxoid from another pathogenic bacterium; an attenuated toxin derivative; a cytokine such as IL-1, IL-1α, and IL-1β; IL-2; IFNγ; IL-10; GM-CSF; or a chemokine such as MIP1α, MIP1β, and RANTES.

91. The immunogenic composition for use of claim 90, wherein the carrier is KLH or a fragment thereof.

92. The immunogenic composition for use of claim 91, wherein the KLH or fragment thereof is coupled to the immunogenic peptide via a maleimide linker, wherein optionally the maleimide linker is N-γ-maleimidobutyryl-oxysulfosuccinimide ester (Sulfo-GMBS).

93. The immunogenic composition for use of any one of claims 73-92, wherein the immunogenic composition further comprises an adjuvant.

94. The immunogenic composition for use of claim 93, wherein the adjuvant comprises an aluminum compound.

95. The immunogenic composition for use of claim 94, wherein the aluminum compound comprises aluminum hydroxide (Al(OH)₃).

96. The immunogenic composition for use of any one of claims 73-95, wherein the immunogenic composition further comprises a phosphate buffer, optionally in a volume of about 0.3 mL.

97. The immunogenic composition for use of any one of claims 73-96, wherein the immunogenic peptide comprises SEQ ID NO: 2.

98. The immunogenic composition for use of any one of claims 73-97, wherein the immunogenic composition comprises an immunogenic peptide comprising SEQ ID NO: 2 coupled via a maleimide linker (optionally Sulfo-GMBS) to KLH, about 0.5 mg aluminum (Al³⁺), and a phosphate buffer, in a volume of about 0.3 mL.

99. The immunogenic composition for use of any one of claims 73-96, wherein the immunogenic peptide comprises SEQ ID NO: 1.

100. The immunogenic composition for use of any one of claims 73-96 and 99, wherein the immunogenic peptide consists of SEQ ID NO: 1 and up to 5 additional amino acids at the C-terminus or N-terminus of the sequence from the adjacent amino acids in tau isoform 2N4R.

101. The immunogenic composition for use of any one of claims 73-98, wherein the immunogenic peptide consists of SEQ ID NO: 2 and up to 5 additional amino acids at the C-terminus or N-terminus of the sequence from the adjacent amino acids in tau isoform 2N4R.

102. The immunogenic composition for use of any one of claims 73-96, wherein the immunogenic peptide consists of SEQ ID NO: 1.

103. The immunogenic composition for use of any one of claims 73-96, wherein the immunogenic peptide consists of SEQ ID NO: 2.

104. An immunogenic composition comprising an immunogenic peptide comprising SEQ ID NO: 1 or SEQ ID NO: 2, for use in treating Alzheimer's Disease (AD) or mild cognitive impairment in a patient in need thereof, comprising:
(a) determining the presence and/or amount of one or more of hsa-let-7a-5p, hsa-miR-15a-5p, hsa-miR191-5p, and hsa-miR-23a-3p in the patient;
(b) administering one or more doses of the immunogenic composition;
(c) after administering the one or more doses, determining the presence and/or amount of one or more of hsa-let-7a-5p, hsa-miR-15a-5p, hsa-miR191-5p, and hsa-miR-23a-3p in the patient;
(d) comparing the presence and/or amount from step (c) after treatment to the presence and/or amount from step (a) before treatment, wherein an altered amount of the one or more of hsa-let-7a-5p, hsa-miR-15a-5p, hsa-miR191-5p, and hsa-miR-23a-3p indicates treatment efficacy; and
(e) administering one or more additional doses of the immunogenic composition when the patient demonstrates treatment efficacy in step (d).

105. An immunogenic composition comprising an immunogenic peptide comprising SEQ ID NO: 1 or SEQ ID NO: 2, for use in treating Alzheimer's Disease (AD) or mild cognitive impairment in a patient in need thereof, comprising:
(a) determining the presence and/or amount of one or more metabolites in the patient;
(b) administering one or more doses of the immunogenic composition;
(c) after administering the one or more doses, determining the presence and/or amount of one or more metabolites in the patient;
(d) comparing the presence and/or amount from step (c) after treatment to the presence and/or amount from step (a) before treatment, wherein an altered amount of neurogranin indicates treatment efficacy; and
(e) administering one or more additional doses of the immunogenic composition when the patient demonstrates treatment efficacy in step (d).

106. A method for diagnosing a patient suffering from Alzheimer's Disease, comprising detecting the presence and/or amount of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p in the patient or a sample from the patient, wherein the presence of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p and/or an altered amount relative to an amount in a control sample or threshold indicates Alzheimer's Disease in the subject; and diagnosing the presence or absence of Alzheimer's Disease in the patient.

107. A method for diagnosing a patient suffering from Alzheimer's Disease, comprising detecting the presence and/or amount of 2,4-dihydroxybutanoic acid, a phospholipid, phosphatidylcholine, sphingomyelin, and/or sterol, in blood plasma, serum, or cerebrospinal fluid (CSF) of the patient, wherein the presence of one or more metabolites and/or an altered amount relative to an amount in a control sample or threshold indicates Alzheimer's Disease in the subject; and diagnosing the presence or absence of Alzheimer's Disease in the patient.

108. The method of claim 106 or 107, further comprising administering an immunogenic composition comprising an immunogenic peptide comprising SEQ ID NO: 1 or SEQ ID NO: 2 to the patient diagnosed with Alzheimer's Disease.

109. A method for selecting a patient suffering from Alzheimer's Disease to treat with an immunogenic composition comprising an immunogenic peptide comprising SEQ ID NO: 1 or SEQ ID NO: 2, comprising:
(a) detecting the presence and/or amount of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p in the patient or a sample from the patient;
(b) comparing to a control sample or threshold, wherein the presence of one or more of hsa-let-7a-5p, hsa-miR-10a-5p, hsa-miR-145-5p, hsa-miR-103a-3p, hsa-miR-191-5p, hsa-miR-374a-5p, hsa-miR-26a-5p, hsa-miR-107, hsa-miR-15a-5p, hsa-miR-126-3p, hsa-miR-224-5p, hsa-miR-18a-5p, hsa-miR-23a-3p, hsa-miR-26b-5p, and hsa-miR-21-5p and/or an altered amount relative to an amount in a control sample or threshold indicates Alzheimer's Disease in the subject; and
(c) selecting the patient for treatment based on step (b).

110. A method for selecting a patient suffering from Alzheimer's Disease to treat with an immunogenic composition comprising an immunogenic peptide comprising SEQ ID NO: 1 or SEQ ID NO: 2, comprising:
(a) detecting the presence and/or amount of 2,4-dihydroxybutanoic acid, a phospholipid, phosphatidylcholine, sphingomyelin, and/or sterol, in blood plasma, serum, or cerebrospinal fluid (CSF) of the patient;
(b) comparing to a control sample or threshold, wherein the presence of one or more metabolites and/or an altered amount relative to an amount in a control sample or threshold indicates Alzheimer's Disease in the subject; and
(c) selecting the patient for treatment based on step (b).

111. The method of claim 109 or claim 110, further comprising administering the immunogenic composition to the selected patient.
